SEQUENCE LISTING (73 sequences)

SEQ ID NO: 1 - protein, 581 aa, Homo sapiens
  source 1..581: note = LRRC15 amino acid sequence GenBank: AAI01066.1
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 2 - DNA, 3191 bp, Homo sapiens
  source 1..3191: mol_type = genomic DNA; note = LRRC15 nucleotide sequence GenBank: BC101065.1
cagccttgagccgtccctcgtcctcctctcaggctccctcttgtccacggcgggcgggcgccgagctgctggctatgccactgaagcattatctccttttgctggtgggctgccaagcctggggtgcagggttggcctaccatggctgccctagcgagtgtacctgctccagggcctcccaggtggagtgcaccggggcacgcattgtggcagtgcccacccctctgccctggaacgccatgagcctgcagatcctcaacacgcacatcactgaactcaatgagtccccgttcctcaatatctcagccctcatcgccctgaggattgagaagaatgagctgtcgcgcatcacgcctggggccttccgaaacctgggctcgctgcgctatctcagcctcgccaacaacaagctgcaggttctgcccatcggcctcttccagggcctggacagcctcgagtctctccttctgtccagtaaccagctgttgcagatccagccggcccacttctcccagtgcagcaacctcaaggagctgcagttgcacggcaaccacctggaatacatccctgacggagccttcgaccacctggtaggactcacgaagctcaatctgggcaagaatagcctcacccacatctcacccagggtcttccagcacctgggcaacctccaggtcctccggctgtatgagaacaggctcacggatatccccatgggcacttttgatgggcttgttaacctgcaggaactggctctgcagcagaaccagattggactgctctcccctggtctcttccacaacaaccacaacctccagagactctacctgtccaacaaccacatctcccagctgccacccagcgtcttcatgcagctgccccagctcaaccgtcttactctctttgggaattccctgaaggagctctctccggggatcttcgggcccatgcccaacctgcgggagctttggctctatgacaaccacatctcttctctacccgacaatgtcttcagcaacctccgccagttgcaggtcctgattcttagccgcaatcagatcagcttcatctccccgggtgccttcaacgggctaacggagcttcgggagctgtccctccacaccaacgcactgcaggacctggacgggaacgtcttccgcatgttggccaacctgcagaacatctccctgcagaacaaccgcctcagacagctcccagggaatatcttcgccaacgtcaatggcctcatggccatccagctgcagaacaaccagctggagaacttgcccctcggcatcttcgatcacctggggaaactgtgtgagctgcggctgtatgacaatccctggaggtgtgactcagacatccttccgctccgcaactggctcctgctcaaccagcctaggttagggacggacactgtacctgtgtgtttcagcccagccaatgtccgaggccagtccctcattatcatcaatgtcaacgttgctgttccaagcgtccatgtccccgaggtgcctagttacccagaaacaccatggtacccagacacacccagttaccctgacaccacatccgtctcttctaccactgagctaaccagccctgtggaagactacactgatctgactaccattcaggtcactgatgaccgcagcgtttggggcatgacccaggcccagagcgggctggccattgccgccattgtaattggcattgtcgccctggcctgctccctggctgcctgcgtcggctgttgctgctgcaagaagaggagccaagctgtcctgatgcagatgaaggcacccaatgagtgttaaagaggcaggctggagcagggctggggaatgatgggactggaggacctgggaatttcatctttctgcctccacccctgggtccatggagctttcccgtgattgctctttctggccccagagaaaggtgagcctacctcttcctgacttgcctgattctcccgtagagaagcaggtcgtgccggaccttcctacaatcaggaagatagatccaactggccatggcaaaagccctggggatttccgattcatacccctgggcttccttcgagagggctcttcctccaaatcctccccacctgtcctccaagaacagccttccctgcgcccaggccccctccgggcctctgtagactcagttagtccacagcctgctcacttcgtgggaatagttctccgctgagatagcccctctcgcctaagtattatgtaagttgatttcccttcttttgtttctcttgtttgtgctacggcttgacccagcatgtcccctcaaatgaaagttctccccttgattttctgctcctgaaggcagggtgagttctctcctcaaagaagacttcaaaccatttaactggtttcttaagagccgtcaatcagcctggttttggggatgctatgaaagagagaaggaaaatcatgccgctcagttcctggagacagaagagccgtcatcagtgtctcacttgtgatttttatctggaaaaggaagaaacaccccagcacagcaagctcagccttttagagaaggatatttccaaactgcaaactttgctttgaaaagtttagccctttaaggaatgaaatcatgtagaattttggacttctaaaaacattaaaatcagcttattaatacgggatagagaaagaaatctggtgcctgggggtccctgtgttcacccctagagtttgttttaaaatttttaattgaagcatgtgaagtgtacgtgcagaaaagtgggaacatgatagtgtatggcttggtggattttcacaaactgaacatacctgtgtaatcagcatctagacccagacccagagcatcacaaatatcccccatcctgggcttttcccagaggagatgggggcttctgaagatggacttacctgggacctgccccccatgagccaggacggtccccccacagtcagcctgtgcaaaggccccgtggccaggggtggaggagaatatgtgggtgtggacaggatgggagactgtggcctgaacaggagattttattatatctggagaccctgagagaccctgagacctggggcaccatggctggccaggtcagaagcatcctgactgcagaggtccgtgcagccacaccctcttccctgccagcaagctgtctgcggctcatcggaggc

SEQ ID NO: 3 - protein, 5 aa, synthetic construct
  source 1..5: note = MDC001 HCDR1 amino acid sequence
TFAMS

SEQ ID NO: 4 - protein, 17 aa, synthetic construct
  source 1..17: note = MDC001 HCDR2 amino acid sequence
VISGTGGSTFYTDSVKG

SEQ ID NO: 5 - protein, 12 aa, synthetic construct
  source 1..12: note = MDC001 HCDR3 amino acid sequence
AVTVNTRGFFDY

SEQ ID NO: 6 - protein, 121 aa, synthetic construct
  source 1..121: note = MDC001 VH amino acid sequence
QVQLLESGGGLVQPGGSLRLSCAASGFTFSTFAMSWVRQAPGKGLDWVSVISGTGGSTFYTDSVKGRFTISRDNSENTLYLEMNSLRAEDTAVYFCAKAVTVNTRGFFDYWGQGTLVTVSS

SEQ ID NO: 7 - protein, 121 aa, synthetic construct
  source 1..121: note = MDC001 VH amino acid sequence with N-terminal pyroglutamate
  MOD_RES 1: note = Pyroglutamate
EVQLLESGGGLVQPGGSLRLSCAASGFTFSTFAMSWVRQAPGKGLDWVSVISGTGGSTFYTDSVKGRFTISRDNSENTLYLEMNSLRAEDTAVYFCAKAVTVNTRGFFDYWGQGTLVTVSS

SEQ ID NO: 8 - DNA, 363 bp, synthetic construct
  source 1..363: mol_type = other DNA; note = MDC001 VH nucleotide sequence
caagtgcagctgctggaatcgggcggcggacttgtgcagccgggcggatccctgaggctgagctgtgctgcctccggattcactttctccaccttcgccatgtcctgggtccgccaagcgcccggaaagggcttggattgggtgtccgtcatttcggggaccgggggtagcactttttacaccgactccgtgaagggccgcttcaccatctcaagagacaactctgaaaacaccctgtacctcgagatgaatagcctccgggccgaggatactgcagtctacttctgcgccaaagccgtgaccgtgaacacccggggatttttcgactattggggacagggtactctcgtgacggtgtcctca

SEQ ID NO: 9 - protein, 11 aa, synthetic construct
  source 1..11: note = MDC001 LCDR1 amino acid sequence
RTSQVIRNDLG

SEQ ID NO: 10 - protein, 7 aa, synthetic construct
  source 1..7: note = MDC001 LCDR2 amino acid sequence
DASSLQS

SEQ ID NO: 11 - protein, 9 aa, synthetic construct
  source 1..9: note = MDC001 LCDR3 amino acid sequence
LQDNNYPWT

SEQ ID NO: 12 - protein, 107 aa, synthetic construct
  source 1..107: note = MDC001 VL amino acid sequence
EIVLTQSPSSLSASVGDRVTITCRTSQVIRNDLGWYQQKPGKAPKLLIYDASSLQSGVPSRFSGSGSGTDFTLTISSLQPEDFATYYCLQDNNYPWTFGQGTKVAIK

SEQ ID NO: 13 - DNA, 321 bp, synthetic construct
  source 1..321: mol_type = other DNA; note = MDC001 VL nucleotide sequence
gagattgtgctcactcaatccccctcctcgctgtcggcctccgtgggggatcgcgtgaccatcacttgccggaccagccaggtcatcagaaacgacctgggatggtaccagcagaagccggggaaggcaccgaagctgctcatctacgacgctagcagcctgcagtccggagtgccatcaaggttctccggctcgggttccggaactgacttcaccttgaccatctcatccctgcaacccgaagatttcgccacctactactgtctgcaagacaacaactatccttggacgtttggacagggcaccaaagtcgcgattaag

SEQ ID NO: 14 - protein, 450 aa, synthetic construct
  source 1..450: note = MDC001 HC amino acid sequence
QVQLLESGGGLVQPGGSLRLSCAASGFTFSTFAMSWVRQAPGKGLDWVSVISGTGGSTFYTDSVKGRFTISRDNSENTLYLEMNSLRAEDTAVYFCAKAVTVNTRGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 451 aa, synthetic construct
  source 1..451: note = MDC001 HC amino acid sequence with C-terminal lysine
QVQLLESGGGLVQPGGSLRLSCAASGFTFSTFAMSWVRQAPGKGLDWVSVISGTGGSTFYTDSVKGRFTISRDNSENTLYLEMNSLRAEDTAVYFCAKAVTVNTRGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 450 aa, synthetic construct
  source 1..450: note = MDC001 HC amino acid sequence with N-terminal pyroglutamate
  MOD_RES 1: note = Pyroglutamate
EVQLLESGGGLVQPGGSLRLSCAASGFTFSTFAMSWVRQAPGKGLDWVSVISGTGGSTFYTDSVKGRFTISRDNSENTLYLEMNSLRAEDTAVYFCAKAVTVNTRGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 214 aa, synthetic construct
  source 1..214: note = MDC001 LC amino acid sequence
EIVLTQSPSSLSASVGDRVTITCRTSQVIRNDLGWYQQKPGKAPKLLIYDASSLQSGVPSRFSGSGSGTDFTLTISSLQPEDFATYYCLQDNNYPWTFGQGTKVAIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 1350 bp, synthetic construct
  source 1..1350: mol_type = other DNA; note = MDC001 HC nucleotide sequence
caagtgcagctgctggaatcgggcggcggacttgtgcagccgggcggatccctgaggctgagctgtgctgcctccggattcactttctccaccttcgccatgtcctgggtccgccaagcgcccggaaagggcttggattgggtgtccgtcatttcggggaccgggggtagcactttttacaccgactccgtgaagggccgcttcaccatctcaagagacaactctgaaaacaccctgtacctcgagatgaatagcctccgggccgaggatactgcagtctacttctgcgccaaagccgtgaccgtgaacacccggggatttttcgactattggggacagggtactctcgtgacggtgtcctcagcatccactaaggggcctagcgtctttccgctggccccgtcctccaagtccacttcgggtggaaccgcggcactggggtgcctcgtgaaggactacttccccgagccggtcaccgtgtcctggaactcgggagccctgacctccggagtgcatactttccctgcggtgctgcagtcctccgggctctactcgctgtcaagcgtggtcaccgtcccgagctcatccctgggtactcagacctacatttgcaacgtgaaccacaaaccttccaacaccaaggtcgacaagagggtggagcctaagagctgcgacaagacccacacctgtcccccgtgtcccgcccctgagctgctgggcggccccagcgtgttcctcttcccgcctaagccgaaggacactctgatgatctcgagaacccctgaagtgacctgtgtggtggtggatgtgtcccacgaggatccggaagtgaagttcaattggtacgtggacggagtggaagtccataacgccaagaccaagccccgcgaggaacagtacaactcaacttaccgggtggtgtcagtgctgaccgtgctgcaccaagattggctgaacgggaaggagtacaagtgcaaagtctccaacaaggcgctgccggcccccattgaaaagaccatcagcaaggctaagggccagccccgggaaccacaggtctacaccttgcccccttcccgggaggaaatgaccaagaaccaagtgtcgctgacgtgcctggtcaagggcttttatccatctgacatcgccgtggagtgggaaagcaacggccagccggaaaacaactacaagactaccccgcctgtgctggactccgacggctcgttcttcctgtattccaagctcaccgtggataagtccagatggcagcagggcaatgtgttcagctgcagcgtgatgcatgaggccctgcacaaccactacactcagaaatcactgtccctttcccccgga

SEQ ID NO: 19 - DNA, 642 bp, synthetic construct
  source 1..642: mol_type = other DNA; note = MDC001 LC nucleotide sequence
gagattgtgctcactcaatccccctcctcgctgtcggcctccgtgggggatcgcgtgaccatcacttgccggaccagccaggtcatcagaaacgacctgggatggtaccagcagaagccggggaaggcaccgaagctgctcatctacgacgctagcagcctgcagtccggagtgccatcaaggttctccggctcgggttccggaactgacttcaccttgaccatctcatccctgcaacccgaagatttcgccacctactactgtctgcaagacaacaactatccttggacgtttggacagggcaccaaagtcgcgattaagcgcaccgtggccgcccctagcgtgtttatcttccctccctcggatgagcagcttaagtcaggcaccgcatccgtggtctgcctgctcaacaacttctacccgagggaagccaaagtgcagtggaaagtggacaacgcgctccagtcgggaaactcccaggagtccgtgaccgaacaggactccaaggacagcacttattccctgtcctccactctgacgctgtcaaaggccgactacgagaagcacaaggtctacgcctgcgaagtgacccatcaggggctttcctcgcccgtgactaagagcttcaatcggggcgaatgc

SEQ ID NO: 20 - DNA, 51 bp, synthetic construct
  source 1..51: mol_type = other DNA; note = MDC001.VK signal peptide nucleotide sequence
atgcgcgcctggatcttcttcctgctttgcctggccggccgggcgctcgcc

SEQ ID NO: 21 - DNA, 51 bp, synthetic construct
  source 1..51: mol_type = other DNA; note = MDC001.VH signal peptide nucleotide sequence
atgcgggcttggatcttcttcctgctgtgcctggccgggagagcgctggcc

SEQ ID NO: 22 - protein, 6 aa, synthetic construct
  source 1..6: note = MDC002 HCDR1 amino acid sequence
SSNWWS

SEQ ID NO: 23 - protein, 20 aa, synthetic construct
  source 1..20: note = MDC002 HCDR2 amino acid sequence
EWIGEIYHSGSTNYNPSLKS

SEQ ID NO: 24 - protein, 11 aa, synthetic construct
  source 1..11: note = MDC002 HCDR3 amino acid sequence
DNWGSFHAFDF

SEQ ID NO: 25 - protein, 120 aa, synthetic construct
  source 1..120: note = MDC002 VH amino acid sequence
QVQLQESGPGLVKPSGTLSLTCAVSGGSISSSNWWSWVRQAPGKGLEWIGEIYHSGSTNYNPSLKSRVTMSVDKSKNQFSLKLTSVTAADTAVYYCARDNWGSFHAFDFWGQGTMVTVSS

SEQ ID NO: 26 - protein, 120 aa, synthetic construct
  source 1..120: note = MDC002 VH amino acid sequence with N-terminal pyroglutamate
  MOD_RES 1: note = Pyroglutamate
EVQLQESGPGLVKPSGTLSLTCAVSGGSISSSNWWSWVRQAPGKGLEWIGEIYHSGSTNYNPSLKSRVTMSVDKSKNQFSLKLTSVTAADTAVYYCARDNWGSFHAFDFWGQGTMVTVSS

SEQ ID NO: 27 - protein, 11 aa, synthetic construct
  source 1..11: note = MDC002 LCDR1 amino acid sequence
RASQSIGSNLH

SEQ ID NO: 28 - protein, 7 aa, synthetic construct
  source 1..7: note = MDC002 LCDR2 amino acid sequence
YTSQSFS

SEQ ID NO: 29 - protein, 9 aa, synthetic construct
  source 1..9: note = MDC002 LCDR3 amino acid sequence
HQSTGLPHT

SEQ ID NO: 30 - protein, 107 aa, synthetic construct
  source 1..107: note = MDC002 VL amino acid sequence
EIVLTQSPDFQSVTPKEKVTITCRASQSIGSNLHWYQQKPDQSPKLLIKYTSQSFSGVPSRFSGSGSGTDFTLTISSLEAEDAATYFCHQSTGLPHTFGGGTKVEIK

SEQ ID NO: 31 - protein, 5 aa, synthetic construct
  source 1..5: note = MDC003 HCDR1 amino acid sequence
DYYMT

SEQ ID NO: 32 - protein, 17 aa, synthetic construct
  source 1..17: note = MDC003 HCDR2 amino acid sequence
DIGSSGGSVDYADSVKG

SEQ ID NO: 33 - protein, 13 aa, synthetic construct
  source 1..13: note = MDC003 HCDR3 amino acid sequence
YNWNDRGYDGFDI

SEQ ID NO: 34 - protein, 122 aa, synthetic construct
  source 1..122: note = MDC003 VH amino acid sequence
QVQLVESGGDLVTPGGSLRLSCAASGFTFSDYYMTWIRQAPGKGLEWISDIGSSGGSVDYADSVKGRFTISRDNAKNSLYLQMSSLRAEDTAVYYCARYNWNDRGYDGFDIWGQGTMVTVSS

SEQ ID NO: 35 - protein, 122 aa, synthetic construct
  source 1..122: note = MDC003 VH amino acid sequence with N-terminal pyroglutamate
  MOD_RES 1: note = Pyroglutamate
EVQLVESGGDLVTPGGSLRLSCAASGFTFSDYYMTWIRQAPGKGLEWISDIGSSGGSVDYADSVKGRFTISRDNAKNSLYLQMSSLRAEDTAVYYCARYNWNDRGYDGFDIWGQGTMVTVSS

SEQ ID NO: 36 - protein, 11 aa, synthetic construct
  source 1..11: note = MDC003 LCDR1 amino acid sequence
RASQGISSALA

SEQ ID NO: 37 - protein, 7 aa, synthetic construct
  source 1..7: note = MDC003 LCDR2 amino acid sequence
DASSLES

SEQ ID NO: 38 - protein, 9 aa, synthetic construct
  source 1..9: note = MDC003 LCDR3 amino acid sequence
QQFNSYPHT

SEQ ID NO: 39 - protein, 107 aa, synthetic construct
  source 1..107: note = MDC003 VL amino acid sequence
EIVLTQSPSSLSASVGDRVTIKCRASQGISSALAWYQQKPGKAPKLLIYDASSLESGVPSGFSGSGSGTDFTLTISSLQPEDFATYYCQQFNSYPHTFGPGTKVDIK

SEQ ID NO: 40 - protein, 4 aa, synthetic construct
  source 1..4: note = MDC004 HCDR1 amino acid sequence
YYWS

SEQ ID NO: 41 - protein, 16 aa, synthetic construct
  source 1..16: note = MDC004 HCDR2 amino acid sequence
YVHHSGGTNYNPSLKS

SEQ ID NO: 42 - protein, 8 aa, synthetic construct
  source 1..8: note = MDC004 HCDR3 amino acid sequence
DTYGPFDY

SEQ ID NO: 43 - protein, 116 aa, synthetic construct
  source 1..116: note = MDC004 VH amino acid sequence
QVQLQESGPGLVKPSETLSLTCTVSGASINIYYWSWIREASGKGLEWIGYVHHSGGTNYNPSLKSRVSISVDTSKNQFSLRLKSVTAADTAVYYCARDTYGPFDYWGQGTLVTVSS

SEQ ID NO: 44 - protein, 116 aa, synthetic construct
  source 1..116: note = MDC004 VH amino acid sequence with N-terminal pyroglutamate
  MOD_RES 1: note = Pyroglutamate
EVQLQESGPGLVKPSETLSLTCTVSGASINIYYWSWIREASGKGLEWIGYVHHSGGTNYNPSLKSRVSISVDTSKNQFSLRLKSVTAADTAVYYCARDTYGPFDYWGQGTLVTVSS

SEQ ID NO: 45 - protein, 11 aa, synthetic construct
  source 1..11: note = MDC004 LCDR1 amino acid sequence
RASQGISNYLA

SEQ ID NO: 46 - protein, 7 aa, synthetic construct
  source 1..7: note = MDC004 LCDR2 amino acid sequence
AASTLQS

SEQ ID NO: 47 - protein, 9 aa, synthetic construct
  source 1..9: note = MDC004 LCDR3 amino acid sequence
QNYYSAPWT

SEQ ID NO: 48 - protein, 107 aa, synthetic construct
  source 1..107: note = MDC004 VL amino acid sequence
EIVLTQSPSSLSASVGDRVTITCRASQGISNYLAWYQQKPGKVPKLLIYAASTLQSGVPSRFSGSGSGTDFTLTVSSLQPEDVATYYCQNYYSAPWTFGQGTKVEIK

SEQ ID NO: 49 - protein, 5 aa, synthetic construct
  source 1..5: note = MDC005 HCDR1 amino acid sequence
TYYWS

SEQ ID NO: 50 - protein, 15 aa, synthetic construct
  source 1..15: note = MDC005 HCDR2 amino acid sequence
YIHYSGGTNYNPSLK

SEQ ID NO: 51 - protein, 8 aa, synthetic construct
  source 1..8: note = MDC005 HCDR3 amino acid sequence
DIYGPFDY

SEQ ID NO: 52 - protein, 116 aa, synthetic construct
  source 1..116: note = MDC005 VH amino acid sequence
QVQLQESGPGLVKPSETLSLTCTVSGGSINTYYWSWIRQPPGMGLEWIGYIHYSGGTNYNPSLKSRVAISADTSRNQFSLKLSFVTAADTAVYYCARDIYGPFDYWGQGTLVTVSS

SEQ ID NO: 53 - protein, 116 aa, synthetic construct
  source 1..116: note = MDC005 VH amino acid sequence with N-terminal pyroglutamate
  MOD_RES 1: note = Pyroglutamate
EVQLQESGPGLVKPSETLSLTCTVSGGSINTYYWSWIRQPPGMGLEWIGYIHYSGGTNYNPSLKSRVAISADTSRNQFSLKLSFVTAADTAVYYCARDIYGPFDYWGQGTLVTVSS

SEQ ID NO: 54 - protein, 11 aa, synthetic construct
  source 1..11: note = MDC005 LCDR1 amino acid sequence
RASQVISNYLA

SEQ ID NO: 55 - protein, 7 aa, synthetic construct
  source 1..7: note = MDC005 LCDR2 amino acid sequence
ASSTLQS

SEQ ID NO: 56 - protein, 9 aa, synthetic construct
  source 1..9: note = MDC005 LCDR3 amino acid sequence
QNYYSVPWT

SEQ ID NO: 57 - protein, 107 aa, synthetic construct
  source 1..107: note = MDC005 VL amino acid sequence
EIVLTQSPSSLSASVGDRVTFTCRASQVISNYLAWYQQKPGKLPKLLIYASSTLQSGVPSRFSGSGSGTDFTLGISSLQPEDVATYYCQNYYSVPWTFGQGTKVEIK

SEQ ID NO: 58 - protein, 5 aa, synthetic construct
  source 1..5: note = MDC006 HCDR1 amino acid sequence
DYYMS

SEQ ID NO: 59 - protein, 17 aa, synthetic construct
  source 1..17: note = MDC006 HCDR2 amino acid sequence
SISNSGGAIYYADSVKG

SEQ ID NO: 60 - protein, 13 aa, synthetic construct
  source 1..13: note = MDC006 HCDR3 amino acid sequence
YNWNDRGYDAFDI

SEQ ID NO: 61 - protein, 122 aa, synthetic construct
  source 1..122: note = MDC006 VH amino acid sequence
QVQLVESGGGLVKPGGSLRLSCAASGFTFSDYYMSWIRQAPRKGLEWVSSISNSGGAIYYADSVKGRFSISRDNAKNSLYLQMNSLRAEDTAVYYCARYNWNDRGYDAFDIWGQGTMVTVSS

SEQ ID NO: 62 - protein, 122 aa, synthetic construct
  source 1..122: note = MDC006 VH amino acid sequence with N-terminal pyroglutamate
  MOD_RES 1: note = Pyroglutamate
EVQLVESGGGLVKPGGSLRLSCAASGFTFSDYYMSWIRQAPRKGLEWVSSISNSGGAIYYADSVKGRFSISRDNAKNSLYLQMNSLRAEDTAVYYCARYNWNDRGYDAFDIWGQGTMVTVSS

SEQ ID NO: 63 - protein, 11 aa, synthetic construct
  source 1..11: note = MDC006 LCDR1 amino acid sequence
RASQGISSALA

SEQ ID NO: 64 - protein, 7 aa, synthetic construct
  source 1..7: note = MDC006 LCDR2 amino acid sequence
DASSLES

SEQ ID NO: 65 - protein, 9 aa, synthetic construct
  source 1..9: note = MDC006 LCDR3 amino acid sequence
QQFNSYPHT

SEQ ID NO: 66 - protein, 107 aa, synthetic construct
  source 1..107: note = MDC006 VL amino acid sequence
DIVMTQSPSSLSASVGDRVTITCRASQGISSALAWYQQKPGKAPKILIYDASSLESGVPSRFSGSGSGTDFTLTISSLLPEDFVTYYCQQFNSYPHTFGPGTKVEIK

SEQ ID NO: 67 - protein, 450 aa, synthetic construct
  source 1..450: note = huM25 huIgG1
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 214 aa, synthetic construct
  source 1..214: note = huM25 hK
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 63 aa, synthetic construct
  source 1..63: note = Anti-LRRC15 antibody signal peptide nucleotide sequence
GAATTCGCCACCATGAGGGCTTGGATCTTCTTTCTGCTCTGCCTGGCCGGGCGCGCCCTCGCA

SEQ ID NO: 70 - protein, 21 aa, synthetic construct
  source 1..21: note = Anti-LRRC15 antibody signal peptide amino acid sequence
EFATMRAWIFFLLCLAGRALA

SEQ ID NO: 71 - DNA, 45 bp, synthetic construct
  source 1..45: mol_type = other DNA; note = appendix nucleotide sequence
tgaggatcccccgacctcgacctctggctaataaaggaaatttat

SEQ ID NO: 72 - protein, 5 aa, synthetic construct
  source 1..5: note = Peptidyl linker
PVGVV

SEQ ID NO: 73 - protein, 4 aa, synthetic construct
GFLG

(19) United States
(12) Patent Application Publication
(10) Pub. No.: US 2026/0144884 A1
(43) Pub. Date: May 28, 2026

(54) ANTI-LRRC15 ANTIBODY DRUG CONJUGATES

(71) Applicant: Bristol-Myers Squibb Company, Princeton, NJ (US)

(72) Inventors: Sayumi YAMAZOE, Princeton, NJ (US); Sonia G. TANLIMCO, Princeton, NJ (US); Lore FLORIN, Princeton, NJ (US); Philip M. VITORINO, Princeton, NJ (US); Qinqin CHENG, Princeton, NJ (US); Christine H. TAN, Princeton, NJ (US); Yuntao ZHANG, Princeton, NJ (US); Sunitha BACHAWAL, Princeton, NJ (US); Hok Yee CHAN, Princeton, NJ (US); Winse Hong MORISHIGE, Princeton, NJ (US); Andrew W. DRAKE, Princeton, NJ (US); Deanne M. LATHERS, Princeton, NJ (US); Jaimee C. WONG, Princeton, NJ (US)

(21) Appl. No.: 19/397,449

(22) Filed: Nov. 21, 2025

Related U.S. Application Data
(60) Provisional application No. 63/724,216, filed on Nov. 22, 2024.
(60) Provisional application No. 63/919,293, filed on Nov. 17, 2025.

(51) Int. Cl. A61K 47/68; A61P 35/00; C07K 16/18

(52) U.S. Cl. CPC A61K 47/68037; A61K 47/68031; A61K 47/6843; A61K 47/6879; A61K 47/6889; A61P 35/00; C07K 16/18

(57) ABSTRACT

Provided herein are antibody drug conjugates of the formula (I) comprising an anti-LRRC15 antibodies, antigen binding portions thereof that is conjugated with a linker and exatecan. The disclosure also provides a method of treating cancer in a subject in need thereof comprising administering to the subject an antibody drug conjugate disclosed herein.

### CROSS-REFERENCE TO RELATED APPLICATIONS

This application claims the benefit of U.S. provisional patent application Ser. No. 63/724,216, filed Nov. 22, 2024, and U.S. provisional patent application Ser. No. 63/919,293, filed Nov. 17, 2025, each of which is incorporated herein by reference in its entirety.

### REFERENCE TO SEQUENCE LISTING SUBMITTED ELECTRONICALLY

The content of the electronically submitted Sequence Listing XML (File Name 3338_3560003_SequenceListing_ST26.xml; Size: 91,909 bytes; and Date of Creation: Nov. 18, 2025) filed with the application is incorporated herein by reference in its entirety.

### FIELD OF THE DISCLOSURE

The disclosure provides antibody drug conjugates (ADCs) comprising antibodies or antigen binding portions that specifically binds to Leucine Rich Repeat Containing 15 (LRRC15) for use in a therapy.

### BACKGROUND OF THE DISCLOSURE

LRRC15 is a transmembrane protein that belongs to the Leucine-Rich Repeat (LRR) superfamily and is involved in cell-cell and cell-ECM interactions (see, e.g., Satoh K et al. Biochem. Biophys. Res. Commun. 2002; 290(2):756-62 and Ray U et al. Cancer Res. 2022; 82(9):1675-81). LRRC15 is found to be highly upregulated in multiple solid tumor indications including breast, head and neck, lung, and pancreatic cancers. Differential expression between tumor and normal tissue was initially identified through bulk tumor mRNA analysis and LRRC15 protein expression was subsequently confirmed in both cancer associated fibroblasts and some tumor cells by immunohistochemistry (IHC). While LRRC15 is relatively broadly expressed on cancer-associated fibroblasts (CAFs) within the stromal compartment of tumors, it is also expressed in subsets of tumors of mesenchymal origin such as glioblastoma, sarcomas, and melanoma (Purcell et al., (2018) Cancer Res., 78(14):4059-4072). Limited LRRC15 expression is observed in normal tissues, differentiating it from other stromal targets, such as FAP (fibroblast activation protein) and EDBFN (extra-domain B of fibronectin) (see, e.g., Purcell J W et al. Cancer Res. 2018; 78(14): 4059-72 and Hooper A T et al. Mol. Cancer Ther. 2022; 21(9):1462-72). LRRC15 selectively marks TGFβ-induced CAFs, also known as myCAFs, which are associated with poor responses to checkpoint inhibitors (Dominguez et al., (2020) Cancer Discov., 10(2):232-253). Depletion of LRRC15+ CAFs have been reported to enhance the activity of checkpoint inhibitors in mice (Krishnamurty et al., (2022) Nature, 611 (7934): 148-154). Though multiple therapeutic approaches targeting LRRC15 in cancer have been explored, there are currently no FDA-approved LRRC15-targeted therapies (Colombo R et al. Cancer Discov. 2024; 14(11):2089-2108).

The present disclosure provides novel stromal targeting ADCs that target LRRC15 comprising a fully human IgG1 monoclonal antibody (mAb) against LRRC15 and topoisomerase I (TOPO1) inhibitors such as exatecan, connected via a cleavable linker-payload system containing a phosphorus (V) moiety (also denoted as “P5”). Exatecan, a clinically explored TOPO1 inhibitor, induces DNA damage and is shown to have enhanced bystander tumor cell killing activity as compared to Deruxtecan, the linker payload system used in Enhertu, a clinically approved ADC (Colombo R et al. Cancer Discov. 2024; 14(11):2089-2108).

Without wishing to be bound by any particular theory, one possible mechanism is that most ADCs target cancer cells directly; however, tumor stroma-targeting anti-LRRC15-P5-TOPO1 ADC binds to and is processed by LRRC15+ fibroblasts in the tumor microenvironment. The freed TOPO1 payload can then diffuse from CAF to neighboring cancer cells, resulting in bystander killing. It was found that LRRC15+ stromal cells are less sensitive to the TOPO1 payload as compared to tumor cells, allowing them to process ADC without being depleted. The ADCs provided herein provide a tumor-antigen independent mechanism for selective delivery of a potent cytotoxic to cancer patients underserved by current standards of care.

### BRIEF SUMMARY OF THE DISCLOSURE

The present disclosure provides an ADC having the formula (I):

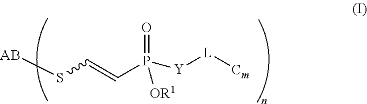

or a pharmaceutically acceptable salt, a stereoisomer or a solvate thereof, wherein:
[symbol] indicates that the configuration of the double bond may be E or Z;
R1 is a polyalkene glycol unit comprising at least 3 alkylene glycol subunits;
Y is NR2;
R2 is H or an optionally substituted aliphatic residue, or an optionally substituted aromatic residue;
L is a linker;
C is a DNA topoisomerase I inhibitor;
m is an integer ranging from 1 to 10;
n is an integer ranging from 1 to 20; and
AB is an anti-LRRC15 antibody, or an antigen binding portion thereof, comprising:
(a) a heavy chain variable region (VH) comprising complementarity determining region (CDR)1, CDR2, and CDR3 regions comprising the amino acid sequences set forth in SEQ ID NOs: 3, 4, and 5, respectively, and a light chain variable region (VL) comprising CDR1, CDR2, and CDR3 regions comprising the amino acid sequences set forth in SEQ ID NOs: 9, 10, and 11, or;
(b) a VH comprising CDR1, CDR2, and CDR3 regions which have at least 80%, 85%, 90%, 95%, 98%, or 99% sequence identity to the amino acid sequences set forth in SEQ ID NOs: 3, 4, and 5, respectively, and a VL comprising CDR1, CDR2, and CDR3 regions which have at least 85%, 90%, 95%, 98%, or 99% sequence identity to the amino acid sequences set forth in SEQ ID NOs: 9, 10, and 11, respectively; or
(c) a VH comprising CDR1, CDR2, and CDR3 regions comprising the amino acid sequences set forth in SEQ ID NOs: 22, 23, and 24, respectively, and a VL comprising CDR1, CDR2, and CDR3 regions comprising the amino acid sequences set forth in SEQ ID NOs: 27, 28, and 29, or;
(d) a VH comprising CDR1, CDR2, and CDR3 regions which have at least 80%, 85%, 90%, 95%, 98%, or 99% sequence identity to the amino acid sequences set forth in SEQ ID NOs: 22, 23, and 24, respectively, and a VL comprising CDR1, CDR2, and CDR3 regions which have at least 85%, 90%, 95%, 98%, or 99% sequence identity to the amino acid sequences set forth in SEQ ID NOs: 27, 28, and 29, respectively, or;
(e) a VH comprising CDR1, CDR2, and CDR3 regions comprising the amino acid sequences set forth in SEQ ID NOs: 31, 32, and 33, respectively, and a VL comprising CDR1, CDR2, and CDR3 regions comprising the amino acid sequences set forth in SEQ ID NOs: 36, 37, and 38, or;
(f) a VH comprising CDR1, CDR2, and CDR3 regions which have at least 80%, 85%, 90%, 95%, 98%, or 99% sequence identity to the amino acid sequences set forth in SEQ ID NOs: 31, 32, and 33, respectively, and a VL comprising CDR1, CDR2, and CDR3 regions which have at least 85%, 90%, 95%, 98%, or 99% sequence identity to the amino acid sequences set forth in SEQ ID NOs: 36, 37, and 38, respectively, or;
(g) a VH comprising CDR1, CDR2, and CDR3 regions comprising the amino acid sequences set forth in SEQ ID NOs: 40, 41, and 42, respectively, and a VL comprising CDR1, CDR2, and CDR3 regions comprising the amino acid sequences set forth in SEQ ID NOs: 45, 46, and 47, or;
(h) a VH comprising CDR1, CDR2, and CDR3 regions which have at least 80%, 85%, 90%, 95%, 98%, or 99% sequence identity to the amino acid sequences set forth in SEQ ID NOs: 40, 41, and 42, respectively, and a VL comprising CDR1, CDR2, and CDR3 regions which have at least 85%, 90%, 95%, 98%, or 99% sequence identity to the amino acid sequences set forth in SEQ ID NOs: 45, 46, and 47, respectively, or;
(i) a VH comprising CDR1, CDR2, and CDR3 regions comprising the amino acid sequences set forth in SEQ ID NOs: 49, 50, and 51, respectively, and a VL comprising CDR1, CDR2, and CDR3 regions comprising the amino acid sequences set forth in SEQ ID NOs: 54, 55, and 56, or;
(j) a VH comprising CDR1, CDR2, and CDR3 regions which have at least 80%, 85%, 90%, 95%, 98%, or 99% sequence identity to the amino acid sequences set forth in SEQ ID NOs: 49, 50, and 51, respectively, and a VL comprising CDR1, CDR2, and CDR3 regions which have at least 85%, 90%, 95%, 98%, or 99% sequence identity to the amino acid sequences set forth in SEQ ID NOs: 54, 55, and 56, respectively, or;
(k) a VH comprising CDR1, CDR2, and CDR3 regions comprising the amino acid sequences set forth in SEQ ID NOs: 58, 59, and 60, respectively, and a VL comprising CDR1, CDR2, and CDR3 regions comprising the amino acid sequences set forth in SEQ ID NOs: 63, 64, and 65, or
(l) a VH comprising CDR1, CDR2, and CDR3 regions which have at least 80%, 85%, 90%, 95%, 98%, or 99% sequence identity to the amino acid sequences set forth in SEQ ID NOs: 58, 59, and 60, respectively, and a VL comprising CDR1, CDR2, and CDR3 regions which have at least 85%, 90%, 95%, 98%, or 99% sequence identity to the amino acid sequences set forth in SEQ ID NOs: 63, 64, and 65, respectively.

In some aspects, the VH of the antibody or antigen binding portion thereof in the ADC comprises CDR1, CDR2, and CDR3 regions comprising the amino acid sequences set forth in SEQ ID NOs: 3, 4, and 5, respectively, and the VL of the antibody or antigen binding portion thereof comprises CDR1, CDR2, and CDR3 regions comprising the amino acid sequences set forth in SEQ ID NOs: 9, 10, and 11.

In some aspects, the VH of the antibody, or antigen binding portion thereof, in the ADC which specifically binds to LRRC15 comprises CDR1, CDR2, and CDR3 regions comprising the amino acid sequences set forth in SEQ ID NOs: 22, 23, and 24, respectively, and a VL comprising CDR1, CDR2, and CDR3 regions comprising the amino acid sequences set forth in SEQ ID NOs: 27, 28, and 29.

In some aspects, the VH of the antibody, or antigen binding portion thereof in the ADC comprises CDR1, CDR2, and CDR3 regions comprising the amino acid sequences set forth in SEQ ID NOs: 31, 32, and 33, respectively, and a VL comprising CDR1, CDR2, and CDR3 regions comprising the amino acid sequences set forth in SEQ ID NOs: 36, 37, and 38.

In some aspects, the VH of the antibody, or antigen binding portion thereof in the ADC comprises CDR1, CDR2, and CDR3 regions comprising the amino acid sequences set forth in SEQ ID NOs: 40, 41, and 42, respectively, and a VL comprising CDR1, CDR2, and CDR3 regions comprising the amino acid sequences set forth in SEQ ID NOs: 45, 46, and 47.

In some aspects, the VH of the antibody, or antigen binding portion thereof in the ADC comprises CDR1, CDR2, and CDR3 regions comprising the amino acid sequences set forth in SEQ ID NOs: 49, 50, and 51, respectively, and a VL comprising CDR1, CDR2, and CDR3 regions comprising the amino acid sequences set forth in SEQ ID NOs: 54, 55, and 56.

In some aspects, the VH of the antibody, or antigen binding portion thereof in the ADC comprises CDR1, CDR2, and CDR3 regions comprising the amino acid sequences set forth in SEQ ID NOs: 58, 59, and 60, respectively, and a VL comprising CDR1, CDR2, and CDR3 regions comprising the amino acid sequences set forth in SEQ ID NOs: 63, 64, and 65.

In some aspects, the VH in the ADC comprises an amino acid sequence having at least 80%, 85%, 90%, 95%, 98%, or 99% sequence identity to the amino acid sequence set forth in SEQ ID NO: 6, SEQ ID NO: 7, SEQ ID NO: 25, SEQ ID NO: 26, SEQ ID NO: 34, SEQ ID NO: 35, SEQ ID NO: 43, SEQ ID NO: 44, SEQ ID NO: 52, SEQ ID NO: 53, SEQ ID NO: 61 or SEQ ID NO: 62. In some aspects, the VH in the ADC comprises an amino acid sequence having at least 80%, 85%, 90%, 95%, 98%, or 99% sequence identity to the amino acid sequence set forth in SEQ ID NO: 6. In some aspects, the VH in the ADC comprises an amino acid sequence having at least 80%, 85%, 90%, 95%, 98%, or 99% sequence identity to the amino acid sequence set forth in SEQ ID NO: 7. In some aspects, the VH comprises an amino acid sequence having at least 80%, 85%, 90%, 95%, 98%, 99%, or 100% sequence identity to the amino acid sequence set forth in SEQ ID NO: 25. In some aspects, the VH comprises an amino acid sequence having at least 80%, 85%, 90%, 95%, 98%, 99%, or 100% sequence identity to the amino acid sequence set forth in SEQ ID NO: 26. In some aspects, the VH comprises an amino acid sequence having at least 80%, 85%, 90%, 95%, 98%, 99% or 100% sequence identity to the amino acid sequence set forth in SEQ ID NO: 34. In some aspects, the VH comprises an amino acid sequence having at least 80%, 85%, 90%, 95%, 98%, 99% or 100% sequence identity to the amino acid sequence set forth in SEQ ID NO: 35. In some aspects, the VH comprises an amino acid sequence having at least 80%, 85%, 90%, 95%, 98%, 99%, or 100% sequence identity to the amino acid sequence set forth in SEQ ID NO: 43. In some aspects, the VH comprises an amino acid sequence having at least 80%, 85%, 90%, 95%, 98%, 99%, or 100% sequence identity to the amino acid sequence set forth in SEQ ID NO: 44. In some aspects, the VH comprises an amino acid sequence having at least 80%, 85%, 90%, 95%, 98%, 99%, or 100% sequence identity to the amino acid sequence set forth in SEQ ID NO: 52. In some aspects, the VH comprises an amino acid sequence having at least 80%, 85%, 90%, 95%, 98%, 99%, or 100% sequence identity to the amino acid sequence set forth in SEQ ID NO: 53. In some aspects, the VH comprises an amino acid sequence having at least 80%, 85%, 90%, 95%, 98%, 99%, or 100% sequence identity to the amino acid sequence set forth in SEQ ID NO: 61. In some aspects, the VH comprises an amino acid sequence having at least 80%, 85%, 90%, 95%, 98%, 99%, or 100% sequence identity to the amino acid sequence set forth in SEQ ID NO: 62.

In some aspects, the VL in the ADC comprises an amino acid sequence having at least 80%, 85%, 90%, 95%, 98%, or 99% sequence identity to an amino acid sequence set forth in SEQ ID NO: 12, SEQ ID NO: 30, SEQ ID NO: 39, SEQ ID NO: 48, SEQ ID NO: 57, or SEQ ID NO: 66. In some aspects, the VL comprises an amino acid sequence having at least 80%, 85%, 90%, 95%, 98%, or 99% sequence identity to an amino acid sequence set forth in SEQ ID NO: 12. In some aspects, the VL comprises an amino acid sequence having at least 80%, 85%, 90%, 95%, 98%, or 99% sequence identity to an amino acid sequence set forth in SEQ ID NO: 30. In some aspects, the VL comprises an amino acid sequence having at least 80%, 85%, 90%, 95%, 98%, or 99% sequence identity to an amino acid sequence set forth in SEQ ID NO: 39. In some aspects, the VL comprises an amino acid sequence having at least 80%, 85%, 90%, 95%, 98%, or 99% sequence identity to an amino acid sequence set forth in SEQ ID NO: 48. In some aspects, the VL comprises an amino acid sequence having at least 80%, 85%, 90%, 95%, 98%, or 99% sequence identity to an amino acid sequence set forth in SEQ ID NO: 57. In some aspects, the VL comprises an amino acid sequence having at least 80%, 85%, 90%, 95%, 98%, or 99% sequence identity to an amino acid sequence set forth in SEQ ID NO: 66.

In some aspects, the VH and the VL of the antibody or antigen binding portion thereof in the ADC have at least 80%, 85%, 90%, 95%, 98%, or 99% sequence identity to:
the amino acid sequence set forth in SEQ ID NO: 6 and the amino acid sequence set forth in SEQ ID NO: 12, respectively; the amino acid sequence set forth in SEQ ID NO: 7 and the amino acid sequence set forth in SEQ ID NO: 12, respectively;
the amino acid sequence set forth in SEQ ID NO: 25 and the amino acid sequence set forth in SEQ ID NO: 30, respectively;
the amino acid sequence set forth in SEQ ID NO: 26 and the amino acid sequence set forth in SEQ ID NO: 30, respectively;
the amino acid sequence set forth in SEQ ID NO: 34 and the amino acid sequence set forth in SEQ ID NO: 39; respectively;
the amino acid sequence set forth in SEQ ID NO: 35 and the amino acid sequence set forth in SEQ ID NO: 39; respectively;
the amino acid sequence set forth in SEQ ID NO: 43 and the amino acid sequence set forth in SEQ ID NO: 48, respectively;
the amino acid sequence set forth in SEQ ID NO: 44 and the amino acid sequence set forth in SEQ ID NO: 48, respectively;
the amino acid sequence set forth in SEQ ID NO: 52 and the amino acid sequence set forth in SEQ ID NO: 57, respectively;
the amino acid sequence set forth in SEQ ID NO: 53 and the amino acid sequence set forth in SEQ ID NO: 57, respectively;
the amino acid sequence set forth in SEQ ID NO: 61 and the amino acid sequence set forth in SEQ ID NO: 66, respectively; or
the amino acid sequence set forth in SEQ ID NO: 62 and the amino acid sequence set forth in SEQ ID NO: 66, respectively.

In some aspects, the VH and the VL of the antibody or antigen binding portion thereof comprises:
the amino acid sequence set forth in SEQ ID NO: 6 and the amino acid sequence set forth in SEQ ID NO: 12, respectively;
the amino acid sequence set forth in SEQ ID NO: 7 and the amino acid sequence set forth in SEQ ID NO: 12, respectively;
the amino acid sequence set forth in SEQ ID NO: 25 and the amino acid sequence set forth in SEQ ID NO: 30, respectively;
the amino acid sequence set forth in SEQ ID NO: 26 and the amino acid sequence set forth in SEQ ID NO: 30, respectively;
the amino acid sequence set forth in SEQ ID NO: 34 and the amino acid sequence set forth in SEQ ID NO: 39; respectively;
the amino acid sequence set forth in SEQ ID NO: 35 and the amino acid sequence set forth in SEQ ID NO: 39; respectively;
the amino acid sequence set forth in SEQ ID NO: 43 and the amino acid sequence set forth in SEQ ID NO: 48, respectively;
the amino acid sequence set forth in SEQ ID NO: 44 and the amino acid sequence set forth in SEQ ID NO: 48, respectively;
the amino acid sequence set forth in SEQ ID NO: 52 and the amino acid sequence set forth in SEQ ID NO: 57, respectively;
the amino acid sequence set forth in SEQ ID NO: 53 and the amino acid sequence set forth in SEQ ID NO: 57, respectively;
the amino acid sequence set forth in SEQ ID NO: 61 and the amino acid sequence set forth in SEQ ID NO: 66, respectively; or
the amino acid sequence set forth in SEQ ID NO: 62 and the amino acid sequence set forth in SEQ ID NO: 66, respectively.

In some aspects, C in formula (I) is exatecan. In some aspects, Y in formula (I) is NH. In some aspects, the polyalkylene glycol unit R¹ in formula (I) comprises 3 to 100 subunits having the structure:

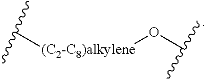

In some aspects, R1 in formula (I) is

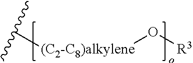

wherein: [symbol] indicates the position of the O;
R³ is selected from the group consisting of H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₃)alkyl and —(C₂-C₁₀)alkyl-N((C₁-C₃)alkyl)₂; and o is an integer ranging from 3 to 100.

In some aspects, the polyethylene glycol unit R¹ in formula (I) comprises 3 to 100 subunits having the structure:

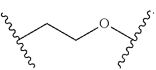 [SMILES: *CCO*]

In some aspects, R¹ in formula (I) is:

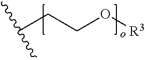

wherein [symbol] indicates the position of the O;
R³ is selected from the group consisting of H, —PO₃H, —(C₁-C₁₀)alkyl, —(C₁-C₁₀)alkyl-SO₃H, —(C₂-C₁₀)alkyl-CO₂H; and o is an integer ranging from 3 to 100. In some aspects, R³ in formula (I) is H.

In some aspects, o is an integer ranging from 8 to 30, e.g., from 8 to 16 or from 20 to 28, e.g., 10, 11, 12, 13 14, 22, 23, 24, 25 or 26.

In some aspects, the linker L in formula (I) has the formula: *-A-W₁₋₈—B₀₋₁-#, wherein A is a first spacer unit; W is an amino acid; B is a second spacer unit; * denotes the attachment point to the —Y—; and # denotes the attachment point to C.

In some aspects, A in formula (I) has the structure:

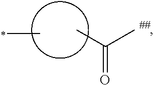

wherein ◯ is a 5- or 6-membered carbocycle; * denotes the attachment point to the —Y; and ## denotes the attachment point to W.

In some aspects,

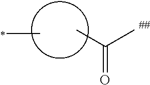

is

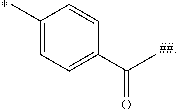

In some aspects, W is a dipeptide (W₂). In some aspects, the dipeptide is selected from the group consisting of valine-citrulline (Val-Cit) and valine-alanine (Val-Ala). In some aspects, the dipeptide is Val-Cit.

In some aspects, the second spacer unit B is a PAB group having the following structure:

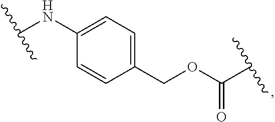 [SMILES: *Nc1ccc(COC(*)=O)cc1 ; ","]

wherein the NH group is bonded to —W— and the C(O) group is bonded to C.

In some aspects, the linker has the structure:

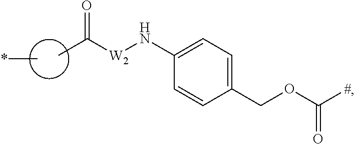

wherein W₂ is Val-Cit.

In some aspects, the linker L in formula (I) is *-A-W₂—B₁-# having the structure:

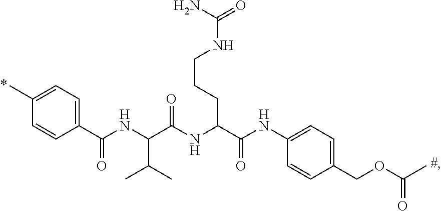

wherein * indicates the attachment point to the Y and # indicates the attachment point to C.

In some aspects, Y is NH; R¹ is a polyalkene glycol unit having the structure:

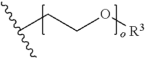

wherein [symbol] indicates the position of the O; R³ is H; o is an integer ranging from 8 to 30; L is a linker having the following structure:

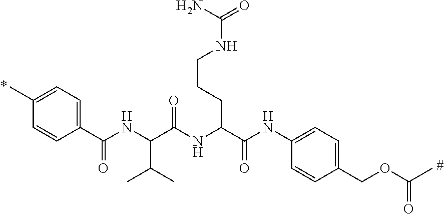

wherein * indicates the attachment point to the Y and # indicates the attachment point to C; C is exatecan; m is 1; and n is an integer ranging from 5 to 10. In some aspects, o is an integer ranging from 8 to 16. In some aspects, o is 10, 11, 12, 13 or 14. In some aspects, o is an integer ranging from 20 to 28. In some aspects, o is 22, 23, 24, 25, or 26. In some aspects, n is an integer ranging from 6 to 8.

In some aspects, the disclosure provides an antibody drug conjugate (ADC) having the formula (II):

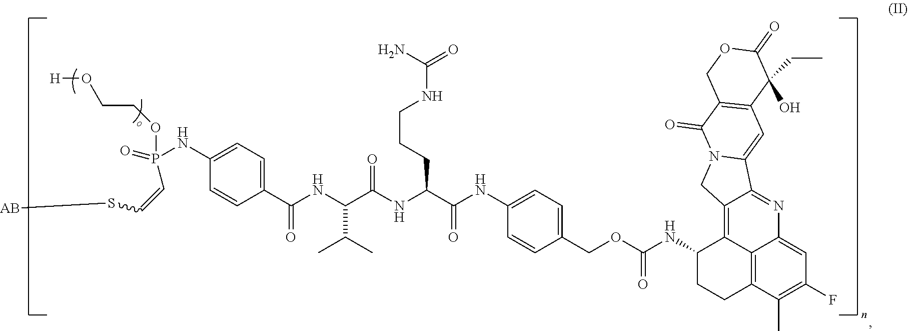

or a pharmaceutically acceptable salt, a stereoisomer, or a solvate thereof, wherein [symbol] indicates that the configuration of the double bond is E or Z; n is an integer ranging from 4 to 8; o is an integer ranging from 10 to 30; AB is an anti-LRRC15 antibody, or antigen binding portion thereof, comprising a VH and a VL which comprise the amino acid sequence set forth in SEQ ID NO: 6 and the amino acid sequence set forth in SEQ ID NO: 12, respectively.

In some aspects, the disclosure provides an antibody drug conjugate (ADC) having the formula (II):

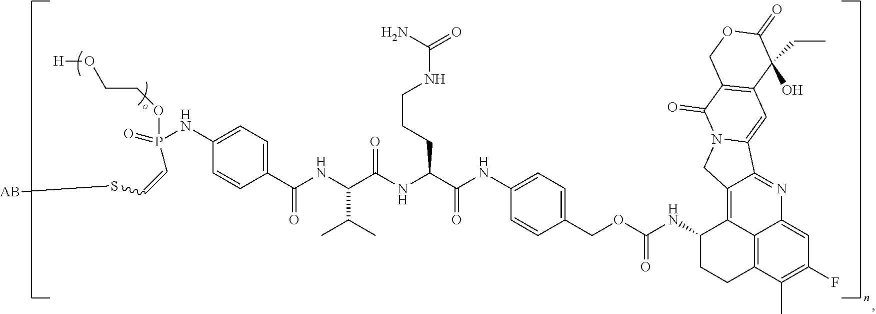

or a pharmaceutically acceptable salt, a stereoisomer, or a solvate thereof, wherein [symbol] indicates that the configuration of the double bond is E or Z; n is an integer ranging from 4 to 8; o is an integer ranging from 10 to 30; AB is an anti-LRRC15 antibody, or antigen binding portion thereof, comprising a VH and a VL which comprise the amino acid sequence set forth in SEQ ID NO: 7 and the amino acid sequence set forth in SEQ ID NO: 12, respectively.

In some aspects, the disclosure provides an antibody drug conjugate (ADC) having the formula (II):

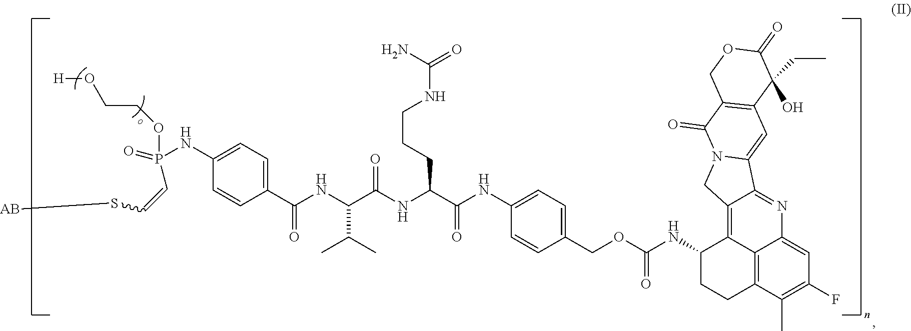

or a pharmaceutically acceptable salt, a stereoisomer, or a solvate thereof, wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 25 and the amino acid sequence set forth in SEQ ID NO: 30; respectively.

In some aspects, the disclosure provides an antibody drug conjugate (ADC) having

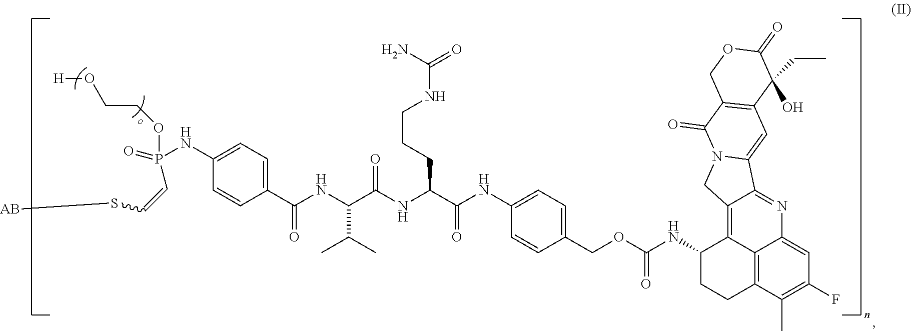

or a pharmaceutically acceptable salt, a stereoisomer, or a solvate thereof, wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 26 and the amino acid sequence set forth in SEQ ID NO: 30; respectively.

In some aspects, the disclosure provides an antibody drug conjugate (ADC) having the formula (II):

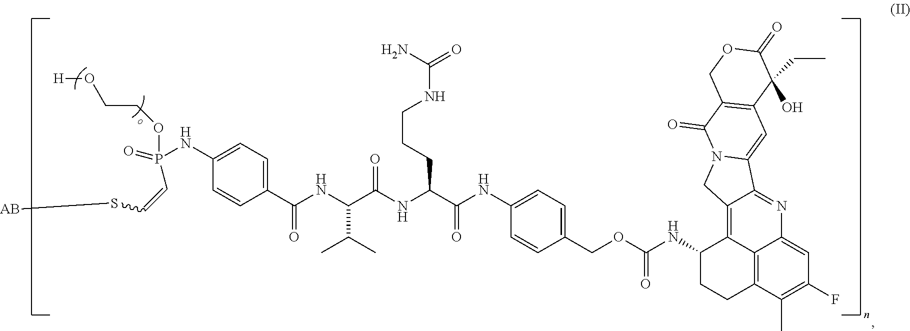

or a pharmaceutically acceptable salt, a stereoisomer, or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 34 and the amino acid sequence set forth in SEQ ID NO: 39, respectively.

In some aspects, the disclosure provides an antibody drug conjugate (ADC) having the formula (II):

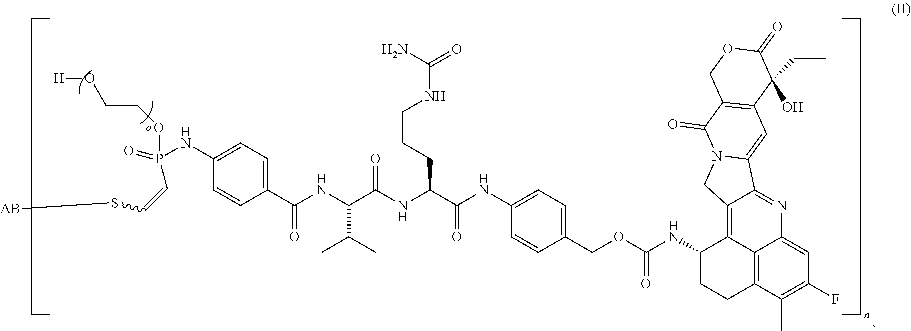

or a pharmaceutically acceptable salt, a stereoisomer, or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 35 and the amino acid sequence set forth in SEQ ID NO: 39, respectively.

In some aspects, the disclosure provides an antibody drug conjugate (ADC) having the formula (II):

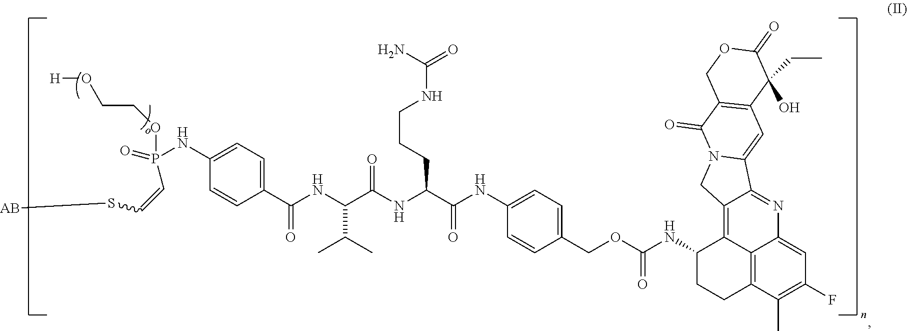

or a pharmaceutically acceptable salt, a stereoisomer or a solvate thereof, wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 43 and the amino acid sequence set forth in SEQ ID NO: 48, respectively.

In some aspects, the disclosure provides an antibody drug conjugate (ADC) having the formula (II):

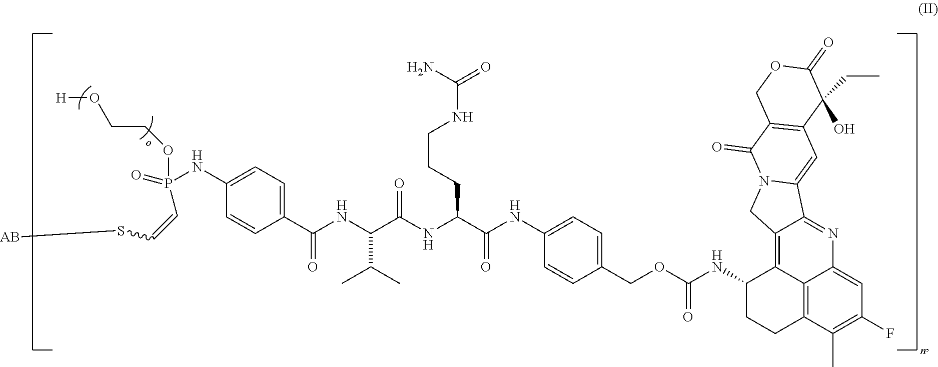

or a pharmaceutically acceptable salt, a stereoisomer or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 44 and the amino acid sequence set forth in SEQ ID NO: 48, respectively.

In some aspects, the disclosure provides an antibody drug conjugate (ADC) having the formula (II):

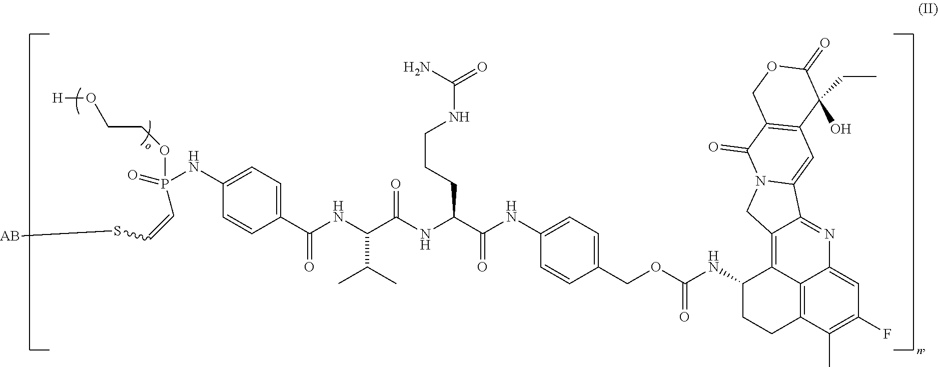

or a pharmaceutically acceptable salt, a stereoisomer, or solvate thereof, wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 52 and the amino acid sequence set forth in SEQ ID NO: 57, respectively.

In some aspects, the disclosure provides an antibody drug conjugate (ADC) having the formula (II):

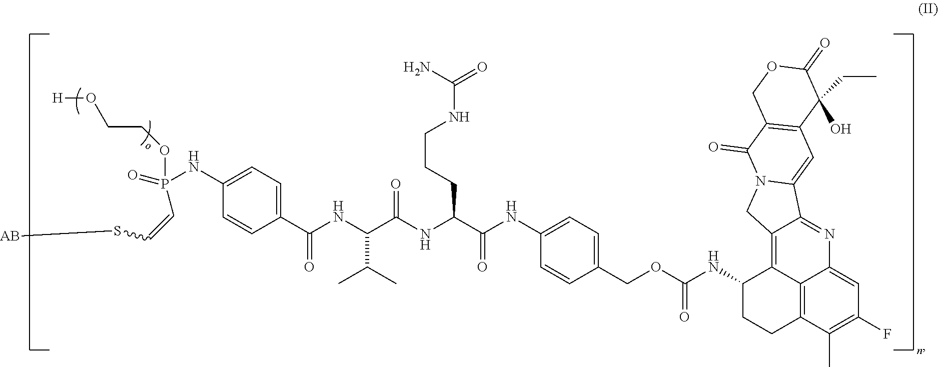

or a pharmaceutically acceptable salt, a stereoisomer, or solvate thereof, wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 53 and the amino acid sequence set forth in SEQ ID NO: 57, respectively.

In some aspects, the disclosure provides an antibody drug conjugate (ADC) having the formula (II):

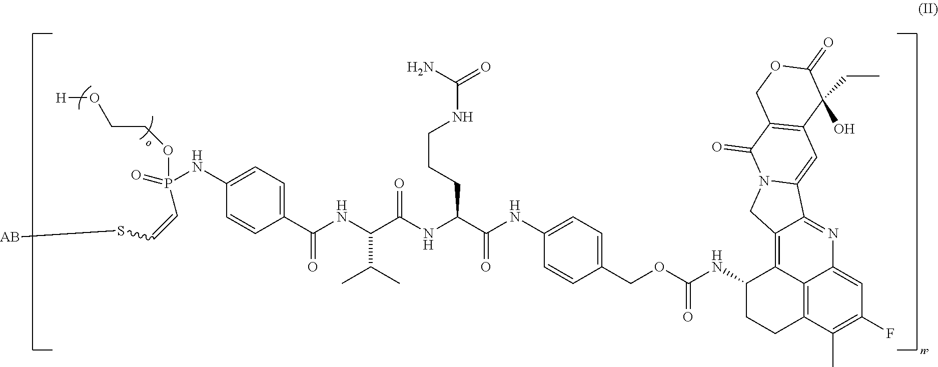

or a pharmaceutically acceptable salt, a stereoisomer, or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 61 and the amino acid sequence set forth in SEQ ID NO: 66, respectively.

In some aspects, the disclosure provides an antibody drug conjugate (ADC) having the formula (II):

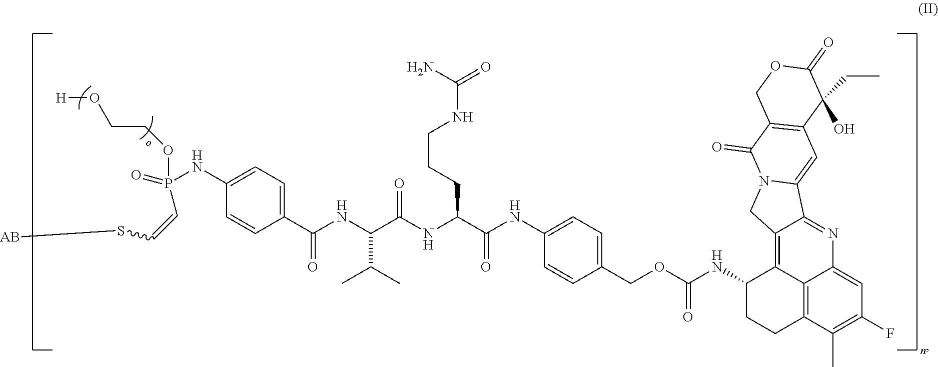

or a pharmaceutically acceptable salt, a stereoisomer, or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a VH and a VL, which comprise the amino acid sequence set forth in SEQ ID NO: 62 and the amino acid sequence set forth in SEQ ID NO: 66, respectively.

In some aspects, n in formula (I) is 4, 5, 6, 7, 8, or 9. In some aspects, n is 6. In some aspects, n is 7. In some aspects, n is 8. In some aspects, n is 9.

In some aspects, AB in formula (I) comprises an IgG1 constant region, IgG2 constant region, IgG3 constant region, IgG4 constant region, or a variant thereof. In some aspects, AB in formula (I) comprises an IgG1 antibody. In some aspects, the AB in formula (I) is a human, humanized, or chimeric antibody. In some aspects, the antigen binding portion thereof in the ADC comprises a Fab, Fab′, (Fab′)2, Fv, or scFv fragment. In some aspects, the AB in formula (I) specifically binds to LRRC15 with a KD less than 1×10⁻⁷ M, less than 1×10⁻⁸ M, less than 1×10⁻⁹ M, or or less than 1×10⁻¹⁰ M. In some aspects, the AB in formula (I) specifically binds to LRRC15 with a KD with about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 5 nM or less, about 3 nM or less, about 1 nM or less, about 0.9 nM or less, about 0.8 nM or less, about 0.7 nM or less, about 0.6 nM or less, about 0.5 nM or less, about 0.4 nM or less, about 0.3 nM or less, about 0.2 nM or less, or about 0.1 nM or less. In some aspects, the AB in formula (I) binds to an epitope of human LRRC15 identified by Carbene Footprinting analysis in Example 5 and FIGS. 4A-4C, for example, the epitope includes all or a portion of amino acids L⁴¹⁵, C⁴¹⁶, E⁴¹⁷, L⁴¹⁸, L⁴²⁰, Y⁴²¹, W⁴²⁵, I⁴³¹, W⁴³⁷, T⁴⁴⁹, C⁴⁵³, and F⁴⁵⁴ of human LRRC15 (SEQ ID NO: 1).

In some aspects, the antibody, or antigen binding portion thereof, in the ADC comprises a bispecific molecule comprising the anti-LRRC15 antibody or antigen binding portion thereof and a second binding region that binds to another antigen. In some aspects, the antibody, or antigen binding portion thereof, in the ADC comprises a multispecific molecule comprising the anti-LRRC15 antibody, or antigen binding portion thereof and at least two binding regions, each of which binds other antigens.

In some aspects, the ADC has an improved bystander effect in vivo compared to a reference ADC comprising the same anti-LRRC15 antibody, or antigen binding portion thereof and a DXd payload (MBP001-Deruxtecan).

In some aspects, the ADC induces in vivo bystander killing of tumor cells which do not express LRRC15.

In some aspects, the disclosure comprises a pharmaceutical composition comprising the ADC disclosed herein and a pharmaceutically acceptable carrier. In some aspects, the pharmaceutical composition further comprises one or more additional therapeutic agents. In some aspects, the disclosure comprises a kit comprising the ADC disclosed herein and instructions for use. In some aspects, the disclosure comprises a method of producing the ADC disclosed herein comprising conjugating the anti-LRRC15 antibody or antigen binding portion thereof with Compound A.

In some aspects, the disclosure comprises a method of inducing or improving a bystander effect of a cancer therapy in a subject in need thereof comprising administering to the subject the ADC disclosed herein or the pharmaceutical composition disclosed herein. In some aspects, the administering results in the killing of LRRC15-negative tumor cells. In some aspects, the LRRC15-negative tumor cells are adjacent to LRRC15-positive cells. In some aspects, the administering results in the killing of LRRC15-negative tumor cells adjacent to LRRC15-positive stromal cells. In some aspects, the LRRC15-positive stromal cells comprise LRRC15-expressing fibroblasts.

In some aspects, the disclosure comprises a method of treating a tumor that expresses LRRC15 in a subject in need thereof comprising administering to the subject a therapeutically effective amount of the ADC disclosed herein or the pharmaceutical composition disclosed herein. In some aspects, the tumor is of a cancer comprising: colorectal cancer, breast cancer, lung cancer, ovarian cancer, pancreatic cancer, bladder cancer, uterine/cervical cancer, prostate cancer, testicular cancer, esophageal cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, or myelodysplastic syndromes.

In some aspects, the methods herein further comprises administering one or more additional therapies. In some aspects, the one or more additional therapies comprises radiation therapy, chemotherapy, immune checkpoint inhibitor therapy, CAR-T therapy, immunosuppressive therapy, immunostimulatory therapy, cell therapy, or any combination thereof. In some aspects, the one or more additional therapies comprise an immune checkpoint inhibitor. In some aspects, the immune checkpoint inhibitor comprises an anti-PD-1 antibody, an anti-PD-L1 antibody, an anti-LAG-3 antibody, an anti-CTLA-4 antibody, an anti-CCR8 antibody, an anti-TIGIT antibody, an anti-TIM3 antibody, or any combination thereof.

In some aspects, the disclosure provides a method of treating cancer that expresses LRRC15 in a stromal cell in a subject in need thereof comprising administering to the subject a therapeutically effective amount of the ADC of formula (I) of any of the preceding claims or the pharmaceutical composition of any one of the preceding claims. In some aspects, the tumor is of a cancer comprising squamous non-small cell lung cancer (SqNSCLC), non-squamous NSCLC, estrogen-receptor (ER) positive breast cancer, EGFR wildtype non-squamous NSCLC, metastatic castration-resistant prostate cancer (mCRPC) bony metastasis, pancreatic cancer, MSS colorectal cancer (CRC), HR/HER-breast cancer, triple-negative breast cancer (TNBC), or head and neck squamous cell carcinoma (HNSCC). In some aspects, the tumor is of a cancer comprising a breast cancer. In some aspects, the breast cancer comprises a HR/HER breast cancer or a triple-negative breast cancer (TNBC).

In some aspects, the method further comprises quantifying the expression of LRRC15 in the tumor using immunohistochemistry (IHC), wherein the quantification comprises calculating an H-score according to the formula: H-score=[(% tumor×1 (low intensity))+(% tumor×2 (medium intensity))+(% tumor×3 (high intensity)]. In some aspects, the tumor expresses LRRC15 by an H-score of at least 1, e.g., 1 to 300. In some aspects, the stromal cells proximal to the tumor express LRRC15 by an H-score of at least 1, e.g., 1 to 300. In some aspects, the tumor and/or the stromal cells express LRRC15 by an H-score of at least 1, e.g., 1 to 300

In some aspects, the disclosure provides an antibody drug conjugate (ADC) having the formula (II):

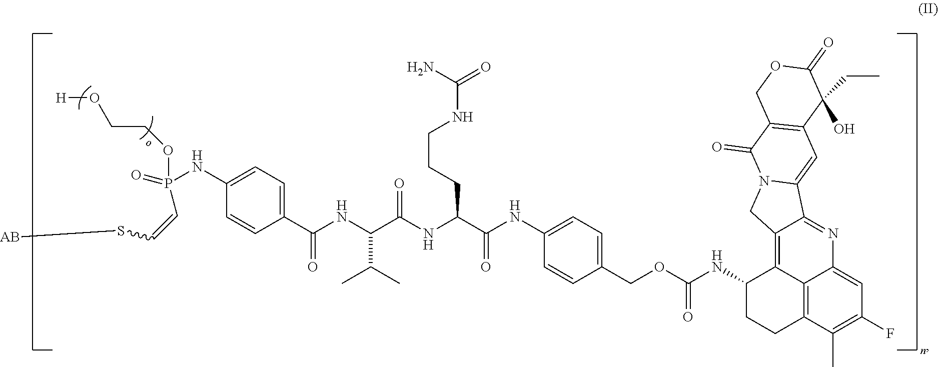

or a pharmaceutically acceptable salt, a stereoisomer, or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a heavy chain variable region (VH) comprising complementarity determining region (CDR)1, CDR2, and CDR3 regions comprising the amino acid sequences set forth in SEQ ID NOs: 3, 4, and 5, respectively, and a light chain variable region (VL) comprising CDR1, CDR2, and CDR3 regions comprising the amino acid sequences set forth in SEQ ID NOs: 9, 10, and 11.

In some aspects, the disclosure provides an antibody drug conjugate (ADC) having the formula (II):

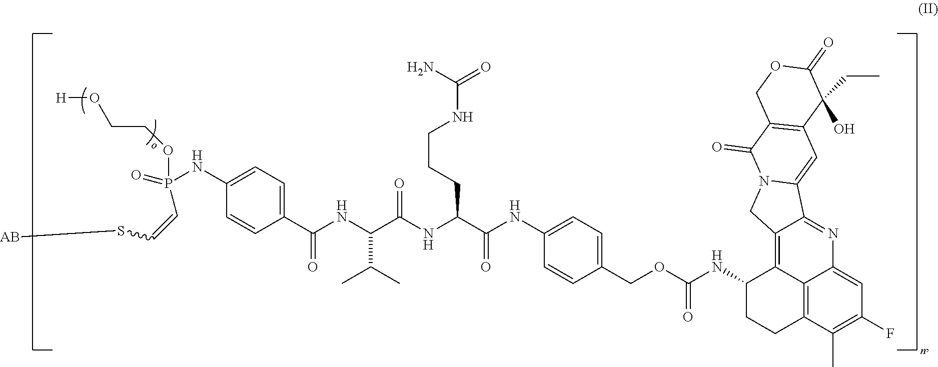

or a pharmaceutically acceptable salt, a stereoisomer, or a solvate thereof; wherein [symbol] indicates that the configuration of the double bond is E or Z; n is 8; o is 24; AB is an anti-LRRC15 antibody or antigen binding portion thereof comprising a heavy chain variable region (VH) comprising the amino acid sequence set forth in SEQ ID NO: 6 or SEQ ID NO: 7, and a light chain variable region (VL) comprising the amino acid sequence set forth in SEQ ID NO: 12.

In some aspects, the disclosure provides an antibody drug conjugate (ADC) having the formula:

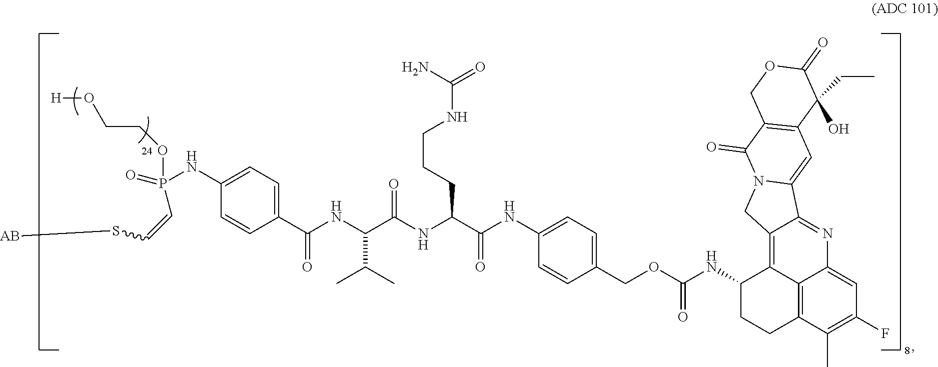

wherein [symbol] indicates that the configuration of the double bond is E or Z; AB is an anti-LRRC15 antibody comprising a heavy chain comprising the amino acid sequence set forth in SEQ ID NO: 14 or SEQ ID NO: 15, and a light chain comprising the amino acid sequence set forth in SEQ ID NO: 17.

In some aspects, the anti-LRRC15 antibody or antigen binding portion thereof consists of two heavy chains and two light chains wherein each heavy chain comprises the amino acid sequence set forth in SEQ ID NO: 14 or SEQ ID NO: 15, and wherein each light chain comprises the amino acid sequence set forth in SEQ ID NO: 17, respectively.

### BRIEF DESCRIPTION OF THE DRAWINGS

FIGS. 1A-1E show cross-species binding by enzyme-linked immunosorbent assay (ELISA) of anti-LRRC15 antibodies, or an isotype control (KLH) at 8 difference concentrations to LRRC15. Binding of the antibodies was assessed to LRRC15 from human (FIG. 1A), cynomolgus (FIG. 1B), mouse (FIG. 1C), rat (FIG. 1D), and canine (FIG. 1E). FIGS. 1A-1C compared the binding of the anti-LRRC15 antibody clones MBP006, MBP003, MBP001, MBP004, MBP005, and huM25. FIGS. 1D and 1E compared the binding of the anti-LRRC15 antibody clones MBP001 and huM25.

FIG. 2 shows an epitope community of antibody hits as described in Example 1.

FIG. 3 shows the binding kinetics for a titration of the anti-LRRC15 antibody MBP001 to human LRRC15.

FIGS. 4A-4D show three-dimensional representations of human LRRC15 (FIG. 4A), with the binding epitope of the anti-LRRC15 antibody MBP001 highlighted (FIG. 4B). The peptic sequence coverage of the human LRRC15 amino acid sequence is highlighted in FIG. 4C, and the epitope interaction between human LRRC15 and the mAb MBP001 is shown in FIG. 4D.

FIGS. 5A-5C show binding of various anti-LRRC15 antibodies to a 3T3 transfectant line expressing human LRRC15 (FIG. 5A), a 3T3 transfectant line expressing mouse LRRC15 (FIG. 5B), and a mesenchymal cancer cell line (U87-MG) expressing endogenous LRRC15 (FIG. 5C) as measured by flow cytometry.

FIGS. 6A-6H show cell surface binding of anti-LRRC15 antibodies to human bone derived marrow mesenchymal stem cells (FIGS. 6A-6G) as measured by flow cytometry. A summary of the antibody binding data for the human bone marrow derived mesenchymal stem cells is shown in FIG. 6H.

FIG. 7 shows the estimated number of LRRC15 receptors in primary breast cancer-associated fibroblasts over multiple passages ex vivo.

FIG. 8 shows the estimated number of LRRC15 receptors in a 3T3 cell line overexpressing mouse LRRC15 alongside a panel of commercially acquired primary human stromal cells at early passage. Bone marrow-derived mesenchymal stem cells, adipose-derived mesenchymal stem cells and primary cancer associated fibroblasts from gastric, ovarian, lung and breast tissue were cultured with or without 10 ng/ml of TGFβ-1.

FIGS. 9A-9C show the concentration-dependent binding of AF647-labeled LRRC15 antibodies to primary human stromal cells by flow cytometry. Bone marrow-derived mesenchymal stem cells (FIGS. 9A-9B) and breast cancer-associated fibroblasts (FIG. 9C) were cultured in the presence of 10 ng/ml of TGFβ-1.

FIGS. 10A-10B show the relative internalization rates of anti-LRRC15 mAbs applied to 3T3 cells expressing either human LRRC15 (FIG. 10A) or mouse LRRC15 (FIG. 10B) after either 3 hours or 24 hours.

FIGS. 11A-11B show U118-MG Promega antibody-dependent cell mediated cytotoxicity (ADCC) assay of the negative control (KLH-hIgG1 mAb), the positive control Cetuximab, MBP001, and MDC001. FIG. 11A shows RLU, and FIG. 11B shows the fold change relative to the negative control.

FIGS. 12A-12B show exatecan payload sensitivity between human primary stromal cells and human cancer cell lines. FIG. 12A shows the exatecan sensitivity represented by EC50 (uM) by Incucyte confluence or nuclear red count. Human primary stromal cells include BM-MSC, Breast CAF, Colon CAF, and Lung CAF. Human cancer cell lines include NCI-H1650, EBC-1, and PANC1 NucRed. FIG. 12B shows exatecan sensitivity between LRRC15+3T3 cells and LRRC15-PANC1 (cancer cells).

FIGS. 13A-13E are a set of graphs and tables showing percent viability of cells treated with anti-LRRC15-P5-exatecan across ADCs against a panel of LRRC15-expressing cell lines. FIG. 13A shows cell surface LRRC15 protein expression, from left to right, of PANC1 cancer cells (LRRC15−), 3T3 parental cells (LRRC15−), and LRRC15 transfectant cell lines 3T3 human LRRC15 clone 1 (LRRC15++), 3T3 mouse LRRC15 2M7 (LRRC15+) and 3T3 mouse LRRC15 1H4 (LRRC15++). FIGS. 13B-13C show the activity of MDC001 on 3T3 human LRRC15 clone 1 (LRRC15++) as compared to parental cells (LRRC15−) (FIG. 13B), and the activity of MDC001 on 3T3 mouse LRRC15 2M7 (LRRC15+) and 3T3 mouse LRRC15 1H4 (LRRC15++) as compared to parental cells (LRRC15−) (FIG. 13C). FIGS. 13D-13E show the activity of ADCs on 3T3 human LRRC15 clone 1 (LRRC15++) (FIG. 13D), and 3T3 mouse LRRC15 1H4 (LRRC15++) (FIG. 13E).

FIGS. 14A and 14B show assessment of direct cell killing activity via fluorescence imaging by an Incucyte SX5 imaging system (Sartorius), plotting Normalized Nuclear Red Cell Count. FIG. 14A shows assessment of direct cell killing activity by various ADCs on the PANC1 NucRed cells plated in monoculture, and FIG. 14B shows assessment of direct cell killing activity by various ADCs on PANC1 NucRed cells in co-culture with LRRC15 negative 3T3 cells (FIG. 14B).

FIGS. 15A-15B are a set of graphs showing bystander killing effect on LRRC15 negative cancer cells through LRRC15 positive cells. FIG. 15A shows dose dependent bystander activity of anti-LRRC15-P5-exatecan ADCs using 3T3 human LRRC15 clone 1, and FIG. 15B shows bystander activity using 3T3 mouse LRRC15 1H4.

FIGS. 16A-16E are a set of graphs showing bystander killing effect on LRRC15 negative cancer cells through LRRC15 positive cells by MDC001 ADC: human LRRC15 clone 1 (FIG. 16A, 4 days), mouse LRRC15 2M7 (FIG. 16B, 4 days), and mouse LRRC15 1H4 (FIG. 16C, 4 days). FIGS. 16D and 16E show the 7-day bystander killing effect on human and mouse LRRC15 negative cancer cells, respectively. Bystander killing was is demonstrated using either a 4:1 or 8:1 ratio of cancer cell to LRRC15+ stromal cells.

FIGS. 17A-17D are a set of Incucyte images of LRRC15 positive and LRRC15 negative cells after treatment with 0.4 nM of MDC001 or an isotype control ADC. FIG. 17A shows minimal activity of MDC001 on 3T3 human LRRC15 clone 1 cells in monoculture and FIG. 17D shows no activity on LRRC15 negative PANC1 NucRed cancer cells alone. Bystander activity as demonstrated by a reduction in the PANC1 NucRed cancer cell counts is evident at day 4 (FIG. 17B) and day 7 (FIG. 17D) in the co-culture condition where the ratio is four LRRC15 negative cancer cells to one LRRC15 positive stromal cell.

FIG. 18 shows Incucyte images of cells after treatment with 0.4 nM of MBP001-Deruxtecan ADC or with 0.4 nM KLH-Deruxtecan ADC. Limited bystander killing is observed as compared to FIG. 17D.

FIGS. 19A-19B show the bystander activity of anti-LRRC15-Deruxtecan compared with MDC001.

FIGS. 20A-20B are a set of graphs showing in vivo efficacy of anti-LRRC15-P5-exatecan ADCs.

FIGS. 21A-21B are a set of graphs showing in vivo efficacy of anti-LRRC15 mAb and anti-LRRC15-P5-exatecan ADC. FIG. 21A is a graph showing in vivo efficacy of MDC001 in the EBC1 model upon treatment with a single dose. FIG. 21B is a graph showing in vivo efficacy of MDC001 in the NCI-H1650 model upon treatment with a single dose at 3 mg/Kg and 10 mg/Kg dose levels when compared to the respective isotype-ADC control groups.

FIGS. 22A-22C show LRRC15 expression in EBC-1 (FIG. 22A) and NCI-H1650 (FIG. 22B) model tumors as assessed by IHC using a mouse backbone anti-LRRC15 antibody. FIGS. 22A-22C show MDC001 induces tumor regression via bystander killing with minimal impact on LRRC15-expressing fibroblasts. FIG. 22C shows that LRRC15 stromal staining is retained post-treatment with MDC001.

FIG. 23 is a Western Blot image showing induction of the pharmacodynamic markers of DNA damage response, including pCHK1 and γH2AX in tumors from the LRRC15+EBC1 model (n=3) at 4, 24, and 96 hours after a single intravenous injection with 1.5 mg/kg of MDC001.

FIGS. 24A-24B are a set of graphs showing anti-LRRC15 huM25-exatecan ADCs result in less direct-killing activity on 3T3 mouse LRRC15 1H4 cells as compared to anti-LRRC15 huM25-MMAE ADC.

FIG. 25 shows a schematic diagram comparing the effects of ADCs targeting a tumor antigen (top) with those targeting an antigen in the stromal region of a tumor (bottom).

FIGS. 26A-26B are a set of graphs showing antigen-dependent and dose-dependent bystander activity of anti-LRRC15 ADCs (i.e., MDC001) on PANC-1 NucRed cancer cells co-cultured with 3T3 cells overexpressing human LRRC15 antigen (FIG. 26A), and the effects on tumor volume of a single IP injection of vehicle, MDC001 (6 mg/kg), or isotype ADC (6 mg/kg) in mice with subcutaneous NCI-H1650 tumors (FIG. 26B).

FIGS. 27A-27F are a set of graphs and images showing in vivo dose dependent efficacy of anti-LRRC15 ADC. Mice with breast cancer PDX tumors CTG-1211 (FIG. 27A) and CTG-1727 (FIG. 27C) were treated with a single dose of MDC001 at 3 or 10 mg/kg and tumor volume was measured over approximately 30 days after dosing. Results are presented as mean values. Representative IHC images of the baseline LRRC15 expression and the corresponding staining intensity is shown for each model (FIGS. 27B and 27D). Quantitative analysis of the LRRC15 expression across the whole tissue (FIG. 27E) or within the stromal compartment (FIG. 27F) of the two breast cancer PDX models is also shown. Statistical analysis was performed using two-way ANOVA, multiple comparisons. Significance is indicated by *(P=0.0292) and **** (P<0.0001).

### DETAILED DESCRIPTION OF THE DISCLOSURE

The present disclosure provides an antibody drug conjugate (ADC) of the formula (I), a pharmaceutical composition comprising the ADC, wherein the ADC comprises an LRRC15 antibody or antigen binding portion thereof, the use of the ADC in a method of treating disease, such as cancer. In particular, the ADC is effective in treating cancer as its LRRC15 antibody targets LRRC15, which is expressed on stroma regions of a tumor. Targeting stroma, or supportive tissue surrounding a tumor, in cancer therapy using antibodies and conjugates can offer several benefits, such as (i) disruption of tumor microenvironment, (ii) reduced tumor growth and spread, (iii) enhanced drug delivery, (iv) inhibition of angiogenesis, (v) reduced resistance to therapy, (vi) direct tumor killing, (vii) immune modulation; and/or (viii) synergistic effects with other therapies.

### Definitions

In order for the following detailed description to be readily understood, certain terms are first defined. Additional definitions are provided throughout.

The term “alkyl” by itself or as part of another term in general refers to a substituted or unsubstituted straight chain or branched, saturated hydrocarbon having the indicated number of carbon atoms; e.g., “—(C₁-C₈)alkyl” or “—(C₁-C₁₀)alkyl” refer to an alkyl group having from 1 to 8 or 1 to 10 carbon atoms, respectively). When the number of carbon atoms is not indicated, the alkyl group may have from 1 to 8 carbon atoms. Representative straight chain —(C₁-C₈)alkyl groups include, but are not limited to, -methyl, -ethyl, -n-propyl, -n-butyl, -n-pentyl, -n-hexyl, -n-heptyl and -n-octyl; branched-(C₁-C₈)alkyl groups include, but are not limited to, -isopropyl, -sec-butyl, -isobutyl, -tert-butyl, -isopentyl, and -2-methylbutyl. In some aspects, an alkyl group may be unsubstituted. Optionally, an alkyl group may be substituted, e.g., with one or more groups.

The term “polyalkene glycol unit,” refers to a repeating alkylene glycol subunit having the formula —[(CH₂)ₙ—O]_{y}—, where n is the number of methylene groups in the subunit and y is the number of subunits in the unit. The oxygen atom of the terminal subunit can be substituted with a hydrogen atom, a protecting group, or any other allowable functionality.

The term “substituted”, “optionally substituted”, “optionally may be substituted” or the like, unless otherwise indicated, in general means that one or more hydrogen atoms can be each independently replaced with a substituent. Typical substituents include, but are not limited to, —X, —R, —O⁻, —OR, —SR, —S⁻, —NR₂, ═NR, —CX₃, —CN, —OCN, —SCN, —N═C—O, —NCS, —NO, —NO₂, ═N₂, —N₃, —NRC(═O)R, —C(═O)R, —C(═O)NR₂, —SO₃, —SO₃H, —S(═O)₂R, —OS(═O)₂OR, —S(═O)₂NR, —S(═O)R, —OP(═O)(OR)₂—P(═O)(OR)₂, —PO₄³⁻, —PO₃H₂, —C(═O)R, —C(═O)X, —C(═S)R, —CO₂R, —CO₂H, —C(═S)OR, —C(═O)SR, —C(═S)SR, —C(═O)NR₂, —C(═S)NR₂, or —C(═NR)NR₂, where each X is independently a halogen: —F, —Cl, —Br, or —I; and each R is independently —H, —(C₁-C₂₀)alkyl (such as e.g. —(C₁-C₁₀)alkyl or —(C₁-C₈)alkyl), —(C₆-C₂₀)aryl, (such as e.g. —(C₆-C₁₀)aryl or, e.g., —C₆-aryl), —(C₃-C₁₄)heterocycle (such as e.g. —(C₃-C₁₀)heterocycle or —(C₃-C₈)heterocycle), a protecting group, or a prodrug moiety. Typical substituents also include (═O).

The term “aliphatic or aromatic residue”, as used herein, in general refers to an aliphatic substituent, such as e.g. but not limited to an alkyl residue, which, however, can be optionally substituted by further aliphatic and/or aromatic substituents. As non-limiting examples an aliphatic residue can be a nucleic acid, an enzyme, a co-enzyme, a nucleotide, an oligonucleotide, a monosaccharide, a polysaccharide, a polymer, a fluorophore, optionally substituted benzene, etc., as long as the direct link of such a molecule to the core structure (in case of R′, e.g., the link to the nitrogen atom of the Y) is aliphatic. An aromatic residue is a substituent, wherein the direct link to the core structure is part of an aromatic system, e.g., an optionally substituted phenyl or triazolyl or pyridyl or nucleotide, as non-limiting example if the direct link of the nucleotide to the core structure is for example via a phenyl-residue. The term “aromatic residue”, as used herein, also includes a heteroaromatic residue.

The term “antibody” as used to herein includes whole antibodies and any antigen binding portions (i.e., “antigen-binding portions”) or single chains thereof. An “antibody” refers, in one aspect, to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. In certain naturally occurring antibodies, the heavy chain constant region is comprised of three domains, CH1, CH2, and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, 10⁻⁸ M or less, 1×10⁻⁹ M or less, 1×10⁻¹⁰ M or less, or 1×10⁻¹¹ M or less. In some aspects, the antibody specifically binds to an antigen with a K_{D} between 10⁻⁸ M and 10⁻¹⁰ M or between 10⁻⁹ M and 10⁻¹¹ M, but does not bind with high affinity to unrelated antigens.

An “antibody” according to the present disclosure includes, but is not limited to, naturally and non-naturally occurring antibodies, monoclonal antibodies, polyclonal antibodies, chimeric antibodies, humanized antibodies, human antibodies, nonhuman antibodies, bivalent antibodies, bispecific antibodies, multispecific antibodies, single chain antibodies, diabodies, and nanobodies.

An “isolated antibody,” as used herein, refers to an antibody which is substantially free of other antibodies having different antigenic specificities.

The phrase “antigen binding portion” of an antibody, as used herein, refers to one or more fragments of an antibody that retain the ability to specifically bind to an antigen (e.g., human and/or cynomolgus LRRC15). It has been shown that the antigen-binding function of an antibody can be performed by fragments of a full-length antibody. Examples of binding fragments of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) an Fd fragment consisting of the V_{H} and CH1 domains; (iv) an Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Antigen-binding portions can be produced by recombinant DNA techniques, or by enzymatic or chemical cleavage of intact immunoglobulins.

Antibody binding portions, e.g., antibody fragments, within the scope of the present disclosure also include F(ab′)2 fragments which may be produced by enzymatic cleavage of an IgG by, for example, pepsin. Fab fragments may be produced by, for example, reduction of F(ab′)2 with dithiothreitol or mercaptoethylamine. A Fab fragment is a VL-CL chain appended to a VH-CH1 chain by a disulfide bridge. A F(ab′)2 fragment is two Fab fragments which, in turn, are appended by two disulfide bridges. The Fab portion of an F(ab′)2 molecule includes a portion of the Fc region between which disulfide bridges are located.

As used herein, “isotype” refers to the antibody class (e.g., IgG (including IgG1, IgG2, IgG3, and IgG4), IgM, IgA (including IgAQ1 and IgA2), IgD, and IgE antibody) that is encoded by the heavy chain constant region genes of the antibody.

An antibody may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids.

As used herein, the term “allotype” refers to naturally occurring variants within a specific isotype group, where the variants differ in a few amino acids. Anti-LRRC15 antibodies described herein can be of any allotype.

As used herein, the term “hypervariable region” (sometimes referred to as the “variable region”) refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al., (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, (1987) J. Mol. Biol. 196:901-917).

As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing. Amino acid residues in antibodies can also be defined using other numbering systems, such as Chothia, enhanced Chothia, IMGT, Kabat/Chothia composite, Honegger (AHo), Contact, or any other conventional antibody numbering scheme.

The term “acceptor human framework” refers to a framework comprising the amino acid sequence of a light chain variable domain (V_{L}) framework, or a heavy chain variable domain (VH) framework derived from a human immunoglobulin framework or a human consensus framework. An acceptor human framework “derived from” a human immunoglobulin framework or a human consensus framework may have the same amino acid sequence as the naturally occurring human immunoglobulin framework or human consensus framework, or it may have amino acid sequence changes compared to wild-type naturally occurring human immunoglobulin framework or human consensus framework. In some aspects, the number of amino acid changes are 10, 9, 8, 7, 6, 5, 4, 3, or 2, or 1. In some aspects, the V_{L} acceptor human framework is identical in sequence to the V_{L} human immunoglobulin framework sequence or human consensus framework sequence.

An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

The term “epitope” or “antigenic determinant” refers to a site on an antigen (e.g., human LRRC15) to which an immunoglobulin or antibody specifically binds. Epitopes can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, or 22 amino acids in a unique spatial conformation.

The term “monoclonal antibody,” as used herein, refers to an antibody that displays a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In one aspect, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell. Monoclonal antibodies include chimeric antibodies, human antibodies, and humanized antibodies and may occur naturally or be produced recombinantly.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). In one aspect, provided herein are single domain antibodies comprising two VH domains with modifications such that single domain antibodies are formed.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain aspects, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A “human” antibody refers to an antibody having variable regions in which both the framework and CDR regions are derived from human germline immunoglobulin sequences. Furthermore, if the antibody contains a constant region, the constant region also is derived from human germline immunoglobulin sequences. Also encompassed are antibodies derived from human germline immunoglobulin sequences that include normal somatic hypermutations which alter the germline immunoglobulin sequences relative to the wild-type germline immunoglobulin sequences.

A “humanized” antibody refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Any additions, deletions, insertions, substitutions or modifications of amino acids are permissible as long as they do not abrogate the ability of the antibody to bind to a particular antigen. A “humanized” antibody may retain an antigenic specificity similar to that of the original antibody.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody which comprises mouse immunoglobulin sequences only.

A “chimeric antibody” refers to an antibody in which the variable regions are derived from one or more species and the constant regions are derived from another species, such as an antibody in which the variable regions are derived from a mouse antibody and the constant regions are derived from a human antibody. See U.S. Pat. No. 4,816,567; and Morrison et al., (1984) Proc. Natl. Acad. Sci. USA 81:6851-6855.

A “domain antibody” or “nanobody” is an immunologically functional immunoglobulin fragment containing only the variable region of a heavy chain or the variable region of a light chain. In some instances, two or more VH regions are covalently joined with a peptide linker to create a bivalent domain antibody. The two Vu regions of a bivalent domain antibody may target the same or different antigens.

A “bivalent antibody” comprises two antigen binding sites. In some instances, the two binding sites have the same antigen specificities. However, bivalent antibodies may be bispecific.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Bifunctional antibodies include, for example, heterodimeric antibody conjugates (e.g., two antibodies or antibody fragments joined together with each having different specificities), antibody/cell surface-binding molecule conjugates (e.g., an antibody conjugated to a non-antibody molecule such as a receptor), and hybrid antibodies (e.g., an antibody having binding sites for two different antigens).

A “multispecific antibody” is an antibody (e.g., bispecific antibodies, tri-specific antibodies) that recognizes two or more different antigens or epitopes.

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker. For a review of scFvs, see Pluckthun (1994) THE PHARMACOLOGY OF MONOCLONAL ANTIBODIES, vol. 113, Rosenburg and Moore eds. Springer-Verlag, New York, pp. 269-315.

As used herein, the term “diabodies” refer to small antibody fragments with two antigen-binding sites in which the fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90:6444-6448. For a review of engineered antibody variants generally see Holliger and Hudson (2005) Nat. Biotechnol. 23:1126-1136.

The term “immune cell engager” or “ICE” is used herein with reference to a multifunctional molecule comprising two or more binding specificities able to redirect immune effector cells against cancer cells. Exemplary immune cell engagers include T-cell engagers (e.g., bispecific T-cell engagers or BiTEs), NK-cell engagers (NKCEs), B-cell engagers, dendritic cell engagers, and macrophage cell engagers.

The term “binds to the same epitope” is used with reference to two or more antibodies that bind to the same segment or same segments of amino acid residues. Techniques for determining whether antibodies bind to the same epitope may be determined by epitope mapping methods described herein. Other methods involve monitoring the binding of the antibody to antigen fragments (e.g., proteolytic fragments) or to mutated variations of the antigen where loss of binding due to a modification of an amino acid residue within the antigen sequence is often considered an indication of an epitope component, such as alanine scanning mutagenesis (Cunningham & Wells (1985) Science 244:1081), yeast display of mutant target sequence variants, or analysis of chimeras. In addition, computational combinatorial methods for epitope mapping can also be employed. These methods rely on the ability of the antibody of interest to affinity isolate specific short peptides from combinatorial phage display peptide libraries. Antibodies having the same V_{H} and V_{L} or the same CDR1, 2 and 3 sequences are expected to bind to the same epitope.

Antibodies that “compete with another antibody for binding to a target” refer to antibodies that inhibit (partially or completely) the binding of another antibody to the target. Whether two antibodies compete with each other for binding to a target, i.e., whether and to what extent one antibody inhibits the binding of the other antibody to a target, may be determined using known binding competition experiments involving surface plasmon resonance (SPR) and bio-layer interferometry (BLI). In certain aspects, an antibody competes with, and inhibits binding of another antibody to a target by at least 50%, 60%, 70%, 80%, 90% or 100%. The level of inhibition or competition may be different depending on which antibody is the “blocking antibody” (i.e., the antibody that when combined with an antigen blocks another immunologic reaction with the antigen). Competition assays can be conducted as described, for example, in Ed Harlow and David Lane, Cold Spring Harb. Protoc. 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope, or to adjacent epitopes (e.g., as evidenced by steric hindrance). Two antibodies “cross-compete” if antibodies block each other both ways by at least 50%, i.e., regardless of whether one or the other antibody is contacted first with the antigen in the competition experiment.

Competitive binding assays for determining whether two antibodies compete or cross-compete for binding include competition for binding to cells expressing LRRC15, e.g., by flow cytometry. Other methods include surface plasmon resonance (SPR) (e.g., BIACORE®), solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) using a predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Any K_{D} greater than about 10⁻⁴ M is generally considered to indicate nonspecific binding.

The term “K_{assoc}” or “kₐ,” as used herein, refers to the association rate of a particular antibody-antigen interaction, whereas the term “k_{dis}” or “k_{d},” as used herein, refers to the dissociation rate of a particular antibody-antigen interaction. The term “K_{D}.” as used herein, is intended to refer to the dissociation constant, which is obtained from the ratio of k_{d} to kₐ (i.e. k_{d}/kₐ) and is expressed as a molar concentration (M). K_{D} values for antibodies can be determined using methods well established in the art. A preferred method for determining the K_{D} of an antibody is by using surface plasmon resonance, for example, using a biosensor system such as a BIACORE®® system or flow cytometry and Scatchard analysis, or bio-layer interferometry.

The term “EC50” or “IC50” in the context of an in vitro or in vivo assay using an antibody or immunoconjugate refers to the concentration of an antibody that induces a response that is 50% of the maximal response, i.e., halfway between the maximal response and the baseline. In pharmacology, the potency of a compound is expressed as the half-maximal effective concentration (EC50), which refers to the concentration of a drug that induces a response halfway between the baseline and maximum. While expressing the potency of a compound by its EC50 value makes sense in a clinical context, it is counterintuitive in the context of bioactivity-guided purification, as the potency of a compound is inversely related to its EC50 value, and the most potent compound is the one with the lowest EC50. Half-maximal inhibitory concentration (IC50) is the most widely used and informative measure of a drug's efficacy. It indicates how much drug is needed to inhibit a biological process by half, thus providing a measure of potency of an antagonist drug in pharmacological research.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, the term “conjugate” is used with reference to an immunoconjugate or antibody drug conjugate comprising an anti-LRRC15 antibody or antigen binding portion thereof described herein linked to a cytotoxic or therapeutic drug described herein.

The term “linker,” as used herein, refers to a chemical moiety comprising a covalent bond and/or any chain of atoms that may be used to covalently attach e.g., a drug to the antibody. Linkers are known in the art and include e.g., disulfide groups, thioether groups, acid labile groups, photolabile groups, peptidase labile groups and esterase labile groups. Conjugation of an antibody of the present disclosure with cytotoxic drugs or other growth inhibitory agents may be performed e.g. using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to an antibody (WO 94/11026).

In certain aspects, the linker is a “cleavable linker,” which may facilitate release of the cytotoxic drug or other growth inhibitory agent inside of or in the vicinity of a cell, e.g., a tumor cell. In some aspects, the linker is a linker cleavable in an endosome of a mammalian cell. For example, an acid-labile linker, a peptidase-sensitive linker, an esterase labile linker, a photolabile linker or a disulfide-containing linker (see e.g., U.S. Pat. No. 5,208,020) may be used.

The term “nucleic acid molecule,” as used herein, is used with reference to DNA molecules and RNA molecules. A nucleic acid molecule may be single-stranded or double-stranded, and may be a cDNA.

The term “isolated nucleic acid molecule,” as used herein in reference to nucleic acids encoding antibodies or antibody binding portions (e.g., V_{H}, V_{L}, CDR3), is intended to refer to a nucleic acid molecule in which the nucleotide sequences are essentially free of other genomic nucleotide sequences, e.g., those encoding antibodies that bind antigens other than LRRC15, which other sequences may naturally flank the nucleic acid in human genomic DNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

Also provided are “conservative sequence modifications” of the sequences set forth herein, e.g., amino acid sequence modifications which do not abrogate the binding of the antibody encoded by the nucleotide sequence or containing the amino acid sequence, to the antigen. Such conservative sequence modifications include conservative nucleotide and amino acid substitutions, as well as nucleotide and amino acid additions and deletions. For example, modifications can be introduced into a sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-LRRC15 antibody can be replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). Alternatively, in another aspect, mutations can be introduced randomly along all or part of an anti-LRRC15 antibody coding sequence, such as by saturation mutagenesis, and the resulting modified anti-LRRC15 antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 80% to 85%, 85% to 90% or 90% to 95%, and at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 80% to 85%, 85% to 90%, 90% to 95%, and at least about 98% to 99.5% of the amino acids.

The percent identity between two sequences is a function of the number of identical positions shared by the sequences (i.e., % homology=# of identical positions/total # of positions×100), considering the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See www.ncbi.nlm.nih.gov.

The term “recombinant host cell” (or simply “host cell”), as used herein, is intended to refer to a cell that comprises a nucleic acid that is not naturally present in the cell and may be a cell into which a recombinant expression vector has been introduced. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

The term “inhibition,” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity.

The term “immunotherapy” as used herein, refers to the treatment of a subject afflicted with, or at risk of contracting or suffering a recurrence of, a disease by a method comprising inducing, enhancing, suppressing or otherwise modifying an immune response.

The terms “immunostimulating therapy” and “immunostimulatory therapy,” as used herein, refer to a therapy that results in an increase (e.g., inducing or enhancing) an immune response in a subject for, e.g., treating cancer.

As used herein, “immune cell” refers to the subset of blood cells known as white blood cells, which include mononuclear cells such as lymphocytes, monocytes, macrophages, and granulocytes.

As used herein, “stroma” refers to the microenvironment surrounding and supporting a tumor. The cell population in tumor stroma often comprises cancer-associated fibroblasts (CAFs), pericytes, mesenchymal stem cells (MSCs) and inflammatory-immune cells. Some cells in tumor stroma are derived from bone marrow and are of hematopoietic lineage, including but not limited to, myeloid cells, granulocytes and neutropils. CAFs have specifically been suggested to derive from bone marrow mesenchymal stem cells (MSCs), or from epithelial normal or transformed cells via epithelial to mesenchymal transition (EMT), or finally from endothelial cells via endothelial to mesenchymal transition (EndMT) (see, e.g., Cirri P et al. Am. J. Cancer Res. 2011; 1(4):482-497).

As used herein, “abnormal” is used in the context of the activity or level or expression of a molecule which is outside of the normal activity or expression level (e.g., overexpressed) as compared to e.g., a control sample or reference sample exhibiting a normal activity/expression profile. The term “normal” is used herein in the context of the activity or level of expression of a protein found in a population of healthy, gender- and age-matched subjects. The minimal size of this healthy population may be determined using standard statistical measures, e.g., the practitioner could consider the incidence of the disease in the general population and the level of statistical certainty desired in the results. In some aspects, the normal range for activity, level or expression of a biomarker is determined from a population of subjects (e.g., at least five, ten or twenty subjects), for example from a population of at least forty or eighty subjects, and from more than 100 subjects.

As used herein, “administering” refers to the physical introduction of a LRRC15 targeting agent, such as an ADC (comprising an anti-LRRC15 antibody or antigen binding portion thereof described herein linked to a cytotoxic moiety described herein via a linker alone or in combination with another therapeutic agent) to a subject, using any of the various methods and delivery systems known to those skilled in the art. Preferred routes of administration for ADCs described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intra-lymphatic, intralesional, intracapsular, intra-orbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, “cancer” refers to a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream, and includes a variety of cancers, including but not limited to e.g., carcinomas, melanomas, sarcomas, leukemias, lymphomas, germ cell tumors, and blastomas. Exemplary cancers for treatment include cancers of the brain, bladder, breast, cervix, colon, head and neck, kidney, lung, non-small cell lung, mesothelioma, ovary, prostate, stomach and uterus, leukemia, and medulloblastoma.

As used herein, the term “small molecule drug” refers to a molecular entity, often organic or organometallic, that is not a polymer, that has medicinal activity, and that has a molecular weight less than about 2 kilodaltons (kDa), less than about 1 kDa, less than about 900 daltons (Da), less than about 800 Da or less than about 700 Da. The term encompasses most medicinal compounds termed “drugs” other than protein or nucleic acids, although a small peptide or nucleic acid analog can be considered a small molecule drug. Examples include chemotherapeutic anticancer drugs and enzymatic inhibitors. Small molecule drugs can be derived synthetically, semi-synthetically (i.e., from naturally occurring precursors), or biologically.

The terms “treat,” “treating,” and “treatment,” as used herein, refer to any type of intervention or process performed on, or administering an active agent (e.g., ADC comprising an anti-LRRC15 antibody or antigen binding portion thereof described herein linked to a cytotoxic moiety via a linker as described herein) to, the subject with the objective of preventing, reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, “adjunctive” or “combined” administration (co-administration) includes simultaneous administration of an ADC (comprising an anti-LRRC15 antibody or antigen binding portion thereof described herein linked to a cytotoxic moiety described herein via a linker) and one or more additional agents and/or compounds in the same or different dosage form, or combined administration in separate dosages concurrently or sequentially. Thus, an ADC (comprising an anti-LRRC15 antibody or antigen binding portion thereof described herein linked to a cytotoxic moiety described herein via a linker), and second, third, or more agents and/or compounds (e.g., small molecules) can be simultaneously administered in a single formulation or formulated for separate administration and are administered concurrently or sequentially.

“Combination” therapy, as used herein, means administration of two or more therapeutic agents in a coordinated fashion, and includes, but is not limited to, concurrent and sequential dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. (See, e.g., Kohrt et al. (2011) Blood 117:2423). For example, the ADC (comprising an anti-LRRC15 antibody linked to a cytotoxic moiety described herein via a linker) can be administered first followed by (e.g., immediately followed by) the administration of a second agent (e.g., an antibody or antigen binding portion thereof), or vice versa. In one aspect, the ADC is administered prior to administration of the second agent (e.g., antibody or antigen binding portion thereof). In another aspect, the ADC is administered, for example, a few minutes (e.g., within about 30 minutes) or at least one hour of the second agent (e.g., antibody or antigen binding portion thereof). Such concurrent or sequential administration can result in both the ADC and the second agent being simultaneously present in treated patients.

The administration of effective amounts of the ADC (comprising an anti-LRRC15 antibody or antigen binding portion thereof described herein linked to a cytotoxic moiety described herein via a linker) alone, or the ADC combined with another compound or agent (e.g., an immune checkpoint inhibitor such as an anti-PD-1 antibody), according to any of the methods provided herein, can result in at least one therapeutic effect, including, for example, reduced tumor growth or size, reduced number of indicia of cancer (e.g., metastatic lesions) appearing over time, complete remission, partial remission, or stable disease. For example, the methods of treatment may produce a comparable clinical benefit rate (CBR=complete remission (CR)+partial remission (PR)+stable disease (SD) lasting ≥6 months) better than that achieved without administration of the ADC, or than that achieved with administration of any one of the ADC and the second agent, e.g., the improvement of clinical benefit rate is about 20% 20%, 30%, 40%, 50%, 60%, 70%, 80% or more.

As used herein, the terms “inhibit” and “block” (e.g., with regard to inhibition/blocking of LRRC15 binding or functional activity) are used interchangeably and encompass both partial and complete inhibition/blocking by the anti-LRRC15 antibody or antigen binding portion thereof in the ADC, or other inhibition/blocking of a functional activity by a therapeutic agent. The degree of inhibition may be at least 20%, 30%, 40%, 50%, 60%, 70%, 80%, 90%, 95%, or 100% (i.e., 2-fold or 2×), 3-fold, 5-fold or 10-fold relative to a control antibody or reference antibody. Additionally, the degree of inhibition may be between 20%-95%, 20%-80%, 20%-50%, 40%-95%, 40%-80%, 40%-60%, 50%-90%, 50%-70%, 75%-95%, 75%-85%, 2-fold to 20-fold, 2-fold to 10-fold, 2-fold to 5-fold, 4-fold to 12-fold, or 4-fold to 8-fold.

The term “effective dose” or “effective dosage” is defined as an amount sufficient to achieve or at least partially achieve a desired effect. A “therapeutically effective amount” or “therapeutically effective dosage” of a drug (e.g., ADC (comprising anti-LRRC15 antibody or antigen binding portion thereof described herein linked to a cytotoxic moiety described herein via a linker)) is any amount of the drug or therapeutic agent that, when used alone or in combination with another therapeutic agent, promotes disease regression evidenced by a decrease in severity of disease symptoms, an increase or therapeutic agent in frequency and duration of disease symptom-free periods, or a prevention of impairment or disability due to the disease affliction. A therapeutically effective amount or dosage of a drug or therapeutic agent includes a “prophylactically effective amount” or a “prophylactically effective dosage,” which is any amount of the drug or therapeutic agent that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, for the treatment of tumors, a therapeutically effective amount or dosage of the drug or therapeutic agent (e.g., ADC (comprising anti-LRRC15 antibody or antigen binding portion thereof described herein linked to a cytotoxic moiety described herein via a linker) inhibits tumor cell growth by at least about 20%, by at least about 30% by at least about 40%, by at least about 50%, by at least about 60%, by at least above 70%, by at least about 80%, or by at least about 90% relative to untreated subjects. In some aspects, a therapeutically effective amount or dosage of the drug or therapeutic agent completely inhibits cell growth or tumor growth, i.e., inhibits cell growth or tumor growth by 100%. The ability of a compound or therapeutic agent, including an antibody, to inhibit tumor growth can be evaluated using the assays described herein. Alternatively, this property of a composition comprising the compound or therapeutic agent can be evaluated by examining the ability of the composition to inhibit cell growth; such inhibition can be measured in vitro by assays known to the skilled practitioner.

As used herein, the term “bystander killing” or “bystander effect” refers to the killing of target-negative cells (e.g., LRRC15 negative tumor cells or tumor cells expressing a low level of LRRC15) by delivering a cytotoxic agent to the cells expressing the target (i.e., target-positive cells (e.g., LRRC15+ stromal cells such as fibroblasts or tumor cells, or fibroblasts or tumor cells expressing an elevated level of LRRC15)). In some aspects, the target positive cells are present in the vicinity of the target-negative cells. In some aspects, killing of target-negative cells is not observed in the absence of target-positive cells. In some aspects, proximity between target-positive and target-negative cells, for example, enables bystander killing. This type of killing is distinguishable from “off-target killing,” which refers to the indiscriminate killing of target-negative cells. “Off-target killing” may be observed in the absence of target-positive cells.

The term “patient” includes human and other mammalian subjects that receive either prophylactic or therapeutic treatment.

As used herein, the term “subject” includes any human or non-human animal. For example, the methods and compositions described herein can be used to treat a subject having cancer. The term “non-human animal” includes all vertebrates, e.g., mammals and non-mammals, such as non-human primates, sheep, cats, dogs, cows, chickens, amphibians, and reptiles.

The term “sample” refers to tissue, bodily fluid, or a cell (or a fraction of any of the foregoing) taken from a patient or a subject. Normally, the tissue or cell will be removed from the patient, but in vivo diagnosis is also contemplated. In the case of a solid tumor, a tissue sample can be taken from a surgically removed tumor and prepared for testing. In the case of lymphomas and leukemias, lymphocytes, leukemic cells, or lymph tissues can be obtained (e.g., leukemic cells from blood) and appropriately prepared. Other samples, including e.g., urine, tears, serum, plasma, cerebrospinal fluid, feces, sputum, and cell extracts can also be useful for particular cancers.

The terms “detection” or “detected”, as used herein refer to qualitative and/or quantitative detection (measuring levels) with or without reference to a control.

The term “diagnosing”, as used herein, means the determination of the nature of a medical condition intended to identify a pathology which affects the subject from a number of collected data.

As used herein, “comprising” is synonymous with “including,” “containing,” “having” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be optionally replaced with either of the other two terms, thus describing alternative aspects of the scope of the subject matter. The disclosure illustratively described herein suitably may be practiced in the absence of any element or elements, limitation or limitations which is not specifically disclosed herein.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The term “about” as used herein when referring to a measurable value such as an amount, a temporal duration and the like, encompasses variations of up to +10% from the specified value. Unless otherwise indicated, all numbers expressing e.g., quantities of ingredients or properties (e.g., molecular weight, reaction conditions) described herein are to be understood as being modified by the term “about.”

As used herein, “and/or” is to be taken as specific disclosure of each of the two specified features or components with or without the other. Thus, the term “and/or” as used in a phrase such as “A and/or B” includes “A and B,” “A or B,” “A” alone, and “B” alone. Likewise, the term “and/or” as used in a phrase such as “A, B, and/or C” encompasses each of the following: A, B, and C; A, B, or C; A or C; A or B; B or C; A and C; A and B; B and C; A alone; B alone; and C alone.

As used herein, recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3% are expressly enumerated in this specification. These are only examples of what is specifically intended, and all possible combinations of numerical values between and including the lowest value and the highest value enumerated are to be considered to be expressly stated in this disclosure.

As used herein, the term “stereoisomer” refers to isomers of identical constitution that differ in the arrangement of their atoms in space. Enantiomers and diastereomers are examples of stereoisomers. Geometric isomers are also examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity. Geometric isomers of C═C double bonds can also be present in the ADCs, and all such stable isomers are contemplated in the present disclosure. Cis- and trans- (or E- and Z-) geometric isomers of the ADCs of the present disclosure are described and may be isolated as a mixture of isomers or as separated isomeric forms.

The term “ADC 101” refers to a compound having the formula:

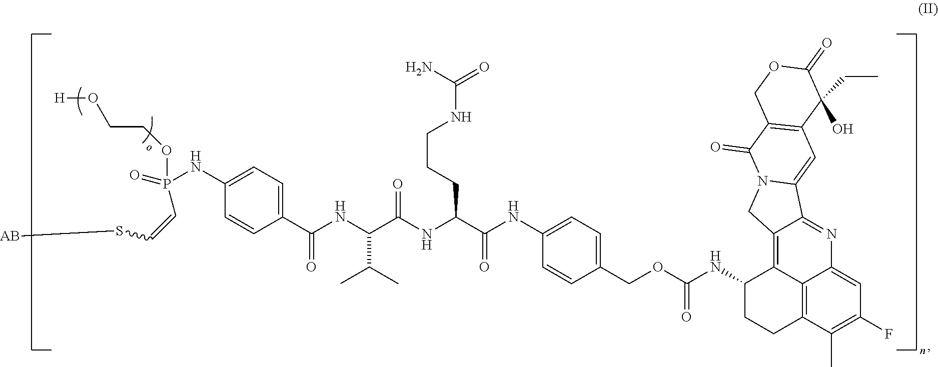

wherein [symbol] indicates that the configuration of the double bond may be E or Z;
AB is an anti-LRRC15 antibody or an antigen binding portion thereof described herein, which is also understood to be identical to the compound having the formula:

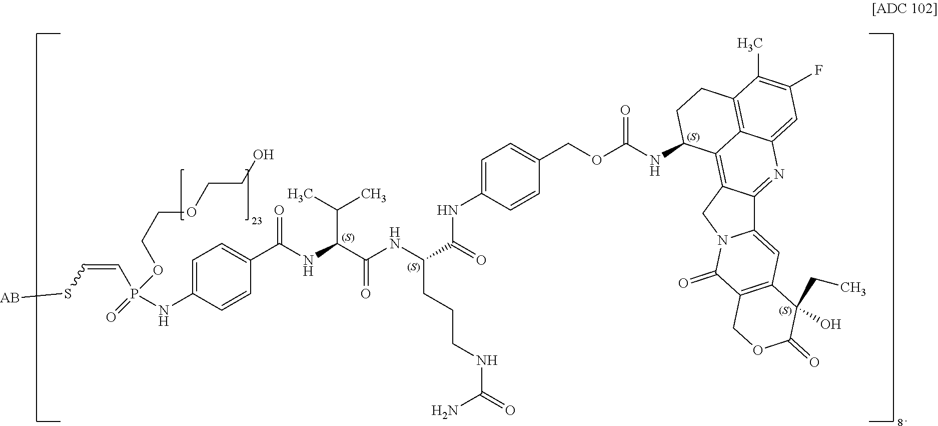

As the two structures are presumed to be identical, the two representations are interchangeable. Either structure can be used to represent the same structure. Various aspects described herein are described in further detail in the following subsections.

### Antibody Drug Conjugates

The present disclosure provides an antibody drug conjugate comprising an anti-LRRC15 antibody or antigen binding portion thereof described herein which is linked or conjugated via a phorphorus (V) moiety (also denoted as “P5”) and a linker to C (a cytotoxic moiety, i.e., camptothecin or derivatives and analogs thereof).

In some aspects, the ADC of the present disclosure has the formula (I):

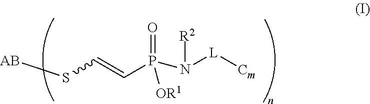

or a pharmaceutically acceptable salt, a stereoisomer, or a solvate thereof, wherein:
AB is an anti-LRRC15 antibody or an antigen-binding portion thereof discloses herein;
[symbol] indicates that the configuration of the double bond may be E or Z;
R¹ is a polyalkene glycol unit comprising at least 3 alkylene glycol subunits;
Y is NR²;
R² is H or an optionally substituted aliphatic residue, or an optionally substituted aromatic residue;
L is a linker;
C is a DNA topoisomerase I inhibitor;
m is an integer ranging from 1 to 10;
n is an integer ranging from 1 to 20, and
wherein the antibody or antigen binding portion thereof comprises:
(a) a heavy chain variable region (VH) comprising complementarity determining region (CDR)1, CDR2, and CDR3 regions comprising the amino acid sequences set forth in SEQ ID NOs: 3, 4, and 5, respectively, and a light chain variable region (VL) comprising CDR1, CDR2, and CDR3 regions comprising the amino acid sequences set forth in SEQ ID NOs: 8, 9, and 10, or;
(b) a VH comprising CDR1, CDR2, and CDR3 regions which have at least 80%, 85%, 90%, 95%, 98%, or 99% sequence identity to the amino acid sequences set forth in SEQ ID NOs: 3, 4, and 5, respectively, and a VL comprising CDR1, CDR2, and CDR3 regions which have at least 85%, 90%, 95%, 98%, or 99% sequence identity to the amino acid sequences set forth in SEQ ID NOs: 9, 10, and 11, respectively; or
(c) a VH comprising CDR1, CDR2, and CDR3 regions comprising the amino acid sequences set forth in SEQ ID NOs: 22, 23, and 24, respectively, and a VL comprising CDR1, CDR2, and CDR3 regions comprising the amino acid sequences set forth in SEQ ID NOs: 27, 28, and 29, or;
(d) a VH comprising CDR1, CDR2, and CDR3 regions which have at least 80%, 85%, 90%, 95%, 98%, or 99% sequence identity to the amino acid sequences set forth in SEQ ID NOs: 22, 23, and 24, respectively, and a VL comprising CDR1, CDR2, and CDR3 regions which have at least 85%, 90%, 95%, 98%, or 99% sequence identity to the amino acid sequences set forth in SEQ ID NOs: 27, 28, and 29, respectively, or;
(e) a VH comprising CDR1, CDR2, and CDR3 regions comprising the amino acid sequences set forth in SEQ ID NOs: 31, 32, and 33, respectively, and a VL comprising CDR1, CDR2, and CDR3 regions comprising the amino acid sequences set forth in SEQ ID NOs: 36, 37, and 38, or;
(f) a VH comprising CDR1, CDR2, and CDR3 regions which have at least 80%, 85%, 90%, 95%, 98%, or 99% sequence identity to the amino acid sequences set forth in SEQ ID NOs: 31, 32, and 33, respectively, and a VL comprising CDR1, CDR2, and CDR3 regions which have at least 85%, 90%, 95%, 98%, or 99% sequence identity to the amino acid sequences set forth in SEQ ID NOs: 36, 37, and 38, respectively, or;
(g) a VH comprising CDR1, CDR2, and CDR3 regions comprising the amino acid sequences set forth in SEQ ID NOs: 40, 41, and 42, respectively, and a VL comprising CDR1, CDR2, and CDR3 regions comprising the amino acid sequences set forth in SEQ ID NOs: 45, 46, and 47, or;
(h) a VH comprising CDR1, CDR2, and CDR3 regions which have at least 80%, 85%, 90%, 95%, 98%, or 99% sequence identity to the amino acid sequences set forth in SEQ ID NOs: 40, 41, and 42, respectively, and a VL comprising CDR1, CDR2, and CDR3 regions which have at least 85%, 90%, 95%, 98%, or 99% sequence identity to the amino acid sequences set forth in SEQ ID NOs: 45, 46, and 47, respectively, or;
(i) a VH comprising CDR1, CDR2, and CDR3 regions comprising the amino acid sequences set forth in SEQ ID NOs: 49, 50, and 51, respectively, and a VL comprising CDR1, CDR2, and CDR3 regions comprising the amino acid sequences set forth in SEQ ID NOs: 54, 55, and 56, or;
(j) a VH comprising CDR1, CDR2, and CDR3 regions which have at least 80%, 85%, 90%, 95%, 98%, or 99% sequence identity to the amino acid sequences set forth in SEQ ID NOs: 49, 50, and 51, respectively, and a VL comprising CDR1, CDR2, and CDR3 regions which have at least 85%, 90%, 95%, 98%, or 99% sequence identity to the amino acid sequences set forth in SEQ ID NOs: 54, 55, and 56, respectively, or;
(k) a VH comprising CDR1, CDR2, and CDR3 regions comprising the amino acid sequences set forth in SEQ ID NOs: 58, 59, and 60, respectively, and a VL comprising CDR1, CDR2, and CDR3 regions comprising the amino acid sequences set forth in SEQ ID NOs: 63, 64, and 65, or
(l) a VH comprising CDR1, CDR2, and CDR3 regions which have at least 80%, 85%, 90%, 95%, 98%, or 99% sequence identity to the amino acid sequences set forth in SEQ ID NOs: 58, 59, and 60, respectively, and a VL comprising CDR1, CDR2, and CDR3 regions which have at least 85%, 90%, 95%, 98%, or 99% sequence identity to the amino acid sequences set forth in SEQ ID NOs: 63, 64, and 65, respectively.

In some aspects, R¹ comprises 3 to 100 subunits having the structure:

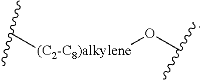

In some aspects, R¹ is

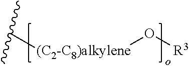

wherein: [symbol] indicates the position of the O; R3 is selected from the group consisting of H, —PO3H, —(C1-C10)alkyl, —(C1-C10)alkyl-SO3H, —(C2-C10)alkyl-CO2H, —(C2-C10)alkyl-OH, —(C2-C10)alkyl-NH2, —(C2-C10)alkyl-NH(C1-C3)alkyl and —(C2-C10)alkyl-N((C1-C3)alkyl)2; and o is an integer ranging from 3 to 100.

In some aspects, R¹ comprises 3 to 50 subunits having the structure:

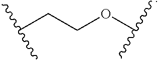 [SMILES: "." ; *CCO*]

In some aspects, R¹ is:

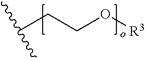

wherein [symbol] indicates the position of the O; R3 is selected from the group consisting of H, —(C1-C10)alkyl, and —(C2-C10)alkyl-OH; and o is an integer ranging from 3 to 50.

In some aspects, R3 is H.

In some aspects, o is an integer ranging from 8 to 30, e.g., from 8 to 16 or from 20 to 28, e.g., 10, 11, 12, 13 14, 22, 23, 24, 25 or 26.

In some aspects, the linker L is cleavable.

In some aspects, the linker L is cleavable by a protease, a glucuronidase, a sulfatase, a phosphatase, an esterase, or by disulfide reduction.

In some aspects, the linker is cleaved under physiological conditions, in particular inside a cell by e.g., a lysosomal or endosomal protease to release the attached cytotoxic moiety. In some aspects, the cleavable linkers are designed to release the free cytotoxic moiety in an unmodified form. Cleavable linkers include, e.g., disulfide linkers, acid labile linkers, photolabile linkers, peptidase labile linkers, and esterase labile linkers. Typically, a peptidyl linker is at least two amino acids long or at least three amino acids long.

Peptidase labile linkers can be used to cleave certain peptides inside or outside cells. In one aspect, the cleavable linker is cleaved under mild conditions, i.e., conditions within a cell under which the activity of the cytotoxic moiety is not affected.

Cleaving agents can include e.g., cathepsins B and D and plasmin, all of which are known to hydrolyze dipeptide drug derivatives resulting in the release of active drug inside target cells. For example, a peptidyl linker that is cleavable by the thiol-dependent protease cathepsin-B, which is highly expressed in cancerous tissue, can be used (e.g., a Phe-Leu or a Gly-Phe-Leu-Gly (SEQ ID NO: 73) linker). In specific aspects, the peptidyl linker cleavable by an intracellular protease is a valine-citrulline (Val-Cit) linker or a phenylalanine-lysine (Phe-Lys) linker. One advantage of using intracellular proteolytic release of the therapeutic agent is that the agent is typically attenuated when conjugated and the serum stabilities of the conjugates are typically high.

A variety of linkers may be used in the conjugates described herein. In some aspects, the linker comprises a peptidyl linker, such as dipeptide valine (Val)-citrulline (Cit) (vc), which can be cleaved by cathepsin inside tumor cells. Additional peptidyl linkers include, but are not limited to Val-Cit, Ala-Val, Val-Ala-Val, Lys-Lys, Pro-Val-Gly-Val-Val (SEQ ID NO: 72), Ala-Asn-Val, Val-Leu-Lys, Ala-Ala-Asn, Cit-Cit, Val-Lys, Lys, Cit, Ser, or Glu. In some aspects, the linker L is cleavable by a protease, for example cathepsins B and D.

In some aspects, the linker L has the formula:

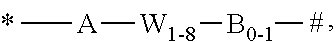

wherein unit A is a first spacer unit; W is an amino acid; B is a second spacer unit, * denotes the attachment point to the —N and # denotes the attachment point to C.

In some aspects, the first spacer unit A has the structure:

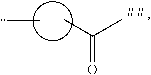

wherein ◯ is a 5- or 6-membered carbocycle; * denotes the attachment point to the —N and ## denotes the attachment point to W. A preferred unit A is

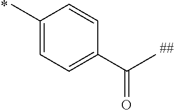

In some aspects, W is a dipeptide (W₂).

In some aspects, the dipeptide is selected from the group consisting of valine-citrulline (Val-Cit) and valine-alanine (Val-Ala).

In some aspects, the second spacer unit B is a PAB group having the following structure:

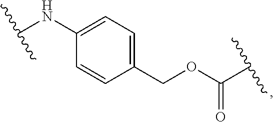 [SMILES: *Nc1ccc(COC(*)=O)cc1 ; ","]

wherein the NH group is bonded to —W— and the C(O) group is bonded to C.

In some aspects, the linker has the following structure:

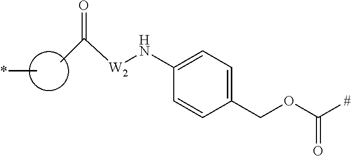

wherein W₂ is a dipeptide,
* indicates the attachment point to the —N, and # indicates the attachment point to C.

In some aspects, the linker L is *-A-W₂—B₁-#, having the structure:

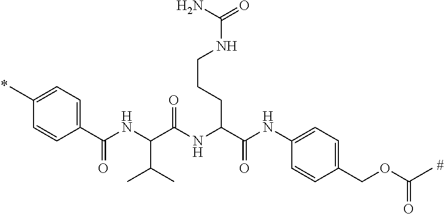

wherein * indicates the attachment point to the —N and # indicates the attachment point to C.

In some aspects, C is a topoisomerase I inhibitor. In some aspects, the topoisomerase I inhibitor is produced by nature, camptothecin. In some aspects, C is a camptothecin derivative, e.g., exatecan. The structure of exatecan is shown below.

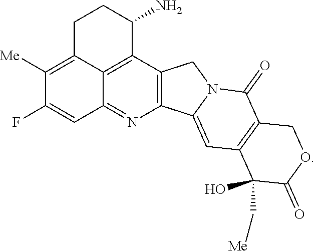 [SMILES: CC[C@@]1(O)C(=O)OCc2c1cc1n(c2=O)Cc2c-1nc1cc(F)c(C)c3c1c2[C@@H](N)CC3]

All stereoisomers of the exatecan are contemplated for the ADCs disclosed herein.

In some aspects, the n in the ADC of the formula (I) is 7 or 8. In some aspects, the n in the ADC is 5 or 6. In some aspects, the n in the ADC is 9 or 10. In some aspects, the n in the ADC is 7. In some aspects, the n in the ADC is 8.

In some aspects, in formula (I), AB is an anti-LRRC15 antibody or an antigen-binding portion thereof discloses herein; n is an integer ranging from 4-8; R1 is a polyalkylene glycol unit having the structure:

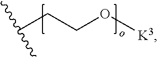

wherein: [symbol] indicates the position of the O;
K³ is H;
is an integer ranging from 8 to 30;
L is a linker having the following structure:

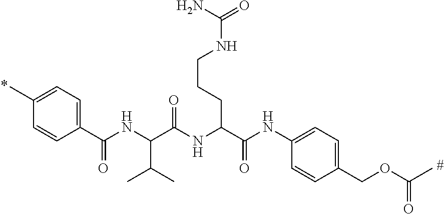

wherein * indicates the attachment point to the —N and # indicates the attachment point to C; C is exatecan; and m is 1.

In some aspects, o is an integer ranging from 8 to 16. In some aspects, o is 10, 11, 12, 13 or 14.

In some aspects, n is an integer ranging from 2 to 10. In some aspects, o is an integer ranging from 20 to 28. In some aspects, o is 22, 23, 24, 25, or 26.

In some aspects, n is an integer ranging from 2 to 10.

In some aspects, the ADC of the present disclosure has the formula (II):

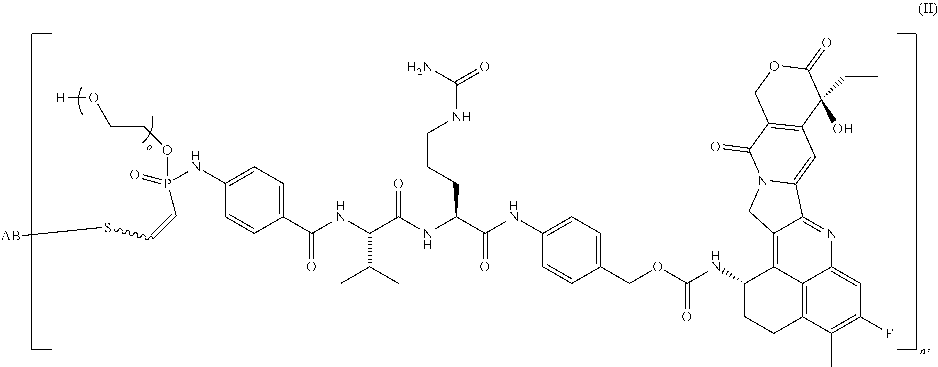

or a pharmaceutically acceptable salt, a stereoisomer, or a solvate thereof, wherein
is an integer from 8 to 30;
n is an integer ranging from 4 to 8;
[symbol] indicates that the configuration of the double bond may be E or Z;
AB is an anti-LRRC15 antibody or antigen binding portion thereof, comprising:
(a) a heavy chain variable region (VH) comprising complementarity determining region (CDR)1, CDR2, and CDR3 regions comprising the amino acid sequences set forth in SEQ ID NOs: 3, 4, and 5, respectively, and a light chain variable region (VL) comprising CDR1, CDR2, and CDR3 regions comprising the amino acid sequences set forth in SEQ ID NOs: 8, 9, and 10, or;
(b) a VH comprising CDR1, CDR2, and CDR3 regions which have at least 80%, 85%, 90%, 95%, 98%, or 99% sequence identity to the amino acid sequences set forth in SEQ ID NOs: 3, 4, and 5, respectively, and a VL comprising CDR1, CDR2, and CDR3 regions which have at least 85%, 90%, 95%, 98%, or 99% sequence identity to the amino acid sequences set forth in SEQ ID NOs: 9, 10, and 11, respectively; or
(c) a VH comprising CDR1, CDR2, and CDR3 regions comprising the amino acid sequences set forth in SEQ ID NOs: 22, 23, and 24, respectively, and a VL comprising CDR1, CDR2, and CDR3 regions comprising the amino acid sequences set forth in SEQ ID NOs: 27, 28, and 29, or;
(d) a VH comprising CDR1, CDR2, and CDR3 regions which have at least 80%, 85%, 90%, 95%, 98%, or 99% sequence identity to the amino acid sequences set forth in SEQ ID NOs: 22, 23, and 24, respectively, and a VL comprising CDR1, CDR2, and CDR3 regions which have at least 85%, 90%, 95%, 98%, or 99% sequence identity to the amino acid sequences set forth in SEQ ID NOs: 27, 28, and 29, respectively, or;
(e) a VH comprising CDR1, CDR2, and CDR3 regions comprising the amino acid sequences set forth in SEQ ID NOs: 31, 32, and 33, respectively, and a VL comprising CDR1, CDR2, and CDR3 regions comprising the amino acid sequences set forth in SEQ ID NOs: 36, 37, and 38, or;
(f) a VH comprising CDR1, CDR2, and CDR3 regions which have at least 80%, 85%, 90%, 95%, 98%, or 99% sequence identity to the amino acid sequences set forth in SEQ ID NOs: 31, 32, and 33, respectively, and a VL comprising CDR1, CDR2, and CDR3 regions which have at least 85%, 90%, 95%, 98%, or 99% sequence identity to the amino acid sequences set forth in SEQ ID NOs: 36, 37, and 38, respectively, or;
(g) a VH comprising CDR1, CDR2, and CDR3 regions comprising the amino acid sequences set forth in SEQ ID NOs: 40, 41, and 42, respectively, and a VL comprising CDR1, CDR2, and CDR3 regions comprising the amino acid sequences set forth in SEQ ID NOs: 45, 46, and 47, or;
(h) a VH comprising CDR1, CDR2, and CDR3 regions which have at least 80%, 85%, 90%, 95%, 98%, or 99% sequence identity to the amino acid sequences set forth in SEQ ID NOs: 40, 41, and 42, respectively, and a VL comprising CDR1, CDR2, and CDR3 regions which have at least 85%, 90%, 95%, 98%, or 99% sequence identity to the amino acid sequences set forth in SEQ ID NOs: 45, 46, and 47, respectively, or;
(i) a VH comprising CDR1, CDR2, and CDR3 regions comprising the amino acid sequences set forth in SEQ ID NOs: 49, 50, and 51, respectively, and a VL comprising CDR1, CDR2, and CDR3 regions comprising the amino acid sequences set forth in SEQ ID NOs: 54, 55, and 56, or;
(j) a VH comprising CDR1, CDR2, and CDR3 regions which have at least 80%, 85%, 90%, 95%, 98%, or 99% sequence identity to the amino acid sequences set forth in SEQ ID NOs: 49, 50, and 51, respectively, and a VL comprising CDR1, CDR2, and CDR3 regions which have at least 85%, 90%, 95%, 98%, or 99% sequence identity to the amino acid sequences set forth in SEQ ID NOs: 54, 55, and 56, respectively, or;
(k) a VH comprising CDR1, CDR2, and CDR3 regions comprising the amino acid sequences set forth in SEQ ID NOs: 58, 59, and 60, respectively, and a VL comprising CDR1, CDR2, and CDR3 regions comprising the amino acid sequences set forth in SEQ ID NOs: 63, 64, and 65, or
(l) a VH comprising CDR1, CDR2, and CDR3 regions which have at least 80%, 85%, 90%, 95%, 98%, or 99% sequence identity to the amino acid sequences set forth in SEQ ID NOs: 58, 59, and 60, respectively, and a VL comprising CDR1, CDR2, and CDR3 regions which have at least 85%, 90%, 95%, 98%, or 99% sequence identity to the amino acid sequences set forth in SEQ ID NOs: 63, 64, and 65, respectively.

In some aspects, o is an integer of 8, 9, 10, 11, 12, 13, 14, 15, 16, 17, 18, 19, 20, 21, 22, 23, 24, 25, 26, 27, 28, 29, or 30. In some aspects, o is an integer between 20 to 30, e.g., 24. In some aspects, o is an integer between 20 and 25, e.g., 24. In some aspects, o is an integer of 24. In some aspects, o is 25. In some aspects, o is 23. In some aspects, o is 22. In some aspects, o is 21. In some aspects, o is 20. In some aspects, o is an integer between 26 and 30. In some aspects, o is 26. In some aspects o is 27. In some aspects, o is 28. In some aspects o is 29. In some aspects, o is 30. In some aspects o is an integer between 8 and 19. In some aspects, o is 8. In some aspects, o is 9. In some aspects, o is 10. In some aspects o is 11. In some aspects o is 12. In some aspects o is 13. In some aspects, o is 14. In some aspects, o is 15. In some aspects o is 16. In some aspects o is 17. In some aspects, o is 18. In some aspects, o is 19.

The present disclosure also provides a compound of the formula (III):

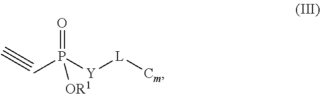

or a pharmaceutically acceptable salt, a stereoisomer, or a solvate thereof, wherein R¹, Y, L, C, and m are as defined in the formula (I).

In some aspects, the present disclosure provides a method of preparing an ADC of the formula (I), comprising reacting a compound of the formula (III) with a thiol containing compound, AB-(SH) n, wherein AB is the anti-LRRC15 antibody or antigen binding portion thereof disclosed herein, n is an integer ranging from 1 and 10, to yield the ADC of the formula (I):

In some aspects, the compounds of the formula (III) have the structure:

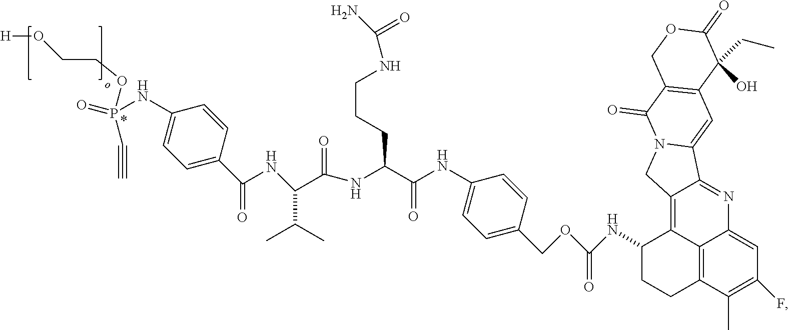

(Compound A), or a pharmaceutically acceptable salt a stereoisomer, or a solvate thereof, wherein o is an integer ranging from 8-25, e.g., 24, * represents a chiral center. All stereoisomers of Compound A are contemplated for the synthesizing the ADCs disclosed herein.

In some aspects, the present disclosure provides a method of preparing an ADC of the formula (II), comprising reacting Compound A with a thiol containing compound, AB-(SH) n, wherein AB is the anti-LRRC15 antibody or antigen binding portion thereof disclosed herein, to obtain the ADC of the formula (II):

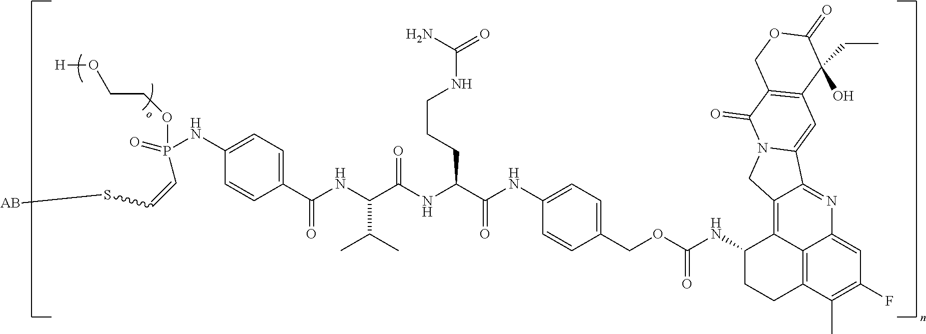

(II), or a pharmaceutically acceptable salt, a stereoisomer, or a solvate thereof, wherein o and n are as defined above.

Methods of selective bioconjugation reaction of ethynylphosphonamideates with cysteine containing compounds have been described in WO2018041985A1 (published Mar. 8, 2018), WO2019170710A2 (published Sep. 12, 2019), WO2022223783A1 (published Oct. 27, 2022), WO2023083900A1 (published May 19, 2023), WO2023083919A1 (published May 19, 2023), each of which is incorporated herein by reference.

The number of cytotoxic moieties linked to the antigen binding moiety of a LRRC15-ADC (drug-to-antibody ratio: DAR) can vary and will be limited only by the number of available attachments sites on the antigen binding moiety and the number of agents linked to a single linker.

The DAR value can vary with the nature of the antigen binding moiety (e.g., any antibody or the antigen-binding portion thereof described herein) and the drug used along with the experimental conditions used for the conjugation (DAR, reaction time, nature of the solvents and/or cosolvents). Thus, the contact between the antibody and the drug may in an ADC lead to a mixture comprising several conjugates differing from one another by different drug-to-antibody ratios and may further include free antibodies and/or aggregates. The DAR that is determined is thus a mean value. DARs may be analyzed by UV spectrometry, monomer content may be analyzed by SEC-HPLC, and free drug content may be analyzed by RP-HPLC.

In some aspects, a linker will link a single cytotoxic moiety to the antigen binding moiety (e.g., any antibody or the antigen-binding portion thereof described herein) of a conjugate. In some aspects where the conjugate include more than one cytotoxic moiety, each moiety may be the same or different. As long as the conjugate does not exhibit unacceptable levels of aggregation under the conditions of use and/or storage, conjugates with DARs of twenty, or even higher, are contemplated. In some aspects, the conjugates described herein may have a DAR in the range of about 1-10, 2-10, 1-8, 2-8, 1-6, 2-6, 1-4, or 2-4. In some specific aspects, the conjugate may have a DAR of 2, 3, 4 or 5. In some aspects, the DAR is 6. In some aspects, the DAR is 7. In some aspects, the DAR is 8. In some aspects, the DAR is 9. In some aspects, the DAR is 6 or 7. In some aspects, the DAR is 7, 7.5 or 8. In some aspects, the DAR is 7-8.

In some aspects, an ADC of the present disclosure has the following structure:

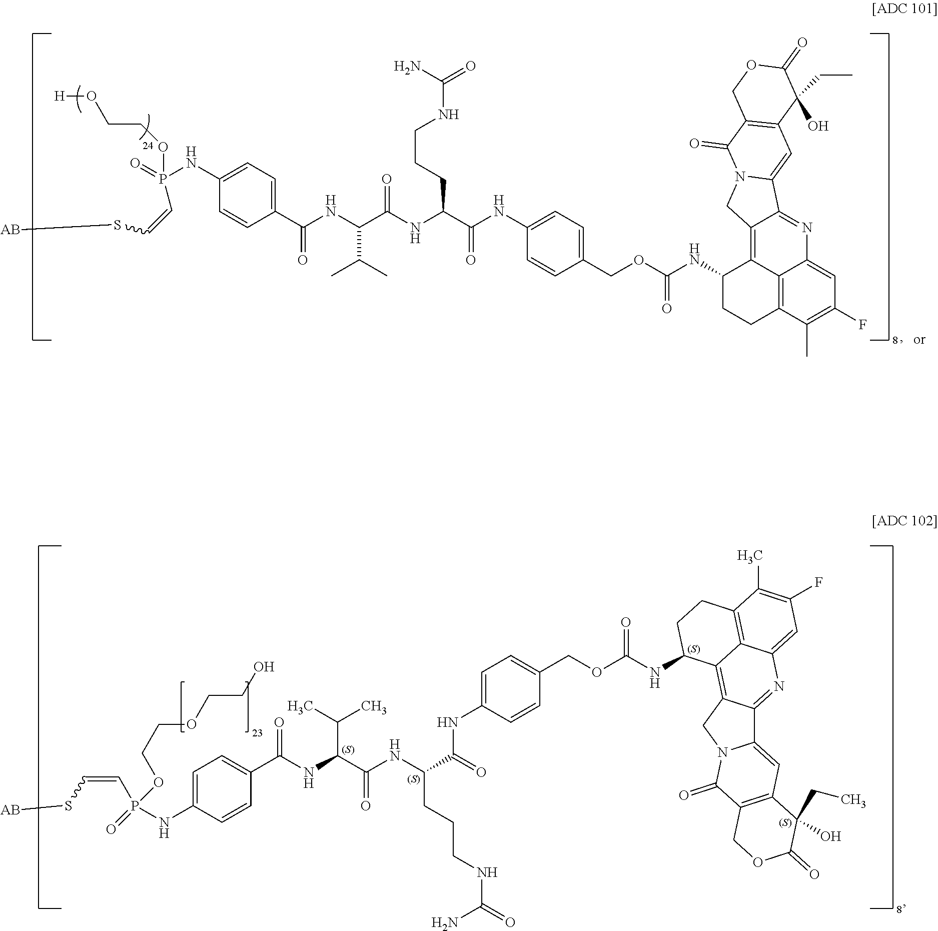

or a pharmaceutically acceptable salt, a stereoisomer, or a solvate thereof, wherein
[symbol] indicates that the configuration of the double bond may be E or Z;
AB is an anti-LRRC15 antibody or an antigen-binding portion, comprising:
(a) a heavy chain variable region (VH) comprising complementarity determining region (CDR)1, CDR2, and CDR3 regions comprising the amino acid sequences set forth in SEQ ID NOs: 3, 4, and 5, respectively, and a light chain variable region (VL) comprising CDR1, CDR2, and CDR3 regions comprising the amino acid sequences set forth in SEQ ID NOs: 8, 9, and 10, or;
(b) a VH comprising CDR1, CDR2, and CDR3 regions which have at least 80%, 85%, 90%, 95%, 98%, or 99% sequence identity to the amino acid sequences set forth in SEQ ID NOs: 3, 4, and 5, respectively, and a VL comprising CDR1, CDR2, and CDR3 regions which have at least 85%, 90%, 95%, 98%, or 99% sequence identity to the amino acid sequences set forth in SEQ ID NOs: 9, 10, and 11, respectively; or
(c) a VH comprising CDR1, CDR2, and CDR3 regions comprising the amino acid sequences set forth in SEQ ID NOs: 22, 23, and 24, respectively, and a VL comprising CDR1, CDR2, and CDR3 regions comprising the amino acid sequences set forth in SEQ ID NOs: 27, 28, and 29, or;
(d) a VH comprising CDR1, CDR2, and CDR3 regions which have at least 80%, 85%, 90%, 95%, 98%, or 99% sequence identity to the amino acid sequences set forth in SEQ ID NOs: 22, 23, and 24, respectively, and a VL comprising CDR1, CDR2, and CDR3 regions which have at least 85%, 90%, 95%, 98%, or 99% sequence identity to the amino acid sequences set forth in SEQ ID NOs: 27, 28, and 29, respectively, or;
(e) a VH comprising CDR1, CDR2, and CDR3 regions comprising the amino acid sequences set forth in SEQ ID NOs: 31, 32, and 33, respectively, and a VL comprising CDR1, CDR2, and CDR3 regions comprising the amino acid sequences set forth in SEQ ID NOs: 36, 37, and 38, or;
(f) a VH comprising CDR1, CDR2, and CDR3 regions which have at least 80%, 85%, 90%, 95%, 98%, or 99% sequence identity to the amino acid sequences set forth in SEQ ID NOs: 31, 32, and 33, respectively, and a VL comprising CDR1, CDR2, and CDR3 regions which have at least 85%, 90%, 95%, 98%, or 99% sequence identity to the amino acid sequences set forth in SEQ ID NOs: 36, 37, and 38, respectively, or;
(g) a VH comprising CDR1, CDR2, and CDR3 regions comprising the amino acid sequences set forth in SEQ ID NOs: 40, 41, and 42, respectively, and a VL comprising CDR1, CDR2, and CDR3 regions comprising the amino acid sequences set forth in SEQ ID NOs: 45, 46, and 47, or;
(h) a VH comprising CDR1, CDR2, and CDR3 regions which have at least 80%, 85%, 90%, 95%, 98%, or 99% sequence identity to the amino acid sequences set forth in SEQ ID NOs: 40, 41, and 42, respectively, and a VL comprising CDR1, CDR2, and CDR3 regions which have at least 85%, 90%, 95%, 98%, or 99% sequence identity to the amino acid sequences set forth in SEQ ID NOs: 45, 46, and 47, respectively, or;
(i) a VH comprising CDR1, CDR2, and CDR3 regions comprising the amino acid sequences set forth in SEQ ID NOs: 49, 50, and 51, respectively, and a VL comprising CDR1, CDR2, and CDR3 regions comprising the amino acid sequences set forth in SEQ ID NOs: 54, 55, and 56, or;
(j) a VH comprising CDR1, CDR2, and CDR3 regions which have at least 80%, 85%, 90%, 95%, 98%, or 99% sequence identity to the amino acid sequences set forth in SEQ ID NOs: 49, 50, and 51, respectively, and a VL comprising CDR1, CDR2, and CDR3 regions which have at least 85%, 90%, 95%, 98%, or 99% sequence identity to the amino acid sequences set forth in SEQ ID NOs: 54, 55, and 56, respectively, or;
(k) a VH comprising CDR1, CDR2, and CDR3 regions comprising the amino acid sequences set forth in SEQ ID NOs: 58, 59, and 60, respectively, and a VL comprising CDR1, CDR2, and CDR3 regions comprising the amino acid sequences set forth in SEQ ID NOs: 63, 64, and 65, or
(l) a VH comprising CDR1, CDR2, and CDR3 regions which have at least 80%, 85%, 90%, 95%, 98%, or 99% sequence identity to the amino acid sequences set forth in SEQ ID NOs: 58, 59, and 60, respectively, and a VL comprising CDR1, CDR2, and CDR3 regions which have at least 85%, 90%, 95%, 98%, or 99% sequence identity to the amino acid sequences set forth in SEQ ID NOs: 63, 64, and 65, respectively.

In some aspects, the disclosure provides an antibody drug conjugate (ADC) having the formula (II):

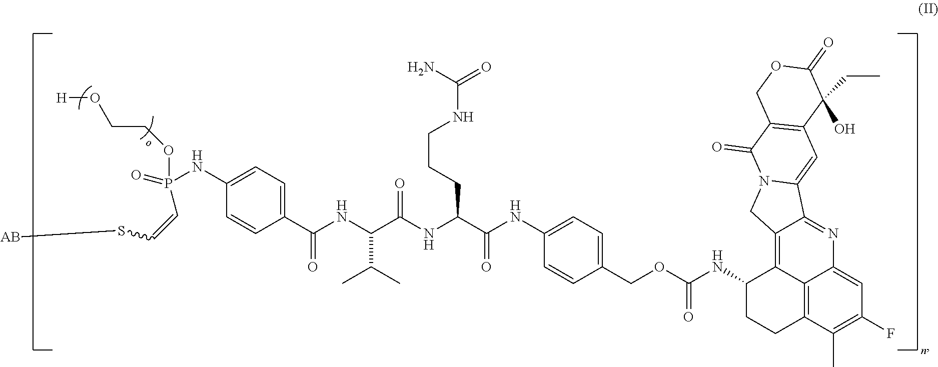

or a pharmaceutically acceptable salt, a stereoisomer, or a solvate thereof; wherein [symbol] indicates that the configuration of the double bond is E or Z; n is an integer ranging from 4 to 8; o is an integer ranging from 10 to 30; AB is an anti-LRRC15 antibody or antigen binding portion thereof comprising a heavy chain variable region (VH) comprising complementarity determining region (CDR)1, CDR2, and CDR3 regions comprising the amino acid sequences set forth in SEQ ID NOs: 3, 4, and 5, respectively, and a light chain variable region (VL) comprising CDR1, CDR2, and CDR3 regions comprising the amino acid sequences set forth in SEQ ID NOs: 9, 10, and 11.

In some aspects, the disclosure provides ADC 101 or ADC 102, or a pharmaceutically acceptable salt thereof, wherein AB is the antibody, or antigen binding portion thereof that binds LRRC15, comprising a VH and a VL, which comprise the amino acid sequences set forth in SEQ ID NOs: 6 and 12, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 7 and the amino acid sequence set forth in SEQ ID NO: 12, respectively.

In some aspects, the disclosure provides ADC 101, or a pharmaceutically acceptable salt thereof, wherein AB is the anti-LRRC15 antibody, or antigen binding portion thereof, comprising a VH and a VL which comprise the amino acid sequence set forth in SEQ ID NO: 25 and the amino acid sequence set forth in SEQ ID NO: 30, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 26 and the amino acid sequence set forth in SEQ ID NO: 30, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 34 and the amino acid sequence set forth in SEQ ID NO: 39, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 35 and the amino acid sequence set forth in SEQ ID NO: 39, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 43 and the amino acid sequence set forth in SEQ ID NO: 48, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 44 and the amino acid sequence set forth in SEQ ID NO: 48, respectively.

In some aspects, the disclosure provides ADC 101, or a pharmaceutically acceptable salt thereof, wherein AB is the anti-LRRC15 antibody, or antigen binding portion thereof, comprising a VH and a VL which comprise the amino acid sequence set forth in SEQ ID NO: 52 and the amino acid sequence set forth in SEQ ID NO: 57, respectively.

In some aspects, the disclosure provides ADC 101 or ADC 102, or a pharmaceutically acceptable salt thereof, wherein AB is the anti-LRRC15 antibody, or antigen binding portion thereof, comprising a VH and a VL which comprise the amino acid sequence set forth in SEQ ID NO: 53 and the amino acid sequence set forth in SEQ ID NO: 57, respectively.

In some aspects, the disclosure provides ADC 101 or ADC 102, or a pharmaceutically acceptable salt thereof, wherein AB is the anti-LRRC15 the antibody, or antigen binding portion thereof, comprising a VH and a VL which comprise the amino acid sequence set forth in SEQ ID NO: 61 and the amino acid sequence set forth in SEQ ID NO: 66, respectively.

In some aspects, the disclosure provides ADC 101 or ADC 102, or a pharmaceutically acceptable salt thereof, wherein AB is the anti-LRRC15 the antibody, or antigen binding portion thereof, comprising a VH and a VL which comprise the amino acid sequence set forth in SEQ ID NO: 62 and the amino acid sequence set forth in SEQ ID NO: 66, respectively.

ADCs can also be used to modify a given biological response, where the cytotoxic moiety should not be construed as limited to classical chemical therapeutic agents. For example, the cytotoxic moiety may be a protein or polypeptide possessing a desired biological activity (e.g., lymphokines, tumor necrosis factor, IFNγ, growth factors).

In some aspects, an ADC of the present disclosure has the following structure:

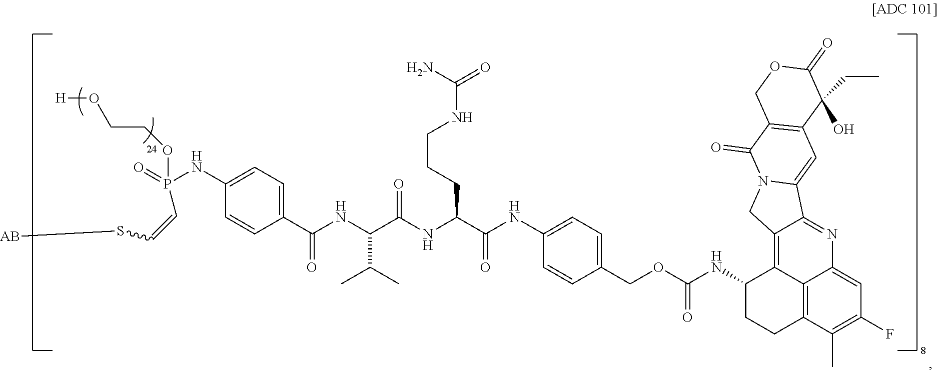

or a pharmaceutically acceptable salt, a stereoisomer, or a solvate thereof, wherein [symbol] represents that the configuration of the double bond may be E or Z; AB is the antibody, or antigen binding portion thereof that binds LRRC15, comprising a heavy chain and a light chain, which comprise the amino acid sequences set forth in SEQ ID NOs: 14 and 17, respectively. In some aspects, the heavy chain comprises the amino acid sequence set forth in SEQ ID NO: 15. In some aspects the heavy chain comprises the amino acid sequence set forth in SEQ ID NO: 16.

In some aspects, an ADC of the present disclosure has the following structure:

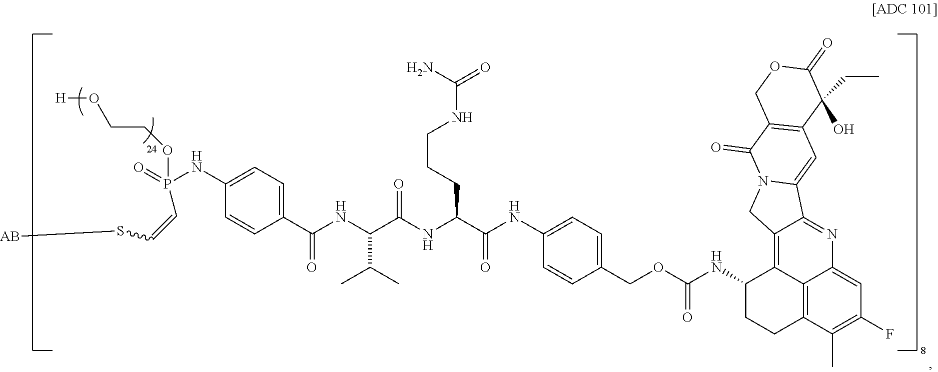

wherein [symbol] represents that the configuration of the double bond is E or Z; AB is the antibody that binds LRRC15, comprising a heavy chain and a light chain, which comprise the amino acid sequences set forth in SEQ ID NOs: 14 and 17, respectively. In some aspects, the heavy chain comprises the amino acid sequence set forth in SEQ ID NO: 15. In some aspects the heavy chain comprises the amino acid sequence set forth in SEQ ID NO: 16.

In some aspects, an ADC of the present disclosure has the following structure:

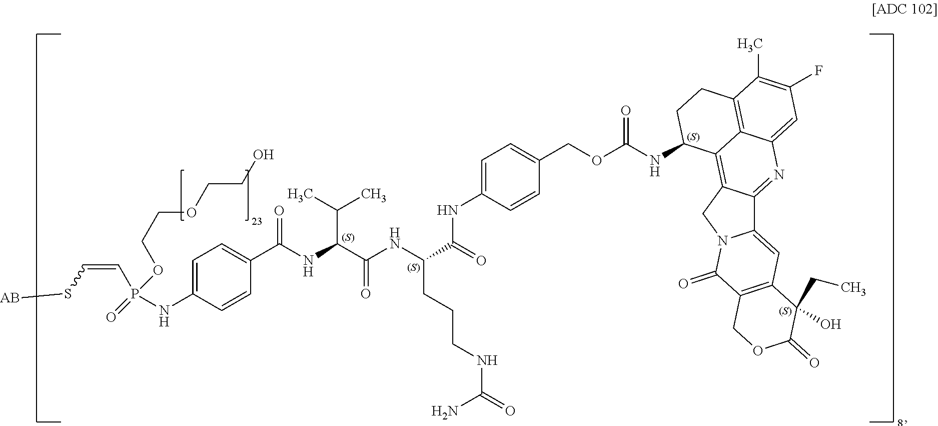

or a pharmaceutically acceptable salt, a stereoisomer, or a solvate thereof, wherein [symbol] represents that the configuration of the double bond may be E or Z; AB is the antibody, or antigen binding portion thereof that binds LRRC15, comprising a heavy chain and a light chain, which comprise the amino acid sequences set forth in SEQ ID NOs: 14 and 17, respectively. In some aspects, the heavy chain comprises the amino acid sequence set forth in SEQ ID NO: 15. In some aspects the heavy chain comprises the amino acid sequence set forth in SEQ ID NO: 16.

In some aspects, an ADC of the present disclosure has the following structure:

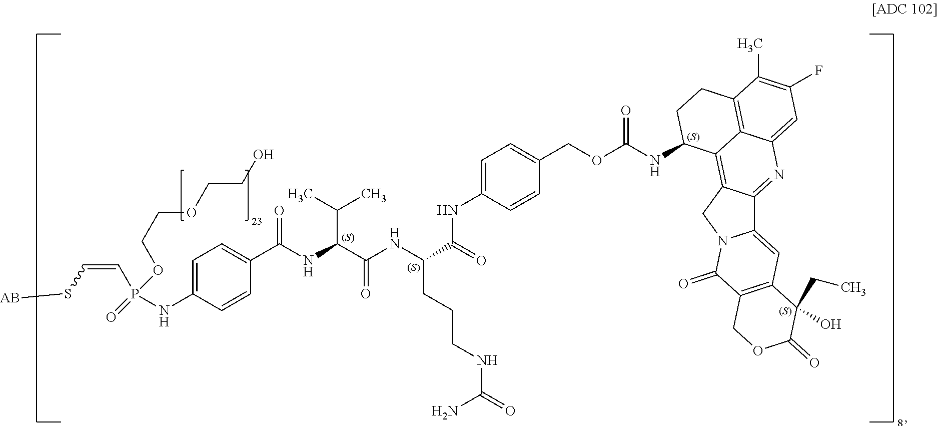

wherein [symbol] represents that the configuration of the double bond is E or Z; AB is the antibody that binds LRRC15, comprising a heavy chain and a light chain, which comprise the amino acid sequences set forth in SEQ ID NOs: 14 and 17, respectively. In some aspects, the heavy chain comprises the amino acid sequence set forth in SEQ ID NO: 15. In some aspects the heavy chain comprises the amino acid sequence set forth in SEQ ID NO: 16.

In some aspects, the disclosure provides an antibody drug conjugate (ADC) having the formula (II):

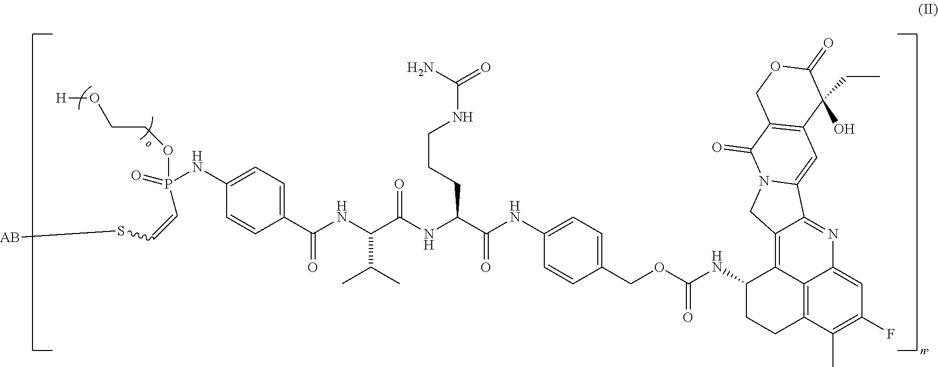

or a pharmaceutically acceptable salt, a stereoisomer, or a solvate thereof; wherein [symbol] indicates that the configuration of the double bond is E or Z; n is 8; o is 24; AB is an anti-LRRC15 antibody or antigen binding portion thereof comprising a heavy chain comprising the amino acid sequence set forth in SEQ ID NO: 14 or SEQ ID NO: 15, and a light chain comprising the amino acid sequence set forth in SEQ ID NO: 17.

In some aspects, the disclosure provides an antibody drug conjugate (ADC) having the formula (II):

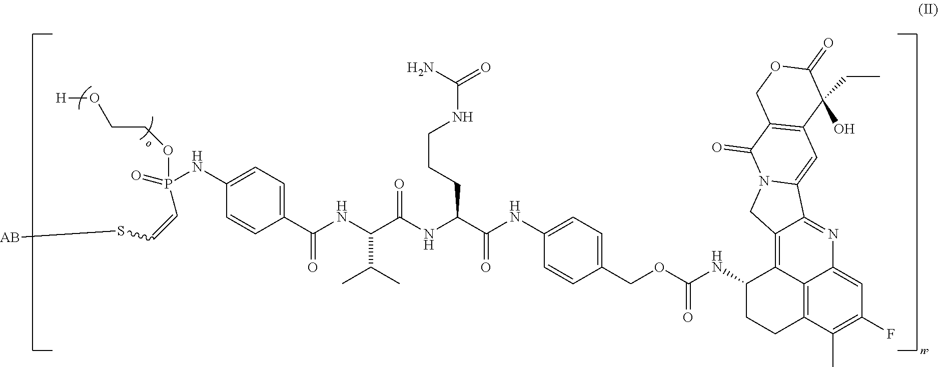

wherein [symbol] indicates that the configuration of the double bond is E or Z; nis 8; o is 24; AB is an anti-LRRC15 antibody comprising a heavy chain comprising the amino acid sequence set forth in SEQ ID NO: 14 or SEQ ID NO: 15, and a light chain comprising the amino acid sequence set forth in SEQ ID NO: 17.

In some aspects, the present ADC targeting LRRC15 is effective in treating disease as LRRC15 is highly expressed on stroma of a tumor. Targeting the stroma, or the supportive tissue surrounding a tumor, in cancer therapy using antibodies can offer several benefits.

In some aspects, stroma provides a protective environment for tumor cells, supplying them with nutrients, growth signals, and resistance to therapies. Targeting the stroma can disrupt this support system, making tumor cells more vulnerable to other treatments like chemotherapy or radiation. Therefore, in some aspects, the ADC comprising an anti-LRRC15 targeting agent, e.g., antibodies or antigen binding portion thereof, can disrupt tumor microenvironment.

In some aspects, tumor-associated stroma cells, such as fibroblasts and immune cells, contribute to tumor growth and metastasis. In some aspects, ADC therapies that target these stromal components, e.g., LRRC15, can slow down or prevent the spread of the tumor by disrupting key signaling pathways.

In some aspects, the dense and abnormal structure of the tumor stroma can act as a barrier to the delivery of therapeutic agents. By targeting and modulating the stroma by an ADC comprising an anti-LRRC15 antibody, the antibodies can help improve the penetration and effectiveness of other treatments.

In some aspects, stroma-targeting ADCs e.g., ADC comprising anti-LRRC15 antibodies, can interfere with angiogenesis, the process by which tumors develop new blood vessels. By disrupting this blood supply, the tumor's growth is limited due to a lack of essential nutrients and oxygen.

In some aspects, tumors often develop resistance to standard therapies like chemotherapy due to the protective role of the stroma. Targeting stromal components, e.g., using an ADC comprising an anti-LRRC15 antibody, can reduce this resistance, making the tumor cells more susceptible to both the immune system and therapeutic agents.

In some aspects, some stroma-targeting ADCs, e.g., ADCs comprising anti-LRRC15 antibody, can recognize specific components in the stroma that are involved in tumor growth and can directly mediate cell death through mechanisms such as antibody-dependent cell-mediated cytotoxicity (ADCC) or complement-dependent cytotoxicity (CDC).

In some aspects, the stroma is often involved in creating an immunosuppressive microenvironment that helps tumors evade the immune system. By targeting the stroma, ADC therapies targeting LRRC15 can potentially restore immune function, enabling the body's immune system to recognize and destroy cancer cells more effectively.

In some aspects, combining stroma-targeting antibodies with other therapies (e.g., checkpoint inhibitors, chemotherapy, radiation, and etc.) can result in synergistic effects, enhancing the overall efficacy of cancer treatment by addressing both the tumor cells and their microenvironment.

Techniques for conjugating toxins or therapeutic moieties to antibodies are known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Antibody Drug Conjugate Bystander Effect

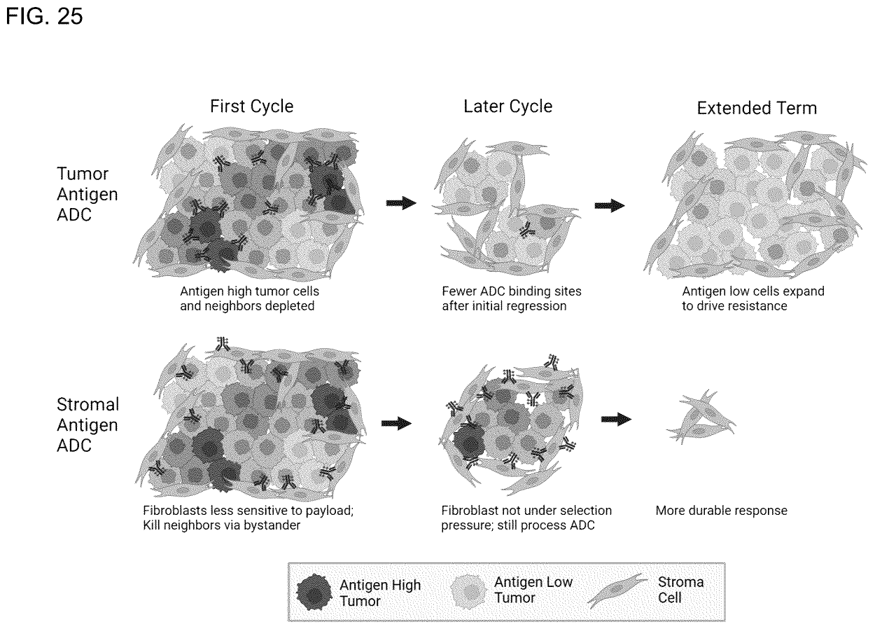

In some aspects, the ADC disclosed herein exhibits a bystander effect. As used herein, the bystander effect refers to the phenomenon in which treatment kills tumor cells by also targeting nearby stromal cells. FIGS. 25-28 show the beneficial effects of the ADCs targeting antigens in a stromal region of a tumor compared to the ADCs targeting antigens directly on tumor cells. In many tumors, LRRC15 expression occurs predominantly on stromal cells, particularly cancer-associated fibroblasts, rather than on the cancer cells themselves. LRRC15 expression on cancer cells may occur in some tumor types (e.g., sarcoma, glioblastoma, and melanoma), where the ADC would provide direct targeting of those cancer cells.

Many factors influence the bystander effect. While not bound by any particular theory, this method aims to target the stroma of the tumor by focusing on proteins that are highly expressed in stromal tissues. By delivering a cytotoxic moiety through an ADC, the moiety is released in the stromal region, thereby exerting a bystander effect on adjacent tumor cells.

Bystander killing (the killing of neighboring cells) may be facilitated through diffusion of the linker-drug and/or the drug alone to neighboring cells. ADCs disclosed herein have several characteristics. When the payload released in the stromal cells is permeable to the neighboring tumor cells or can remain within the membrane of the stromal cells, it induces bystander effects that enhance the killing of the neighboring tumor cells by the payload. The anti-LRRC15 ADC targets LRRC15-expressing stromal cells, which are retained following ADC treatment. In some aspects, the cytotoxic payload released from the ADC can diffuse to neighboring LRRC15-negative tumor cells, killing them through the bystander effect. As shown in the schematics (FIG. 25), the traditional ADCs that target tumor cells directly (i.e., antigens expressed on the tumor) may result in resistance to the ADC therapy: after the initial ADC therapy targeting the tumor directly, fewer ADC binding sites remain in a later cycle following the initial regression and over time (i.e., extended term), antigen low cells can expand to drive resistance. Unlike the ADC therapy that targets the tumor antigen directly, the LRRC15-positive stromal cells remain viable while the surrounding cancer cells are eliminated. Moreover, the bystander effect of these drugs may also alter the tumor microenvironment, which in turn may further enhance the killing effect of ADCs.

In some aspects, ADCs may contain a cleavable linker, which can be cleaved at a defined pH range or by specific proteases to release the free drug. Although the resulting free drug can directly kill the target cell, it can diffuse out of the target cell to cause bystander killing depending on the drug type and its physicochemical properties.

In other aspects, the linker is designed to facilitate bystander killing (the killing of neighboring cells) through diffusion of the linker-drug and/or the drug alone to neighboring cells. In other aspects, the linker promotes cellular internalization.

In some aspects, the anti-LRRC15 antibody or its antigen-binding portion can be conjugated with a cytotoxic moiety that facilitates the bystander effect, thereby enhancing its efficacy.

Bystander killing activity may be determined, e.g., by an assay employing two cell lines, one positive for a target antigen and one negative for a target antigen. In some aspects, the design of the assay allows tracking of only target negative cells. In some aspects, cells are plated under three conditions: (i) target negative cells alone; (ii) target positive cells alone; and (iii) co-culture of target negative cells and target positive cells. In some aspects, nuclear labeled cancer cells (e.g., PANC1 cells) are used to enumerate the cancer cell component of the bystander cultures, making the assessment unambiguous as to which cells are being evaluated. Cells are then treated with an ADC and subsequently monitored for cytotoxicity. Killing of the target-negative cells when mixed with target-positive cells is indicative of bystander killing, whereas killing of the target-negative cells in the absence of the target-positive cells is indicative of off-target killing.

In some aspects, the ADC disclosed herein has an improved bystander effect in vivo compared to a reference ADC comprising the same anti-LRRC15 antibody, or antigen binding portion thereof and a DXd payload (MBP001-Deruxtecan).

In some aspects, the ADC induces in vivo bystander killing of tumor cells that do not express LRRC15. In some aspects, the ADC results in the killing of LRRC15-negative tumor cells. In some aspects, the LRRC15-negative tumor cells are adjacent to LRRC15-positive cells.

In some aspects, the ADC results in the killing of LRRC15-negative tumor cells adjacent to LRRC15-positive stromal cells. In some aspects, the LRRC15-positive stromal cells comprise LRRC15-expressing fibroblasts.

Depending on the linker design, membrane permeable (lipophilic) toxins that are released inside target positive cells can pass the cell membrane and kill other cells that are in close proximity, including neighboring cancer cells that lack antigen expression (bystander effect) (Kovtun, Y. V. et al. (2006) Cancer Res. 66 (6), 3214-3221). The ability of such cytotoxic drugs to mediate local bystander killing is one selection criterion for the ADCs according to the present disclosure.

In some aspects, the disclosed ADCs also demonstrate bystander killing activity with low off-target cytotoxicity. Without being bound by theory, the bystander killing activity of an ADC may be particularly beneficial where its penetration into a solid tumor is limited and/or target antigen expression among tumor cells is heterogeneous. In some aspects, an ADC comprising a cleavable linker is particularly effective at bystander killing and/or demonstrates improved bystander killing activity, relative to comparable treatment with an ADC comprising a non-cleavable linker. In some aspects, the ADCs disclosed herein exhibit improved permeability and target cell penetrance over the drug moieties on their own. The improved permeability of, e.g., exatecan, allows for improved bystander activity when used as a payload.

### Anti-LRRC15 Antibodies and Antigen Binding Portions

In some aspects, the anti-LRRC15 antibody (e.g., recombinant humanized, chimeric, or human antibody) or antigen binding portion thereof useful for the present ADC is described herein in Table 3.

Anti-LRRC15 antibodies useful for ADC include all known forms of antibodies and other protein scaffolds with antibody-like properties. For example, the antibody can be a monoclonal antibody, a humanized antibody, a human antibody, a bispecific antibody, an immunoconjugate, a chimeric antibody, or a protein scaffold with antibody-like properties, such as fibronectin or ankyrin repeats. The antibody also can be a Fab, F(ab′)2, scFv, AFFIBODY, avimer, nanobody, single chain antibody, or a domain antibody. The antibody also can have any isotype or allotype, including any of the following isotypes: IgG1, IgG2, IgG3, IgG4, IgM, IgA1, IgA2, secretory IgA (SIgA), IgD, IgE, and allotypes thereof. Full-length antibodies can be prepared from VH and VL sequences using standard recombinant DNA techniques and nucleic acid encoding the desired constant region sequences to be operatively linked to the variable region sequences.

In some aspects, the anti-LRRC15 antibody, or antigen binding portion thereof useful for the present ADC disclosed herein comprises a heavy chain variable region (VH) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 3, 4, and 5, respectively, and a light chain variable region (VL) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 9, 10, and 11, respectively. In some aspects, the anti-LRRC15 antibody, or antigen binding portion thereof useful for the present ADC, which specifically binds to LRRC15 comprises: a VH comprising CDR1, CDR2, and CDR3 regions which comprises the amino acid sequences set forth in SEQ ID NOs: 3, 4, and 5, respectively, and a VL comprising CDR1, CDR2, and CDR3 regions which comprise the amino acid sequences set forth in SEQ ID NOs: 9, 10, and 11, respectively.

In some aspects, the anti-LRRC15 antibody, or antigen binding portion thereof, useful for the present ADC comprises a heavy chain variable region (VH) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 22, 23, and 24, respectively, and a light chain variable region (VL) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 27, 28, and 29, respectively. In some aspects, the anti-LRRC15 antibody, or antigen binding portion thereof, which specifically binds to LRRC15 comprises: a VH comprising CDR1, CDR2, and CDR3 regions which comprises the amino acid sequences set forth in SEQ ID NOs: 22, 23, and 24, respectively, and a VL comprising CDR1, CDR2, and CDR3 regions which comprise the amino acid sequences set forth in SEQ ID NOs: 27, 28, and 29, respectively.

In some aspects, the anti-LRRC15 antibody, or antigen binding portion thereof, useful for the present ADC comprises a heavy chain variable region (VH) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 31, 32, and 33, respectively, and a light chain variable region (VL) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 36, 37, and 38, respectively. In some aspects, the anti-LRRC15 antibody, or antigen binding portion thereof, which specifically binds to LRRC15 comprises: a VH comprising CDR1, CDR2, and CDR3 regions which comprises the amino acid sequences set forth in SEQ ID NOs: 31, 32, and 33, respectively, and a VL comprising CDR1, CDR2, and CDR3 regions which comprise the amino acid sequences set forth in SEQ ID NOs: 36, 37, and 38, respectively.

In some aspects, the anti-LRRC15 antibody, or antigen binding portion thereof, useful for the present ADC comprises a heavy chain variable region (VH) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 40, 41, and 42, respectively, and a light chain variable region (VL) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 45, 46, and 47, respectively. In some aspects, the anti-LRRC15 antibody, or antigen binding portion thereof, which specifically binds to LRRC15 comprises: a VH comprising CDR1, CDR2, and CDR3 regions which comprises the amino acid sequences set forth in SEQ ID NOs: 40, 41, and 42, respectively, and a VL comprising CDR1, CDR2, and CDR3 regions which comprise the amino acid sequences set forth in SEQ ID NOs: 45, 46, and 47, respectively.

In some aspects, the anti-LRRC15 antibody, or antigen binding portion thereof, useful for the present ADC comprises a heavy chain variable region (VH) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 49, 50, and 51, respectively, and a light chain variable region (VL) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 54, 55, and 56, respectively. In some aspects, the anti-LRRC15 antibody, or antigen binding portion thereof, which specifically binds to LRRC15 comprises: a VH comprising CDR1, CDR2, and CDR3 regions which comprises the amino acid sequences set forth in SEQ ID NOs: 49, 50, and 51, respectively, and a VL comprising CDR1, CDR2, and CDR3 regions which comprise the amino acid sequences set forth in SEQ ID NOs: 54, 55, and 56, respectively.

In some aspects, the anti-LRRC15 antibody, or antigen binding portion thereof, useful for the present ADC comprises a heavy chain variable region (VH) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 58, 59, and 60, respectively, and a light chain variable region (VL) comprising CDR1, CDR2, and CDR3 regions which have at least 60%, at least 70%, at least 80%, at least 85%, at least 90%, at least 95%, at least 98%, or at least 99% sequence identity to the amino acid sequences set forth in SEQ ID NOs: 63, 64, and 65, respectively. In some aspects, the anti-LRRC15 antibody, or antigen binding portion thereof, which specifically binds to LRRC15 comprises: a VH comprising CDR1, CDR2, and CDR3 regions which comprises the amino acid sequences set forth in SEQ ID NOs: 58, 59, and 60, respectively, and a VL comprising CDR1, CDR2, and CDR3 regions which comprise the amino acid sequences set forth in SEQ ID NOs: 63, 64, and 65, respectively.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NOs: 6, 7, 25, 26, 34, 35, 43, 44, 52, 53, 61, or 62, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID Nos: 12, 30, 39, 48, 57, or 66.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 6, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 12.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 7, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 12.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 25, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 30.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 26, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 30.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 34, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 39.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 35, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 39.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 43, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 48.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 44, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 48.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 52, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 57.

In some aspects, the anti-LRRC15 antibody, or antigen binding portion thereof, useful for the present ADC comprises a VH comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3 sequences, respectively, in SEQ ID NO: 53, and a VL comprising CDR1, CDR2, and CDR3 regions which have at least 60%, at least 70%, at least 80%, at least 85%, at least 90%, at least 95%, at least 98%, at least 99%, or 100% sequence identity to the CDR1, CDR2, and CDR3, respectively, in SEQ ID NO: 57.

In some aspects, the anti-LRRC15 antibody, or antigen binding portion thereof, useful for the present ADC comprises a VH comprising an amino acid sequence at least 60%, at least 70%, at least 80%, at least 85%, at least 90%, at least 95%, at least 98%, at least 99%, or 100% sequence identity to the amino acid sequence set forth in SEQ ID NO: 61 and a VL comprising an amino acid sequence at least 60%, at least 70%, at least 80%, at least 85%, at least 90%, at least 95%, at least 98%, at least 99%, or 100% sequence identity to the amino acid sequence set forth in SEQ ID NO: 66.

In some aspects, the anti-LRRC15 antibody, or antigen binding portion thereof, useful for the present ADC comprises a VH comprising an amino acid sequence at least 60%, at least 70%, at least 80%, at least 85%, at least 90%, at least 95%, at least 98%, at least 99%, or 100% sequence identity to the amino acid sequence set forth in SEQ ID NO: 62 and a VL comprising an amino acid sequence at least 60%, at least 70%, at least 80%, at least 85%, at least 90%, at least 95%, at least 98%, at least 99%, or 100% sequence identity to the amino acid sequence set forth in SEQ ID NO: 66.

In some aspects, the anti-LRRC15 antibody, or antigen binding portion thereof, useful for the present ADC comprises a VH and a VL comprising the amino acid sequence set forth in SEQ ID NO: 6 and the amino acid sequence set forth in SEQ ID NO: 12, respectively.

In some aspects, the anti-LRRC15 antibody, or antigen binding portion thereof, useful for the present ADC comprises a VH and a VL comprising the amino acid sequence set forth in SEQ ID NO: 7 and the amino acid sequence set forth in SEQ ID NO: 12, respectively.

In some aspects, the anti-LRRC15 antibody, or antigen binding portion thereof, useful for the present ADC comprises a VH and a VL comprising the amino acid sequence set forth in SEQ ID NO: 25 and the amino acid sequence set forth in SEQ ID NO: 30, respectively.

In some aspects, the anti-LRRC15 antibody, or antigen binding portion thereof, useful for the present ADC comprises a VH and a VL comprising the amino acid sequence set forth in SEQ ID NO: 26 and the amino acid sequence set forth in SEQ ID NO: 30, respectively.

In some aspects, the anti-LRRC15 antibody, or antigen binding portion thereof, useful for the present ADC comprises a VH and a VL comprising the amino acid sequence set forth in SEQ ID NO: 34 and the amino acid sequence set forth in SEQ ID NO: 39, respectively.

In some aspects, the anti-LRRC15 antibody, or antigen binding portion thereof, useful for the present ADC comprises a VH and a VL comprising the amino acid sequence set forth in SEQ ID NO: 35 and the amino acid sequence set forth in SEQ ID NO: 39, respectively.

In some aspects, the anti-LRRC15 antibody, or antigen binding portion thereof, useful for the present ADC comprises a VH and a VL comprising the amino acid sequence set forth in SEQ ID NO: 43 and the amino acid sequence set forth in SEQ ID NO: 48, respectively.

In some aspects, the anti-LRRC15 antibody, or antigen binding portion thereof, useful for the present ADC comprises a VH and a VL comprising the amino acid sequence set forth in SEQ ID NO: 44 and the amino acid sequence set forth in SEQ ID NO: 48, respectively.

In some aspects, the anti-LRRC15 antibody, or antigen binding portion thereof, useful for the present ADC comprises a VH and a VL comprising the amino acid sequence set forth in SEQ ID NO: 52 and the amino acid sequence set forth in SEQ ID NO: 57, respectively.

In some aspects, the anti-LRRC15 antibody, or antigen binding portion thereof, useful for the present ADC comprises a VH and a VL comprising the amino acid sequence set forth in SEQ ID NO: 53 and the amino acid sequence set forth in SEQ ID NO: 57, respectively.

In some aspects, the anti-LRRC15 antibody, or antigen binding portion thereof, useful for the present ADC comprises a VH and a VL comprising the amino acid sequence set forth in SEQ ID NO: 61 and the amino acid sequence set forth in SEQ ID NO: 66, respectively.

In some aspects, the anti-LRRC15 antibody, or antigen binding portion thereof, useful for the present ADC comprises a VH and a VL comprising the amino acid sequence set forth in SEQ ID NO: 62 and the amino acid sequence set forth in SEQ ID NO: 66, respectively.

In some aspects, the anti-LRRC15 antibody, or antigen binding portion thereof, useful for the present ADC comprises a heavy chain (HC) comprising an amino acid sequence at least 60%, at least 70%, at least 80%, at least 85%, at least 90%, at least 95%, at least 98%, at least 99%, or 100% sequence identity to the amino acid sequence set forth in any one of SEQ ID NO: 14-16 and a light chain (LC) comprising an amino acid sequence at least 60%, at least 70%, at least 80%, at least 85%, at least 90%, at least 95%, at least 98%, at least 99%, or 100% sequence identity to the amino acid sequence set forth in SEQ ID NO: 17. In some aspects, the anti-LRRC15 antibody, or antigen binding portion thereof, useful for the present ADC comprises a HC and a LC comprising the amino acid sequence set forth in any one of SEQ ID NO: 14-16 and the amino acid sequence set forth in SEQ ID NO: 17, respectively. In some aspects, the anti-LRRC15 antibody, or antigen binding portion thereof, useful for the present ADC comprises a HC and a LC comprising the amino acid sequence set forth in any one of SEQ ID NO: 15 and the amino acid sequence set forth in SEQ ID NO: 17, respectively. In some aspects, the anti-LRRC15 antibody, or antigen binding portion thereof, useful for the present ADC comprises a HC and a LC comprising the amino acid sequence set forth in any one of SEQ ID NO: 16 and the amino acid sequence set forth in SEQ ID NO: 17, respectively.

In some aspects, the anti-LRRC15 antibody, or antigen binding portion thereof, useful for the present ADC specifically binds to LRRC15 with a KD less than 1×10⁻⁶M. In some aspects, the anti-LRRC15 antibody, or antigen binding portion thereof, useful for the present ADC specifically binds to LRRC15 with a KD less than 1×10⁻⁷ M. In some aspects, the anti-LRRC15 antibody, or antigen binding portion thereof, useful for the present ADC specifically binds to LRRC15 with a KD less than 1×10⁻⁸ M. In some aspects, the anti-LRRC15 antibody, or antigen binding portion thereof, useful for the present ADC specifically binds to LRRC15 with a KD less than 5×10⁻⁹M. In some aspects, the anti-LRRC15 antibody, or antigen binding portion thereof, useful for the present ADC specifically binds to LRRC15 with a KD less than 1×10⁻⁹ M. In some aspects, the anti-LRRC15 antibody, or antigen binding portion thereof, useful for the present ADC specifically binds to LRRC15 with a KD less than 5×10⁻¹⁰ M.

### Antibody Binding

The anti-LRRC15 antibodies or antigen binding portions thereof useful for the present ADC bind to LRRC15 (e.g., human LRRC15) in solution, to LRRC15 attached to a solid surface, such as a microtiter plate, and/or to LRRC15 (e.g., human LRRC15) anchored to the membrane of a cell. In some aspects, the anti-LRRC15 antibody or antigen binding portion thereof useful for the present ADC binds to human LRRC15, cyno LRRC15, or both.

In some aspects, the anti-LRRC15 antibody or antigen binding portion thereof useful for the present ADC binds to human and/or cynomolgus LRRC15 with a KD of 100 nM or less, such as 90 nM or less, 80 nM or less, 70 nM or less, 60 nM or less, 50 nM or less, such as 40 nM or less, 30 nM or less, 20 nM or less, 10 nM or less, 5 nM or less, 3 nM or less, 1 nM or less, 0.9 nM or less, 0.8 nM or less, 0.7 nM or less, 0.6 nM or less, 0.5 nM or less, 0.4 nM or less, 0.3 nM or less, 0.2 nM or less, 0.1 nM or less, as measured by any detection method known in the art or described herein, for example, in Example 4.

In some aspects, the anti-LRRC15 antibody or antigen binding portion thereof useful for the present ADC binds to human and/or cynomolgus LRRC15 with a KD between 0.1 nM and 100 nM, between 0.1 nM and 50 nM, between 0.1 nM and 25 nM, between 0.1 nM and 10 nM, between 0.1 nM and 5 nM, between 0.1 nM and 2 nM, between 0.1 nM and 1 nM, between 0.1 nM and 0.5 nM, between 1 nM and 100 nM, between 1 nM and 50 nM, between 1 nM and 25 nM, between 1 nM and 10 nM, between 1 nM and 5 nM, between 1 nM and 2 nM, between 5 nM and 100 nM, between 5 nM and 50 nM, between 5 nM and 25 nM, between 5 nM and 10 nM, between 10 nM and 100 nM, between 10 nM and 50 nM, between 10 nM and 25 nM, between 25 nM and 100 nM, between 25 nM and 50 nM, or between 50 nM and 100 nM, as measured by any detection method known in the art or described herein.

Binding of an anti-LRRC15 antibody or antigen binding portion thereof (and absence of binding) may be assessed qualitatively or quantitatively by any method known in the art. Exemplary binding methodologies include immunohistochemistry, flow cytometry using, e.g., LRRC15-overexpressing cells (e.g., 3T3-LRRC15), surface plasmon resonance (SPR) using, e.g., BIACORE® system (Cytiva), or bio-layer interferometry (BLI) using, e.g., the Octet platform (ForteBio).

In some aspects, the LRRC15 antibody or antigen binding portion thereof useful for the present ADC does not bind to or does not cross-react with other leucine-rich repeat-containing protein family, including, but not limited to LRRC26, LRRC38, LRRC52, LRRC55, LRRC8A, LRRC8B, LRRC8C, LRRC8D, and LRRC8E, e.g., as assessed by, e.g., flow cytometry using cells that overexpress one of the foregoing LRRCs, or by SPR or BLI. For example, in some aspects, the anti-LRRC15 antibody or antigen binding portion thereof useful for the present ADC binds to one of the foregoing LRRCs with a signal or affinity that is not significantly above the signal seen with a control antibody (e.g., isotype control) or the signal seen in the absence of the anti-LRRC15 antibody.

In some aspects, the anti-LRRC15 antibody or antigen binding portion thereof useful for the present ADC binds to all or a portion of amino acids of human LRRC15 (SEQ ID NO: 1) as described in Example 5.

In some aspects, the anti-LRRC15 antibody or antigen binding portion thereof useful for the present ADC binds to (or is determined to bind to) LRRC15-overexpressing cancer cell lines or tumor cells. In some aspects, the anti-LRRC15 antibody or antigen binding portion thereof useful for the present ADC binds to LRRC15 on these cells as assessed e.g., by flow cytometry. For example, in some aspects, at least 5%, at least 10%, at least 20% at least 50%, at least 75%, or at least 90% of LRRC15-expressing cells can be detected by binding of the anti-LRRC15 antibody (e.g., display a signal above that seen with an isotype control antibody) by any detection method known in the art or described herein.

In some aspects, the anti-LRRC15 antibody or antigen binding portion thereof useful for the present ADC binds to LRRC15 expressed on cells (e.g., human and/or murine LRRC15 expressed on, e.g., 3T3 cells) with an EC50 of 1000 ng/ml or less, 500 ng/ml or less, 200 ng/ml or less, 150 ng/ml or less, 100 ng/ml or less, 50 ng/ml or less, 25 ng/ml or less, 10 ng/ml or less, 5 ng/ml or less, 2 ng/ml or less, or 1 ng/ml or less, as measured by any detection method known in the art or described herein.

In some aspects, the anti-LRRC15 antibody or antigen binding portion thereof useful for the present ADC binds to LRRC15 expressed on cells with an EC50 between about 1 ng/ml and about 1000 ng/ml, between about 1 ng/ml and about 500 ng/ml, between about 1 ng/ml and about 200 ng/ml, between about 1 ng/ml and about 100 ng/ml, between about 1 ng/ml and about 50 ng/ml, between about 1 ng/ml and about 25 ng/ml, between about 1 ng/ml and about 10 ng/ml, between about 1 ng/ml and about 5 ng/ml, between about 5 ng/ml and about 500 ng/ml, between about 5 ng/ml and about 200 ng/ml, between about 5 ng/ml and about 100 ng/ml, between about 5 ng/ml and about 50 ng/ml, between about 5 ng/ml and about 25 ng/ml, between about 5 ng/ml and about 10 ng/ml, between about 10 ng/ml and about 500 ng/ml, between about 10 ng/ml and about 200 ng/ml, between about 10 ng/ml and about 100 ng/ml, between about 10 ng/ml and about 50 ng/ml, between about 10 ng/ml and about 25 ng/ml, between about 25 ng/ml and about 500 ng/ml, between about 25 ng/ml and about 200 ng/ml, between about 25 ng/ml and about 100 ng/ml, between about 25 ng/ml and about 50 ng/ml, between about 50 ng/ml and about 500 ng/ml, between about 50 ng/ml and about 200 ng/ml, between about 50 ng/ml and about 100 ng/ml, between about 100 ng/ml and about 500 ng/ml, or between about 100 ng/ml and about 200 ng/ml, as measured by any detection method known in the art or described herein.

The binding of the anti-LRRC15 antibody or antigen binding portion thereof useful for the present ADC to LRRC15 may also be defined using quantitative immunofluorescence by flow cytometry, which allows the number of antibody molecules bound per cell or the number of LRRC15-expressing cells to be quantified. In some aspects, the number of LRRC15 molecules expressed per cell or number of LRRC15-expressing cells in a cell line or tumor sample may be quantified by quantitative immunofluorescence using an anti-LRRC15 antibody or antigen binding portion thereof described herein.

An anti-LRRC15 antibody or antigen binding portion thereof useful for the present ADC binds to soluble or membrane-bound human and/or cynomolgus LRRC15 with high affinity, for example, with a KD of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, as measured by, e.g., surface plasmon resonance or other art-recognized methods.

In some aspects, the anti-LRRC15 antibody or antigen binding portion thereof useful for the present ADC binds to soluble or membrane-bound human and/or cynomolgus LRRC15 with a KD of between 10⁻⁷ M and 10⁻¹² M, between 10⁻⁷ M and 10⁻¹¹ M, between 10⁻⁷ M and 10⁻¹⁰ M, between 10⁻⁷ M and 10⁻⁹ M, between 10⁻⁷ M and 10⁻⁸ M, between 10⁻⁸ M and 10⁻¹² M, between 10⁻⁸ M and 10⁻¹¹ M, between 10⁻⁸ M and 10⁻¹¹ M, between 10⁻⁸ M and 10⁻⁹ M, between 10⁻⁹ M and 10⁻¹² M, between 10⁻⁹ M and 10⁻¹¹ M, between 10⁻⁹ M and 10⁻¹⁰ M, between 10⁻¹⁰ M and 10⁻¹² M, between 10⁻¹⁰ M and 10⁻¹¹ M, or between 10⁻¹¹ M and 10⁻¹² M, as measured by, e.g., surface plasmon resonance or other art-recognized methods.

In some aspects, the anti-LRRC15 antibody or antigen binding portion thereof binds to soluble or membrane-bound human and/or cynomolgus LRRC15 with a K_{D} of between 10⁻⁷ M and 10⁻¹² M, between 10⁻⁷ M and 10⁻¹¹ M, between 10⁻⁷ M and 10⁻¹⁰ M, between 10⁻⁷ M and 10⁻⁹ M, between 10⁻⁷ M and 10⁻⁸ M, between 10⁻⁸ M and 10⁻¹² M, between 10⁻⁸ M and 10⁻¹¹ M, between 10⁻⁸ M and 10⁻¹¹ M, between 10⁻⁸ M and 10⁻⁹ M, between 10⁻⁹ M and 10⁻¹² M, between 10⁻⁹ M and 10⁻¹¹ M, between 10⁻⁹ M and 10⁻¹⁰ M, between 10⁻¹⁰ M and 10⁻¹² M, between 10⁻¹⁰ M and 10⁻¹¹ M, or between 10⁻¹¹ M and 10⁻¹² M, as measured by, e.g., surface plasmon resonance or other art-recognized methods.

Competing Antibodies and Antibodies that Bind to the Same Epitope

The anti-LRRC15 antibodies and antigen binding portions described herein are distinguished by the characteristic epitope(s) (i.e., site(s) on LRRC15) to which they bind, e.g., Example 5. The epitope(s) to which the antibody or antigen binding portion binds can be determined using art-recognized methods. An anti-LRRC15 antibody or antigen binding portion thereof is considered to bind to the same epitope as a reference anti-LRRC15 antibody (for example, MBP001) if it, e.g., contacts one or more of the same residues on human LRRC15 as the reference antibody or contacts all of the same residues at all of the same regions of human LRRC15 as the reference antibody.

Antibodies sharing common epitope binding characteristics may considered to fall within a common “epitope bin.” In some cases, a LRRC15-binding “test antibody” may be determined to fall within a common “epitope bin” by comparison to the sequence of a given “reference” antibody (e.g., MBP001) known to fall within a particular epitope bin. In other cases, epitope binning experiments may be performed to determine whether a test antibody falls into the same “bin” as an antibody based on common binding characteristics with a reference antibody. Antibodies that reduce binding of the antibodies disclosed herein by sequence to, e.g., an immobilized LRRC15 protein or protein fragment, particularly at roughly stoichiometric concentrations, are likely to bind at the same, overlapping, or adjacent epitopes, and thus may share the desirable functional properties as one or more of the antibodies disclosed herein.

In some aspects, antibodies falling into the same epitope bin are determined by assaying for antibodies that compete for binding to LRRC15 with particular anti-LRRC15 antibodies described herein. Methods of determining antibody competition are known in the art.

In some aspects, BIACORE analysis can be used to assess the ability of the antibodies to compete. The ability of a test antibody to inhibit the binding of an anti-LRRC15 antibody described herein to LRRC15 demonstrates that the test antibody can compete with the antibody for binding to LRRC15.

Inhibition or blocking by one antibody relative to another may be carried out by performing any suitable competitive inhibition experiment using art-recognized methods or those described herein, including but not limited to surface plasmon resonance (SPR) using e.g., the BIACORE® system (Cytiva), bio-layer interferometry (BLI) using e.g., the Octet platform (ForteBio), enzyme-linked immunoassay (ELISA), and flow cytometry. In some aspects, epitope binning of the anti-LRRC15 antibodies may be performed using a recombinant LRRC15 protein or fragment, which is biotinylated and captured onto, e.g., Streptavidin biosensors which are bound by the first antibody until saturation is achieved. In some aspects, epitope binning may be carried out using a cell-based competition binding FACS assay.

Unless otherwise indicated, an antibody will be considered to compete with an anti-LRRC15 antibody if it reduces binding of the selected antibody to human LRRC15 (SEQ ID NO: 1), cynomolgus LRRC15, or fragment thereof by at least 20% when used at a roughly equal molar concentration with the selected antibody, as measured in competition ELISA experiments as outlined in the preceding two paragraphs.

In some aspects, the anti-LRRC15 antibodies or antigen binding portions thereof comprise a linear epitope. In some aspects, the anti-LRRC15 antibodies or antigen binding portions thereof comprise a conformational epitope.

In some aspects, the anti-LRRC15 antibodies are screened for high affinity binding to human LRRC15, and selected antibodies therefrom are studied, e.g., using yeast display assays in which sequence variants of LRRC15 are presented on the surface of yeast cells, MS-based protein footprinting, such as HDX-MS and Fast Photochemical Oxidation of Proteins (FPOP), and structural methods, such as X-ray crystal structure determination, molecular modeling, and nuclear magnetic resonance (NMR) spectroscopy, including NMR determination of the H-D exchange rates of labile amide hydrogens in LRRC15 when free and when bound in a complex with an antibody of interest. Such methods can provide atomic resolution of the precise epitope bound by the antibody. In recent years, SP-cryo-EM has emerged as a complementary technique to crystallography and NMR for determining near-atomic level structures suitable for application in drug discovery (Renaud et al. Nat Rev Drug Discov 2018; 17:471-92; Scapin et al. Cell Chem Biol 2018; 25:1318-25; Ceska et al. Biochemical Society Transactions 2019: p. BST20180267).

Anti-LRRC15 antibodies which bind to and compete for the same or similar epitopes to the antibodies disclosed herein may be raised using immunization protocols similar to those described herein, for example, in Example 1. In some aspects the immunization may be carried out with a construct containing the epitope bound by the anti-LRRC15 antibodies disclosed herein. The resulting antibodies can be screened for high affinity binding to human LRRC15 by FACS, ELISA, or SPR and/or screened for the ability to block binding of a reference antibody disclosed herein as determined by ELISA or by blocking their ability to bind to cells expressing LRRC15 on their surface, e.g., by FACS or SPR. A test antibody can be contacted with a LRRC15 protein, protein fragment, or LRRC15-expressing cell prior to, at the same time as, or after the addition of the reference antibody.

Alternatively, variants of anti-LRRC15 antibodies or antigen binding portion described herein can be obtained by mutagenesis of cDNA sequences encoding the heavy and light chains of the antibody.

In some aspects, variants of anti-LRRC15 antibodies or antigen binding portion thereof has one or more properties of the following: (i) disruption of tumor microenvironment, (ii) reduced tumor growth and spread, (iii) enhanced drug delivery, (iv) inhibition of angiogenesis, (v) reduced resistance to therapy, (vi) direct tumor killing, (vii) immune modulation; and (viii) synergistic effects with other therapies.

### Antibody Internalization

In another aspect, provided herein is an anti-LRRC15 antibody, or antigen binding portion thereof, which binds to human and/or cynomolgus LRRC15 and induces internalization of the anti-LRRC15 antibody or antigen binding portion thereof in accordance with, e.g., the conditions and results described in, e.g., Example 10, or is linked to a cytotoxin for killing LRRC15-expressing cells in accordance with the conditions and results described in, e.g., Example 10.

The identification of internalizing anti-LRRC15 antibodies, or antigen binding portions thereof, disclosed herein is important for development of effective antibody-drug conjugates (ADCs). An anti-LRRC15 antibody, or antigen binding portion thereof, in accordance with the present disclosure can be evaluated for its ability to internalize into cells as determined by any well-known method in the art, including but not limited to use of the IncuCyte live-cell analysis system, Amnis IMAGESTREAM® Imaging Flow Cytometry Analysis, or laser scanning confocal microscopy.

The internalizing anti-LRRC15 antibody, or antigen binding portion thereof, disclosed herein can be characterized or ranked in terms of their “degree of internalization” or “level of internalization,” which can relate to the degree (e.g., cell percentage) or level of internalization (total amount of internalized antibodies) at a given antibody concentration (e.g., 100 nM) or following a given period of time (e.g., 2 minutes, 5 minutes, 10 minutes or 30 minutes) relative to a control antibody (e.g., MBP001), such as a non-internalizing antibody, control IgG, or other control antibody (e.g., benchmark antibody).

In some aspects, the anti-LRRC15 antibody, or antigen binding portion thereof, disclosed herein internalizes into at least about 20%, 30%, 40%, 50%, 60%, 70%, 80%, 90% or 100% of LRRC15-expressing cells in a cell population. In some aspects, the anti-LRRC15 antibody, or antigen binding portion thereof, of the present disclosure internalizes into a LRRC15-expressing cell or population of LRRC15-expressing cells at a level at least 2-fold, at least 5-fold, at least 20-fold, at least 100-fold, at least 500-fold, or at least 2,000-fold greater level than a control antibody (e.g., non-internalizing antibody, control IgG, other antibody, benchmark antibody).

In some aspects, the level of internalization of an anti-LRRC15 antibody, or antigen binding portion thereof, disclosed herein into LRRC15-expressing cells (e.g., 3T3-hu/mu LRRC15) is determined by comparing area under time-course (AUC) immunofluorescence levels relative to a reference antibody, e.g., as described in Example 10. In some aspects, the anti-LRRC15 antibody, or antigen binding portion thereof, disclosed herein internalizes into a LRRC15-expressing cell or population of LRRC15-expressing cells at antibody/cell concentrations resulting in an AUC immunofluorescence level that is at least 50%, at least 75%, at least 2-fold, at least 3-fold, at least 5-fold, at least 10-fold, at least 25-fold, at least 50-fold, or at least 100-fold greater compared to a control antibody, as described herein. In some aspects, the anti-LRRC15 antibody, or antigen binding portion thereof, internalizes into a LRRC15-expressing cell e.g., in accordance with the conditions and results set forth in Example 10.

In some aspects, the anti-LRRC15 antibody, or antigen binding portion thereof, disclosed herein may be characterized by its “rate of internalization” represented, e.g., by its T1/2 of internalization, which is defined as the time at which half of the maximal internalization is achieved, as measured from the time the antibody is added to the cells. In some aspects, the T1/2 of internalization for the anti-LRRC15 antibody, or antigen binding portion thereof, disclosed herein may be enhanced or increased by at least 10%, 30%, 50%, 75%, 2-fold, 3-fold, 5-fold or more, resulting in a reduction of the T1/2 by at least 10%, 30%, 50%, 75%, 2-fold, 3-fold, 5-fold or more compared to a control antibody, as described herein. For example, instead of having a T1/2 of 10 minutes, the anti-LRRC15 antibody, or antigen binding portion thereof, may exhibit an increased rate of internalization and thereby reduce the T1/2 to 5 minutes (i.e., a two-fold increase in rate of internalization or a two-fold decrease in T1/2). In some aspects, the T1/2 is reduced by at least 10 minutes, 30 minutes, or 1 hour.

In some aspects, the anti-LRRC15 antibody, or antigen binding portion thereof, disclosed herein may be characterized by its maximal level of internalization into a LRRC15-expressing cell or population of LRRC15-expressing cells, where the maximal level of internalization is represented by the level of internalization at the plateau of a graph representing the internalization plotted against antibody concentrations or times. In some aspects, the anti-LRRC15 antibody, or antigen binding portion thereof, disclosed herein exhibits a maximal level of internalization, which is at least 10%, 30%, 50%, 75%, 2-fold, 3-fold, 5-fold or more relative to a control antibody, as described herein.

Another way to compare internalization efficacies of the anti-LRRC15 antibody, or antigen binding portion thereof, disclosed herein is to compare their level of internalization at a given antibody concentration (e.g., 100 nM) and/or at a given time (e.g., 2 minutes, 5 minutes, 10 minutes or 30 minutes).

In some aspects, the anti-LRRC15 antibody, or antigen binding portion thereof, disclosed herein may be characterized by level of internalization, which ability to internalize may be determined using area under time-course (AUC) immunofluorescence analysis representing the antibody concentration at which 50% of the maximum level of internalization is obtained, as measured from the time the antibody is added to the cells, for example, as described in Example 10.

In some aspects, the anti-LRRC15 antibody, or antigen binding portion thereof, disclosed herein exhibits an EC50 binding value less than 50 nM, less than 40 nM, less than 30 nM, less than 25 nM, less than 20 nM, less than 15 nM, less than 10 nM, less than 8 nM, less than 6 nM, less than 4 nM or less than 3 nM. In some aspects, the anti-LRRC15 antibody, or antigen binding portion thereof, disclosed herein exhibits an EC50 internalization value between 1 nM and 50 nM, between 4 nM and 50 nM, between 10 nM and 50 nM, between 20 nM and 50 nM, between 30 nM and 50 nM, between 4 nM and 40 nM, between 4 nM and 30 nM, between 4 nM and 20 nM, between 8 nM and 40 nM, between 8 nM and 30 nM, between 8 nM and 20 nM, between 12 nM and 40 nM, between 12 nm and 30 nM, or between 12 nM and 25 nM.

In some aspects, the level of binding of the anti-LRRC15 antibody, or antigen binding portion thereof, disclosed herein can be defined relative to that of a given control antibody, as described herein, and expressed as a percentage of the EC50 value obtained compared to the control antibody. In some aspects, the extent of binding reflected in the EC50 value can be enhanced by at least 10%, 30%, 50%, 75%, 2-fold, 3-fold, 5-fold or more, as compared to a control antibody.

In some aspects, the anti-LRRC15 antibody, or antigen binding portion thereof, disclosed herein comprises a modified constant region conferring increased internalization relative to the same anti-LRRC15 antibody, or antigen binding portion thereof, without the modified constant region, or relative to a control antibody, as described herein. Modified constant regions for use in these aspects are described in U.S. Pat. No. 10,653,791, the contents of which are herein incorporated by reference in their entirety. For example, in some aspects, the anti-LRRC15 antibody, or antigen binding portion thereof, disclosed herein comprises an IgG2 hinge or a substitution of a non-IgG2 hinge with an IgG2 hinge. In some aspects, the anti-LRRC15 antibody, or antigen binding portion thereof, disclosed herein comprises a hinge and/or a CH1 domain that is not an IgG2 hinge and/or IgG2 CH1 domain is replaced with an IgG2 hinge and/or IgG2 CH1 domain.

In some aspects, the anti-LRRC15 antibody, or antigen binding portion thereof, disclosed herein comprising the modified constant region has a rate of internalization (as measured by T1/2) that is increased by at least 10%, 30%, 50%, 75%, 2 fold, 3 fold, 5 fold or more, resulting in a reduction of the T1/2 by at least 10%, 30%, 50%, 75%, 2 fold, 3 fold, 5 fold or more relative to the same anti-LRRC15 antibody, or antigen binding portion thereof, without the modified constant region, or relative to a control antibody, as described herein.

### Antibody Physical Properties

Each antibody or antigen binding portion thereof will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. In addition, each antibody or antigen binding portion thereof will have a characteristic melting temperature, with a higher melting temperature indicating greater overall stability in vivo (Krishnamurthy R and Manning M C (2002) Curr Pharm Biotechnol 3:361-71). In general, the TM1 (the temperature of initial unfolding) may be greater than 60° C., greater than 65° C., or greater than 70° C. The melting point of an antibody or antigen binding portion can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9). In a further aspect, the antibodies and antigen binding portions thereof are selected that do not degrade rapidly. Degradation of an antibody or antigen binding portion thereof can be measured using capillary electrophoresis (CE) and MALDI-MS (Alexander AJ and Hughes DE (1995) Anal Chem 67:3626-32).

In some aspects, the anti-LRRC15 antibody or antigen binding portion thereof has minimal aggregation effects, which can otherwise lead to the triggering of an unwanted immune response and/or altered or unfavorable pharmacokinetic properties. Generally, antibodies and antigen binding portions thereof are acceptable with aggregation of 25% or less, 20% or less, 15% or less, 10% or less, or 5% or less. Aggregation can be measured by several techniques, including size-exclusion column (SEC), high performance liquid chromatography (HPLC), and light scattering.

### Immunoconjugates for LRRC15 Detection

In some aspects, the immunoconjugate comprises a detectable label for LRRC15 detection and/or cancer diagnosis. The detectable labels may be attached to the anti-LRRC15 antibodies, antigen binding portions thereof, or bispecific antibodies described herein according to the coupling methods described herein. The immunoconjugate detection agents of the present disclosure may be used for detecting LRRC15 and/or diagnosing LRRC15-associated cancers in accordance with the methods described herein.

For diagnostic purposes, detectable labels may include, for example, radioisotopes for whole body imaging, and radioisotopes, enzymes, fluorescent labels, and other suitable antibody tags for sample testing. The detectable labels can be any of the various types used currently in the field of in vitro diagnostics, including particulate labels including metal sols such as colloidal gold, isotopes such as I¹²⁵ or Tc⁹⁹ presented for instance with a peptidic chelating agent of the N2S2, N3S or N4 type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase, and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-STAR® or other art-recognized luminescent substrates including, for example, chelates of suitable lanthanides, such as Terbium (III) and Europium (III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

### Engineered Cells

In some aspects, provided herein is an engineered cell, e.g., a chimeric antigen receptor (CAR) expressing cell, in which expresses an engineered polypeptide comprising: (1) a LRRC15 binding domain, (2) a transmembrane domain, and (3) at least one cytoplasmic signaling domain. The LRRC15 binding domain includes one or more LRRC15 antigen binding portions including, but not limited to, CDRs, variable heavy domains, variable light domains, Fab regions, heavy chains, light chains, single-chain variable fragments (scFvs), CH1, CH2, and/or CH3 domains, Fc regions, fragments thereof, and combinations thereof. In certain aspects, the LRRC15 antigen binding portion is an scFv that specifically binds to LRRC15.

The transmembrane domain can be any transmembrane domain derived or obtained from any molecule. In some aspects, the transmembrane domain is fused to the LRRC15 binding domain of the CAR. The transmembrane domain may be derived from either a natural or synthetic source. In some aspects, the transmembrane domain can be derived from any membrane-bound or transmembrane protein. In some aspects, the transmembrane (TM) domain is selected from a group including, but not limited to, the alpha, beta, or zeta chain of the T cell receptor, CD3-epsilon, CD3-zeta, CD4, CD5, CD8, CD9, CD16, CD22, CD28, CD33, CD37, CD45, CD64, CD80, CD86, CD134, CD137, or CD154.

The CAR also comprises at least one signaling domain, which can also be referred to as the intracellular signaling domain and/or the cytoplasmic co-stimulatory signaling domain of the CAR. The cytoplasmic signaling domain is responsible for activation of at least one of the normal effector functions of the T cell and is required for an efficient response of lymphocytes to an antigen. The term “effector function” refers to a specialized function of a cell. Effector function of a T cell, for example, may be cytolytic activity or helper activity including the secretion of cytokines. Thus, the term “cytoplasmic costimulatory signaling domain” refers to the portion of a protein which transduces the effector function signal and directs the cell to perform a specialized function. While the entire intracellular signaling domain can be employed, in many cases it is not necessary to use the entire chain (i.e., the signaling domain can be derived from the entire protein). To the extent that a truncated portion of the intracellular signaling domain is used, such truncated portion may be used in place of the intact chain as long as it transduces the effector function signal.

The intracellular signaling domain can be derived from and include any truncated portion of the intracellular signaling domain sufficient to transduce the effector function signal. In some aspects, the intracellular signaling domain is selected from the cytoplasmic sequences of the T cell receptor (TCR) and co-receptors that initiate signal transduction following antigen receptor engagement. In some aspects, the intracellular signaling domain comprises a domain derived from CD2, CD3-zeta, CD3-gamma, CD3-delta, CD3-epsilon, CD5, CD7, CD22, CD27, CD28, CD30, CD40, CD66d, CD79a, CD79b, 4-1BB (CD137), 0×40, PD-1, ICOS, lymphocyte function-associated antigen-1 (LFA-1), LIGHT, NKG2C, B7-H3, FcR-gamma, FcR-beta, TCR-zeta, or any combination thereof. In some aspects, the intracellular signaling domain is derived from CD3-zeta, CD28, and/or 4-1BB. In some aspects, the CAR comprises two or three cytoplasmic signaling domains, such as the cytoplasmic signaling domains are derived from CD28, CD3, and/or 4-1BB.

In some aspects, the provided herein are CAR-T cell-derived effector cells for use in patients with cancer. In some aspects, the CAR-T cell-derived effector cells are a population of activated T cells expressing a CAR engineered polypeptide comprising a LRRC15 binding domain, a transmembrane domain, and at least one cytoplasmic signaling domain, as described herein.

T cells used in the methods disclosed herein can be isolated. Sources for the T cells include, but are not limited to, peripheral blood, umbilical cord blood, bone marrow, or other sources of hematopoietic cells. Methods for separating, enriching, and expanding the desired T cells can be employed. In some aspects, the T cells are expanded by culturing in the presence of IL-2. In other aspects, the T cells are expanded by culturing in the presence of anti-CD3 antibodies and/or anti-CD28 antibodies. In other aspects, the T cells are expanded by culturing in the presence of IL-2, and by culturing in the presence of anti-CD3 antibodies and/or anti-CD28 antibodies.

Procedures for separation of cells include, but are not limited to, density gradient centrifugation, coupling to particles that modify cell density, magnetic separation with antibody-coated magnetic beads, affinity chromatography; cytotoxic agents joined to or used in conjunction with a mAb, including, but not limited to, complement and cytotoxins, and panning with an antibody attached to a solid matrix, for example, a plate or chip, elutriation, flow cytometry, or any other convenient techniques.

The isolated T cells can be autologous or non-autologous to the subject to which they are administered in the methods of treatment disclosed herein. Autologous cells are isolated from the subject to which the population of activated T cells comprising the CAR are to be administered. In some aspects, autologous cells are isolated from the subject to which the isolated and expanded cells recombinantly expressing a CAR are to be administered. In some aspects, the cells can be obtained by leukapheresis, where leukocytes are selectively removed from withdrawn blood, made recombinant, and then re-transfused into the donor subject. Alternatively, allogeneic cells from a non-autologous donor that is not the subject can be used. In the case of a non-autologous donor, the cells are typed and matched for human leukocyte antigen (HLA) to determine an appropriate level of compatibility. For both autologous and non-autologous cells, the cells can optionally be cryopreserved until ready to be used for genetic manipulation and/or administration to a subject.

Because cytokine release is a necessary consequence of T cell activation and efficacy, for effective CAR-T cell-based therapy, it is preferred that at least a portion of the activated T cells produce one or more cytokines, such as one or more cytokines selected from the group consisting of IL-1, IL-2, TNF-α, and IFN-γ. Additionally, at least a portion of the population of activated T cells express one or more surface markers comprising CD2, CD28, CTLA4, CD40 ligand (gp39), CD18, CD25, CD69, CD16/CD56, MHC Class I, MHC Class II, CD8, CD4, CD3/TcR, CD54, LFA-1, VLA-4, or any combination thereof.

### Antibody Engineering

The anti-LRRC15 antibody, or antigen binding portion thereof, bispecific antibody, multispecific antibody, immunoconjugate, antibody drug conjugate (ADC), or engineered cells such as CAR as disclosed herein can be modified or engineered to improve their physical and functional properties.

Antibodies useful for ADC produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine. In some aspects, the antibody useful for ADC of the present disclosure comprises a Lys at the C-terminus of the heavy chain. In some aspects, an example of the antibody with Lys comprises a heavy chain comprising the sequence as set forth in SEQ ID NO: 15 and the light chain comprising the sequence as set forth in SEQ ID NO: 17.

Antibodies useful for ADC may be modified as part of the production process in certain host cells or through metabolism in vivo. An antibody or antibody region amino acid sequence herein is intended to encompass not only the specific amino acid sequence, but also that sequence as post-translationally modified, for instance, including side chain modifications and cleavages. Such a post-translational modification can occur, for instance, as a result of production of the antibody in a host cell and/or as a result of post-translational modification in vivo in an animal (e.g., a human).

In some aspects, an antibody useful for ADC disclosed herein comprises a post-translational modification (e.g., one or more post-translational modifications). Post-translational modifications can include, e.g., ubiquitination, phosphorylation, acetylation, hydroxylation, methylation, glycyosylation, AMPylation, prenylation, deamidation, elimylation, citrullination, pyroglutamation, and carbamoylation. In some aspects, the antibody useful for ADC is not post-translationally modified. In some aspects, the antibody useful for ADC of the present disclosure comprises a pyroglutamate at the N terminus of the heavy chain. In some aspects, an example of the pyroglutamate modified antibody comprises a heavy chain comprising the sequence as set forth in SEQ ID NO: 16 and the light chain comprising the sequence as set forth in SEQ ID NO: 17.

As noted above, antibodies useful for ADC can undergo post-translational cleavage of one or more, particularly one or two, amino acids from the C-terminus of the heavy chain, often a Gly-Lys. This cleavage can occur, for instance, as a result of the process of production of the antibody in a host cell. An antibody useful for ADC produced by expression of a specific nucleic acid molecule encoding a full-length heavy chain can include the full-length heavy chain, or it can include a cleaved variant of the full-length heavy chain, such as a heavy chain lacking a C-terminal Lys or a C-terminal Gly-Lys. In some aspects, the antibody useful for ADC of the present disclosure comprises no Lys at the C-terminus of the heavy chain. In some aspects, an example of the antibody useful for ADC without any Lys comprises a heavy chain comprising the sequence as set forth in SEQ ID NO: 14 and the light chain comprising the sequence as set forth in SEQ ID NO: 17.

In some aspects, the antibody useful for ADC of the present disclosure comprises Lys at the C-terminus of the heavy chain and pyroglutamate at the N-terminus of the heavy chain.

In some aspects, the pharmaceutical composition comprising the antibody useful for ADC of the present disclosure comprises two or more antibodies in combination wherein two or more antibodies comprise (i) an antibody comprising a heavy chain comprising the sequence as set forth in SEQ ID NO: 14 and the light chain comprising the sequence as set forth in SEQ ID NO: 17; (2) an antibody comprising a heavy chain comprising the sequence as set forth in SEQ ID NO: 15 and the light chain comprising the sequence as set forth in SEQ ID NO: 17; (3) an antibody comprising a heavy chain comprising the sequence as set forth in SEQ ID NO: 16 and the light chain comprising the sequence as set forth in SEQ ID NO: 17; or (4) any combination thereof.

Other types of post-translational modifications can occur during production of antibodies useful for ADC, or otherwise in vivo, such as the modification of an amino acid side chain. For instance, an N-terminal Glu or Gln residue on an antibody chain can be post-translationally modified to an N-terminal pyroglutamate (also known as pyrrolidine carboxylate; abbreviated pE).

### Antibody Engineering of the Fc Region

The anti-LRRC15 antibody, or antigen binding portion thereof or the bispecific antibody, multispecific antibody, immunoconjugate, antibody drug conjugate (ADC), or engineered cells such as CAR that comprises the anti-LRRC15 antibody, or antigen binding portion thereof described herein may include modifications to their respective Fc regions, typically to alter one or more of their physical or functional properties, such as effector function (e.g., antigen-dependent cellular cytotoxicity), Fc receptor binding, serum half-life, and complement fixation). Furthermore, anti-LRRC15 antibodies and antigen binding portions thereof disclosed herein can be chemically modified (e.g., one or more chemical moieties can be attached to the antibody) or be modified to alter its glycosylation, again to alter one or more properties of the antibody or antigen binding portion. In the context of Fc region modifications, the numbering of residues in the Fc region is that of the EU index of Kabat.

The antibodies and antigen binding portions thereof disclosed herein also include antibodies and antigen binding portions with modified (or blocked) Fc regions to provide altered effector functions as described in e.g., U.S. Pat. No. 5,624,821; U.S. Patent Publication numbers US2009/280114 and US2011/142858; and PCT Publication Number WO2006/0057702. Such modifications can further include alterations to enhance or suppress various reactions of the immune system, with possible beneficial effects in diagnosis and therapy.

### Altered Effector Functions

In some aspects, the anti-LRRC15 antibody, or antigen binding portion thereof comprises a variant Fc region that is modified (e.g., by amino acid substitution, deletion and/or insertion) relative to a parent Fc sequence (e.g., an unmodified Fc polypeptide that is subsequently modified to generate a variant) to increase or reduce the ability of the antibody or antigen binding portion to mediate one or more effector function(s) and/or to increase or decrease its binding to the Fc-gamma receptors (FcγRs), while retaining its antigen binding ability. Thus, in exemplary aspects, the anti-LRRC15 antibody, or antigen binding portion thereof may include one or more amino acid changes altering affinity for an effector ligand, such as an Fc receptor or the C1 component of complement. This approach is described in further detail in U.S. Pat. Nos. 5,624,821 and 5,648,260.

The interaction between the constant region of an antigen binding protein (such as a anti-LRRC15 antibody, or antigen binding portion thereof,) and various Fc receptors (FcR), including FcγRI (CD64), FcγRII (CD32) and FcγRIII (CD16), is believed to mediate the effector functions, such as ADCC and CDC, of the antigen binding protein. The Fc receptor is also important for antibody cross-linking, which can be important for anti-tumor immunity. In exemplary aspects, modifications can be made in the Fc region in order to generate an Fc variant promoting (a) increased or decreased antibody-dependent cell-mediated cytotoxicity (ADCC), (b) increased or decreased complement mediated cytotoxicity (CDC), (c) increased or decreased affinity for C1q, (d) increased or decreased affinity for a Fc receptor relative to the parent Fc, and/or (e) increased or decreased pharmacokinetic stability.

Alterations of the Fc region may include amino acid changes, such as substitutions, deletions, insertions, glycosylation, deglycosylation, and/or addition of multiple Fc regions. Combining amino acid modifications may be particularly desirable. For example, the variant Fc region may include two, three, four, five, or more substitutions therein, e.g., of the specific Fc region positions identified herein. In some aspects, the Fc region is altered by replacing at least one amino acid residue with a different amino acid residue such that the antibody has an altered affinity for an effector ligand but retains the antigen-binding ability of the parent antibody. For example, one or more amino acids selected from amino acid residues 234, 235, 236, 237, 297, 318, 320 and 322 can be replaced with a different amino acid residue. The effector ligand to which affinity is altered can be, for example, an Fc receptor or the C1 component of complement. This approach is described in further detail in U.S. Pat. Nos. 5,624,821 and 5,648,260, both by Winter et al. In some aspects, the C1q binding site may be removed from the Fc region by deleting or substituting, for example, the EKK sequence of human IgG1. In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al. In another example, one or more amino acid residues within amino acid positions 231 and 239 are altered to change the ability of the antibody to fix complement. This approach is described further in U.S. Pat. No. 6,180,377.

The Fc region of an antibody can be engineered to modulate effector functions, such as enhancing antigen-dependent cell-mediated cytotoxicity (ADCC) and/or complement-dependent cytotoxicity (CDC), or altering binding to Fc receptors. This can be achieved by introducing one or more amino acid substitutions in the Fc region. Alternatively, cysteine residues can be introduced to allow inter-chain disulfide bond formation, resulting in a homodimeric antibody with improved internalization capability and/or increased complement-mediated cell killing and ADCC (see, e.g., Caron P C et al., J. Exp. Med. 1992; 176 (4): 1191-5; Shopes B. J. Immunol. 1992; 148 (9): 2918-22).

In some aspects, an antibody or antigen binding portion thereof comprises a modified amino acid sequence that reduces or eliminates binding to most Fcγ receptors, thereby reducing uptake and toxicity in normal cells and tissues expressing these receptors, such as macrophages and liver sinusoidal cells. For example, an antibody with leucine (L) residues substituted by alanine (A) at positions 234 and 235 (LALA) has been shown to reduce Fc binding to FcγRs, consequently decreasing ADCC and complement binding/activation. In another example, an antibody comprises the substitution P329G in addition to the LALA double substitution (PG-LALA or the PG-LALA set of substitutions).

In some aspects, the anti-LRRC15 antibodies may be engineered to have different affinities and selectivities for Fc gamma receptors (FcγRs) by mutating the heavy chain constant region, including the hinge and Fc domains. Mutations can be introduced to either enhance or reduce FcγR binding. These mutations can increase or decrease FcγR-mediated cross-linking and/or signaling. For therapeutic targets, such as LRRC15, FcγR-mediated cross-linking of anti-LRRC15 antibodies have the potential to provide undesirable agonist signaling and potential for toxicity absent the introduction of certain modification to obviate this problem.

Binding sites on human IgG1 for FcγRI, FcγRII, FcγRIII and FcRn have been mapped and variants with improved binding have been described (see Shields, R. L. et al. (2001) Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/1332E and S239D/1332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/1332E variant showed an enhanced capacity to deplete B cells in monkeys (Lazar et al., 2006).

In some aspects, the anti-LRRC15 antibodies may be engineered for reduced FcγR binding and potential for cross-linking and/or signaling, specifically, reduced engagement of the “low affinity” FcγRs hCD32a/FcγRIIa, hCD32b/FcγRIIb, hCD16a/FcγRIIIa, and hCD16b/FcγRIIIb. Engagement of the “high affinity” receptor CD64/FcγRI is generally believed to be of lower concern due to saturation of this receptor with serum IgG. Therefore, in some aspects, the anti-LRRC15 antibodies may comprise an IgG1.3 Fc region, which is essentially devoid of binding to CD16, CD32a, CD32b and CD64 and lacks ADCC, ADCP and CDC functions (see U.S. Pat. No. 10,077,306 and U.S. Patent Publication No. US2022/0106400).

In some aspects, the Fc region may be engineered for increased antibody dependent cellular cytotoxicity (ADCC) and/or increased FcγR binding by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (as described e.g., in U.S. Pat. No. 6,737,056) wherein the numbering of the residues in the Fc region is that of the EU index as in Kabat. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

In some aspects, the Fc region is modified to decrease the ability of the anti-LRRC15 antibody or antigen binding portion thereof described herein to mediate effector function and/or to increase anti-inflammatory properties by modifying residues 243 and 264. In one aspect, the Fc region of the anti-LRRC15 antibody or antigen binding portion thereof is modified by changing the residues at positions 243 and 264 to alanine. In another aspect, the Fc region is modified to decrease the ability of the anti-LRRC15 antibody or antigen binding portion thereof to mediate effector function and/or to increase anti-inflammatory properties by modifying residues 243, 264, 267 and 328.

Other Fc modifications to the Fc region include those for reducing or ablating binding to FcγRs and/or complement proteins, thereby reducing or ablating Fc-mediated effector functions, such as ADCC, ADCP, and CDC. Modifications for altering binding to FcγRllb include one or more substitutions, insertions, and deletions at positions 234, 235, 236, 237, 239, 266, 267, 268, 269, 325, 326, 327, 328, and 332, wherein numbering is according to the EU index. In one aspect, the Fc variants provide selectively enhanced affinity to FcγRllb relative to one or more activating receptors. Exemplary substitutions include but are not limited to 234G, 235G, 236R, 237K, 267R, 269R, 325L, and 328R. Other Fc variants for enhancing binding to FcγRllb include 235Y/267E, 236D/267E, 236R/328R, 239D/268D, 239D/267E, 267E/268D, 267E/268E, and 267E/328F. Other modifications for reducing FcγR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

In certain aspects, the Fc region may be modified to remove an ADCC site. ADCC sites can be found, for example, in Molec. Immunol. 29 (5): 633-9 (1992) with regard to ADCC sites in IgG1. In addition, IgG1 mutants containing L235V, F243L, R292P, Y300L and P396L mutations were found to exhibit enhanced binding to FcγRIIIa and concomitantly enhanced ADCC activity in transgenic mice expressing human FcγRIIIa in models of B cell malignancies and breast cancer (Stavenhagen et al., 2007; Nordstrom et al., 2011). Other Fc mutants that may be used include: S298A/E333A/L334A, S239D/1332E, S239D/1332E/A330L, L235V/F243L/R292P/Y300L/P396L, and M428L/N434S. Specific examples of variant Fc domains are disclosed for example, in U.S. Pat. No. 6,096,871 and PCT Publication number WO 97/34631.

Optionally, the Fc region may comprise a non-naturally occurring amino acid residue at additional and/or alternative positions (see, e.g., U.S. Pat. Nos. 5,624,821; 6,277,375; 6,737,056; 6,194,551; 7,317,091; 8,101,720; PCT Patent Publication numbers WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

In one aspect, the hinge region of Fc is modified such that the number of cysteine residues in the hinge region is altered, e.g., increased or decreased. For example, in one aspect, the number of cysteine residues in the hinge region of CH1 is increased to provide increased the stability of the antibody or decreased to provide enhanced assembly of the light and heavy chains or as described in U.S. Pat. No. 5,677,425.

In some aspects, the, changes to the Fc region may be made to increase the biological half-life of the anti-LRRC15-directed therapeutic antibodies and immunoconjugates so as to facilitate less frequent dosing, with the concomitant increase convenience and decreases use of material (Presta (2005) J. Allergy Clin. Immunol. 116:731 at 734-35). Various approaches may be employed. For example, in certain aspects, this may be achieved by increasing the binding affinity of the Fc region for the neonatal Fc receptor (FcRn). For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

Other Fc variants for increased binding to FcRn and/or improved pharmacokinetic properties include substitutions at positions 259, 308, 428, and 434, including for example 259I, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 31 1A, 312A, 376A, 378Q, 380A, 382A, 434A (Shields et al, Journal of Biological Chemistry, 2001, 276 (9): 6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/311S (Dall'Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

In another aspect, the Fc hinge region may be mutated to decrease the biological half-life of the antibody or fragment. For example, one or more amino acid mutations may be introduced into the CH2-CH3 domain interface region of the Fc hinge fragment such that the antibody or fragment has impaired Staphylococcal protein A (SpA) binding relative to native Fc-hinge domain SpA binding as described in U.S. Pat. No. 6,165,745.

In certain aspects, hybrid IgG isotypes with particular biological characteristics may be used. For example, in certain aspects, one or more regions and/or mutations from an IgG2 or IgG4. In one aspect, the anti-LRRC15 antibody or antigen binding portion thereof described herein is an IgG4 isotype antibody or fragment comprising a serine to proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al. supra; position 241 is based on the Kabat numbering system). When using an IgG4 constant domain, it is usually preferable to include the substitution S228P, which mimics the hinge sequence in IgG1 and thereby stabilizes IgG4 molecules.

In another aspect, an IgG1/IgG3 hybrid variant may be constructed by substituting IgG1 positions in the CH2 and/or CH3 region with the amino acids from IgG3 at positions where the two isotypes differ. Thus, a hybrid variant IgG antibody may be constructed that comprises one or more substitutions, e.g., 274Q, 276K, 300F, 339T, 356E, 358M, 384S, 392N, 397M, 4221, 435R, and 436F. In other aspects described herein, an IgG1/IgG2 hybrid variant may be constructed by substituting IgG2 positions in the CH2 and/or CH3 region with amino acids from IgG1 at positions where the two isotypes differ. Thus, a hybrid variant IgG antibody may be constructed that comprises one or more substitutions, e.g., one or more of the following amino acid substitutions: 233E, 234L, 235L, 236G (referring to an insertion of a glycine at position 236), and 327A.

In some aspects, the variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other aspects, the Fc region may be modified to make it more compatible with a selected host cell. For example, one may remove the PA sequence near the N-terminus of a typical native Fc region, which may be recognized by a digestive enzyme in E. coli such as proline iminopeptidase.

The anti-LRRC15 antibody or antigen binding portion thereof disclosed herein may contain one or more glycosylation sites. Such glycosylation sites may result in increased immunogenicity of the antibody or fragment or an alteration of the pK of the antibody due to altered antigen-binding (Marshall et al. (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence.

Therefore, in some aspects, the glycosylation properties of the anti-LRRC15 antibody or antigen binding portion thereof described herein may be modified. For example, one or more glycosylation sites within the Fc domain may be modified or removed. Residues that are typically glycosylated (e.g., asparagine) may confer a cytolytic response. Such residues may be deleted or substituted with unglycosylated residues (e.g., alanine) to produce an aglycosylated antibody. In certain aspects, glycosylation can be altered to, for example, increase the affinity of the antibody for antigen. For example, one or more amino acid substitutions can be made that result in elimination of one or more variable region framework glycosylation sites to thereby eliminate glycosylation at that site. The resulting aglycosylation may increase the affinity of the antibody for antigen. Such an approach is described in further detail in U.S. Pat. Nos. 5,714,350 and 6,350,861 by Co et al. Glycosylation of the constant region on N297 may be prevented by mutating the N297 residue to another residue, e.g., N297A, and/or by mutating an adjacent amino acid, e.g., 298 to thereby reduce glycosylation on N297.

Additionally, or alternatively, an anti-LRRC15 antibody or antigen binding portion thereof described herein can be engineered with an altered type of glycosylation, such as a hypofucosylated antibody having reduced amounts of fucosyl residues or an antibody having increased bisecting GlcNac structures. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. Such carbohydrate modifications can be accomplished by, for example, expressing the antibody in a host cell with altered glycosylation machinery.

Cells with altered glycosylation machinery can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase (i.e., alpha-1,6-fucosyltransferase), such that antibodies expressed in such a cell line exhibit hypofucosylation. Recombinant host cells which have been genetically modified to inactivate the FUT8 gene encoding an alpha-1,6-fucosyltransferase are available. See, e.g., the POTELLIGENT™ technology system available from BioWa, Inc. (Princeton, N.J.) in which CHOKISV cells lacking a functional copy of the FUT8 gene produce monoclonal antibodies having enhanced antibody dependent cell mediated cytotoxicity (ADCC) activity that is increased relative to an identical monoclonal antibody produced in a cell with a functional FUT8 gene. Aspects of the POTELLIGENT™ technology system are described in U.S. Pat. Nos. 7,214,775 and 6,946,292, and PCT Publication numbers WO00/61739 and WO02/31240.

PCT Publication number WO 03/035835 by Presta describes a variant CHO cell line, Lec13 cells, with reduced ability to attach fucose to Asn (297)-linked carbohydrates, also resulting in hypofucosylation of antibodies expressed in that host cell (see also Shields, R. L. et al. (2002) J. Biol. Chem. 277:26733-26740). PCT Publication number WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of the antibodies described herein is pegylation. In some aspects, the anti-LRRC15 antibody or antigen binding portion described herein is pegylated to, for example, increase the biological (e.g., serum) half-life of the antibody. To pegylate an antibody, the antibody, or antigen binding portion thereof, typically is reacted with polyethylene glycol (PEG), such as a reactive ester or aldehyde derivative of PEG, under conditions in which one or more PEG groups become attached to the antibody or antigen binding portion thereof. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain aspects, the antibody to be pegylated is an aglycosylated antibody. Methods for pegylating proteins can be applied to the antibodies described herein. See for example, European patent number EP 0 154 316 by Nishimura et al. and European patent number EP 0 401 384 by Ishikawa et al.

Effector functions can be measured in a number of ways including for example via binding of the FcγRIII to Natural Killer cells or via FcγRI to monocytes/macrophages to measure for ADCC effector function. For example, an antigen binding protein of the present invention can be assessed for ADCC effector function in a Natural Killer cell assay. Examples of such assays can be found in Shields et al., 2001 J. Biol. Chem., Vol. 276, p 6591-6604; Chappel et al., 1993 J. Biol. Chem., Vol 268, p 25124-25131; Lazar et al., 2006 PNAS, 103; 4005-4010.

The affinities and binding properties of an Fc region for its ligand may be determined by a variety of in vitro assay methods (biochemical or immunological based assays) including, but not limited to, equilibrium methods (e.g., enzyme-linked immunosorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

With regard to the above-described modifications for increasing or decreasing one or more of the functional properties described herein (e.g., biochemical, immunochemical, cellular, physiological or other biological activities, as determined using methods known to the art and described herein), the resulting increase in a given parameter may represent a statistically significant increase of at least 10% of the measured parameter, more preferably at least 20%, 30%, 40%, 50%, 60%, 70%, 80%, 90%, 95%, or 100% (i.e., 2-fold), 3-fold, 5-fold or 10-fold. Conversely, the resulting decrease in a measured parameter may represent a statistically significant decrease of at least 10% of the measured parameter, e.g., at least 20%, 30%, 40%, 50%, 60%, 70%, 80%, 90%, 95%, or 100%, 3-fold, 5-fold or 10-fold.

Any of the above-described modifications may be employed alone or in combination with any of the above-described modifications or those described in the next section in order to further enhance or decrease effector functions or other desirable properties (e.g., stability, expression).

### Antibody Engineering of Variable Regions

In some aspects, the anti-LRRC15 antibodies or antigen binding portions thereof are engineered with modifications to framework residues within the variable domains of the parental antibody, e.g., to improve the properties of the antibody or antigen binding portion thereof. Typically, such framework modifications are made to decrease the immunogenicity of the anti-LRRC15 antibodies or antigen binding portions thereof. This is usually accomplished by replacing non-CDR residues in the variable domains (i.e., framework residues) in a parental (e.g., rodent) antibody with analogous residues from the immune repertoire of the species in which the antibody is to be used, e.g., human residues in the case of human therapeutics One approach is to “back-mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. Another approach is to revert to the original parental (e.g., rodent) residue at one or more positions of the engineered (e.g., humanized) antibody, e.g., to restore binding affinity that may have been lost in the process of replacing the framework residues. (See, e.g., U.S. Pat. Nos. 5,693,762, 5,585,089 and 5,530,101.)

In certain aspects, the anti-LRRC15 antibodies and antigen binding portions thereof are engineered (e.g., humanized) to include modifications in the framework and/or CDRs to improve their properties. Such engineered changes can be based on molecular modeling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, Md.; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al., (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice, the potential antigen binding regions based on model differ from the conventional “CDR”s or “hyper variable” loops. Commercial scientific software such as MOE (Chemical Computing Group) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases, certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689. In certain aspects, one or more glycosylation sites in either the light or heavy chain immunoglobulin variable regions, such as the framework regions, may be modified or removed to reduce immunogenicity. In particular aspects, it will be desirable to change certain amino acids containing exposed side-chains to another amino acid residue in order to provide for greater chemical stability of the final antibody, so as to avoid deamidation or isomerization. The deamidation of asparagine may occur on NG, DG, NG, NS, NA, NT, QG or QS sequences and result in the creation of an isoaspartic acid residue that introduces a kink into the polypeptide chain and decreases its stability (isoaspartic acid effect). Isomerization can occur at DG, DS, DA or DT sequences. In certain aspects, the antibodies provided herein do not contain deamidation or asparagine isomerism sites. For example, an asparagine (Asn) residue may be changed to Gln or Ala to reduce the potential for formation of isoaspartate at any Asn-Gly sequences, particularly within a CDR.

A similar problem may occur at an Asp-Gly sequence. Reissner and Aswad (2003) Cell. Mol. Life Sci. 60:1281. Isoaspartate formation may debilitate or completely abrogate binding of an antibody to its target antigen. See, Presta (2005) J. Allergy Clin. Immunol. 116:731 at 734.

In various aspect, the asparagine is changed to glutamine (Gln). It may also be desirable to alter an amino acid adjacent to an asparagine (Asn) or glutamine (Gln) residue to reduce the likelihood of deamidation, which occurs at greater rates when small amino acids occur adjacent to asparagine or glutamine. See, Bischoff & Kolbe (1994) J. Chromatog. 662:261. In addition, any methionine residues (typically solvent exposed Met) in CDRs may be changed to Lys, Leu, Ala, or Phe or other amino acids in order to reduce the possibility that the methionine sulfur would oxidize, which could reduce antigen-binding affinity and also contribute to molecular heterogeneity in the final antibody preparation. Id. Additionally, in order to prevent or minimize potential scissile Asn-Pro peptide bonds, it may be desirable to alter any Asn-Pro combinations found in a CDR to Gln-Pro, Ala-Pro, or Asn-Ala. Antibodies with such substitutions are subsequently screened to ensure that the substitutions do not decrease the affinity or specificity of the antibody for LRRC15, or other desired biological activity to unacceptable levels. See Table 1 for exemplary stabilizing CDR variants.

TABLE 1
Exemplary stabilizing CDR variants

| CDR Residue | Stabilizing Variant Sequence |
|---|---|
| Asn-Gly (N-G) | Gln-Gly, Ala-Gly, or Asn-Ala (Q-G), (A-G), or (N-A) |
| Asp-Gly (D-G) | Glu-Gly, Ala-Gly or Asp-Ala (E-G), (A-G), or (D-A) |
| Met (M) | Lys, Leu, Ala, or Phe (K), (L), (A), or (F) |
| Asn (N) | Gln or Ala |
| | (Q) or (A) |
| Asn-Pro (N-P) | Gln-Pro, Ala-Pro, or Asn-Ala (Q-P), (A-P), or (N-A) |

### Nucleic Acid Molecules

Also provided herein are nucleic acids, or a set of nucleic acids, that encode the anti-LRRC15 antibodies or antigen binding portions thereof. The nucleic acids include nucleotide sequences encoding heavy and/or light chain variable regions, antibody heavy or light chain sequences, antigen binding portions thereof, bispecific antibodies, or multispecific antibodies including sequence fragments thereof.

The nucleic acids may be present in whole cells, in a cell lysate, or in a partially purified or substantially pure form. A nucleic acid described herein can be, for example, DNA or RNA and may or may not contain intronic sequences. In certain aspects, the nucleic acid is a cDNA molecule. The nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques.

In some aspects, provided herein are nucleic acid molecules that encode the VH and/or VL sequences, or heavy and/or light chain sequences, of any of the anti-LRRC15 antibodies or antigen binding portions thereof. Host cells comprising the nucleotide sequences (e.g., nucleic acid molecules) described herein are encompassed herein. Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment, such as an antibody constant region or a flexible linker, whereby the two DNA fragments are joined such that the amino acid sequences encoded by the two DNA fragments remain in-frame.

An isolated DNA encoding the VH region can be converted to a full-length heavy chain gene by operatively linking the VH-encoding DNA to another DNA molecule encoding heavy chain constant regions (hinge, CH1, CH2 and/or CH3). Any sequences of human heavy chain constant region genes can be used for the purpose of the present disclosure (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

An isolated DNA encoding the VL region can be converted to a full-length light chain gene (as well as a Fab light chain gene) by operatively linking the VL-encoding DNA to another DNA molecule encoding the light chain constant region, CL. Any sequences of human light chain constant region genes can be used for the present disclosure (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

In some aspects, the nucleic acid molecules with conservative substitutions do not alter the resulting amino acid sequence upon translation of the nucleic acid molecule. In other aspects, the LRRC15 coding regions in the nucleic acid molecules are codon-optimized for improved expression.

In another aspect, provided herein are host cells transformed with the nucleic acids or expression vectors encoding the anti-LRRC15 antibodies or antigen binding portions of the present disclosure. The host cells can be any eukaryotic or prokaryotic cell capable of expressing the LRRC15 antibodies or antigen binding portions of the present disclosure, including immunoglobulin heavy and light chains thereof. The host cells may be used in methods for producing the LRRC15 antibodies described herein as further described herein.

### Methods of Production

The anti-LRRC15 antibodies or antigen binding portion thereof provided herein can be prepared using a wide variety of techniques including the use of hybridoma, recombinant, and phage display technologies, or a combination thereof.

Various methods for making monoclonal antibodies described herein are available in the art. For example, the monoclonal antibodies can be made using the hybridoma method first described by Kohler et al., Nature, 256:495 (1975), or any later developments thereof, or by recombinant DNA methods (U.S. Pat. No. 4,816,567). For example, monoclonal antibodies can be produced using hybridoma techniques including those known in the art and taught, for example, in Harlow et al, Antibodies: A Laboratory Manual, (Cold Spring Harbor Laboratory Press, 2nd ed., 1988); Hammer-ling, et al., in: Monoclonal Antibodies and T-Cell Hybridomas 563-681 (Elsevier, N.Y., 1981). Methods for producing and screening for specific antibodies using hybridoma technology are known in the art. In another example, antibodies useful in the methods and compositions described herein can also be generated using various art-recognized phage display methods, such as isolation from antibody phage libraries generated using the techniques described in McCafferty et al., Nature, 348:552-554 (1990). Clackson et al., Nature, 352:624-628 (1991) and Marks et al., J. Mol. Biol, 222:581-597 (1991) describe the isolation of murine and human antibodies, respectively, using phage libraries. Subsequent publications describe the production of high affinity (e.g., nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Human antibodies can be made by a variety of methods known in the art, including phage display methods using antibody libraries derived from human immunoglobulin sequences. See also, U.S. Pat. Nos. 4,444,887 and 4,716,111; and PCT publication numbers WO 98/46645, WO 98/50433, WO 98/24893, WO 98/16654, WO 96/34096, WO 96/33735, and WO 91/10741, the contents of which are herein incorporated by reference in their entireties. Human antibodies can also be produced using transgenic mice which express human immunoglobulin genes, and upon immunization are capable of producing a full repertoire of human antibodies in the absence of endogenous immunoglobulin production. For an overview of this technology for producing human antibodies, see, Lonberg and Huszar, 1995, Int. Rev. Immunol. 13:65-93. Phage display technology (McCafferty et al., Nature 348:552-553 (1990)) also can be used to produce human antibodies and antigen binding portions in vitro, from immunoglobulin variable (V) domain gene repertoires from unimmunized donors. Human antibodies can also be generated by in vitro activated B cells (see U.S. Pat. Nos. 5,567,610 and 5,229,275, the contents of which are herein incorporated by reference in their entireties). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1994, Bio/technology 12:899-903).

Chimeric antibodies can be prepared based on the sequence of a murine monoclonal antibody. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.).

Humanized forms of anti-LRRC15 antibodies (e.g., humanized forms of mouse anti-LRRC15 antibodies) are chimeric antibodies which contain minimal sequence derived from non-human immunoglobulin. Humanized antibodies are typically human immunoglobulins (recipient antibody) in which residues from a CDR or hypervariable region of the recipient are replaced by residues from a CDR or hypervariable region of a non-human species (donor antibody), such as mouse, rat, rabbit or nonhuman primate having the desired specificity, affinity, and capacity. In some instances, Fv framework region (FR) residues of the human immunoglobulin are replaced by corresponding non-human residues. Humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin consensus sequence. The humanized antibody optionally also will comprise at least a portion of an immunoglobulin constant region (Fc), typically that of a human immunoglobulin. For further details, see Jones et al., Nature 321: 522-525 (1986); Riechmann et al., Nature 332:323-329 (1988); and Presta, Curr. Op. Struct. Biol. 2:593-596 (1992).

The framework and CDR regions of a humanized antibody need not correspond precisely to the parental sequences, e.g., the donor antibody CDR or the consensus framework can be mutagenized by substitution, insertion and/or deletion of at least one amino acid residue so that the CDR or framework residue at that site does not correspond exactly to either the donor antibody or the consensus framework. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see e.g., Winnaker, From Genes to Clones (Veriagsgesellschaft, Weinheim, Germany 1987). In a family of immunoglobulins, each position in the consensus sequence is occupied by the amino acid occurring most frequently at that position in the family. Where two amino acids occur equally frequently, either can be included in the consensus sequence. As used herein, “Vernier zone” refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and can impact on the structure of CDRs and the affinity of the antibody. Human immunoglobulin (lg) sequences that can be used as a recipient are known in the art.

Framework residues in the human framework regions can be substituted with the corresponding residue from the CDR donor antibody to alter, preferably improve, antigen binding. These framework substitutions are identified by methods known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, including, but not limited to, those described in Jones et al., Nature 321:522 (1986); Verhoeyen et al., Science 239:1534 (1988), Sims et al., J. Immunol. 151:2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28 (4/5): 489-498 (1991); Studnicka et al., Protein Engineering 7 (6): 805-814 (1994); Roguska. et al., PNAS 91:969-973 (1994); PCT publication number WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592, 106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530, 101, 5,585,089, 5,225,539; 4,816,567, each incorporated by reference herein.

In one aspect, the antibodies described herein are human monoclonal antibodies. Such human monoclonal antibodies directed against LRRC15 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb MOUSE® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368 (6474): 856-859). Accordingly, the mice exhibit reduced expression of mouse IgM or K, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13:65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. et al. (1993) EMBO J. 12:821-830; Tuaillon et al. (1994) J. Immunol. 152:2912-2920; Taylor, L. et al. (1994) International Immunology 6:579-591; and Fishwild, D. et al. (1996) Nature Biotechnology 14:845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In certain aspects, antibodies described herein are raised using a mouse that carries human immunoglobulin sequences on transgenes and transchomosomes, such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise anti-LRRC15 antibodies described herein. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-LRRC15 antibodies described herein. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-LRRC15 antibodies described herein.

Additional mouse systems described in the art for raising human antibodies, e.g., human anti-LRRC15 antibodies, include (i) the VELOCIMMUNE® mouse (Regeneron Pharmaceuticals, Inc.), in which the endogenous mouse heavy and light chain variable regions have been replaced, via homologous recombination, with human heavy and light chain variable regions, operatively linked to the endogenous mouse constant regions, such that chimeric antibodies (human V/mouse C) are raised in the mice, and then subsequently converted to fully human antibodies using standard recombinant DNA techniques; and (ii) the MEMOR mouse (Merus Biopharmaceuticals, Inc.), in which the mouse contains unrearranged human heavy chain variable regions but a single rearranged human common light chain variable region. Such mice, and methods for raising antibodies, are described in, for example, WO 2009/15777, US 2010/0069614, WO 2011/072204, WO 2011/097603, WO 2011/163311, WO 2011/163314, WO 2012/148873, US 2012/0070861 and US 2012/0073004.

In some aspects, the anti-LRRC15 antibodies or antigen binding portions thereof, bispecific molecules (e.g., bispecific antibodies), and/or multispecific molecules (e.g., multispecific antibodies) are produced by culturing suitable host cells transformed with one or more nucleic acids or expression vectors encoding the LRRC15 antibodies or antigen binding portions thereof, bispecific molecules (e.g., bispecific antibodies), and/or multispecific molecules (e.g., multispecific antibodies) described in the present disclosure under conditions allowing for small-scale or large-scale production and purification of the antibodies or antigen binding portions thereof, bispecific molecules, and/or multispecific molecules.

In some aspects, a method for producing antibodies comprises culturing a cell transiently or stably expressing one or more constructs encoding one or more polypeptide chains in the antibody; and purifying the antibodies produced from the cultured cells. Any cell capable of producing a functional antibody may be used.

In some aspects, cells are stably transformed with DNAs encoding partial or full-length light and heavy chains obtained by standard molecular biology techniques (e.g., PCR amplification or cDNA cloning using a hybridoma that expresses the antibody of interest) inserted into expression vectors such that the genes are operatively linked to transcriptional and translational control sequences to facilitate their intended function of transcribing and translating the antibody gene(s) or antigen binding portions thereof. The expression vector and expression control sequences are chosen to be compatible with the expression host cell used. The antibody light chain and heavy chain genes or fragments thereof can be inserted into the same or separate expression vectors by standard methods. In some aspects, the light and heavy chain variable regions of the antibodies or antigen binding portions thereof described herein can be used to create full-length antibody genes of any antibody isotype by inserting them into expression vectors already encoding heavy chain constant and light chain constant regions of the desired isotype such that the VH segment is operatively linked to the CH segment(s) within the vector and the VL segment is operatively linked to the CL segment within the vector. Additionally, the expression vector can encode a signal peptide that facilitates secretion of the antibody chain from a host cell, whereby the antibody chain genes or antigen binding portions thereof are cloned into the vector such that the signal peptide is linked in-frame to the amino terminus of the antibody chain gene or antigen binding portion thereof. The signal peptide can be an immunoglobulin signal peptide or a heterologous signal peptide (i.e., a signal peptide from a non-immunoglobulin protein).

Mammalian host cells for expressing the recombinant antibodies or antigen binding portions described herein include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In other aspects, the cell is a yeast cell, an insect cell or a bacterial cell programmed to express the antibodies or antigen binding portions.

The antibodies in the present disclosure may be isolated from antibody expressing cells following culture and maintenance in any appropriate culture medium, such as RPMI, DMEM, and AIM VR. The antibodies can be purified using protein purification methodologies (e.g., affinity purification, chromatography), including the use of Protein-A or Protein-G immunoaffinity purification. Typically, the antibodies are engineered for secretion into culture supernatants for isolation therefrom.

An anti-LRRC15 antibody, antigen binding portion thereof, bispecific molecule, multispecific molecule, immunoconjugate, or antibody drug conjugate (ADC), as disclosed herein can be tested for desired properties, including particular binding specificities, binding affinities, targeted cell populations, described in the Examples, for example, protein-protein binding assays, biochemical screening assays, immunoassays, and/or cell-based assays. An aspect of the disclosure provides molecules that may be used to screen for antibodies or antigen binding portions thereof that bind LRRC15. Exemplary assays include, but are not limited to, fluorescense-activated cell sorting (FACS), enzyme-linked immunoabsorbent assay (ELISA), surface plasmon resonance (SPR) analysis, bio-layer interferometry (e.g., ForteBio assay), and Scatchard analysis.

In some aspects, the antibodies or antigen binding portions thereof are tested for specific binding to LRRC15 (e.g., human LRRC15). Methods for analyzing binding affinity, cross-reactivity, and binding kinetics of various anti-LRRC15 antibodies or antigen binding portions thereof include standard assays known in the art, for example, BIACORE™ surface plasmon resonance (SPR) analysis using a BIACORE™ 2000 SPR instrument (Biacore AB, Uppsala, Sweden) or bio-layer interferometry (e.g., ForteBio assay), as described in the Examples.

In some aspects, functional assays may be employed. Antibodies or antigen binding portions thereof can also be tested for their ability to inhibit the proliferation or viability of cells (either in vivo or in vitro), such as tumor cells, using various methods (e.g., 3H-thymidine incorporation, immunohistochemistry with proliferation markers, animal cancer models). For example, the antibodies or antigen binding portions thereof can be tested for their anti-tumor activity in vivo (e.g., as monotherapy or combination therapy), using in tumor xenograft models.

In some aspects, the functional activity of the LRRC15 antibodies or antigen binding portions thereof are assayed for their ability to reduce LRRC15 activation or function. In some aspects, the anti-LRRC15 antibody, or antigen binding portion thereof, inhibits (or is determined to inhibit) LRRC15 activation or function by, for example, 10% or more, for example, 20% or more, 30% or more, 40% or more, 50% or more, 60% or more, 70% or more, 80% or more, 90% or more, or 95% or more, relative to a control (e.g., a control antibody), as measured by ELISA.

Exemplary criteria for determining whether an anti-LRRC15 antibody or antigen binding portion thereof exhibits certain properties (e.g., binding, inhibition of activation, activation) are shown in Table 2.

TABLE 2
Exemplary criteria for anti-LRRC15 antibody properties

| Antibody Property | Positive |
|---|---|
| Binding to LRRC15, as | ≥2 SD above the mean of a negative control |
| assessed by ELISA | |
| Binding to LRRC15, as | ≥2 SD above the mean (median fluorescent |
| assessed by flow cytometry | intensity, MFI, on a homogeneous cell line |
| | or cell population) of a negative control |

### Pharmaceutical Compositions

Also provided herein are pharmaceutical compositions comprising an ADC as disclosed herein and a carrier (e.g., pharmaceutically acceptable carrier). Such compositions are useful for various therapeutic applications, such as cancer treatment.

In some aspects, the pharmaceutical compositions may further include other compounds, drugs, and/or agents for various therapeutic applications. Such compounds, drugs, and/or agents can include, for example, an anti-cancer agent, a chemotherapeutic agent, an immunosuppressive agent, an immunostimulatory agent, an immune checkpoint inhibitor, and/or an anti-inflammatory agent. Exemplary compounds, drugs, and agents that can be formulated together or separately with the ADC described in the next section.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition described herein may also include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. Except insofar as any media or agent is incompatible with the active compound, use thereof in the pharmaceutical compositions described herein is contemplated. A pharmaceutical composition may comprise a preservative or may be devoid of a preservative. Supplementary active compounds can be incorporated into the compositions.

A composition described herein can be administered via one or more routes of administration using one or more of a variety of methods. The route and/or mode of administration can vary depending upon the desired results. Routes of administration for the ADC described herein include e.g., intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an ADC described herein can be administered via a non-parenteral route, such as a topical, epidermal or mucosal route of administration, for example, intranasally, orally, vaginally, rectally, sublingually or topically.

### Uses and Methods

The ADC described herein have numerous in vitro and in vivo utilities as described herein.

### Cancer Treatment

In one aspect, provided herein is a method of treating cancer comprising administering to a subject in need thereof an ADC in an effective amount so that the growth of a cancerous tumor is inhibited or reduced and/or that regression and/or that prolonged survival is achieved.

In some aspects, provided herein is a method of treating a tumor in a subject in need thereof comprising administering to the subject an ADC disclosed herein in an effective amount so that the growth of the tumor is inhibited or reduced and/or that regression and/or that prolonged survival is achieved.

In some aspects, provided herein is a method of treating a tumor that expresses LRRC15 in a stromal cell in a subject in need thereof comprising administering to the subject an effective amount of an ADC described herein or the pharmaceutical composition described herein.

In some aspects, provided herein is a method of treating a tumor that does not express LRRC15 in a subject in need thereof comprising administering to the subject an effective amount of an ADC described herein or the pharmaceutical composition described herein.

In some aspects, provided herein is a method of treating a tumor that does not express LRRC15 in a subject in need thereof comprising administering to the subject an effective amount of an ADC described herein or the pharmaceutical composition described herein, wherein stromal cells expressing LRRC15 are present in the vicinity of the tumor cells.

In some aspects, the tumor is of a cancer comprising: colorectal cancer, breast cancer, lung cancer, ovarian cancer, pancreatic cancer, bladder cancer, uterine/cervical cancer, prostate cancer, testicular cancer, esophageal cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, or myelodysplastic syndromes. In some aspects, the tumor is of a cancer comprising a breast cancer.

In some aspects, provided herein is a method of inducing or improving a bystander effect of a cancer therapy in a subject in need thereof comprising administering to the subject an ADC described herein or the pharmaceutical composition described herein. In some aspects, the administering results in the killing of LRRC15-negative tumor cells. In some aspects, the LRRC15-negative tumor cells are adjacent to LRRC15-positive cells. In some aspects, the administering results in the killing of LRRC15-negative tumor cells adjacent to LRRC15-positive stromal cells. In some aspects, the LRRC15-positive stromal cells comprise LRRC15-expressing fibroblasts. In some aspects, the LRRC15-positive stromal cells comprise LRRC15-expressing mesenchymal stem cells.

In some aspects, the ADC described herein may be administered in combination with additional cytotoxic or therapeutic agent(s), for example as described herein.

Cancers that express LRRC15 whose growth may be inhibited using the ADC described herein include, but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer (e.g. estrogen-receptor positive breast cancer; estrogen-receptor positive breast cancer HER2-positive breast cancer; triple negative breast cancer); cancer of the peritoneum; cervical cancer; cholangiocarcinoma; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; liver cancer (e.g., hepatocellular carcinoma; hepatoma); intra-epithelial neoplasm; kidney or renal cancer; larynx cancer; leukemia; lung cancer (e.g., small-cell lung cancer, non-small cell lung cancer (NSCLC), squamous non-small cell lung cancer (SqNSCLC), non-squamous NSCLC, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; non-squamous cell cancer; teratocarcinoma; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblasts leukemia; and post-transplant lymphoproliferative disorder (PTLD), as well as abnormal vascular proliferation associated with phakomatoses, edema (such as that associated with brain tumors), tumors of primitive origins and Meigs' syndrome.

Additional cancers which express LRRC15 and can be treated using the ADC described herein include metastatic pancreatic cancer, metastatic adenocarcinoma of the pancreas, stomach cancer, fibrotic cancer, glioma, malignant glioma, diffuse intrinsic pontine glioma, recurrent childhood brain neoplasm renal cell carcinoma, clear-cell metastatic renal cell carcinoma, metastatic castration resistant prostate cancer, stage IV prostate cancer, metastatic melanoma, malignant melanoma, recurrent melanoma of the skin, melanoma brain metastases, malignant melanoma of head and neck, squamous cell non-small cell lung cancer, metastatic breast cancer, follicular lymphoma, advanced B-cell NHL, HL including diffuse large B-cell lymphoma (DLBCL), multiple myeloma, chronic myeloid leukemia, adult acute myeloid leukemia in remission, adult acute myeloid leukemia with Inv(16)(p13.1q22), CBFB-MYH11, adult acute myeloid leukemia with t(16:16) (p13.1:q22), CBFB-MYH11, adult acute myeloid leukemia with t(8:21)(d22:q22), RUNX1-RUNX1T1, adult acute myeloid leukemia with t(9:11)(p22:q23), MLLT3-MLL, adult acute promyelocytic leukemia with tO15:17)(q22:q12), PML-RARA, alkylating agent-related acute myeloid leukemia, Richter's syndrome, adult glioblastoma, adult gliosarcoma, recurrent glioblastoma, recurrent childhood rhabdomyosarcoma, recurrent Ewing sarcoma/peripheral primitive neuroectodermal tumor, recurrent neuroblastoma, recurrent osteosarcoma, colorectal cancer, MSI positive colorectal cancer, MSI negative colorectal cancer, nasopharyngeal nonkeratinizing carcinoma, recurrent nasopharyngeal undifferentiated carcinoma, cervical adenocarcinoma, cervical adenosquamous carcinoma; cervical squamous cell carcinoma, recurrent cervical carcinoma, anal canal squamous cell carcinoma, metastatic anal canal carcinoma, recurrent anal canal carcinoma, recurrent head and neck cancer, squamous cell of head and neck, head and neck squamous cell carcinoma (HNSCC), ovarian carcinoma, colon cancer, advanced GI cancer, gastric adenocarcinoma, gastroesophageal junction adenocarcinoma, bone neoplasms, soft tissue sarcoma, bone sarcoma, thymic carcinoma, urothelial carcinoma, Merkel cell carcinoma, recurrent Merkel cell carcinoma, mycosis fungoides, Sezary syndrome, neuroendocrine cancer, nasopharyngeal cancer, basal cell skin cancer, squamous cell skin cancer, dermatofibrosarcoma trotuberans, glioma, mesothelioma, myelodysplastic syndromes (MDS), myelofibrosis (MF), myeloproliferative neoplasms, acute myeloid leukemia (AML), or any combination thereof.

Cancers may be, e.g., metastatic or primary cancers; desmoplastic or non-desmoplastic cancers; or recurrent cancers.

In some aspects, the cancer is associated with fibrosis. In some aspects, the cancer is associated with infiltration of CD4+ regulatory T cells. In some aspects, the cancer is associated with infiltration of CD8+ regulatory T cells. In some aspects, the cancer is associate with infiltration of regulatory B cells. In some aspects, the cancer is associated with infiltration of myeloid-derived suppressor cells. In some aspects, the cancer is associated with infiltration of tumor-associated macrophages. In some aspects, the cancer is associated with infiltration of innate lymphoid cells. In some aspects, the cancer is associated with infiltration of cancer-associated fibroblasts. In some aspects, the cancer is associated with a radiation-related increase in the above cell types.

In some aspects, the cancer comprises a breast cancer. In some aspects, the breast cancer comprises a HR/HER breast cancer or a triple-negative breast cancer (TNBC).

In some aspects, the ADCs described herein are used to treat myelodysplastic syndromes (MDSs). MDSs are a diverse group of malignant disorders marked by bone marrow failure due to defective hematopoiesis and production of dysplastic cells. TGF-β is a primary driver in MDS (Geyh et al., Haematologica 2018; 103:1462-71) and agents that inhibit the function of TGF-β have been proposed as therapeutics (Mies et al., Curr Hematol Malig Rep 2016; 11:416-24). Furthermore, MDSCs are known to be dysregulated in MDS (Chen et al., JCI 2013; 123:4595-611) and agents that reduce MDSC levels in the bone marrow are potential therapeutics.

In some aspects, the cancer is resistant to checkpoint inhibitor(s). In some aspects, the cancer is intrinsically refractory or resistant (e.g., resistant to a PD-1 pathway inhibitor, PD-1 pathway inhibitor, or CTLA-4 pathway inhibitor). In some aspects, the resistance or refractory state of the cancer is acquired. In some aspects, the ADC described herein can be used in combination with checkpoint inhibitors to overcome resistance of the cancer to the checkpoint inhibitors. In some aspects, the ADC described herein can be used to treat tumors with a mesenchymal and/or EMT signature together with checkpoint inhibitors in combination or sequentially with agents that induce a mesenchymal phenotype, such as MAPK pathway inhibitors.

In some aspects, the ADCs described herein are used to enhance the viability of immune cells ex vivo, e.g., in adoptive NK cell transfer. Accordingly, in some aspects, ADCs are used in combination with adoptively transferred NK cells to treat cancer. In some aspects, the ADC described herein are used to treat tumors with MHC loss or MHC down-regulation, as monotherapy or in combination with NK activating or enhancing treatment.

In some aspects, the method described herein further comprises quantifying the expression of LRRC15 in the tumor cells and/or in the stromal cells using immunohistochemistry (IHC), wherein the quantification comprises calculating an H-score according to the formula: H-score=[(% tumor×1 (low intensity))+(% tumor×2 (medium intensity))+(% tumor×3 (high intensity)]. In some aspects, the tumor expresses LRRC15 by an H-score of at least 1. In some aspects, the tumor expresses LRRC15 by an H-score of greater than 1. In some aspects, the stromal cells proximal to the tumor express LRRC15 by an H-score of at least 1 or greater than 1.

To determine the histoscore, the pathologist estimates the percentage of stained cells in each intensity category within a specimen. Because expression of most biomarkers is heterogeneous the histoscore is a truer representation of the overall expression. The final histoscore ranges from 0 (no expression) to 300 (maximum expression), wherein an H score of 0 is considered a negative expression, and an H score of 1 to 300 is considered a positive expression.

### Combination Therapy

The ADCs described herein can be used in combination with various treatments or agents (or in the context of a multispecific antibody or bifunctional partner) known in the art for the treatment of disease or condition, as described herein.

In some aspects, a method of treating cancer comprises administering to a subject in need thereof an effective amount of an ADC described herein in combination with an additional therapeutic agent, such as a second antibody, a therapeutic protein or a small molecule drug. In some aspects, the therapeutic protein is a checkpoint inhibitor. In some aspects, the small molecule drug is a chemotherapeutic agent as described herein. In some aspects, the additional therapeutic agent comprises an anti-cancer agent.

Suitable anti-cancer agents for use in combination therapy with the ADC described herein include, but are not limited to, surgery, chemotherapeutic agents, growth inhibitory agents, cytotoxic agents, radiotherapy and agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC (Imatinib Mesylate)), COX-2 inhibitors (e.g., celecoxib), interferons, and cytokines; antagonists (e.g., neutralizing antibodies) that bind to and/or neutralize the activity of one or more of the following targets: PD-1, PD-L1, PD-L2 (e.g., pembrolizumab; nivolumab; MK-3475; AMP-224; MPDL3280A; MEDI0680; MSB0010718C; and/or MEDI4736); CTLA4 (e.g., tremelimumab (PFIZER) and ipilimumab); LAG3 (e.g., BMS-986016); CD 103; TIM-3 and/or other TIM family members; anti-VEGF antibodies (e.g., Bevacizumab); CEACAM1, CEACAM6, and/or other CEACAM family members; ErbB2, ErbB3, ErbB4, PDGFR-beta, BlyS, APRIL, BCMA or VEGF receptor(s), TRAIL/Apo2, PARP inhibitors (e.g., AZD-2281, Lynparza OCEACAM5arib, Rubraca Rucaparib; (Zejula) niraparib), DNA damage repair inhibitors (e.g., ATMi, ATRi, DNAPKi), and other bioactive and organic chemical agents, including those described in section VII. Combinations thereof are also specifically contemplated for the methods described herein.

In some aspects, the ADC is administered with an anti-cancer agent, such as an EGFR inhibitor; a HER2 inhibitor; a histone deacetylase inhibitor; a hormone; a mitotic inhibitor; a phosphatidylinositol-3-kinase (PI3K) inhibitor; an Akt inhibitor; a mammalian target of rapamycin (mTOR) inhibitor; a proteasomal inhibitor; a poly(ADP-ribose) polymerase (PARP) inhibitor; a Ras/MAPK pathway inhibitor; a centrosome declustering agent; a multi-kinase inhibitor; a serine/threonine kinase inhibitor; a tyrosine kinase inhibitor; a VEGF/VEGFR inhibitor; a microtubule targeting drug; a topoisomerase poison drug; or a combination thereof.

In some aspects, the ADC is administered along with an immune checkpoint inhibitor. Exemplary immune checkpoint inhibitors include, but are not limited to, agents (e.g., antibodies) that bind to PD-1, PD-L1, PD-L2, LAG-3, CTLA4, CCR8, TIGIT, ICOS, OX40, PVR, PVRIG, VISTA, TIM3, SIRPα, ILT2, ILT3, ILT4, or ILT5.

Any anti-PD-1 antibody can be used in combination with the ADC in the presently described methods. Various human monoclonal antibodies that bind specifically to PD-1 with high affinity have been disclosed in U.S. Pat. No. 8,008,449.

In some aspects, the anti-PD-1 antibody is pembrolizumab, nivolumab, cemiplimab, spartalizumab, camrelizumab, sintilimab, tislelizumab, toripalimab, dostarlimab, retifanlimab, pimivalimab, dostarlimab, serplulimab, zimberelimab, acrixolimab, MEDI-0680, AM-0001, STI-1110, AGEN2034, BCD-100, sasanlimab, BI 754091, or SSI-361.

In some aspects, the anti-PD-1 antibody used in combination with the ADC comprises the CDRH1, CDRH2, CDRH3, CDRL1, CDRL2, CDRL3, the VH and the VL, and/or the heavy and light chains of any of pembrolizumab, nivolumab, cemiplimab, spartalizumab, camrelizumab, sintilimab, tislelizumab, toripalimab, dostarlimab, retifanlimab, pimivalimab, MEDI-0680, GLS-010, AM-0001, STI-1110, AGEN2034, BCD-100, sasanlimab, BI 754091, or SSI-361.

In some aspects, the anti-PD-1 antibody used in combination with the ADC is selected from the group consisting of nivolumab (OPDIVO®; formerly designated 5C4, BMS-936558, MDX-1106, or ONO-4538), pembrolizumab (KEYTRUDA®; formerly designated lambrolizumab and MK-3475; see WO 2008/156712A1), PDR001 (see WO 2015/112900), MEDI-0680 (formerly designated AMP-514; see WO 2012/145493), REGN-2810 see WO 2015/112800), JS001 (see Liu and Wu, 2017), BGB-A317 (see WO 2015/035606 and US 2015/0079109), INCSHR1210 (SHR-1210; see WO 2015/085847; Liu and Wu, 2017), TSR-042 (ANB011; see WO 2014/179664), GLS-010 (WBP3055; see Liu and Wu, 2017), AM-0001 (see WO 2017/123557), STI-1110 (see WO 2014/194302), AGEN2034 (see WO 2017/040790), and MGD013 (see WO 2017/106061).

In some aspects, the anti-PD-1 antibody used in combination with the ADC is pembrolizumab (Merck; also known as KEYTRUDA®, lambrolizumab, and MK-3475; see, for example, WO 2008/156712). Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

In some aspects, the anti-PD-1 antibody used in combination with the ADC comprises nivolumab (also known as OPDIVO®, 5C4, BMS-936558, MDX-1106, and ONO-4538). Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (see, for example, U.S. Pat. No. 8,008,449; Wang et al., 2014 Cancer Immunol Res. 2 (9): 846-56).

In some aspects, the anti-PD-1 antibody used in combination with the ADC is cemiplimab (Regeneron; also known as LIBTAYO or REGN-2810; see, for example, WO 2015/112800 and U.S. Pat. No. 9,987,500).

In some aspects, the anti-PD-1 antibody used in combination with the ADC is spartalizumab (Novartis; also known as PDR001; see, for example, WO 2015/112900 and U.S. Pat. No. 9,683,048).

In some aspects, the anti-PD-1 antibody used in combination with the ADC is camrelizumab (Jiangsu Hengrui Medicine; also known as SHR-1210 or INCSHR1210; see, for example, WO 2015/085847; Si-Yang Liu et al., J. Hematol. Oncol. 10:136 (2017)).

In some aspects, the anti-PD-1 antibody used in combination with the ADC is MEDI-0680 (AstraZeneca; also known as AMP-514; see, for example, WO 2012/145493). In some aspects, the anti-PD-1 antibody is pimivalimab (also known as JTX-4014; see, for example, Papadopoulos, et al., 2022, IOTECH, Vol. 16, Supplement 1, 100284). In some aspects, the anti-PD-1 antibody is toripalimab (TAIZHOU JUNSHI PHARMA; also known as JS001; see, for example, Si-Yang Liu et al., J. Hematol. Oncol. 10:136 (2017)). In some aspects, the anti-PD-1 antibody is tislelizumab (Beigene; also known as BGB-A317; see, for example, WO 2015/35606 and US 2015/0079109). In some aspects, the anti-PD-1 antibody is dostarlimab (Tesaro Biopharmaceutical; also known as ANB011 or TSR-042; see, for example, WO2014/179664). In some aspects, the anti-PD-1 antibody is GLS-010 (Wuxi/Harbin Gloria Pharmaceuticals; also known as WBP3055; see, for example, Si-Yang Liu et al., J. Hematol. Oncol. 10:136 (2017)). In some aspects, the anti-PD-1 antibody is AM-0001 (Armo BioSciences).

In some aspects, the anti-PD-1 antibody is STI-1110 (Sorrento Therapeutics; see, for example, WO 2014/194302). In some aspects, the anti-PD-1 antibody is AGEN2034 (Agenus; see, for example, WO 2017/040790). In some aspects, the anti-PD-1 antibody is retifanlimab (Macrogenics, also known as MGA012, AEX-1188, and INCMGA-00012; see, for example, WO 2017/19846). In some aspects, the anti-PD-1 antibody is BCD-100 (Biocad; see, for example, Kaplon et al., mAbs 10 (2): 183-203 (2018). In some aspects, the anti-PD-1 antibody is sintilimab (Innovent; also known as IBI308; see, for example, WO 2017/024465, WO 2017/025016, WO 2017/132825, and WO 2017/133540). In some aspects, the anti-PD-1 antibody is sasanlimab (Pfizer; also known as PF-06801591; see, for example, US 2016/0159905). In some aspects, the anti-PD-1 antibody is BI 754091 (Boehringer Ingelheim; see, for example, Zettl M et al., Cancer. Res. (2018); 78 (13 Suppl): Abstract 4558). In some aspects, the anti-PD-1 antibody is SSI-361 (Lyvgen Biopharma Holdings Limited, see, for example, US 2018/0346569).

Other anti-PD-1 monoclonal antibodies suitable for the methods of the present disclosure have been described in, for example, U.S. Pat. Nos. 6,808,710, 7,488,802, 8,168,757, 8,354,509, and 9,205,148, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Examples of anti-PD-L1 antibodies useful in combination with the ADC according to the methods of the present disclosure include the antibodies disclosed in U.S. Pat. No. 9,580,507. In some aspects, the anti-PD-L1 antibody is atezolizumab, durvalumab, avelumab, envafolimab, cosibelimab, BMS-936559, STI-1014, CX-072, LY3300054, FAZ053, CS-1001, SHR-1316, CBT-502, KN035, or BGB-A333.

In some aspects, the anti-PD-L1 antibody is BMS-936559 (also known as 12A4, MDX-1105; see, e.g., U.S. Pat. No. 7,943,743 and WO 2013/173223).

In some aspects, the anti-PD-L1 antibody is STI-1014 (Sorrento; see, for example, WO 2013/181634). STI-104 is designated H6 in U.S. Pat. No. 9,175,082. In some aspects, the anti-PD-L1 antibody is CX-072 (Cytomx; see, for example, WO 2016/149201). In some aspects, the anti-PD-L1 antibody is LY3300054 (Eli Lilly Co.; see, e.g., WO 2017/034916). In some aspects, the anti-PD-L1 antibody is FAZ053 (Novartis). In some aspects, the anti-PD-L1 antibody is CK-301 (Checkpoint Therapeutics; see, for example, Gorelik et al., AACR: Abstract 4606 (April 2016)). CK-301 is also referred to as cosibelimab. In some aspects, the anti-PD-L1 antibody is CS-1001. See, for example, Zhou et al., Journal of Clinical Oncology, Meeting Abstract, 2020 ASCO Annual Meeting I, Lung Cancer-Non-Small Cell Metastatic, e21687, and Zhang et al., Cancer Research, 2020, 80 (16_Supplement): 3260. In some aspects, the anti-PD-L1 antibody is SHR-1316. See, for example, Mu et al., Thorac Cancer, 2021 May; 12 (9): 1373-1381, and Wu et al., Anals of Oncology, Abstract, Vol. 33, Supplement 2, S72, April 2022. In some aspects, the anti-PD-L1 antibody is CBT-502 (also known as TQB2450; see, for example, Wei et al., Mol Cancer Ther (2018) 17 (1_Supplement): A200). In some aspects, the anti-PD-L1 antibody is KN035 (3D Med/Alphamab; also referred to as envafolimab; see, for example, Zhang et al., Cell Discov. 7:3 (March 2017) and Shimizu et al., Invest New Drugs, 2022 October; 40 (5): 1021-1031).

In some aspects, the anti-PD-L1 antibody is BGB-A333 (BeiGene; see, for example, Desai et al., JCO 36 (15suppl):TPS3113 (2018) and Desai et al., 2023, British Journal of Cancer 128, 1418-1428). In certain aspects, the PD-L1 antibody is atezolizumab. Atezolizumab is a fully humanized IgG1 monoclonal anti-PD-L1 antibody. Atezolizumab (Roche) is also known as TECENTRIQ®; MPDL3280A, RG7446. See, for example, U.S. Pat. No. 8,217,149 and Herbst et al. (2013) J. Clin. Oncol. 31 (suppl): 3000). Atezolizumab is designated YW243.55S70 in U.S. Pat. No. 8,217,149. In certain aspects, the PD-L1 antibody is durvalumab. Durvalumab is a human IgG1 kappa monoclonal anti-PD-L1 antibody. Durvalumab (AstraZeneca) is also known as IMFINZI® or MEDI-4736. Durvalumab is designated 2.14H9OPT in U.S. Pat. No. 8,779,108. See, for example, WO 2011/066389. In certain aspects, the PD-L1 antibody is avelumab. Avelumab is a human IgG1 lambda monoclonal anti-PD-L1 antibody. Avelumab (Pfizer) is also known as BAVENCIO® or MSB0010718C. Avelumab is designated A09-246-2 in U.S. Pat. No. 9,624,298. See, for example, WO 2013/079174.

In some aspects, the anti-CTLA-4 antibody useful in combination with the ADC is tremelimumab, ipilimumab, botensilimab, BMS-986218, BMS-986288, BMS-986249, IBI310, MK-1308 (quavonlimab), AGEN-1884 (zalifrelimab), ONC-392, ADG116, or CS1002.

In some aspects, the anti-CTLA-4 antibody useful in combination with the ADC is MK-1308. MK-1308 is also known as quavonlimab. See, for example, Perets et al. 2021, Ann Oncol 32 (3): 395-403.

In some aspects, the anti-CTLA-4 antibody useful in combination with the ADC is AGEN-1884. AGEN-1884 is also known as zalifrelimab. See, for example, WO 2016/196237.

In some aspects, the anti-CTLA-4 antibody useful in combination with the ADC is tremelimumab. Tremelimumab, sold under the brand name IMJUDO®, is a fully human monoclonal antibody used for the treatment of hepatocellular carcinoma and non-small cell lung cancer. Tremelimumab (AstraZeneca) is also known as ticilimumab, CP-675,206; see WO 2000/037504 and Ribas, Update Cancer Ther. 2(3): 133-39 (2007)).

In some aspects, the anti-CTLA-4 antibody useful in combination with the ADC is ipilimumab. Ipilimumab (sold under the brand name YERVOY®, which was first approved for the treatment of metastatic melanoma, has since been approved for use in other cancers. Hoos et al. (2010) Semin. Oncol. 37:533; Hodi et al. (2010) N. Engl. J. Med. 363:711; Pardoll (2012) Nat. Immunol. 13(12):1129. In 2011, ipilimumab is a human antibody, which has an IgG1 constant region, was approved in the US and EU for the treatment of unresectable or metastatic melanoma based on an improvement in overall survival in a phase III trial of previously treated patients with advanced melanoma. Hodi et al. (2010) N. Engl. J. Med. 363:711. Tumor regressions and disease stabilization were frequently observed. Ipilimumab is also known as MDX-010 and 10D1. See U.S. Pat. No. 6,984,720.

In some aspects, the anti-CTLA-4 antibody is an activatable anti-CTLA-4 antibody, such as an activatable anti-CTLA-4 antibody wherein the light chains of the antibody comprise a cleavable moiety and a masking moiety at the amino termini. The masking moiety interferes with binding to CTLA-4, but is preferentially released in the tumor microenvironment after cleavage of the cleavable moiety by proteases that are more prevalent and/or active in tumors than in peripheral tissues (see, in particular, WO 2018/085555). Such preferential cleavage in the tumor microenvironment enables full CTLA-4 blocking, promoting anti-tumor immune response, while minimizing CTLA-4 blockade in normal tissue, thereby reducing the risk of potential systemic toxicity of an anti-CTLA-4 antibody. In some aspects, the activatable anti-CTLA-4 antibody is an activatable form of ipilimumab, such as an antibody comprising light chains modified to comprise a masking moiety and a cleavable moiety, as disclosed, for example, in WO 2018/085555. An example of an activatable anti-CTLA-4 antibody that has entered human clinical trials is BMS-986249 (NCT03369223: “A Study of BMS-986249 Alone and in Combination with Nivolumab in Advanced Solid Tumors”). In some aspects, the anti-CTLA-4 antibody is BMS-986249.

In some aspects, the anti-CTLA-4 antibody shows an enhanced Fcγ receptor (CD16) binding. Whether an anti-CTLA-4 antibody shows an enhanced Fcγ receptor binding is assessed by comparison with the Fcγ receptor binding of ipilimumab. Anti-CTLA-4 antibodies with enhanced Fcγ receptor (CD 16) binding have been proposed as therapeutic agents for treatment of cancer through depletion of Treg cells. See, in particular, WO 2014/089113. In some aspects, the anti-CTLA-4 antibody shows an Fcγ receptor (CD16) binding that is at least two-fold enhanced when compared to the Fcγ receptor binding of ipilimumab.

Examples of anti-CTLA-4 antibodies that show enhanced Fcγ receptor (i.e., FcγRIIIA or CD16) binding are nonfucosylated anti-CTLA-4 antibodies. In some aspects, the anti-CTLA-4 antibody is a nonfucosylated anti-CTLA-4 antibody. Non-fucosylated anti-CTLA-4 antibodies lack fucose residues in its N-linked glycans. In some aspects, the non-fucosylated anti-CTLA-4 antibody is produced by expressing the chains of the antibody in a mammalian cell under conditions that prevent fucosylation, including but not limited to use of mammalian cells with genetic modifications preventing fucosylation, or growth of the cells expressing the antibody in medium containing one or more chemical compounds that inhibit fucosylation. In some aspects, the genetic modification that prevents fucosylation is inactivation, e.g. knock-out, of the FUT8 gene. In some aspects, the anti-CTLA-4 antibody is a hypofucosylated anti-CTLA-4 antibody.

An exemplary nonfucosylated anti-CTLA-4 antibody that has entered human clinical trials is BMS-986218 (e.g., NCT03110107: “First-In-Human Study of Monoclonal Antibody BMS-986218 by Itself and in Combination with Nivolumab in Participants with Advanced Solid Tumors”). BMS-986218 is a nonfucosylated antibody developed to increase the effects of CTLA-4 blockade by enhancing binding to Fcγ receptor, thus promoting APC-mediated T cell priming. In some aspects, the anti-CTLA-4 antibody is BMS-986218. See, for example, PCT/US18/19868.

In some aspects, the Fc region of the anti-CTLA-4 antibody contains amino acid substitutions in the antibody constant region to enhance binding to activating Fcγ receptors. Exemplary substitutions are G236A, S239D, A330L and 1332E (all residue numbering per the EU numbering system). In some aspects, the anti-CTLA-4 antibody comprises a human IgG1 constant domain with S239D, A330L and 1332E mutations.

In some aspects, the anti-CTLA-4 antibody is an activatable and nonfucosylated anti-CTLA-4 antibody.

Human monoclonal antibodies that bind specifically to CTLA-4 with high affinity that are suitable for the methods of the present disclosure have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety.

Any anti-CCR8 antibody can be used in combination with the ADC in the presently described methods. In some aspects, the anti-CCR8 antibody is GS-1811, S-531011, HBM1022, DT-7012, RO7502175, LM-108, IPG0521, 14S15, 14S15h, or imzokitug. In some aspects, the anti-CCR8 antibody used in combination with the ADC comprises the CDRH1, CDRH2, CDRH3, CDRL1, CDRL2, CDRL3, the VH and the VL, and/or the heavy and light chains of any of GS-1811, S-531011, HBM1022, DT-7012, RO7502175, LM-108, IPG0521, CHS-114, 14S15, 14S15h, or imzokitug.

Human monoclonal antibodies that bind specifically to CCR8 with high affinity that are suitable for the methods of the present disclosure have been disclosed in, for example, International Publication Nos. WO 2023/230473, WO 2021/194942, WO 2022/146947, and WO 2025/145207, each of which is incorporated by reference herein in its entirety.

In some aspects, the anti-LAG-3 antibody useful in combination with the LRRC15 targeting agent according to the methods of the present disclosure is relatlimab (BMS-986016), IMP731 (H5L7BW), MK4280 (28G-10, favezelimab), REGN3767 (fianlimab), GSK2831781, humanized BAP050, IMP-701 (LAG525, ieramilimab), aLAG-3 (0414), aLAG-3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, ABL501

In some aspects, the anti-LAG-3 antibody useful in combination with the LRRC15 targeting agent comprises the CDRH1, CDRH2, CDRH3, CDRL1, CDRL2, CDRL3, the VH and the VL, and/or the heavy and light chains of any of relatlimab (BMS-986016), IMP731 (H5L7BW), MK4280 (28G-10, favezelimab), REGN3767 (fianlimab), GSK2831781, humanized BAP050, IMP-701 (LAG525, ieramilimab), aLAG-3 (0414), aLAG-3 (0416), Sym022, TSR-033, TSR-075, XmAb841 (XmAb22841), MGD013 (tebotelimab), BI754111, FS118, P 13B02-30, AVA-017, 25F7, AGEN1746, RO7247669, INCAGN02385, IBI-110, EMB-02, IBI-323, LBL-007, or ABL501.

In some aspects, the anti-LAG-3 antibody useful in combination with the ADC comprises relatlimab (BMS-986016). In some aspects, the anti-LAG-3 antibody comprises IMP731 (H5L7BW). In some aspects, the anti-LAG-3 antibody comprises MK4280 (28G-10, favezelimab). MK-4280 (28G-10, favezelimab) described in WO2016028672 and U.S. Publication No. 2020/0055938. In some aspects, the anti-LAG-3 antibody comprises REGN3767 (fianlimab). REGN3767 (fianlimab) is described, for example, in Burova E, et al., J. Immunother. Cancer (2016); 4 (Supp. 1): P195 and U.S. Pat. No. 10,358,495. In some aspects, the anti-LAG-3 antibody comprises GSK2831781. In some aspects, the anti-LAG-3 antibody comprises humanized BAP050. Humanized BAP050 is described, for example, in WO2017/019894. In some aspects, the anti-LAG-3 antibody comprises IMP-701 (LAG525, ieramilimab) IMP-701 (LAG525; ieramilimab) is described, for example, in U.S. Pat. No. 10,711,060 and U.S. Publ. No. 2020/0172617. In some aspects, the anti-LAG-3 antibody comprises aLAG-3 (0414). In some aspects, the anti-LAG-3 antibody comprises aLAG-3 (0416). In some aspects, the anti-LAG-3 antibody comprises Sym022. In some aspects, the anti-LAG-3 antibody comprises TSR-033. In some aspects, the anti-LAG-3 antibody comprises TSR-075. In some aspects, the anti-LAG-3 antibody comprises XmAb841 (XmAb22841). In some aspects, the anti-LAG-3 antibody comprises MGD013 (tebotelimab). In some aspects, the anti-LAG-3 antibody comprises BI754111. In some aspects, the anti-LAG-3 antibody comprises FS118. In some aspects, the anti-LAG-3 antibody comprises P 13B02-30. In some aspects, the anti-LAG-3 antibody comprises AVA-017. In some aspects, the anti-LAG-3 antibody comprises 25F7. 25F7 is described, for example, in U.S. Publ. No. 2011/0150892. In some aspects, the anti-LAG-3 antibody comprises AGEN1746. In some aspects, the anti-LAG-3 antibody comprises RO7247669. In some aspects, the anti-LAG-3 antibody comprises INCAGN02385. In some aspects, the anti-LAG-3 antibody comprises IBI-110. In some aspects, the anti-LAG-3 antibody comprises EMB-02. In some aspects, the anti-LAG-3 comprises IBI-323. In some aspects, the anti-LAG-3 antibody comprises LBL-007. In some aspects, the anti-LAG-3 antibody comprises ABL501.

In general, any anti-LAG-3 antibody useful in combination with the ADC can be used. Antibodies that bind to LAG-3 have been disclosed in Int'l Publ. No. WO/2015/042246 and U.S. Publ. Nos. 2014/0093511 and 2011/0150892, each of which is incorporated by reference herein in its entirety. Disclosure relating to the anti-LAG-3 antibodies described herein and other anti-LAG-3 antibodies useful in the methods of the present disclosure can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

Several experimental treatment protocols involve ex vivo activation and expansion of antigen specific T cells and adoptive transfer of these cells into recipients in order to generate antigen-specific T cells against tumors. Ex vivo activation in the presence of the anti-LRRC15 antibodies described herein with or without an additional immunostimulating therapy (e.g., an immune checkpoint inhibitor) can be expected to increase the frequency and activity of the adoptively transferred T cells.

In some aspects, the ADC described herein may also be administered with a standard of care treatment, or another treatment, such as radiation, surgery, or chemotherapy. The ADC may be combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90:3539-43).

### V. Kits

Also provided are kits comprising an ADC described herein and instructions for use.

In some aspects, the kits comprise the ADC in unit dosage form, such as in a single dose vial or a single dose pre-loaded syringe, optionally contained in a single vial or container, along with e.g., instructions for use in treating a cancer as described herein.

The present disclosure is further illustrated by the following examples, which should not be construed as further limiting. The contents of all figures and all references, Genbank sequences, issued patents, and published patent applications cited throughout this disclosure are expressly incorporated herein by reference.

### EXAMPLES

Commercially available reagents referred to in the Examples below were used according to manufacturer's instructions unless otherwise indicated. Unless otherwise noted, the present disclosure uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

The following Examples describe the isolation and characterization of anti-LRRC15 monoclonal antibodies. The CDR sequences, variable region sequences, and full-length heavy and light chain sequences of anti-LRRC15 antibodies are provided below, for example in Table 3.

TABLE 3
Summary table of amino acid sequences and nucleic acid sequences

| Description | Amino acid sequence or nucleic acid sequence | SEQ ID NO. |
|---|---|---|
| H. sapiens | MPLKHYLLLLVGCQAWGAGLAYHGCPSECTCSRASQVECTGARIVAVPTPLPW | 1 |
| LRRC15 | NAMSLQILNTHITELNESPFLNISALIALRIEKNELSRITPGAFRNLGSLRYL | |
| amino acid | SLANNKLQVLPIGLFQGLDSLESLLLSSNQLLQIQPAHFSQCSNLKELQLHGN | |
| sequence | HLEYIPDGAFDHLVGLTKLNLGKNSLTHISPRVFQHLGNLQVLRLYENRLTDI | |
| GenBank: | PMGTFDGLVNLQELALQQNQIGLLSPGLFHNNHNLQRLYLSNNHISQLPPSVF | |
| AAI01066.1 | MQLPQLNRLTLFGNSLKELSPGIFGPMPNLRELWLYDNHISSLPDNVFSNLRQ | |
| (Signal Peptide | LQVLILSRNQISFISPGAFNGLTELRELSLHTNALQDLDGNVFRMLANLQNIS | |
| Underlined) | LQNNRLRQLPGNIFANVNGLMAIQLQNNQLENLPLGIFDHLGKLCELRLYDNP | |
| | WRCDSDILPLRNWLLLNQPRLGTDTVPVCFSPANVRGQSLIIINVNVAVPSVH | |
| | VPEVPSYPETPWYPDTPSYPDTTSVSSTTELTSPVEDYTDLTTIQVTDDRSVW | |
| | GMTQAQSGLAIAAIVIGIVALACSLAACVGCCCCKKRSQAVLMQMKAPNEC | |
| H. sapiens | CAGCCTTGAGCCGTCCCTCGTCCTCCTCTCAGGCTCCCTCTTGTCCACGGCGG | 2 |
| LRRC15 | GCGGGCGCCGAGCTGCTGGCTATGCCACTGAAGCATTATCTCCTTTTGCTGGT | |
| nucleotide | GGGCTGCCAAGCCTGGGGTGCAGGGTTGGCCTACCATGGCTGCCCTAGCGAGT | |
| sequence | GTACCTGCTCCAGGGCCTCCCAGGTGGAGTGCACCGGGGCACGCATTGTGGCA | |
| GenBank: | GTGCCCACCCCTCTGCCCTGGAACGCCATGAGCCTGCAGATCCTCAACACGCA | |
| BC101065.1 | CATCACTGAACTCAATGAGTCCCCGTTCCTCAATATCTCAGCCCTCATCGCCC | |
| | TGAGGATTGAGAAGAATGAGCTGTCGCGCATCACGCCTGGGGCCTTCCGAAAC | |
| | CTGGGCTCGCTGCGCTATCTCAGCCTCGCCAACAACAAGCTGCAGGTTCTGCC | |
| | CATCGGCCTCTTCCAGGGCCTGGACAGCCTCGAGTCTCTCCTTCTGTCCAGTA | |
| | ACCAGCTGTTGCAGATCCAGCCGGCCCACTTCTCCCAGTGCAGCAACCTCAAG | |
| | GAGCTGCAGTTGCACGGCAACCACCTGGAATACATCCCTGACGGAGCCTTCGA | |
| | CCACCTGGTAGGACTCACGAAGCTCAATCTGGGCAAGAATAGCCTCACCCACA | |
| | TCTCACCCAGGGTCTTCCAGCACCTGGGCAACCTCCAGGTCCTCCGGCTGTAT | |
| | GAGAACAGGCTCACGGATATCCCCATGGGCACTTTTGATGGGCTTGTTAACCT | |
| | GCAGGAACTGGCTCTGCAGCAGAACCAGATTGGACTGCTCTCCCCTGGTCTCT | |
| | TCCACAACAACCACAACCTCCAGAGACTCTACCTGTCCAACAACCACATCTCC | |
| | CAGCTGCCACCCAGCGTCTTCATGCAGCTGCCCCAGCTCAACCGTCTTACTCT | |
| | CTTTGGGAATTCCCTGAAGGAGCTCTCTCCGGGGATCTTCGGGCCCATGCCCA | |
| | ACCTGCGGGAGCTTTGGCTCTATGACAACCACATCTCTTCTCTACCCGACAAT | |
| | GTCTTCAGCAACCTCCGCCAGTTGCAGGTCCTGATTCTTAGCCGCAATCAGAT | |
| | CAGCTTCATCTCCCCGGGTGCCTTCAACGGGCTAACGGAGCTTCGGGAGCTGT | |
| | CCCTCCACACCAACGCACTGCAGGACCTGGACGGGAACGTCTTCCGCATGTTG | |
| | GCCAACCTGCAGAACATCTCCCTGCAGAACAACCGCCTCAGACAGCTCCCAGG | |
| | GAATATCTTCGCCAACGTCAATGGCCTCATGGCCATCCAGCTGCAGAACAACC | |
| | AGCTGGAGAACTTGCCCCTCGGCATCTTCGATCACCTGGGGAAACTGTGTGAG | |
| | CTGCGGCTGTATGACAATCCCTGGAGGTGTGACTCAGACATCCTTCCGCTCCG | |
| | CAACTGGCTCCTGCTCAACCAGCCTAGGTTAGGGACGGACACTGTACCTGTGT | |
| | GTTTCAGCCCAGCCAATGTCCGAGGCCAGTCCCTCATTATCATCAATGTCAAC | |
| | GTTGCTGTTCCAAGCGTCCATGTCCCCGAGGTGCCTAGTTACCCAGAAACACC | |
| | ATGGTACCCAGACACACCCAGTTACCCTGACACCACATCCGTCTCTTCTACCA | |
| | CTGAGCTAACCAGCCCTGTGGAAGACTACACTGATCTGACTACCATTCAGGTC | |
| | ACTGATGACCGCAGCGTTTGGGGCATGACCCAGGCCCAGAGCGGGCTGGCCAT | |
| | TGCCGCCATTGTAATTGGCATTGTCGCCCTGGCCTGCTCCCTGGCTGCCTGCG | |
| | TCGGCTGTTGCTGCTGCAAGAAGAGGAGCCAAGCTGTCCTGATGCAGATGAAG | |
| | GCACCCAATGAGTGTTAAAGAGGCAGGCTGGAGCAGGGCTGGGGAATGATGGG | |
| | ACTGGAGGACCTGGGAATTTCATCTTTCTGCCTCCACCCCTGGGTCCATGGAG | |
| | CTTTCCCGTGATTGCTCTTTCTGGCCCCAGAGAAAGGTGAGCCTACCTCTTCC | |
| | TGACTTGCCTGATTCTCCCGTAGAGAAGCAGGTCGTGCCGGACCTTCCTACAA | |
| | TCAGGAAGATAGATCCAACTGGCCATGGCAAAAGCCCTGGGGATTTCCGATTC | |
| | ATACCCCTGGGCTTCCTTCGAGAGGGCTCTTCCTCCAAATCCTCCCCACCTGT | |
| | CCTCCAAGAACAGCCTTCCCTGCGCCCAGGCCCCCTCCGGGCCTCTGTAGACT | |
| | CAGTTAGTCCACAGCCTGCTCACTTCGTGGGAATAGTTCTCCGCTGAGATAGC | |
| | CCCTCTCGCCTAAGTATTATGTAAGTTGATTTCCCTTCTTTTGTTTCTCTTGT | |
| | TTGTGCTACGGCTTGACCCAGCATGTCCCCTCAAATGAAAGTTCTCCCCTTGA | |
| | TTTTCTGCTCCTGAAGGCAGGGTGAGTTCTCTCCTCAAAGAAGACTTCAAACC | |
| | ATTTAACTGGTTTCTTAAGAGCCGTCAATCAGCCTGGTTTTGGGGATGCTATG | |
| | AAAGAGAGAAGGAAAATCATGCCGCTCAGTTCCTGGAGACAGAAGAGCCGTCA | |
| | TCAGTGTCTCACTTGTGATTTTTATCTGGAAAAGGAAGAAACACCCCAGCACA | |
| | GCAAGCTCAGCCTTTTAGAGAAGGATATTTCCAAACTGCAAACTTTGCTTTGA | |
| | AAAGTTTAGCCCTTTAAGGAATGAAATCATGTAGAATTTTGGACTTCTAAAAA | |
| | CATTAAAATCAGCTTATTAATACGGGATAGAGAAAGAAATCTGGTGCCTGGGG | |
| | GTCCCTGTGTTCACCCCTAGAGTTTGTTTTAAAATTTTTAATTGAAGCATGTG | |
| | AAGTGTACGTGCAGAAAAGTGGGAACATGATAGTGTATGGCTTGGTGGATTTT | |
| | CACAAACTGAACATACCTGTGTAATCAGCATCTAGACCCAGACCCAGAGCATC | |
| | ACAAATATCCCCCATCCTGGGCTTTTCCCAGAGGAGATGGGGGCTTCTGAAGA | |
| | TGGACTTACCTGGGACCTGCCCCCCATGAGCCAGGACGGTCCCCCCACAGTCA | |
| | GCCTGTGCAAAGGCCCCGTGGCCAGGGGTGGAGGAGAATATGTGGGTGTGGAC | |
| | AGGATGGGAGACTGTGGCCTGAACAGGAGATTTTATTATATCTGGAGACCCTG | |
| | AGAGACCCTGAGACCTGGGGCACCATGGCTGGCCAGGTCAGAAGCATCCTGAC | |
| | TGCAGAGGTCCGTGCAGCCACACCCTCTTCCCTGCCAGCAAGCTGTCTGCGGC | |
| | TCATCGGAGGC | |
| MDC001 | TFAMS | 3 |
| HCDR1 amino | | |
| acid sequence | | |
| MDC001 | VISGTGGSTFYTDSVKG | 4 |
| HCDR2 amino | | |
| acid sequence | | |
| MDC001 | AVTVNTRGFFDY | 5 |
| HCDR3 amino | | |
| acid sequence | | |
| MDC001 | QVQLLESGGGLVQPGGSLRLSCAASGFTFSTFAMSWVRQAPGKGLDWVSVIS | 6 |
| VH amino acid | GTGGSTFYTDSVKGRFTISRDNSENTLYLEMNSLRAEDTAVYFCAKAVTVNT | |
| sequence | RGFFDYWGQGTLVTVSS | |
| MDC001 | (pE)VQLLESGGGLVQPGGSLRLSCAASGFTFSTFAMSWVRQAPGKGLDWVS | 7 |
| VH amino acid | VISGTGGSTFYTDSVKGRFTISRDNSENTLYLEMNSLRAEDTAVYFCAKAVT | |
| sequence with N- | VNTRGFFDYWGQGTLVTVSS | |
| terminal | | |
| pyroglutamate | | |
| MDC001 | CAAGTGCAGCTGCTGGAATCGGGCGGCGGACTTGTGCAGCCGGGCGGATCCCT | 8 |
| VH nucleotide | GAGGCTGAGCTGTGCTGCCTCCGGATTCACTTTCTCCACCTTCGCCATGTCCT | |
| sequence | GGGTCCGCCAAGCGCCCGGAAAGGGCTTGGATTGGGTGTCCGTCATTTCGGGG | |
| | ACCGGGGGTAGCACTTTTTACACCGACTCCGTGAAGGGCCGCTTCACCATCTC | |
| | AAGAGACAACTCTGAAAACACCCTGTACCTCGAGATGAATAGCCTCCGGGCCG | |
| | AGGATACTGCAGTCTACTTCTGCGCCAAAGCCGTGACCGTGAACACCCGGGGA | |
| | TTTTTCGACTATTGGGGACAGGGTACTCTCGTGACGGTGTCCTCA | |
| MDC001 | RTSQVIRNDLG | 9 |
| LCDR1 amino | | |
| acid sequence | | |
| MDC001 | DASSLQS | 10 |
| LCDR2 amino | | |
| acid sequence | | |
| MDC001 | LQDNNYPWT | 11 |
| LCDR3 amino | | |
| acid sequence | | |
| MDC001 | EIVLTQSPSSLSASVGDRVTITCRTSQVIRNDLGWYQQKPGKAPKLLIYDAS | 12 |
| VL amino acid | SLQSGVPSRFSGSGSGTDFTLTISSLQPEDFATYYCLQDNNYPWTFGQGTKV | |
| sequence | AIK | |
| MDC001 | GAGATTGTGCTCACTCAATCCCCCTCCTCGCTGTCGGCCTCCGTGGGGGATCG | 13 |
| VL nucleotide | CGTGACCATCACTTGCCGGACCAGCCAGGTCATCAGAAACGACCTGGGATGGT | |
| sequence | ACCAGCAGAAGCCGGGGAAGGCACCGAAGCTGCTCATCTACGACGCTAGCAGC | |
| | CTGCAGTCCGGAGTGCCATCAAGGTTCTCCGGCTCGGGTTCCGGAACTGACTT | |
| | CACCTTGACCATCTCATCCCTGCAACCCGAAGATTTCGCCACCTACTACTGTC | |
| | TGCAAGACAACAACTATCCTTGGACGTTTGGACAGGGCACCAAAGTCGCGATT | |
| | AAG | |
| MDC001 | QVQLLESGGGLVQPGGSLRLSCAASGFTFSTFAMSWVRQAPGKGLDWVSVIS | 14 |
| HC amino acid | GTGGSTFYTDSVKGRFTISRDNSENTLYLEMNSLRAEDTAVYFCAKAVTVNT | |
| sequence | RGFFDYWGQGTLVTVSSASTKGPSVFPLAPSSKSTSGGTAALGCLVKDYFPE | |
| | PVTVSWNSGALTSGVHTFPAVLQSSGLYSLSSVVTVPSSSLGTQTYICNVNH | |
| | KPSNTKVDKRVEPKSCDKTHTCPPCPAPELLGGPSVFLFPPKPKDTLMISRT | |
| | PEVTCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSREEMTK | |
| | NQVSLTCLVKGFYPSDIAVEWESNGQPENNYKTTPPVLDSDGSFFLYSKLTV | |
| | DKSRWQQGNVFSCSVMHEALHNHYTQKSLSLSPG | |
| MDC001 | QVQLLESGGGLVQPGGSLRLSCAASGFTFSTFAMSWVRQAPGKGLDWVSVIS | 15 |
| HC amino acid | GTGGSTFYTDSVKGRFTISRDNSENTLYLEMNSLRAEDTAVYFCAKAVTVNT | |
| sequence with C- | RGFFDYWGQGTLVTVSSASTKGPSVFPLAPSSKSTSGGTAALGCLVKDYFPE | |
| terminal lysine | PVTVSWNSGALTSGVHTFPAVLQSSGLYSLSSVVTVPSSSLGTQTYICNVNH | |
| | KPSNTKVDKRVEPKSCDKTHTCPPCPAPELLGGPSVFLFPPKPKDTLMISRT | |
| | PEVTCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSREEMTK | |
| | NQVSLTCLVKGFYPSDIAVEWESNGQPENNYKTTPPVLDSDGSFFLYSKLTV | |
| | DKSRWQQGNVFSCSVMHEALHNHYTQKSLSLSPGK | |
| MDC001 | (pE)VQLLESGGGLVQPGGSLRLSCAASGFTFSTFAMSWVRQAPGKGLDWVS | 16 |
| HC amino acid | VISGTGGSTFYTDSVKGRFTISRDNSENTLYLEMNSLRAEDTAVYFCAKAVT | |
| sequence with N- | VNTRGFFDYWGQGTLVTVSSASTKGPSVFPLAPSSKSTSGGTAALGCLVKDY | |
| terminal | FPEPVTVSWNSGALTSGVHTFPAVLQSSGLYSLSSVVTVPSSSLGTQTYICN | |
| pyroglutamate | VNHKPSNTKVDKRVEPKSCDKTHTCPPCPAPELLGGPSVFLFPPKPKDTLMI | |
| | SRTPEVTCVVVDVSHEDPEVKFNWYVDGVEVHNAKTKPREEQYNSTYRVVSV | |
| | LTVLHQDWLNGKEYKCKVSNKALPAPIEKTISKAKGQPREPQVYTLPPSREE | |
| | MTKNQVSLTCLVKGFYPSDIAVEWESNGQPENNYKTTPPVLDSDGSFFLYSK | |
| | LTVDKSRWQQGNVFSCSVMHEALHNHYTQKSLSLSPG | |
| MDC001 | EIVLTQSPSSLSASVGDRVTITCRTSQVIRNDLGWYQQKPGKAPKLLIYDAS | 17 |
| LC amino acid | SLQSGVPSRFSGSGSGTDFTLTISSLQPEDFATYYCLQDNNYPWTFGQGTKV | |
| sequence | AIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC | |
| MDC001 | CAAGTGCAGCTGCTGGAATCGGGCGGCGGACTTGTGCAGCCGGGCGGATCCCT | 18 |
| HC nucleotide | GAGGCTGAGCTGTGCTGCCTCCGGATTCACTTTCTCCACCTTCGCCATGTCCT | |
| sequence | GGGTCCGCCAAGCGCCCGGAAAGGGCTTGGATTGGGTGTCCGTCATTTCGGGG | |
| | ACCGGGGGTAGCACTTTTTACACCGACTCCGTGAAGGGCCGCTTCACCATCTC | |
| | AAGAGACAACTCTGAAAACACCCTGTACCTCGAGATGAATAGCCTCCGGGCCG | |
| | AGGATACTGCAGTCTACTTCTGCGCCAAAGCCGTGACCGTGAACACCCGGGGA | |
| | TTTTTCGACTATTGGGGACAGGGTACTCTCGTGACGGTGTCCTCAGCATCCAC | |
| | TAAGGGGCCTAGCGTCTTTCCGCTGGCCCCGTCCTCCAAGTCCACTTCGGGTG | |
| | GAACCGCGGCACTGGGGTGCCTCGTGAAGGACTACTTCCCCGAGCCGGTCACC | |
| | GTGTCCTGGAACTCGGGAGCCCTGACCTCCGGAGTGCATACTTTCCCTGCGGT | |
| | GCTGCAGTCCTCCGGGCTCTACTCGCTGTCAAGCGTGGTCACCGTCCCGAGCT | |
| | CATCCCTGGGTACTCAGACCTACATTTGCAACGTGAACCACAAACCTTCCAAC | |
| | ACCAAGGTCGACAAGAGGGTGGAGCCTAAGAGCTGCGACAAGACCCACACCTG | |
| | TCCCCCGTGTCCCGCCCCTGAGCTGCTGGGCGGCCCCAGCGTGTTCCTCTTCC | |
| | CGCCTAAGCCGAAGGACACTCTGATGATCTCGAGAACCCCTGAAGTGACCTGT | |
| | GTGGTGGTGGATGTGTCCCACGAGGATCCGGAAGTGAAGTTCAATTGGTACGT | |
| | GGACGGAGTGGAAGTCCATAACGCCAAGACCAAGCCCCGCGAGGAACAGTACA | |
| | ACTCAACTTACCGGGTGGTGTCAGTGCTGACCGTGCTGCACCAAGATTGGCTG | |
| | AACGGGAAGGAGTACAAGTGCAAAGTCTCCAACAAGGCGCTGCCGGCCCCCAT | |
| | TGAAAAGACCATCAGCAAGGCTAAGGGCCAGCCCCGGGAACCACAGGTCTACA | |
| | CCTTGCCCCCTTCCCGGGAGGAAATGACCAAGAACCAAGTGTCGCTGACGTGC | |
| | CTGGTCAAGGGCTTTTATCCATCTGACATCGCCGTGGAGTGGGAAAGCAACGG | |
| | CCAGCCGGAAAACAACTACAAGACTACCCCGCCTGTGCTGGACTCCGACGGCT | |
| | CGTTCTTCCTGTATTCCAAGCTCACCGTGGATAAGTCCAGATGGCAGCAGGGC | |
| | AATGTGTTCAGCTGCAGCGTGATGCATGAGGCCCTGCACAACCACTACACTCA | |
| | GAAATCACTGTCCCTTTCCCCCGGA | |
| MDC001 | GAGATTGTGCTCACTCAATCCCCCTCCTCGCTGTCGGCCTCCGTGGGGGATCG | 19 |
| LC nucleotide | CGTGACCATCACTTGCCGGACCAGCCAGGTCATCAGAAACGACCTGGGATGGT | |
| sequence | ACCAGCAGAAGCCGGGGAAGGCACCGAAGCTGCTCATCTACGACGCTAGCAGC | |
| | CTGCAGTCCGGAGTGCCATCAAGGTTCTCCGGCTCGGGTTCCGGAACTGACTT | |
| | CACCTTGACCATCTCATCCCTGCAACCCGAAGATTTCGCCACCTACTACTGTC | |
| | TGCAAGACAACAACTATCCTTGGACGTTTGGACAGGGCACCAAAGTCGCGATT | |
| | AAGCGCACCGTGGCCGCCCCTAGCGTGTTTATCTTCCCTCCCTCGGATGAGCA | |
| | GCTTAAGTCAGGCACCGCATCCGTGGTCTGCCTGCTCAACAACTTCTACCCGA | |
| | GGGAAGCCAAAGTGCAGTGGAAAGTGGACAACGCGCTCCAGTCGGGAAACTCC | |
| | CAGGAGTCCGTGACCGAACAGGACTCCAAGGACAGCACTTATTCCCTGTCCTC | |
| | CACTCTGACGCTGTCAAAGGCCGACTACGAGAAGCACAAGGTCTACGCCTGCG | |
| | AAGTGACCCATCAGGGGCTTTCCTCGCCCGTGACTAAGAGCTTCAATCGGGGC | |
| | GAATGC | |
| MDC001.VK | ATGCGCGCCTGGATCTTCTTCCTGCTTTGCCTGGCCGGCCGGGCGCTCGCC | 20 |
| signal peptide | | |
| nucleotide | | |
| sequence | | |
| MDC001.VH | ATGCGGGCTTGGATCTTCTTCCTGCTGTGCCTGGCCGGGAGAGCGCTGGCC | 21 |
| signal peptide | | |
| nucleotide | | |
| sequence | | |
| MDC002 | SSNWWS | 22 |
| HCDR1 amino | | |
| acid sequence | | |
| MDC002 | EWIGEIYHSGSTNYNPSLKS | 23 |
| HCDR2 amino | | |
| acid sequence | | |
| MDC002 HCDR3 | DNWGSFHAFDF | 24 |
| amino acid | | |
| sequence | | |
| MDC002 | QVQLQESGPGLVKPSGTLSLTCAVSGGSISSSNWWSWVRQAPGKGLEWIGEIYH | 25 |
| VH amino acid | SGSTNYNPSLKSRVTMSVDKSKNQFSLKLTSVTAADTAVYYCARDNWGSFHAFD | |
| sequence | FWGQGTMVTVSS | |
| MDC002 | (pE)VQLQESGPGLVKPSGTLSLTCAVSGGSISSSNWWSWVRQAPGKGLEWIGE | 26 |
| VH amino acid | IYHSGSTNYNPSLKSRVTMSVDKSKNQFSLKLTSVTAADTAVYYCARDNWGSFH | |
| sequence with N- | AFDFWGQGTMVTVSS | |
| terminal | | |
| pyroglutamate | | |
| MDC002 | RASQSIGSNLH | 27 |
| LCDR1 amino acid | | |
| sequence | | |
| MDC002 | YTSQSFS | 28 |
| LCDR2 amino acid | | |
| sequence | | |
| MDC002 LCDR3 | HQSTGLPHT | 29 |
| amino acid | | |
| sequence | | |
| MDC002 | EIVLTQSPDFQSVTPKEKVTITCRASQSIGSNLHWYQQKPDQSPKLLIKYTSQS | 30 |
| VL amino acid | FSGVPSRFSGSGSGTDFTLTISSLEAEDAATYFCHQSTGLPHTFGGGTKVEIK | |
| sequence | | |
| MDC003 | DYYMT | 31 |
| HCDR1 amino | | |
| acid sequence | | |
| MDC003 HCDR2 | DIGSSGGSVDYADSVKG | 32 |
| amino acid | | |
| sequence | | |
| MDC003 HCDR3 | YNWNDRGYDGFDI | 33 |
| amino acid | | |
| sequence | | |
| MDC003 | QVQLVESGGDLVTPGGSLRLSCAASGFTFSDYYMTWIRQAPGKGLEWISDIGSS | 334 |
| VH amino acid | GGSVDYADSVKGRFTISRDNAKNSLYLQMSSLRAEDTAVYYCARYNWNDRGYDG | |
| sequence | FDIWGQGTMVTVSS | |
| MDC003 | (pE)VQLVESGGDLVTPGGSLRLSCAASGFTFSDYYMTWIRQAPGKGLEWISDI | 335 |
| VH amino acid | GSSGGSVDYADSVKGRFTISRDNAKNSLYLQMSSLRAEDTAVYYCARYNWNDRG | |
| sequence with N- | YDGFDIWGQGTMVTVSS | |
| terminal | | |
| pyroglutamate | | |
| MDC003 LCDR1 | RASQGISSALA | 36 |
| amino acid | | |
| sequence | | |
| MDC003 LCDR2 | DASSLES | 37 |
| amino acid | | |
| sequence | | |
| MDC003 LCDR3 | QQFNSYPHT | 38 |
| amino acid | | |
| sequence | | |
| MDC003 | EIVLTQSPSSLSASVGDRVTIKCRASQGISSALAWYQQKPGKAPKLLIYDASSL | 39 |
| VL amino acid | ESGVPSGFSGSGSGTDFTLTISSLQPEDFATYYCQQFNSYPHTFGPGTKVDIK | |
| sequence | | |
| MDC004 | YYWS | 40 |
| HCDR1 amino | | |
| acid sequence | | |
| MDC004 | YVHHSGGTNYNPSLKS | 41 |
| HCDR2 amino | | |
| acid sequence | | |
| MDC004 | DTYGPFDY | 42 |
| HCDR3 amino | | |
| acid sequence | | |
| MDC004 | QVQLQESGPGLVKPSETLSLTCTVSGASINIYYWSWIREASGKGLEWIGYVHHS | 43 |
| VH amino acid | GGTNYNPSLKSRVSISVDTSKNQFSLRLKSVTAADTAVYYCARDTYGPFDYWGQ | |
| sequence | GTLVTVSS | |
| MDC004 | (pE)VQLQESGPGLVKPSETLSLTCTVSGASINIYYWSWIREASGKGLEWIGYV | 44 |
| VH amino acid | HHSGGTNYNPSLKSRVSISVDTSKNQFSLRLKSVTAADTAVYYCARDTYGPFDY | |
| sequence with N- | WGQGTLVTVSS | |
| terminal | | |
| pyroglutamate | | |
| MDC004 | RASQGISNYLA | 45 |
| LCDR1 amino acid | | |
| sequence | | |
| MDC004 | AASTLQS | 46 |
| LCDR2 amino acid | | |
| sequence | | |
| MDC004 | QNYYSAPWT | 47 |
| LCDR3 amino acid | | |
| sequence | | |
| MDC004 | EIVLTQSPSSLSASVGDRVTITCRASQGISNYLAWYQQKPGKVPKLLIYAASTL | 48 |
| VL amino acid | QSGVPSRFSGSGSGTDFTLTVSSLQPEDVATYYCQNYYSAPWTFGQGTKVEIK | |
| sequence | | |
| MDC005 | TYYWS | 49 |
| HCDR1 amino | | |
| acid sequence | | |
| MDC005 | YIHYSGGTNYNPSLK | 50 |
| HCDR2 amino | | |
| acid sequence | | |
| MDC005 | DIYGPFDY | 51 |
| HCDR3 amino | | |
| acid sequence | | |
| MDC005 | QVQLQESGPGLVKPSETLSLTCTVSGGSINTYYWSWIRQPPGMGLEWIGYIHYS | 52 |
| VH amino acid | GGTNYNPSLKSRVAISADTSRNQFSLKLSFVTAADTAVYYCARDIYGPFDYWGQ | |
| sequence | GTLVTVSS | |
| MDC005 | (pE)VQLQESGPGLVKPSETLSLTCTVSGGSINTYYWSWIRQPPGMGLEWIGYI | 53 |
| VH amino acid | HYSGGTNYNPSLKSRVAISADTSRNQFSLKLSFVTAADTAVYYCARDIYGPFDY | |
| sequence with N- | WGQGTLVTVSS | |
| terminal | | |
| pyroglutamate | | |
| MDC005 | RASQVISNYLA | 54 |
| LCDR1 amino acid | | |
| sequence | | |
| MDC005 | ASSTLQS | 55 |
| LCDR2 amino acid | | |
| sequence | | |
| MDC005 | QNYYSVPWT | 56 |
| LCDR3 amino acid | | |
| sequence | | |
| MDC005 | EIVLTQSPSSLSASVGDRVTFTCRASQVISNYLAWYQQKPGKLPKLLIYASSTL | 57 |
| VL amino acid | QSGVPSRFSGSGSGTDFTLGISSLQPEDVATYYCQNYYSVPWTFGQGTKVEIK | |
| sequence | | |
| MDC006 | DYYMS | 58 |
| HCDR1 amino | | |
| acid sequence | | |
| MDC006 HCDR2 | SISNSGGAIYYADSVKG | 59 |
| amino acid | | |
| sequence | | |
| MDC006 HCDR3 | YNWNDRGYDAFDI | 60 |
| amino acid | | |
| sequence | | |
| MDC006 | QVQLVESGGGLVKPGGSLRLSCAASGFTFSDYYMSWIRQAPRKGLEWVSSISNS | 61 |
| VH amino acid | GGAIYYADSVKGRFSISRDNAKNSLYLQMNSLRAEDTAVYYCARYNWNDRGYDA | |
| sequence | FDIWGQGTMVTVSS | |
| MDC006 | (pE)VQLVESGGGLVKPGGSLRLSCAASGFTFSDYYMSWIRQAPRKGLEWVSSI | 62 |
| VH amino acid | SNSGGAIYYADSVKGRFSISRDNAKNSLYLQMNSLRAEDTAVYYCARYNWNDRG | |
| sequence with N- | YDAFDIWGQGTMVTVSS | |
| terminal | | |
| pyroglutamate | | |
| MDC006 LCDR1 | RASQGISSALA | 63 |
| amino acid | | |
| sequence | | |
| MDC006 LCDR2 | DASSLES | 64 |
| amino acid | | |
| sequence | | |
| MDC006 LCDR3 | QQFNSYPHT | 65 |
| amino acid | | |
| sequence | | |
| MDC006 | DIVMTQSPSSLSASVGDRVTITCRASQGISSALAWYQQKPGKAPKILIYDASSL | 66 |
| VL amino acid | ESGVPSRFSGSGSGTDFTLTISSLLPEDFVTYYCQQFNSYPHTFGPGTKVEIK | |
| sequence | | |
| huM25 huIgG1 | EVQLVQSGAEVKKPGASVKVSCKASGYKFSSYWIEWVKQAPGQGLEWIGEILPG | 67 |
| amino acid | SDTTNYNEKFKDRATFTSDTSINTAYMELSRLRSDDTAVYYCARDRGNYRAWFG | |
| sequence | YWGQGTLVTVSSASTKGPSVFPLAPSSKSTSGGTAALGCLVKDYFPEPVTVSWN | |
| | SGALTSGVHTFPAVLQSSGLYSLSSVVTVPSSSLGTQTYICNVNHKPSNTKVDK | |
| | KVEPKSCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGKEYKC | |
| | KVSNKALPAPIEKTISKAKGQPREPQVYTLPPSREEMTKNQVSLTCLVKGFYPS | |
| | DIAVEWESNGQPENNYKTTPPVLDSDGSFFLYSKLTVDKSRWQQGNVFSCSVMH | |
| | EALHNHYTQKSLSLSPGK | |
| huM25 hK amino | DIQMTQSPSSLSASVGDRVTITCRASQDISNYLNWYQQKPGGAVKFLIYYTSRL | 68 |
| acid sequence | HSGVPSRFSGSGSGTDYTLTISSLQPEDFATYFCQQGEALPWTFGGGTKVEIKR | |
| | TVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSFNRGEC | |
| Anti-LRRC15 | GAATTCGCCACCATGAGGGCTTGGATCTTCTTTCTGCTCTGCCTGGCCGGGCGC | 69 |
| antibody signal | GCCCTCGCA | |
| peptide nucleotide | | |
| sequence | | |
| Anti-LRRC15 | EFATMRAWIFFLLCLAGRALA | 70 |
| antibody signal | | |
| peptide amino acid | | |
| sequence | | |
| 3′ appendix | TGAGGATCCCCCGACCTCGACCTCTGGCTAATAAAGGAAATTTAT | 71 |
| nucleotide | | |
| sequence | | |

### Example 1: Generation of Anti-LRRC15 Antibodies

### Antibody Discovery

Anti-LRRC15 antibodies were identified from immunization of human BCR transgenic mice with recombinant human and mouse LRRC15 protein antigen, and LRRC15 mRNA-lipid nanoparticle (LNP). Following immunization of the mice, the B cells were harvested, and antibodies binding LRRC15 were isolated via hybridoma fusion, single B-cell cloning, sequencing and recombinant expression as human IgG1/kappa antibodies. Antibodies were screened by flow cytometry for binding to cells expressing human, cyno and mouse LRRC15 and functional profiling for internalization and potency in ADC cytotoxicity assays in vitro and in vivo.

### Immunization and Antibody Selection

Antibodies were tested by single-point FACS assays to evaluate binding to cell lines over-expressing full-length human LRRC15, cynomolgus (cyno) LRRC15, mouse LRRC15 and parental cell lines. Antibodies that displayed binding to human, cyno and mouse LRRC15 were selected for further characterization.

### Example 2: Binding Specificity of Anti-LRRC15 Antibodies by ELISA

The cell binding specific of the anti-LRRC15 antibody mAbs (e.g., MBP006, MBP003, MBP001, MBP004, MBP005), compared to a positive control anti-LRRC15 antibody clone huM25, and a isotype control antibody (KLH). The ELISA plate was immobilized with neutravidin (Thermo Fisher, cat #31000) at 2 μg/ml in a carbonate-bicarbonate buffer and incubated overnight at 4° C. to allow the antigen to adhere to the wells. The plate was then blocked with ELISA diluent containing BSA to prevent non-specific binding. After washing, biotinylated human, mouse, cyno, rat, and canine LRRC15 antigens were added to the wells and incubated for 1 hour at room temperature to allow the antigens to bind to neutravidin (rat and canine LRRC15 were only tested with MBP001, huM25, and KLH). Following washing, anti-LRRC15 antibodies were added to the wells starting at 6.67 nM and diluted 3-fold for an 8-pt titration curve and incubated for 1 hour at room temperature to allow antibodies to bind to the antigens. Unbound antibodies were subsequently washed away and a horseradish peroxidase (HRP) conjugated goat anti-human IgG Fc (Jackson ImmunoResearch, cat #109-036-098) secondary antibody for detection was added. Following incubation and washing to remove unbound secondary antibodies, TMB substrate for HRP (Surmodics, cat #ABTS-1000-01) was added and allowed to develop. Absorbance was then measured at 405 nm using an Envision microplate reader.

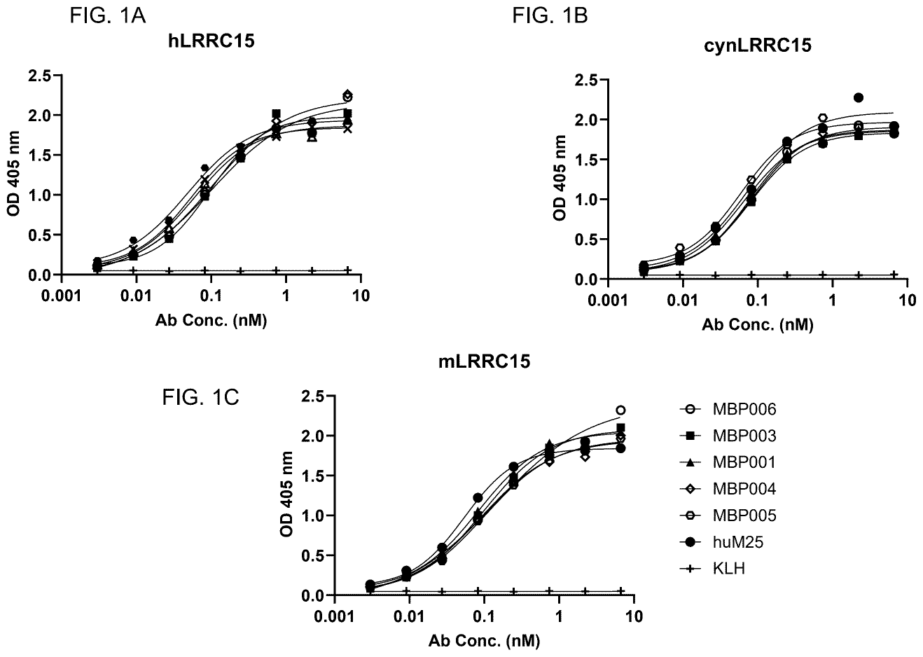

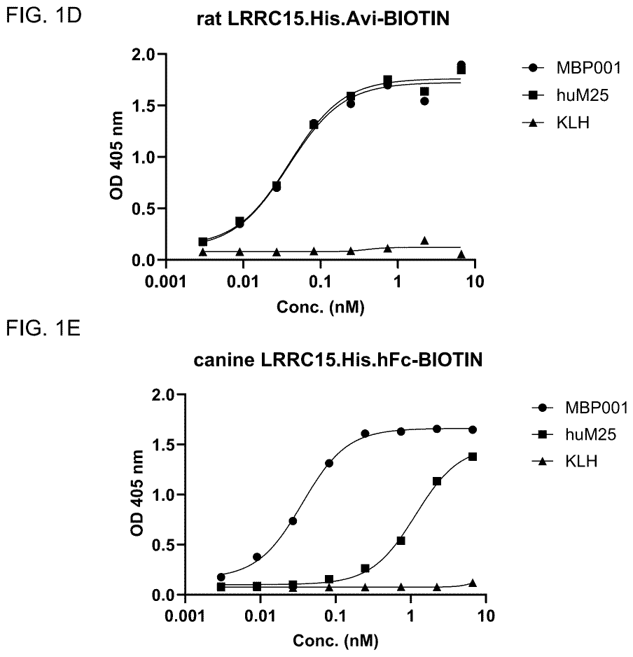

As shown in Table 4 and FIGS. 1A-1C, the anti-LRRC15 mAbs exhibited specific binding to human (FIG. 1A), cyno (FIG. 1B), and mouse (FIG. 1C) LRRC15, comparable with the huM25 mAb control.

TABLE 4
Binding specificity of anti-LRRC15 mAbs to human, cyno, and mouse LRRC15

| | MDC006 | MDC003 | MDC001 | MDC004 | MDC005 | huM25 |
|---|---|---|---|---|---|---|
| | EC50 (nM) | EC50 (nM) | EC50 (nM) | EC50 (nM) | EC50 (nM) | EC50 (nM) |
| hLRRC15 | 0.101 | 0.094 | 0.059 | 0.102 | 0.048 | 0.054 |
| cynLRRC15 | 0.077 | 0.079 | 0.065 | 0.083 | 0.059 | 0.073 |
| mLRRC15 | 0.142 | 0.099 | 0.083 | 0.091 | 0.100 | 0.054 |

As shown in FIGS. 1D-1E, the anti-LRRC15 mAb MBP001 also exhibited specific binding to both rat (FIG. 1D) and canine (FIG. 1E) LRRC15. MBP001 bound to rat and canine LRRC15 with an EC50 of 0.039 nM and 0.036 nM, respectively, while huM25 bound to rat and canine LRRC15 with an EC50 of 0.040 nM and 1.152 nM, respectively.

### Example 3: Epitope Community of Binder Hits

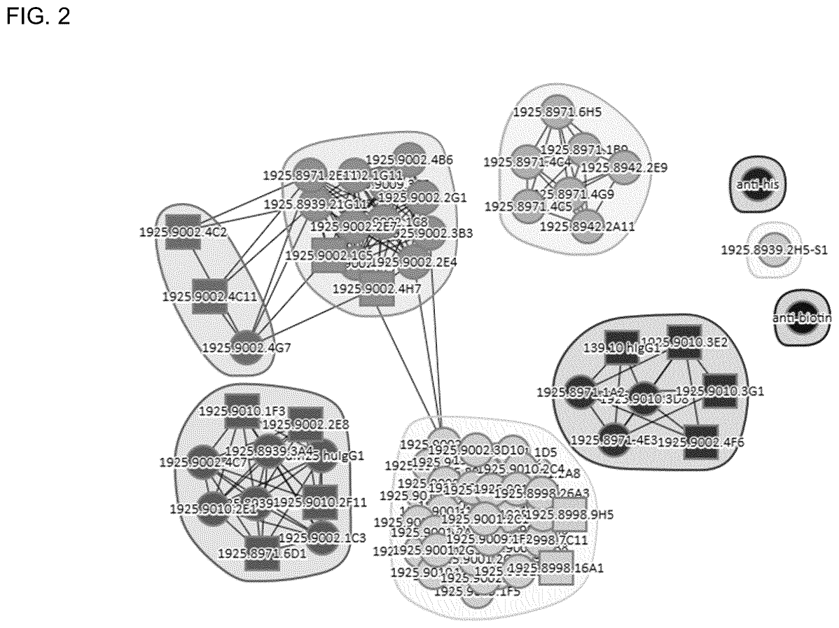

Eight epitope communities were identified (FIG. 2), six clones from five different epitope bins were selected for further characterization based on FACS binding, binding kinetics and sequence diversity. Six clones were tested by surface plasmon resonance (SPR) to determine binding kinetics to human, cyno and mouse LRRC15. This experiment was done using a Biacore 8K+ instrument where an anti-human IgG Fc capture reagent was directly immobilized to a carboxymethyl dextran chip surface, followed by multiple cycles of a dilution series of LRRC15 analyte injected over each antibody captured to the anti-human Fc capture surface.

### Example 4: Binding Kinetics of LRRC15 mAb

Analytical binding studies were carried out by surface plasmon resonance (SPR) to characterize the binding kinetics of the anti-LRRC15 mAb MBP001 to human LRRC15 (Acro Biosystems Cat. #LR5-H52H3).

Hits from single-point screening were then evaluated in a high-throughput epitope binning experiment using the Carterra LSA platform, to determine which mAbs share common binding epitopes. A set of pairwise competition injections were set up by immobilizing the panel of mAbs to a polycarboxylate chip surface, then injections of full-length human LRRC15 over the immobilized mAbs were followed immediately by injections of competitor mAb. These competing antibody relationships allowed the panel of mAbs to be grouped into distinct epitope communities.

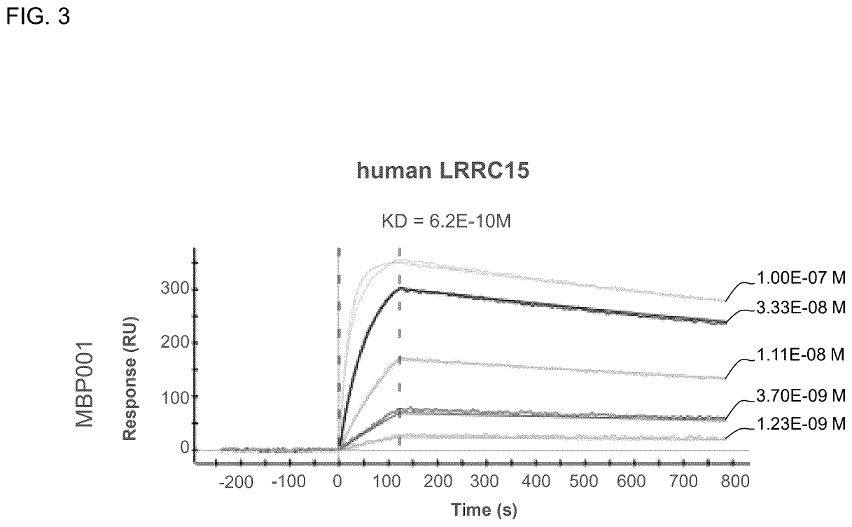

The kinetic data for anti-LRRC15 mAb: LRRC15 binding were fit to a 1:1 Langmuir binding with Rmax to provide estimates of the kinetic and affinity values for the corresponding interactions. The binding kinetics and affinities for MBP001 to human LRRC15 are shown in FIG. 3 and Table 5, and indicate that MBP001 has a KD of 7.4×10⁻¹⁰ M.

TABLE 5
Binding kinetics of anti-LRRC15
mAb MBP001 to human LRRC15

(Acro) human LRRC15 (37° C.)

| | Avg. | Avg. | Avg. | Avg. |
|---|---|---|---|---|
| | ka (1/Ms) | kd (1/s) | t_{1/2} (min) | KD (M) |
| MBP001 | 6.5E+05 | 4.3E−04 | 28.1 | 7.4E−10 |
| | 95% C.I. | 95% C.I. | 95% C.I. | 95% C.I. |
| | ±2.8E5 | ±7.4E−5 | ±3.8 | ±1.4E−10 |
| | n = 6 | n = 6 | n = 6 | n = 6 |

### Example 5: Carbene Footprinting

### Epitope Mapping

Complexes of a non-native LRRC15 antigen and anti-LRRC15 mAb MBP001 were prepared using 5 μg of antigen and the necessary equal molar amount of antibody amount (12.30 ug) to ensure capture of the free antigen. Samples were prepared on an Agilent U96 PP 0.5 ml round bottom 96 well plate by incubating the mixture of antibody, LRRC15, and Leu-Enkephalin internal standard at 25° C. for 25 min, followed by incubation with diazirine reagent at 25° C. for 35 min. The samples were then snap frozen in liquid nitrogen. Labeling occurred via incubation in a UV incubator CL-3000L to reach a total exposure of 6000 mJ/cm². All samples were then thawed to room temperature and transferred to individual 1.5 ml tubes. Deglycosylation with PNGase F was conducted by incubating at 50° C. overnight.

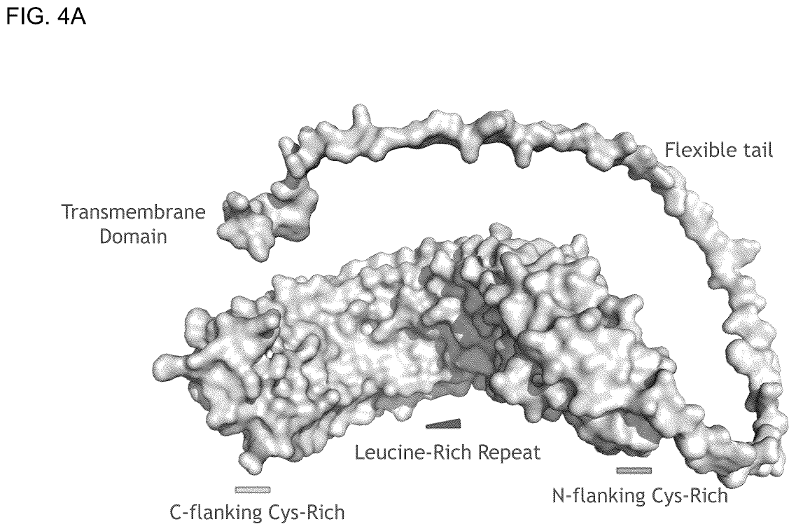

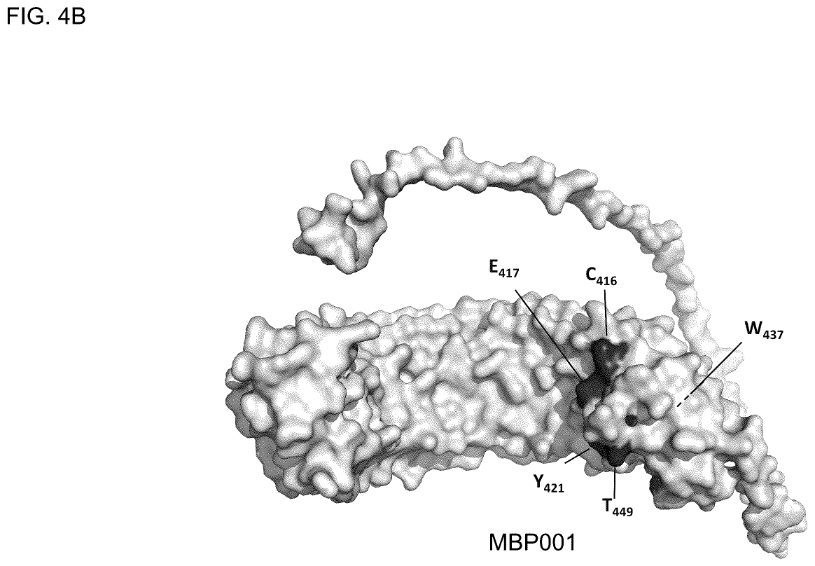

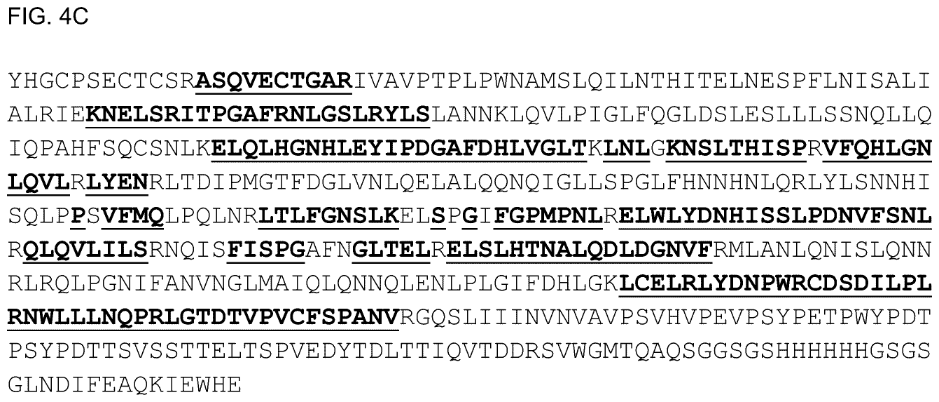

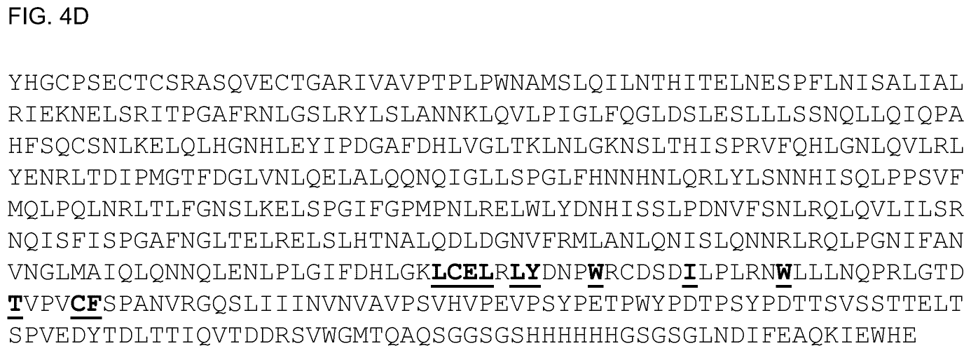

Aliquots of all samples containing 5 μg of antigen were evaporated to dryness in a lyophilizer and resuspended in 7.66 M Urea and 100 mM Tris pH 7.5 buffer, followed by reduction with dithiolthreitol at 37° C. for 1 hour. Alkylation of the samples with iodoacetamide was performed at room temperature protected from light for 30 min. Then samples were diluted with 100 mM Tris pH7.5 buffer and digested with Trypsin. After trypsin digestion, the digested samples were analyzed on an Orbitrap Fusion Lumos mass spectrometer coupled with a Waters M-Class UPLC using an Acquity CSH C18 column (0.3×150 mm, 1.7 um) over a 60 min gradient from 0.5 to 35% B at 10 μl/min. Analysis was performed by LC-MS. Mass spectrometry data was analyzed using protein metrics, and the residues that experience protection upon antibody binding are shown in FIG. 4D. The residues that experience carbine labeling are shown in FIG. 4C. FIG. 4A shows a three-dimensional representation of human LRRC15, identifying the transmembrane domain, flexible tail, N-flanking cys-rich region, leucine-rich repeat region, and C-flanking cys-rich region. As shown in FIG. 4B, the epitope for mAb MBP001 binding includes residues C416, E417, Y421, W437, and T449 on native human LRRC15 (corresponding to residues C395, E396, Y400, W416 and T428 of the non-native LRRC15 antigen used in this experiment).

### Example 6: In Vitro Binding of LRRC15 mAbs to Cell Lines Expressing LRRC15

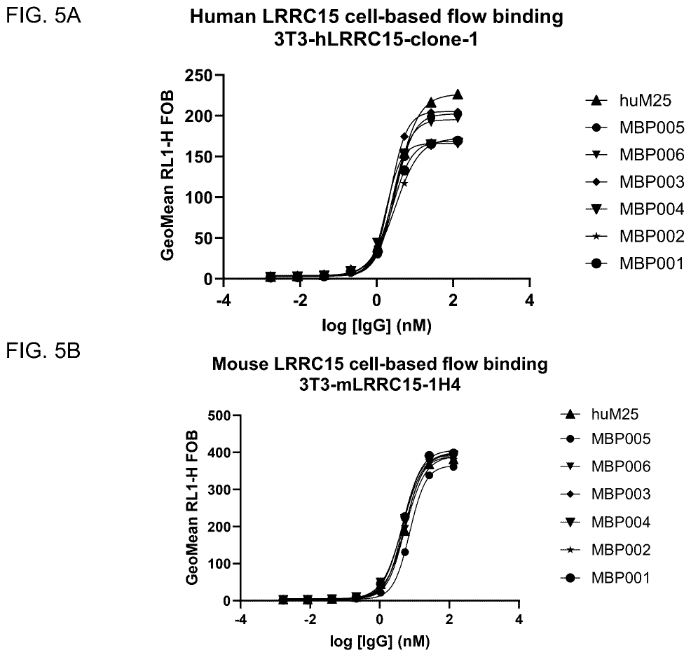

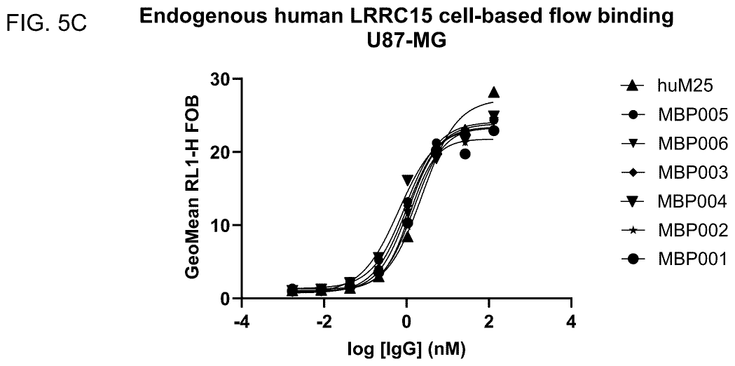

FIGS. 5A-5C show binding of anti-LRRC15 antibodies to a transfected 3T3 cell line expressing human LRRC15 (FIG. 5A), a transfected 3T3 cell line expressing mouse LRRC15 (FIG. 5B), and a mesenchymal cancer cell line (U87-MG) expressing endogenous LRRC15 (FIG. 5C).

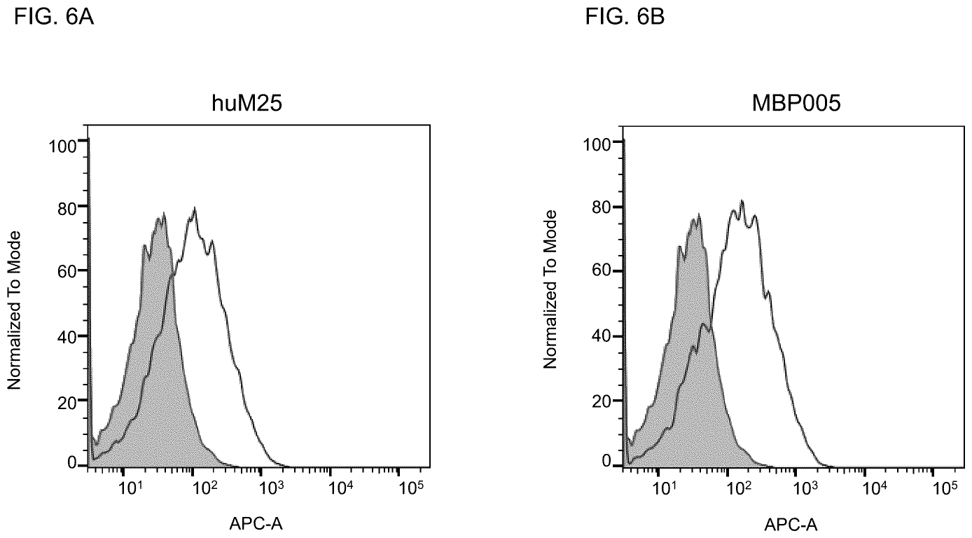

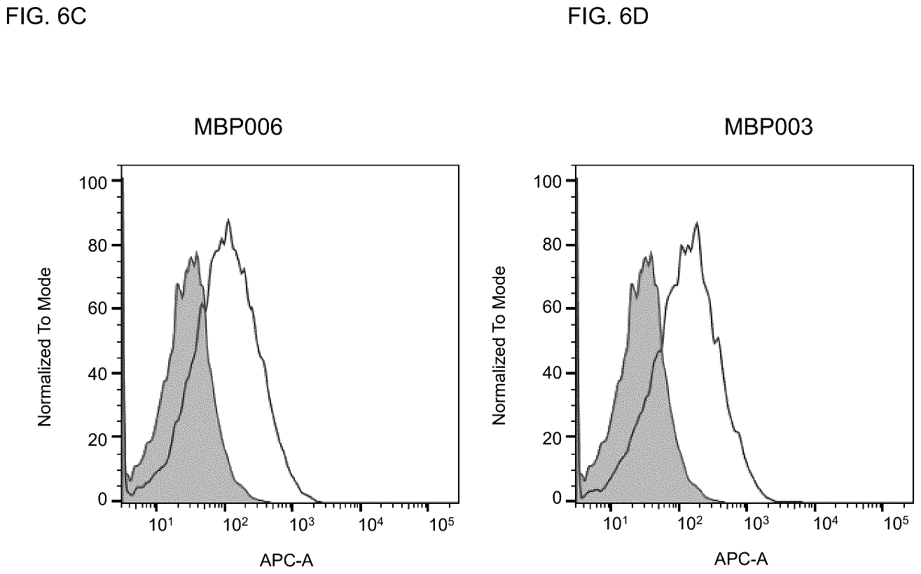

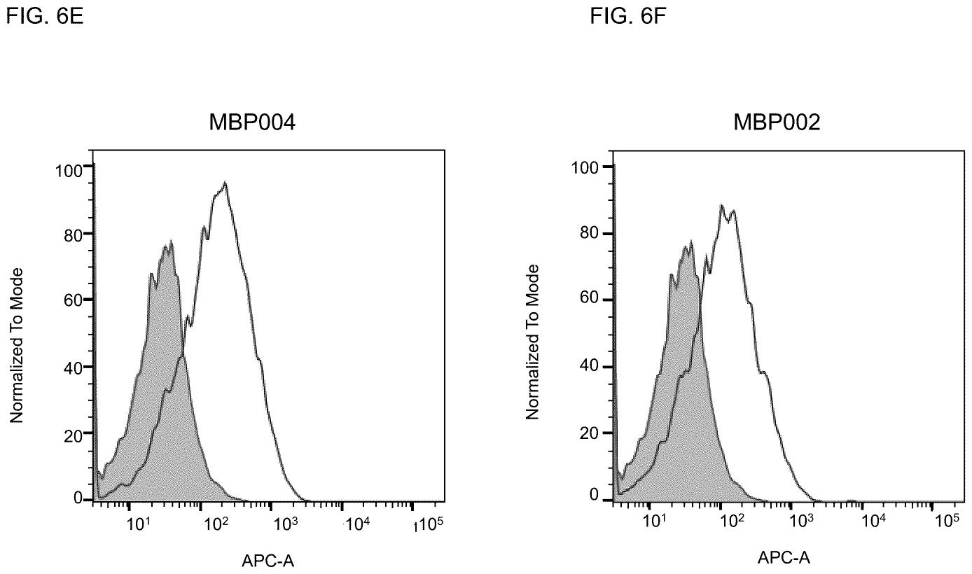

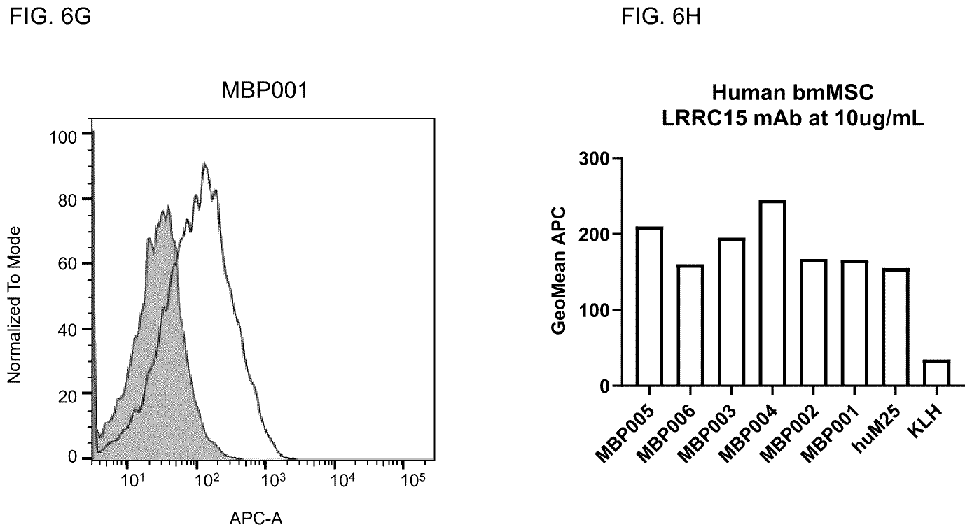

FIGS. 6A-6H show cell surface binding of anti-LRRC15 antibodies to human bone derived marrow mesenchymal stem cells (FIGS. 6A-6G) as measured by flow cytometry. Anti-LRRC15 antibodies were used at 10 ug/ml and an AF647-conjugated secondary antibody (Jackson Immunoresearch, 109-136-098) was used for detection by flow cytometry. As shown in FIG. 6H, the anti-LRRC15 antibodies MBP005, MBP006, MBP003, MBP004, MBP002, MBP001, and huM25 showed higher cell surface binding than the KLH control to human bone marrow derived mesenchymal stem cells.

### Example 7: Expression of LRRC15 on Primary Breast Cancer Associated Fibroblasts Over Multiple Passages

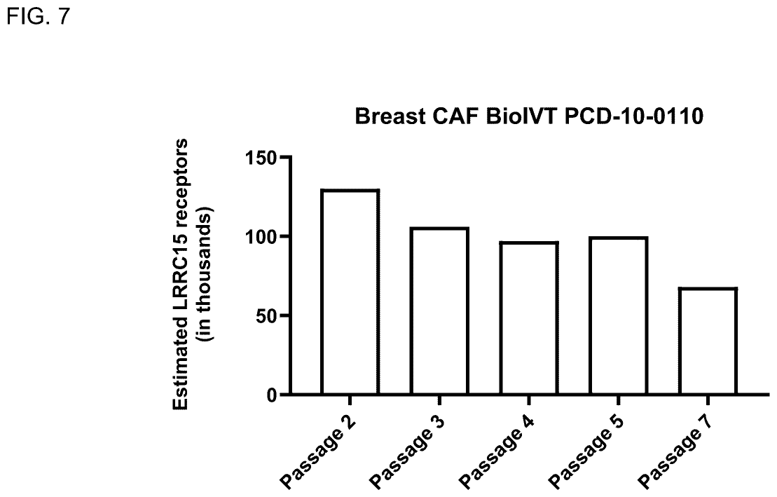

FIG. 7 shows decreasing LRRC15 expression of primary Cancer Associated Fibroblasts (CAFs) over multiple passages ex vivo. CAFs derived from breast tissue (BioIVT #PCD-10-0110) were found to express LRRC15 by flow cytometry, with a decrease in the estimated LRRC15 receptor copy number seen as the number of passages increased. Receptor copy numbers were estimated using Quantum Simply Cellular anti-Human IgG Bead kit (Bangs Laboratories #816A).

### Example 8: Expression of LRRC15 on Stromal Cells and Cancer Associated Fibroblasts Treated With TGFb

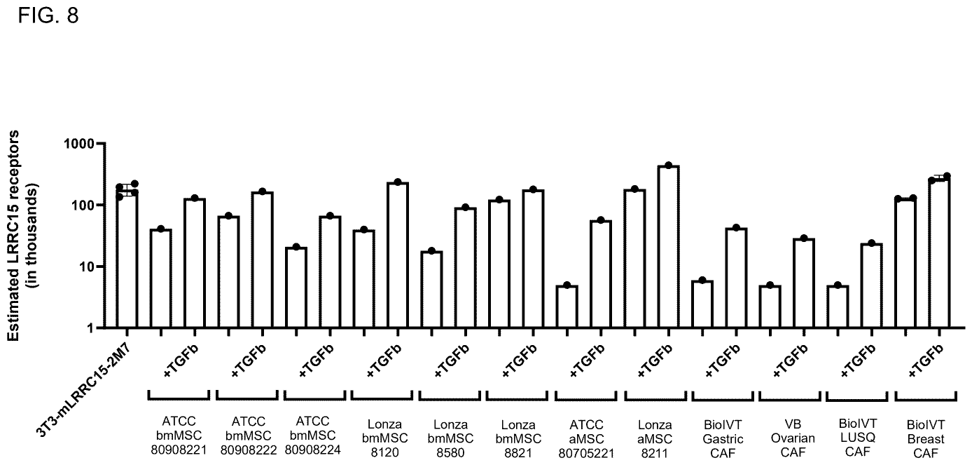

FIG. 8 shows LRRC15 expression of a 3T3 line overexpressing mouse LRRC15 alongside a panel of commercially acquired primary human stromal cells at an early passage. Bone marrow derived mesenchymal stem cells (bmMSC) (ATCC #PCS-500-012 and Lonza #PT-2501), adipose derived mesenchymal stem cells (ATCC #PCS-500-011 and Lonza #PT-5006) and primary Cancer Associated Fibroblasts from Gastric, Ovarian, Lung and Breast tissue (BioIVT #PCD-10-0210, #PCD-10-0410, #HUMANCAF-0003058 and VitroBiopharma #CAF-02) were cultured with or without 10 ng/ml of TGFβ-1 (R&D Systems #240-B). Receptor copy numbers were estimated using the Quantum Simply Cellular anti-Human IgG Bead kit (Bangs Laboratories #816A). As shown in FIG. 8, the estimated LRRC15 receptor copy number increased in each cell line tested with the addition of TGFβ-1.

### Example 9: In Vitro Flow Binding of LRRC15 mAbs to Endogenous Stromal Cells

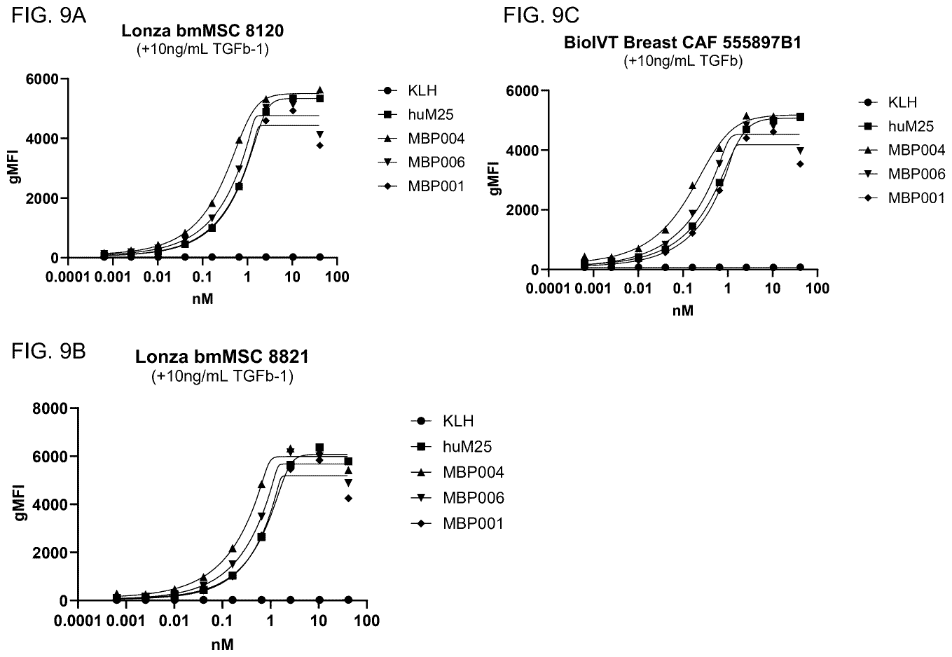

FIGS. 9A-9C shows concentration-dependent binding of AF647-labeled LRRC15 antibodies to primary human stromal cells by flow cytometry. Two bone marrow derived mesenchymal stem cells (bmMSC) clones were acquired from Lonza (#PT-2501) and Breast cancer-associated fibroblasts (CAF) were acquired from BioIVT (#PCD-10-0110.) Comparable anti-LRRC15 antibody binding of mAb clones huM25, MBP004, MBP006, and MBP001 was seen in both bmMSC lines (FIGS. 9A-9B), as in the breast CAF cells (FIG. 9C), with the KLH isotype control antibody showing no binding.

### Example 10: LRRC15 mAb Internalization by Incucyte Imaging

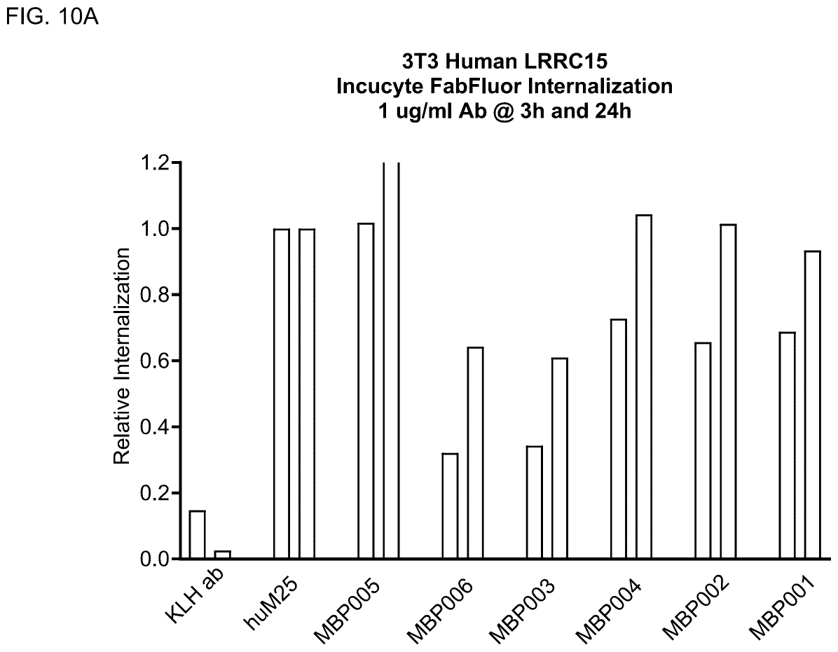

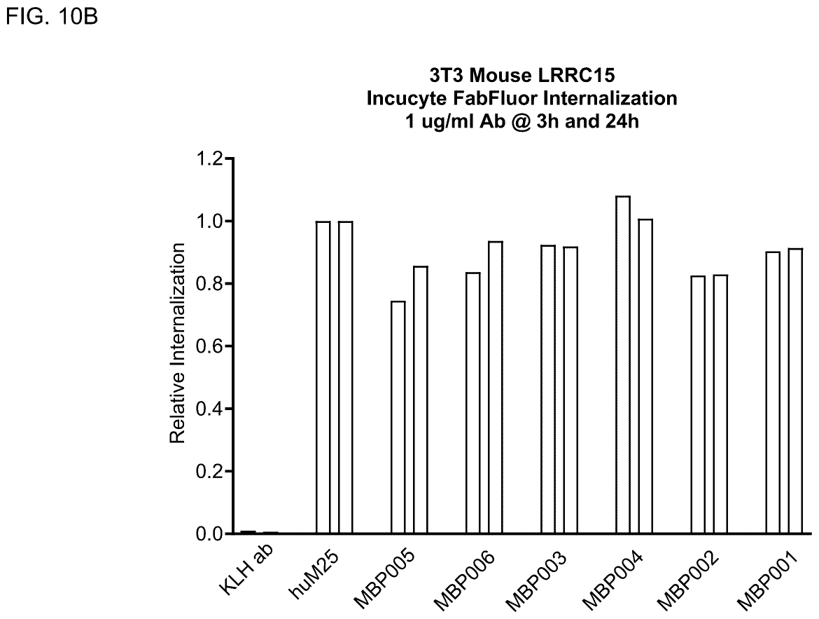

FIGS. 10A-10B show the relative internalization rates of anti-LRRC15 mAbs in 3T3 cells expressing either human LRRC15 (FIG. 10A) or mouse LRRC15 (FIG. 10B), representing 5 epitope communities. 3T3 transfectant cells lines expressing human or mouse LRRC15 were seeded at 10,000 cells per well. Following the vendor protocol, hIgG1 mAbs were pre-incubated with the Incucyte Human Fabfluor-pH Red Antibody Labeling Dye (Sartorius, Cat #4722) at 37 C. The Fabfluor-antibody mix was then added to the cell plate and Phase/Red images were captured every 30 minutes using the Incucyte SX5 system. Data plotted shows the relative internalization of antibody clones at 1 μg/ml, as assessed by Red Integrated Intensity normalized to confluence at 3 hours and at 24 hours.

### Example 11: Anti-LRRC15 MBP001 Antibody has Lower Polyreactivity Compared to Anti-LRRC15 huM25 Antibody

Baculovirus particles (BVP, 25690; Curia Bio) stock was diluted to a 1% suspension with 50 mM sodium carbonate (pH 9.6) and incubated on ELISA plates at 4° C. overnight. The next day, unbound BVPs were aspirated from the wells. All remaining steps were performed at room temperature. One hundred microliters of blocking buffer (PBS with 0.5% BSA) was added and incubated for 1 hour, followed by three washes with 50 μL of PBS. Next, 50 μL of 1 uM testing antibodies in blocking buffer was added to the wells and incubated for 1 hour, followed by three washes with 100 μL of PBS. Fifty microliters of diluted anti-human IgG-HRP conjugate (109-035-008; Jackson Immuno Research) was then added to the wells and incubated for 1 hour, followed by six washes as before. Finally, 100 μL of TMB substrate (TMBW-1000-01; BioFX) was added to each well and incubated for 15 minutes. The reactions were stopped by adding 100 μL of stop solution (STPR; BioFX). The absorbance was read at 450 nm, and the BVP score was determined by normalizing the absorbance to control wells with no test antibody.

The BVP score of anti-LRRC15 MBP001 antibody was found to be 3.72, which is significantly lower than the score of huM25 (7.48). The data suggests that MBP001 antibody has a lower level of polyreactivity compared to anti-LRRC15 huM25 antibody.

### Example 12: Conjugation Reaction with Compound A′ and Anti-LRRC15 mAbs to Yield DAR8 ADCs

This example describes the conjugation of LRRC15 antibodies, e.g., MBP001 antibodies, to Compound A′. Table 6 lists materials used for the conjugation method.

TABLE 6
Reagents and consumables

| Description | Manufacturer | Catalog number |
|---|---|---|
| Zeba Spin desalting columns 7 kDa MWCO | Thermo Fisher Scientific | 89893 |
| Amicon Ultra - 1 5 PLTK Ultracel-PL | Merck Millipore | UFC903024 |
| centrifugal filters Regenerated Cellulose | | |
| (30 kDa MWCO) | | |
| TCEP HCl, 0.5 M, pH 7.0 | Sigma-Aldrich | 646547 |

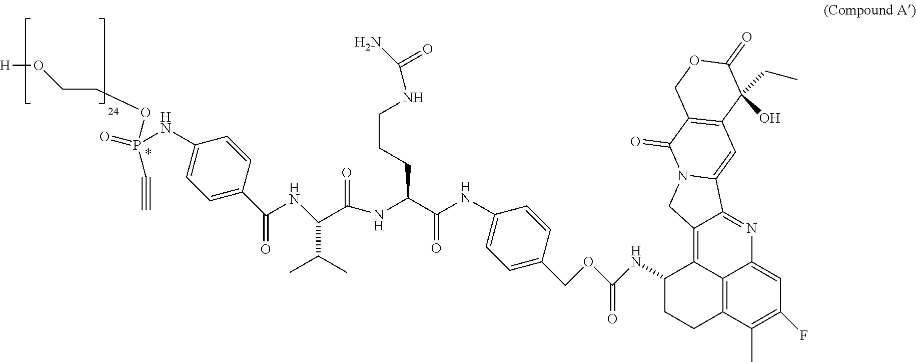

Firstly, each mAb was buffer-exchanged into P5 conjugation buffer (50 mM Tris, 100 mM NaCl, 1 mM EDTA, pH 8.3 at 25° C.) and adjusted to a concentration of 10 mg/ml. Next, Zeba Spin desalting columns were utilized. The columns were equilibrated with P5 conjugation buffer according to the manufacturers' instructions. Recovery-yields were usually >95%.

After buffer exchange, the mAb concentration was measured with a nanophotometer. P5 conjugation buffer was utilized as the blank. Finally, the mAb concentration was adjusted to 1˜5 mg/ml with P5 conjugation buffer.

Next the mAb was transferred into Eppendorf tube. The conjugation reaction was performed protected from light. Conjugation with Compound A′ was performed with a molar ratio of 7 eq. TCEP and 10 eq. of Compound A′ per 1 equivalent of mAb. Typically, a 40 mM Compound A′ stock in DMSO and a 10 mM TCEP working solution were used. The 10 mM TCEP working solution was prepared by combining 20 μl of 0.5 M TECP-HCl pH 7.0 with 980 μl of P5 conjugation buffer. Both reagents were vortexed prior to use.

The calculated amount of TCEP working solution was added to the solution of the mAb and mixed by gentle swirling. The calculated amount of Compound A′ stock was added immediately afterwards. The mixture was then incubated overnight at 23° C. in amber 50 ml tubes and spun at a speed of 300 rpm.

Conjugation efficiency was evaluated by LC-MS analysis. The conjugate samples were reduced by TCEP. The samples were analyzed by LC-MS using an Agilent 1290 Infinity UPLC coupled to a 6530 Accurate-Mass Q-TOF mass spectrometer (Agilent, Santa Clara, CA). The analytical column used was a Waters BEH C4 column, 1.7 μm, 2.1 mm×50 mm, held at 60° C. The mobile phase consisted of 0.1% formic acid in water (A) and 0.1% formic acid in acetonitrile (B). The system was operated at a flow rate of 200 μL/min. The gradient condition was as follows: 0-2 min, held at 27% B; 2-9 min, slow ramp from 27% to 37% B; 9-9.5 min, linear ramp from 37% to 90% B; 9.5-12.3 min, held at 90% B. The MS settings were as follows: Polarity=Positive, Capillary Voltage=4.2 kV, Sample Cone=40 V, Source Offset=15 V, Source Temperature=140° C., Desolvation Temperature=325° C. The data acquisition range was 900-3200 m/z. Deconvolution was performed using Agilent MassHunter Walkup. The process yielded DAR8 ADCs, MBP005-P5-exatecan (MDC005) ADC, MBP006-P5-exatecan (MDC006) ADC, MBP003-P5-exatecan (MDC003) ADC, MBP004-P5-exatecan (MDC004) ADC, MBP002-P5-exatecan (MDC002) ADC, and MBP001-P5-exatecan (MDC001) ADC.

### Example 13: Antibody-Dependent Cell-Mediated Cytotoxicity (ADCC) Characterization of LRRC15 mAb and LRRC15-P5-Exatecan ADC

Antibody-dependent cell-mediated cytotoxicity was assessed using an ADCC reporter assay (Promega, G7010). Target cells U118-MG expressing both EGFR and LRRC15 were incubated with a dose titration of LRRC15 mAb, LRRC15-P5-exatecan ADC, or Cetuximab (anti-EGFR mAb) as a positive control. Jurkat-FcγRIIIa effector cells were then added. NFAT activation, reflecting ADCC response, was assessed by determining luciferase activity six hours later.

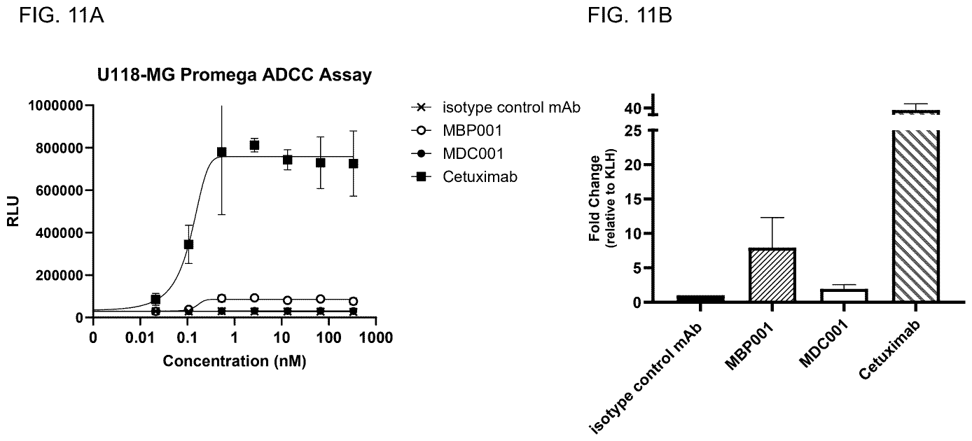

FIGS. 11A and 11B show that relative to the positive control Cetuximab, MBP001 mAB and MDC001 ADC show minimal antibody-dependent cell mediated cytotoxicity.

### Example 14: Exatecan Payload Sensitivity Showing Fibroblast Vs Cancer Cell Differential Susceptibility

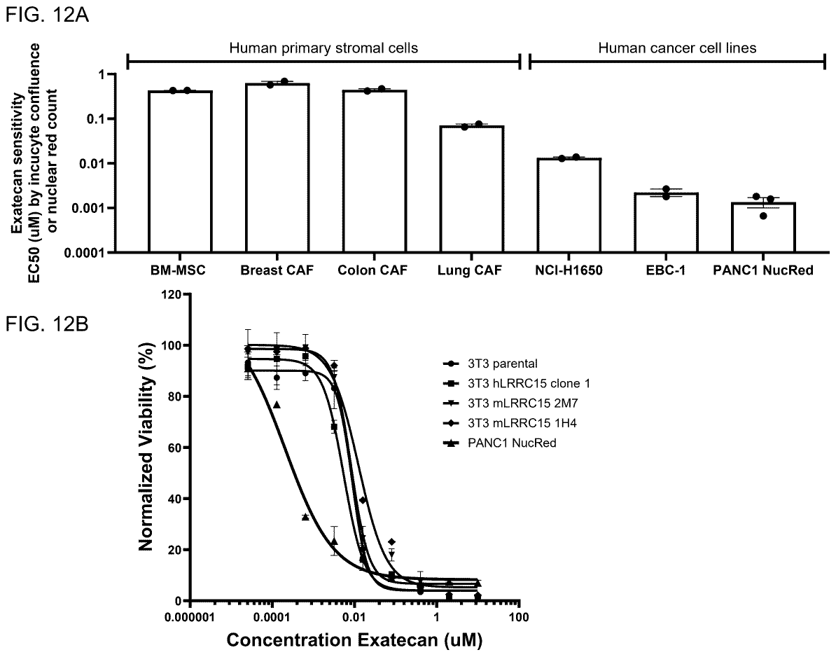

This example shows the intrinsic sensitivity of various cell lines to exatecan. A panel of human primary stromal cells and human cancer cell lines were treated with exatecan at concentrations ranging from 10 μM to 2.56 pM. Bone marrow derived mesenchymal stem cells were purchased from Lonza, and cancer associated fibroblasts (CAFs) from breast, colon and lung tissue were purchased from BioIVT and VitroBiopharma. Cells were cultured with exatecan for four days and the Incucyte SX5 imaging system (Sartorius) was used to assess cell confluence or nuclear red cell count. The sensitivity of each cell line to exatecan is plotted as half-maximal effective concentration (EC₅₀) values in FIG. 12A, showing that human primary stromal cells are less sensitive than PANC1 cancer cells to exatecan. 3T3 mouse fibroblast cells and 3T3 LRRC15 overexpressing transfectant cell lines are similarly less sensitive than PANC1 cancer cells in the exatecan full dose response curves shown in FIG. 12B.

### Example 15: Monoculture Viability Assay Using Anti-LRRC15-P5-Exatecan ADCs

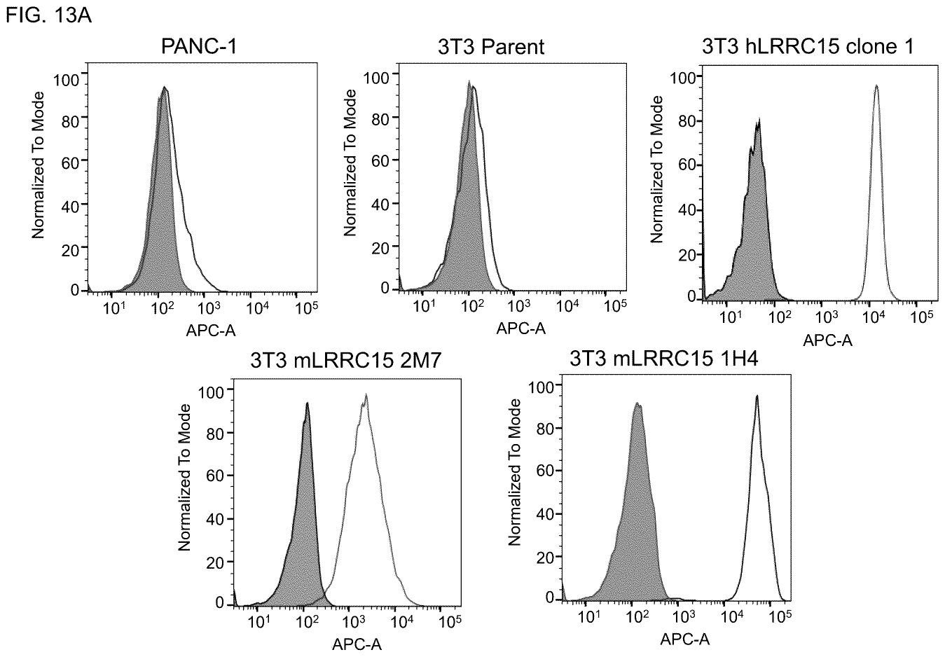

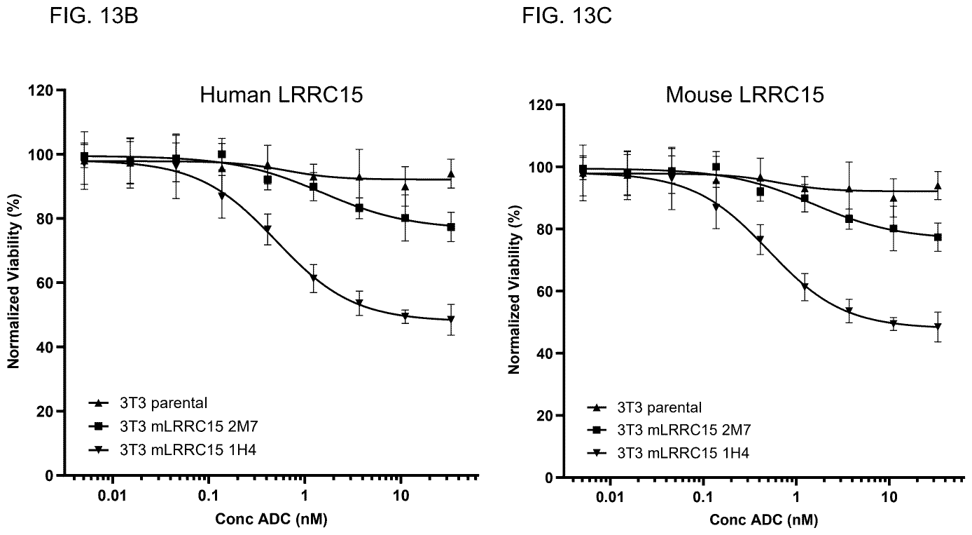

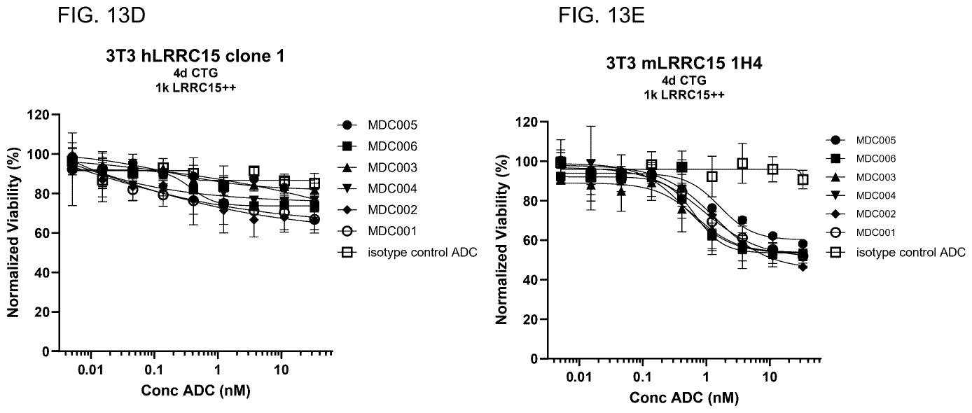

FIG. 13A shows cell surface LRRC15 protein expression of PANC1 cancer cells (LRRC15−), 3T3 parental cells (LRRC15−), and LRRC15 transfectant cell lines 3T3 human LRRC15 clone 1 (LRRC15++), 3T3 mouse LRRC15 2M7 (LRRC15+) and 3T3 mouse LRRC15 1H4 (LRRC15++).

Monocultures of 3T3 parental and LRRC15 transfectant cell lines were assessed for direct ADC activity. 3T3 parental cells (LRRC15−), 3T3 human LRRC15 clone 1 (LRRC15++), 3T3 mouse LRRC15 2M7 (LRRC15+) and 3T3 mouse LRRC15 1H4 (LRRC15++) were seeded at 500 or 1000 cells per well. ADCs were tested in a 3-fold titration range covering 33.33 nM to 0.005 nM ADC. KLH 2F5-P5-exatecan (hIgG1 isotype control) and a panel of anti-LRRC15-P5-exatecan ADCs were used. Viability at day 4 was assessed by Luminescence using CellTiterGlo (Promega, G7573) and by confluence using phase imaging on an Incucyte SX5 system.

Anti-LRRC15-P5-exatecan ADCs show dose-dependent and antigen dependent activity on 3T3 transfectant cell lines expressing high levels of LRRC15, but activity is limited to cell stasis (FIGS. 13B, 13C, 13D, and 13E). Minimal activity is seen using the lower level expressing 3T3 mLRRC15 2M7 transfectant line (FIG. 13C). 3T3 parental cells which do not express the LRRC15 antigen are not impacted by the anti-LRRC15-P5-exatecan ADCs (FIGS. 13B-13C).

### Example 16: Monoculture and Co-Culture Viability Assays Using Anti-LRRC15-P5-Exatecan ADCs on LRRC15 Negative Cancer Cell Line PANC-1

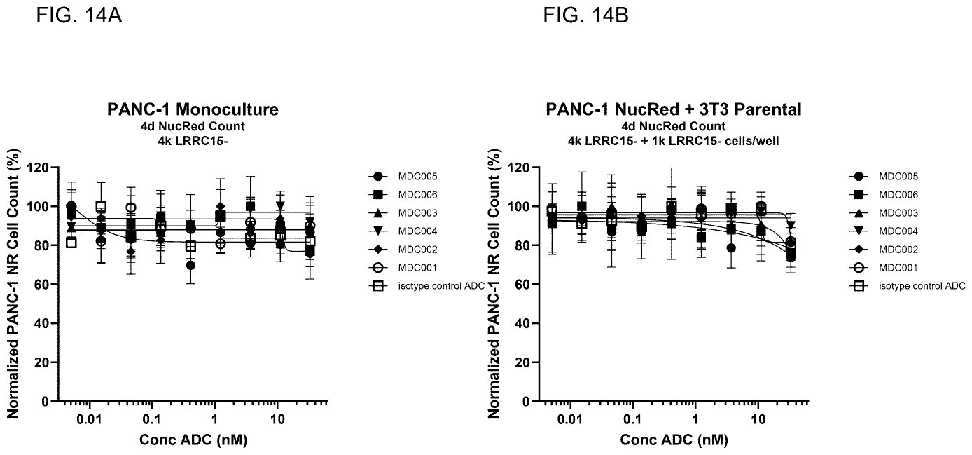

PANC-1 negative for LRRC15, were fluorescently labeled using the Nuclight Red Lenti kit from Sartorius (Cat 4625.) 4000 PANC-1 NucRed cells or 4000 PANC-1 NucRed cells co-cultured with 1000 3T3 parental cells were treated with KLH 2F5-P5-exatecan (hIgG1 isotype control) or a panel of LRRC15-P5-exatecan ADCs. Fluorescence imaging was performed using an Incucyte SX5 imaging system (Sartorius) and Normalized Nuclear Red Cell Counts at Day 4 are plotted. In the absence of LRRC15 antigen on the PANC1 cancer cells, anti-LRRC15-P5-exatecan ADCs do not have direct cell killing activity on the PANC1 NucRed cells plated in monoculture (FIG. 14A), or on PANC1 NucRed cells in co-culture with LRRC15 negative 3T3 cells (FIG. 14B).

### Example 17: Co-Culture Bystander Assay Using Anti-LRRC15-P5-Exatecan ADCs

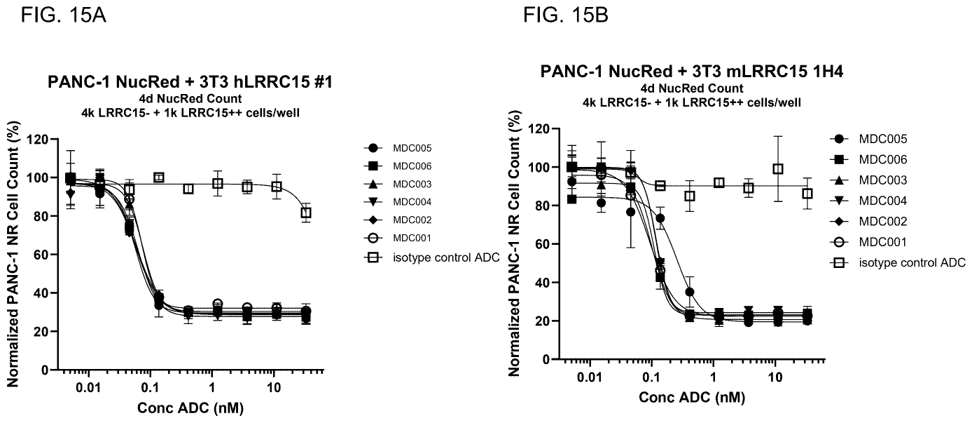

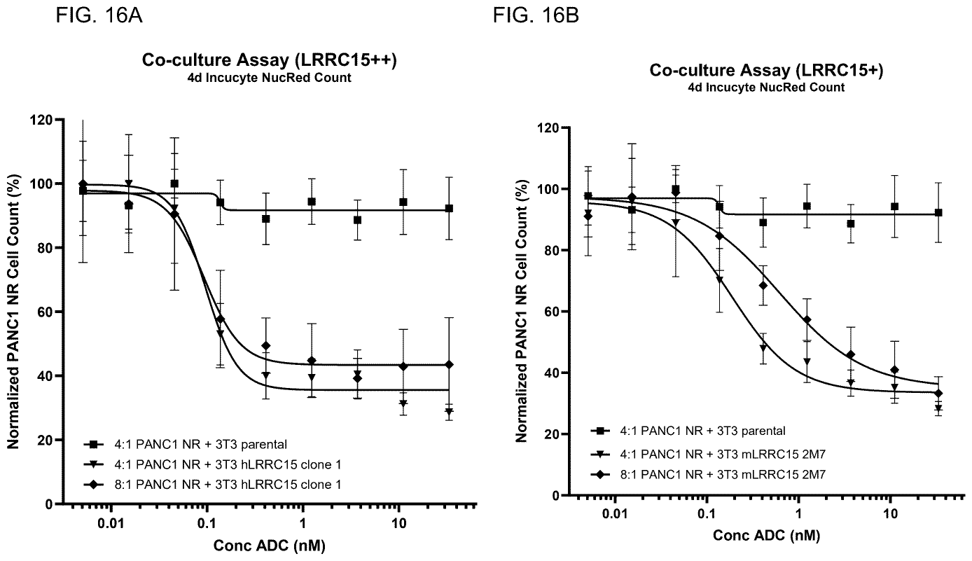

Anti-LRRC15-P5-exatecan ADCs were assessed in bystander assays consisting of LRRC15 negative cancer cells and 3T3 stromal cells. Cell lines used for the co-culture assays included: PANC1 NucRed cancer cells (LRRC15−), 3T3 parental cells (LRRC15−), 3T3 human LRRC15 clone 1 (LRRC15++), 3T3 mouse LRRC15 2M7 (LRRC15+) and 3T3 mouse LRRC15 1H4 (LRRC15++.) ADCs were tested in a 3-fold titration range covering 33.33 nM to 0.005 nM ADC. Fluorescence imaging was performed using an Incucyte SX5 imaging system (Sartorius) and normalized nuclear red cell counts at Day 4 are plotted. FIG. 15A shows dose dependent bystander activity of anti-LRRC15-P5-exatecan ADCs using 3T3 human LRRC15 clone 1, and FIG. 15B shows bystander activity using 3T3 mouse LRRC15 1H4.

### Example 18: Co-Culture Bystander Assay Using Different Ratios of LRRC15 Positive Stromal Cells and LRRC15 Negative Cancer Cells

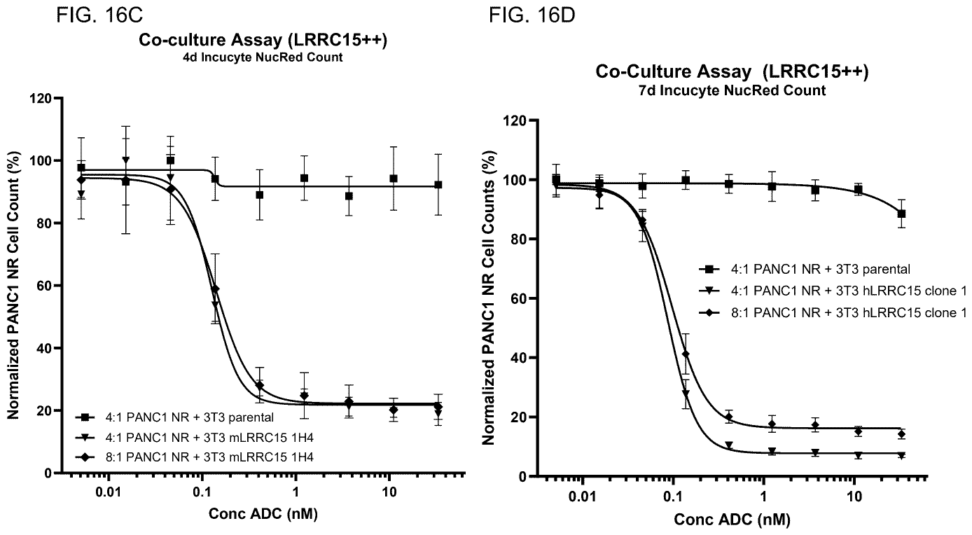

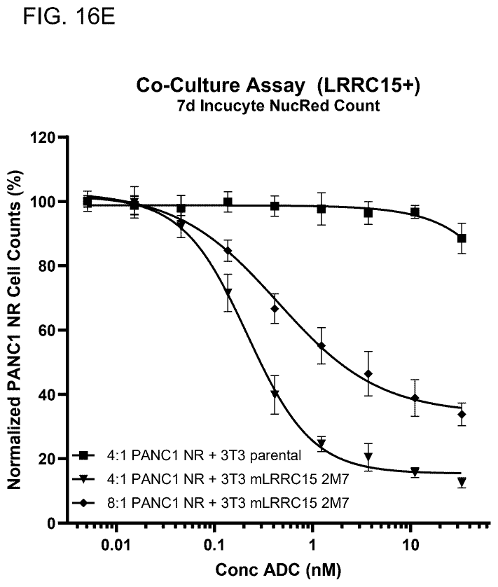

Bystander activity of MDC001 was assessed using transfectant cell lines with three levels of antigen density. Cell lines used for the co-culture assays included: PANC1 NucRed cancer cells (LRRC15−), 3T3 parental cells (LRRC15−), 3T3 human LRRC15 clone 1 (LRRC15++), 3T3 mouse LRRC15 2M7 (LRRC15+) and 3T3 mouse LRRC15 1H4 (LRRC15++.) Antigen dependent bystander activity was tested using both a 4:1 ratio and an 8:1 ratio of LRRC15 negative cancer cells to 3T3 stromal cells. Fluorescence imaging was performed using an Incucyte SX5 imaging system (Sartorius) and normalized nuclear red cell counts of the PANC1 NucRed cancer cells at Day 4 (FIGS. 16A, 16B, and 16C) or Day 7 (FIGS. 16D and 16E) are plotted.

The MDC001 activity is demonstrated to be antigen dependent as shown by the lack of bystander activity in the co-culture condition using 3T3 parental cells, as well as with a non-targeting isotype control P5-exatecan ADC. Increased bystander activity was observed with increasing LRRC15 antigen presence in the co-cultures, tested both by increased per-cell surface expression using overexpressing cell lines, and by the altered ratio of LRRC15 expressing cells at 8:1 ratio vs 4:1 ratio.

Notably, in the co-culture assay using 3T3 mouse LRRC15 2M7 clone (FIG. 16B), which has a receptor copy number comparable to that of primary human stromal cells expressing LRRC15 (Example 8), MBP001 shows robust bystander activity at both the 4:1 and the 8:1 ratio of cancer cell to stromal cell. In the bystander assay, MDC001 binds to the target stromal cell, gets internalized and processed, and it is the liberated cell-permeable exatecan payload which exerts its cytotoxic activity on the LRRC15 negative PANC1 cancer cell line.

### Example 19: Incucyte Images of Monoculture and Co-Cultures Treated with MDC001

At doses of MDC001 which result in bystander killing (e.g., 0.4 nM ADC), imaging data shows that 3T3 stromal cells remain intact and viable.

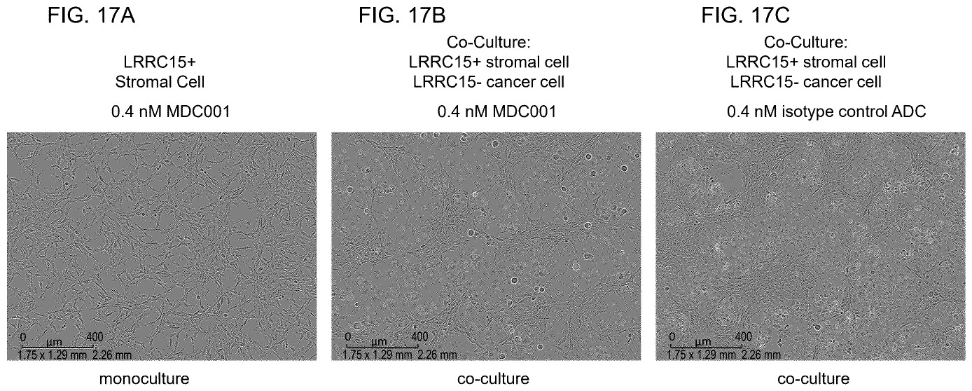

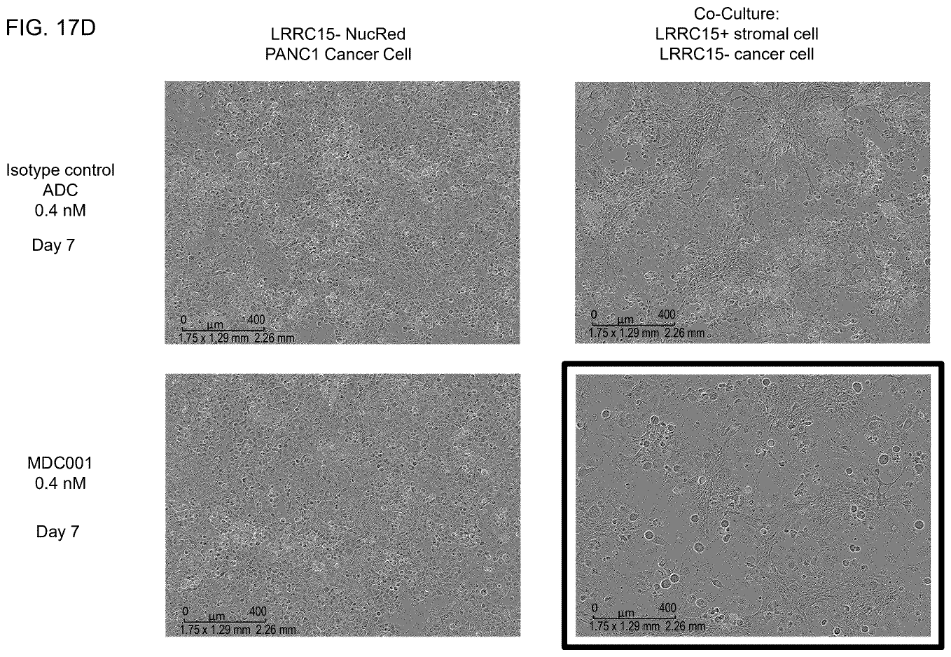

FIGS. 17A-17D shows Incucyte images of cells after treatment with 0.4 nM of MDC001 and with 0.4 nM KLH-P5-exatecan ADC. PANC1 cancer cells are demarcated using a nuclear fluorescence mask (FIGS. 17B-17C). 3T3 human clone 1 stromal cells remain intact at doses where bystander activity of the MDC001 is observed (FIGS. 17A-17B). FIG. 17C shows co-cultured PANC1 NucRed cancer cells treated with the isotype control. Relative to the control, PANC1 NucRed cancer cell counts are significantly reduced upon treatment with MDC001 (FIG. 17B). LRRC15 positive stromal cells process the LRRC15-P5-exatecan ADCs, and the liberated exatecan payload results in a reduction of the LRRC15 negative cancer cells.

FIG. 17D shows minimal activity of MDC001 on LRRC15 negative PANC1 NucRed cancer cells alone. Bystander activity of MDC001, as demonstrated by a reduction in the PANC1 NucRed cancer cell counts in the co-culture assay, is evident at day 7 (FIG. 17D).

### Example 20: Incucyte Images Comparing Two Topoisomerase 1 Inhibitor Payloads: Deruxtecan and P5-Exatecan

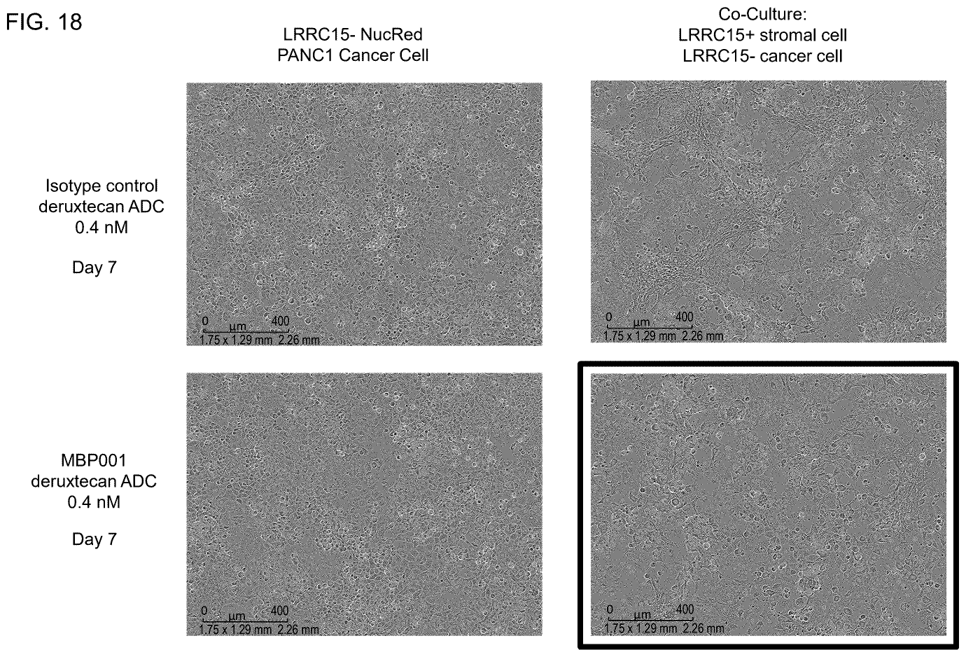

FIG. 18 shows Incucyte images of cells after treatment with 0.4 nM of MBP001-Deruxtecan ADC or with 0.4 nM KLH-Deruxtecan ADC. LRRC15 positive stromal cells process the LRRC15-Deruxtecan ADC, and the liberated ADC payload is released. The co-culture images show that Deruxtecan ADC results in only a modest reduction of PANC1 NucRed cell counts at day 7. Though both anti-LRRC15 Deruxtecan ADC and anti-LRRC15-P5-exatecan ADC use a Topoisomerase 1 inhibitor payload, the MDC001 (FIG. 17D) ADC results in significantly more robust bystander activity as compared to MBP001 Deruxtecan (FIG. 18).

### Example 21: Co-Culture Bystander Assay Comparing MBP001-Deruxtecan with MDC001

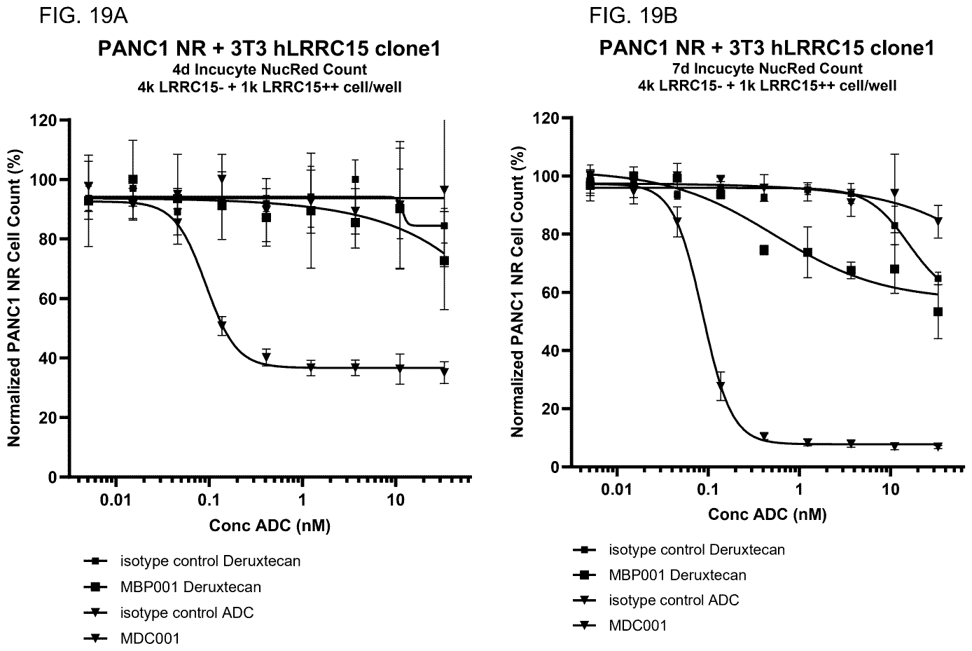

FIGS. 19A-19B compare the bystander activity of MDC001 and MBP001-deruxtecan ADC. PANC1 NucRed cancer cells were co-cultured at a 4:1 ratio with 3T3 human LRRC15 overexpressing stromal cells. ADCs were tested in a 3-fold titration range covering 33.33 nM to 0.005 nM ADC. At day 4 post treatment, robust bystander activity was observed using MDC001 (FIG. 19A), and this bystander activity appears more pronounced at day 7 (FIG. 19B). In contrast, only modest bystander activity is observed using MBP001-deruxtecan ADC at day 7 (FIG. 19B).

Exatecan is reported in literature to be more potent than deruxtecan in small molecule assessments and is reported to have increased bystander potential due to enhanced cell permeability. FIGS. 19A-19B shows that while Deruxtecan and P5-exatecan are both Topoisomerase 1 inhibitor-based ADC linker payloads, P5-exatecan ADCs appear to result in enhanced bystander activity in a co-culture system where the cell expressing the target is a stromal cell, and the cancer cells themselves are negative for the antigen. Since the LRRC15 antigen expressing stromal cell is not directly killed by the MDC001, a steady supply of LRRC15 antigen is maintained in the co-culture system.

### Example 22: In Vivo Anti-Tumor Efficacy Using 6 Different Anti-LRRC15-P5-Exatecan ADCs

In vivo anti-tumor efficacy of six anti-LRRC15 clones was investigated after conjugating them with the P5-exatecan linker payload in EBC-1 cell line-derived xenograft model. 2.5×10⁶ EBC-1 cells (Matrigel to media 1:1) in an injection volume of 0.1 mL were implanted into female CB-17 SCID mice (CB17/Icr-Prkdcscid/IcrIcoCrl, strain #236, Charles River Laboratory). Animals with tumor volumes approximately between 100 mm³˜200 mm³ were randomized into treatment groups (n=10/group) with mean tumor volume of approximately ˜150 mm³. Tumor bearing animals were treated with a single intraperitoneal injection of Isotype ADC or with anti-LRRC15-P5-exatecan ADCs at 1.5 mg/Kg (Isotype antibody, MDC001, MDC002, MDC003, MDC004, MDC005, and MDC006). Body weight (data not shown) and tumor length (L) and width (W) were measured twice a week post tumor implantation. Tumor volumes were calculated using the formulation L*(W\hat{ }2)/2.

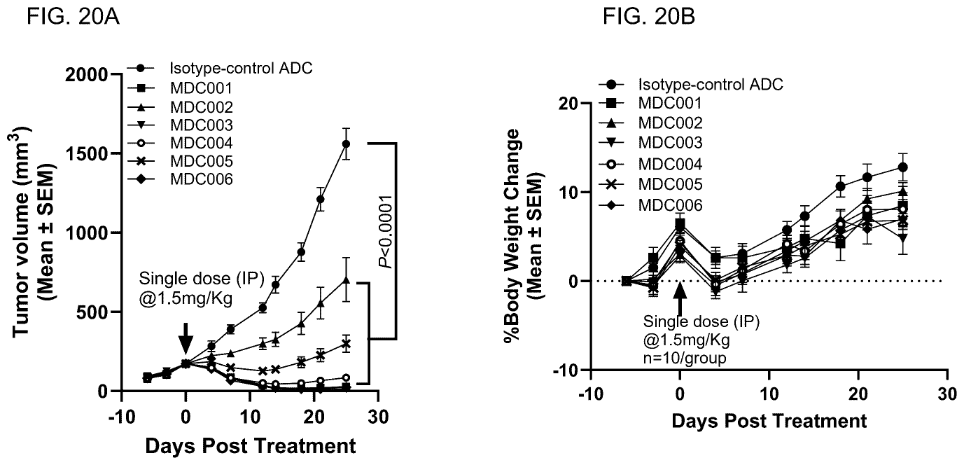

FIG. 20A demonstrates that treatment with a single dose of 1.5 mg/Kg of MDC001, MDC002, MDC003, MDC004, MDC005, and MDC006 resulted in a significant tumor growth suppression of the EBC1 tumor model when compared to isotype-P5-exatecan. Among the six ADCs, MDC001, MDC004, MDC006, and MDC003 demonstrated robust tumor regression.

FIG. 20B shows no significant loss in body weight of animals upon treatment with MDC001, MDC002, MDC003, MDC004, MDC005, and MDC006, indicating that these compounds were well tolerated at doses tested.

### Example 23: In Vivo Data Showing Anti-LRRC15 mAb and MDC001 Activity

In vivo anti-tumor efficacy of MBP001 was investigated after conjugating the lead clone with the P5-exatecan linker payload in EBC-1 and NCI-H1650 cell line-derived xenograft model. 2.5×10⁶ EBC-1 or 5×10⁶ NCI-H1650 cells (Matrigel to media 1:1) in an injection volume 0.1 ml were implanted into female CB-17 SCID mice (CB17/Icr-Prkdcscid/IcrIcoCrl, strain #236, Charles River Laboratory). Animals tumor volumes approximately between 100 mm³˜200 mm³ were randomized into treatment groups (n=8-9/group) with mean tumor volume of 150 mm³. Tumor bearing animals were treated with a single intraperitoneal injection of naked antibody (MBP001), Isotype-control ADC, or MDC001 at the indicated dose level. Body weight (data not shown) and tumor length (L) and width (W) were measured twice a week post tumor implantation. Tumor volumes were calculated using the formulation L*(W\hat{ }2)/2.

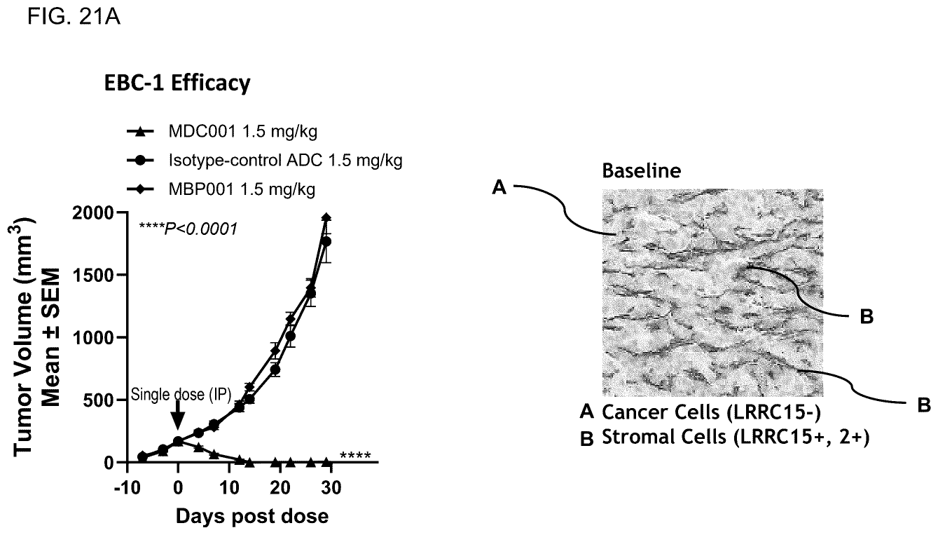

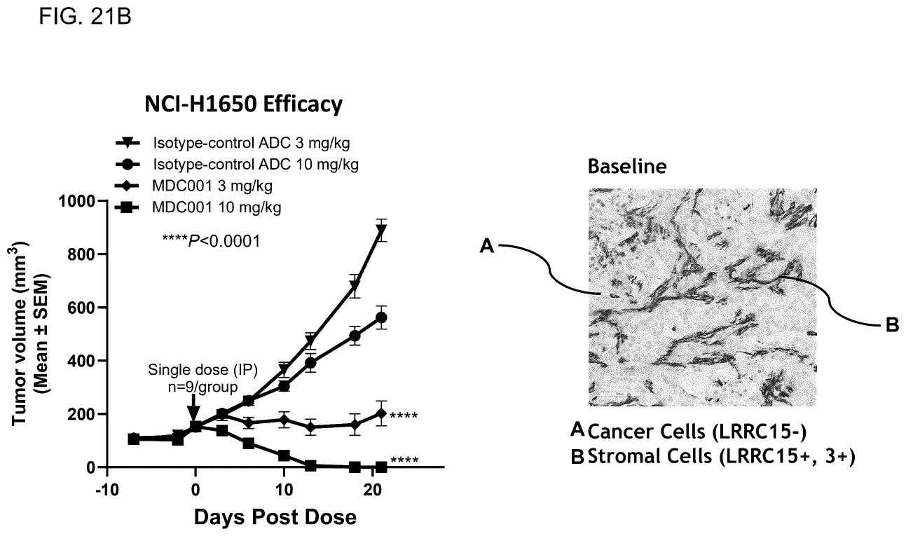

FIG. 21A demonstrates robust tumor growth regression in the EBC1 model upon treatment with a single dose of MDC001 at 1.5 mg/kg when compared to the isotype-P5-exatecan ADC. Notably, since treatment with the naked anti-LRRC15 mAb did not show activity in this model, the MDC001 efficacy observed is due to activity derived from the ADC payload.

FIG. 21B demonstrates significant dose-dependent tumor growth regression in the NCI-H1650 model upon treatment with single dose of MDC001 at 3 mg/kg and 10 mg/kg dose levels when compared to the respective isotype-ADC control groups. At the 10 mg/kg dose level, complete regression of all the tumors across all the animals was observed.

### Example 24: LRRC15 Expression by Immunohistochemistry

This Example analyzed the expression of LRRC15 in mouse models used to assess the in vivo anti-tumor efficacy of MDC001 in cell line derived xenograft models (EBC-1 and NCI-H1650). Animals were randomized into treatment groups at various timepoints from baseline, 4, 24, 96, and 168 hours. EBC-1 mice were treated with 1.5 mg/Kg of MDC001 and NCI-H1650 mice were treated with 3 mg/Kg of MDC001. Tumors were collected into media and transferred to the histology lab. Samples were then embedded into O.C.T. compound (Sakura Finetek; Cat #: 4583) within cryomolds (Sakura Finetek; Cat #: 4566), which were then floated on isopentane (2-methylbutane) (Thermo Fisher; Cat #: 019387-AP) that is kept on dry ice until the OCT and sample were evenly frozen. Samples were then sectioned using the cryostat (Leica CM 1950) at 5 uM. Prior to IHC (immunohistochemistry) staining, sections are prepped with 10% NBF for 10 minutes for 3 times and then wash in PBS for 5 minutes for 3 times.

LRRC15 expression in EBC-1 and NCI-H1650 model tumors was assessed by IHC using a mouse backbone anti-LRRC15 antibody diluted in Leica Bond Diluent (Leica; Cat #: AR9352) to a concentration of 2 μg/mL to test on fresh frozen tumor tissues. IHC was performed using the Leica Bond RX autostainer with Peroxidase Block, DAB Chromogen, and Hematoxylin reagents from the Leica Bond Refine Kit (Leica; Cat #: DS9800) and Rodent Block M (Biocare; Cat #: RBM961L) blocker and Mouse-on-Mouse (Biocare Cat #: MM620L) detection kit reagents from Biocare.

After IHC staining, the slides were reviewed by a pathologist.

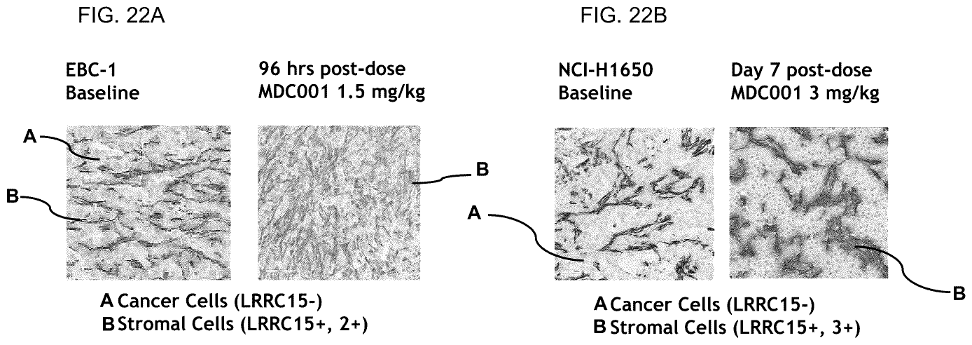

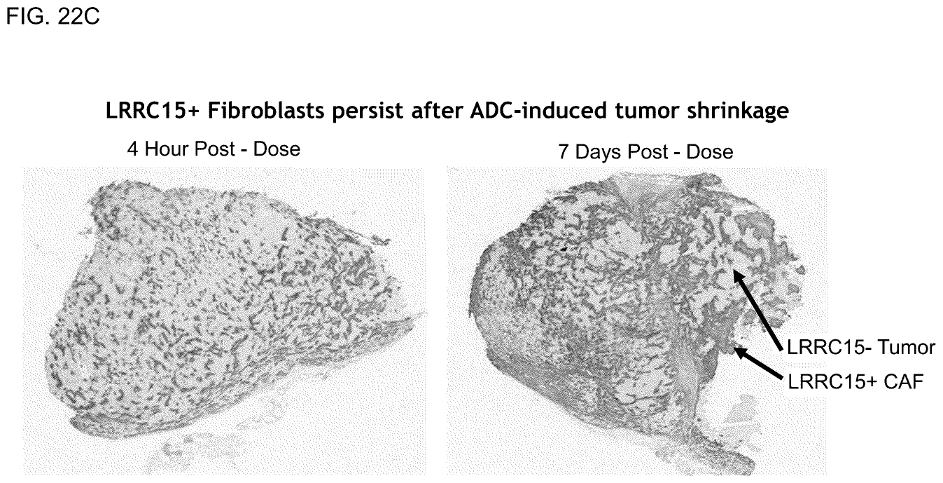

FIG. 22A shows that at baseline, EBC-1 tumors are cancer cell LRRC15 negative, but mouse stromal cell positive for LRRC15. After treatment with 1.5 mg/Kg MDC001, EBC-1 tumors are found to retain LRRC15 stromal expression.

FIG. 22B shows that at baseline, NCI-H1650 tumors are cancer cell LRRC15 negative, but stromal cell LRRC15 positive. IHC images (FIGS. 22B and 22C) show that post 3 mg/Kg MDC001 treatment, stromal LRRC15 is retained.

FIGS. 21A-21B and 22A-22B demonstrate that MDC001 induces tumor regression via bystander killing with minimal impact on LRRC15-expressing fibroblasts. LRRC15 stromal staining is retained post-treatment with MDC001.

### Example 25: Analysis of the Pharmacodynamic Markers of DDR Pathway in Tumors from Mice Treated with MDC001

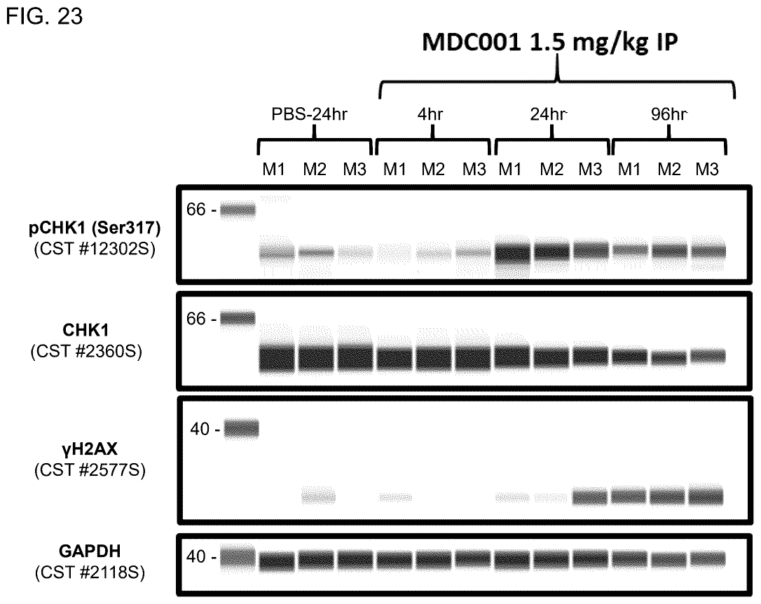

This Example evaluated the DNA damage response pathway elicited by treatment with MDC001. EBC1 tumors in mice (n=3) were treated with 1.5 mg/kg of the ADC. Expression of DNA damage response markers pCHK1 Ser317 (CST #12302S) and γH2AX (CST #2577S) were assessed in tumor samples harvested from animals at 4, 24, or 96 hours post a single intraperitoneal injection of MDC001. Total CHK1 (CST #2360S) and loading control GAPDH (CST #2118S) are shown for reference. DNA damage markers pCHK1 and γH2AX were induced within 24 to 96 hours post-dosing in the stromal LRRC15+EBC1 model (FIG. 23).

### Example 26: Anti-LRRC15-P5-Exatecan ADCs Result in Less Direct-Killing Activity on 3T3 Mouse LRRC15 1H4 Cells as Compared to Anti-LRRC15 huM25-MMAE ADC

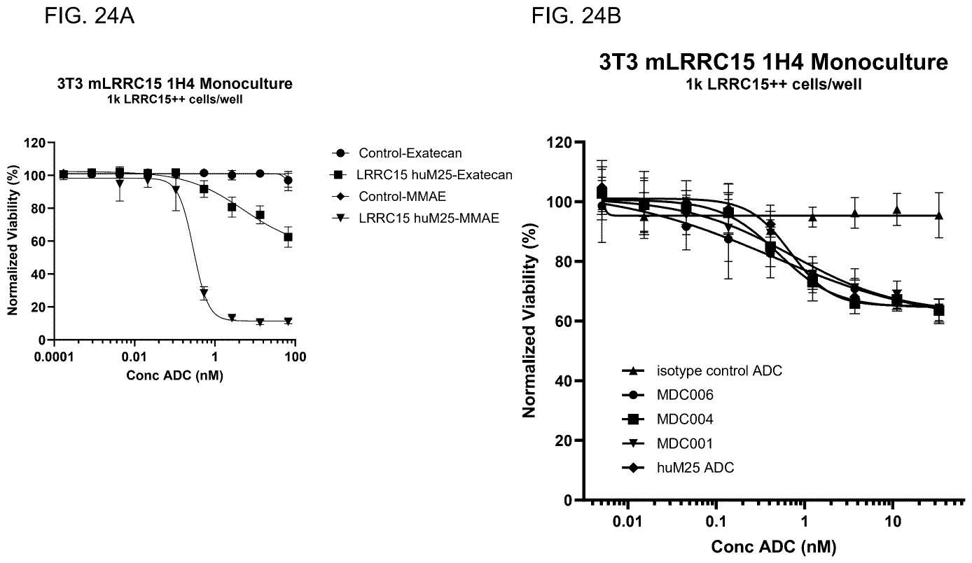

3T3 mouse LRRC15 1H4 cells, which express high levels of LRRC15, were treated with irrelevant hIgG1 isotype control ADC or a panel of anti-LRRC15 ADCs in a dose titration. Normalized cell viability on day 4 are plotted. FIG. 24A shows that anti-LRRC15 huM25-MMAE ADC (MMAE is a microtubule inhibitor-based payload) results in robust direct-targeting activity 3T3 cells overexpressing LRRC15. Anti-LRRC15 huM25-Exatecan ADC showed significantly less direct-targeting activity on LRRC15 overexpressing cells. Using the same LRRC15 overexpressing 3T3 cell line, a panel of anti-LRRC15 clones conjugated with P5-exatecan linker and payload similarly showed only modest direct-targeting activity of fibroblasts, even at the highest doses tested. Despite having robust bystander activity in co-culture assays (Examples 16-17), topoisomerase I inhibitor based P5-exatecan ADCs appear to spare 3T3 fibroblast cells in vitro, unlike microtubule inhibitor-based ADCs like huM25-MMAE (FIG. 24A).

Due to the differential sensitivity of fibroblasts and cancer cells to exatecan payload (FIG. 24B), LRRC15 expressing fibroblasts are able to persist in the tumor microenvironment, even as cancer cells are being depleted. Compared to MMAE based ADCs, it appears that exatecan based ADCs are particularly better suited for the stromal bystander mechanism, as exatecan based ADCs appear to be fibroblast-sparing. Retention of LRRC15 positive fibroblasts after MDC001 treatment enables sustained ADC payload processing resulting in increased bystander killing of neighboring cancer cells. Anti-LRRC15-P5-exatecan ADCs result in less direct-killing activity on 3T3 mouse LRRC15 1H4 cells as compared to anti-LRRC15 huM25-MMAE ADC, and thus, have the potential of offering more durable bystander killing response.

FIG. 25 illustrates that when a tumor-antigen targeting ADC is administered, cells which have high expression of the tumor antigen are targeted for depletion. While this tumor-antigen targeting approach initially results in tumor regression, a reduction of antigen binding sites is a consequence of therapeutic intervention. With fewer ADC binding sites available upon subsequent cycles of treatment, cancer cells expressing low levels of antigen expand over time and contribute to therapeutic resistance.

Unlike antibody drug conjugates which target cancer cells directly, Anti-LRRC15-P5-exatecan ADCs act on cancer cells via a bystander mechanism. Tumor-localized anti-LRRC15-P5-exatecan ADCs are processed by LRRC15 positive fibroblasts in the tumor microenvironment, and the localized release of free exatecan payload exerts cytotoxic activity on neighboring cancer cells.

Due to the differential sensitivity of fibroblasts and cancer cells, LRRC15 expressing fibroblasts persist in the tumor microenvironment, even as cancer cells are being depleted and tumor regression is observed. Sustained processing of anti-LRRC15-P5-exatecan ADCs by LRRC15 positive fibroblasts not under selection pressure circumvents the problem of tumor antigen heterogeneity and offers the potential for more durable responses.

### Example 27: MDC001 Demonstrates Greater Bystander Activity as Compared to Anti-LRRC15 ADCs Carrying Alternate Payloads

MDC001 was compared to two other anti-LRRC15 ADCs (MBP001-Deruxtecan and huM25-MMAE E2) for bystander killing activity in in vivo and in vitro models.

Generation of LRRC15 M25-MMAE DAR2 (huM25-MMAE E2) ADC: Antibody at approximately 6 mg/mL was reduced with 2 equivalents of tris (2-carboxyethyl) phosphine (TCEP) (20 mM in water) and incubated overnight at 4° C. Subsequently, 4 equivalents of valine-citrulline monomethyl auristatin E (VcMMAE) (10 mM in DMSO) were added, and the reaction was incubated for 1 hour. The reaction progress was monitored using analytical hydrophobic interaction chromatography (HIC), followed by purification using preparative scale HIC with GE Butyl Sepharose-HP resin (5 mL) equilibrated with Buffer A [1.5M ammonium sulfate, 20 mM sodium phosphate, pH 7]. After loading and washing to baseline, a linear gradient with Buffers A and B (Buffer B=20 mM sodium phosphate, pH 7+25% isopropanol) was run over 20 column volumes. The pooled fractions were concentrated using 10 kDa molecular weight cut-off spin filters and desalted with PD-10 columns. The ADC was further purified by HIC using GE Butyl Sepharose-HP resin (5 mL) equilibrated with Buffer A [1.5M ammonium sulfate, 20 mM sodium phosphate, pH 7], followed by a linear gradient with Buffers A and B (Buffer B=20 mM sodium phosphate, pH 7+25% isopropanol) over 20 column volumes to enrich the DAR2 variant.

Generation of MBP001-Deruxtecan ADC: An antibody stock solution at 5.2 mg/mL in 50 mM Tris pH 8.4, 100 mM NaCl, and 0.05 mM DTPA was adjusted to 2 mM EDTA using a 0.5 M EDTA stock solution. Antibody was reduced using 20 equivalents of tris (2-carboxyethyl) phosphine (TCEP) (20 mM in water) for 30 minutes at room temperature. The reduced antibody was then desalted using PD-10 columns into PBS with 2 mM EDTA. To the reduced antibody, 10 equivalents of linker-payload (LP) as a 10 mM stock in DMSO were added. The reaction mixture was incubated at 37° C. for 30 minutes, followed by 2 hours at room temperature, and monitored by liquid chromatography-mass spectrometry (LC-MS). The ADC was purified using PD-10 columns, yielding the desired DAR8 ADC.

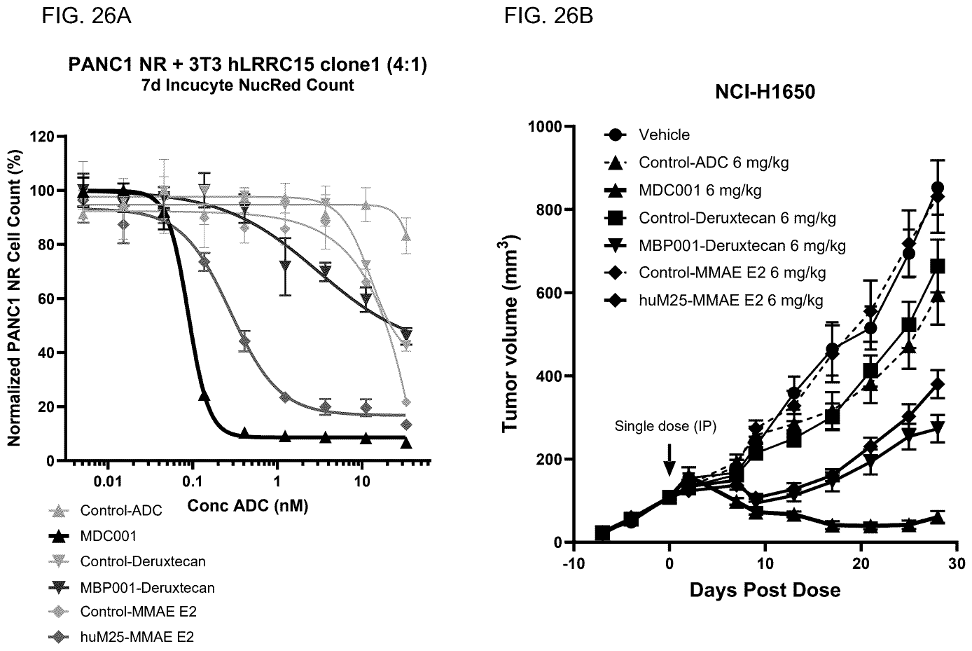

Antigen-dependent and dose-dependent bystander activity of the anti-LRRC15 ADCs on LRRC15-negative PANC-1 NucRed cancer cells grown in co-culture with LRRC15 positive stromal cells was assessed. As shown in FIG. 26A, comparison of three anti-LRRC15 ADCs carrying different payloads showed that MDC001, which carries an exatecan cytotoxic payload, exhibited increased bystander activity compared to an ADC carrying a DXd payload (MBP001-Deruxtecan) and an ADC carrying an MMAE payload (huM25-MMAE E2).

In addition, mice (n=10/group) with subcutaneous NCI-H1650 tumors were treated with a single IP injection of vehicle, MDC001 (6 mg/kg), or isotype ADC (6 mg/kg). Tumor volume was measured over 28 days throughout the duration of study. As shown in FIG. 26B, MDC001 at 6 mg/kg demonstrates greater bystander activity in vivo as compared to MBP001-Deruxtecan at 6 mg/kg in the NCI-H1650 model. In the NCI-H1650 model, a single dose of MDC001 at 6 mg/kg resulted in greater tumor regression as compared to huM25-MMAE E2 at 6 mg/kg.

These studies demonstrated that MDC001 demonstrates greater bystander killing activity as compared to anti-LRRC15 ADCs carrying alternate payloads. In the absence of LRRC15 antigen, MDC001 did not impact cell viability in vitro.

### Example 28: Antitumor Efficacy of MDC001 in Breast Cancer PDX Models

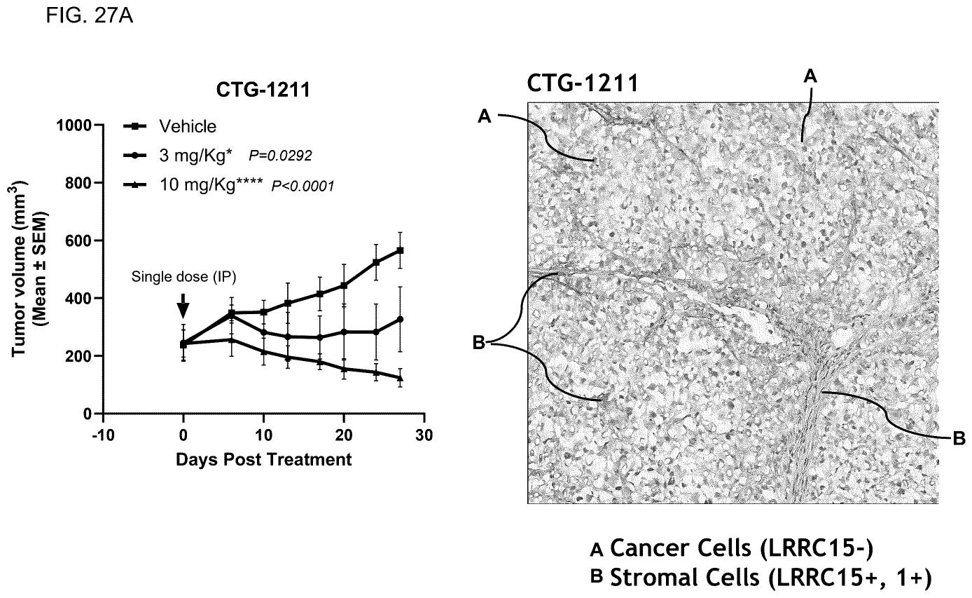

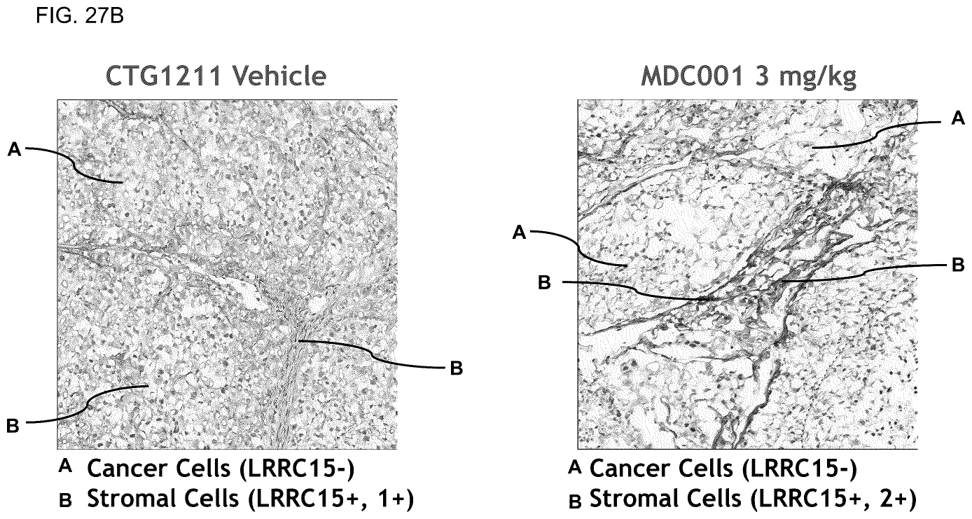

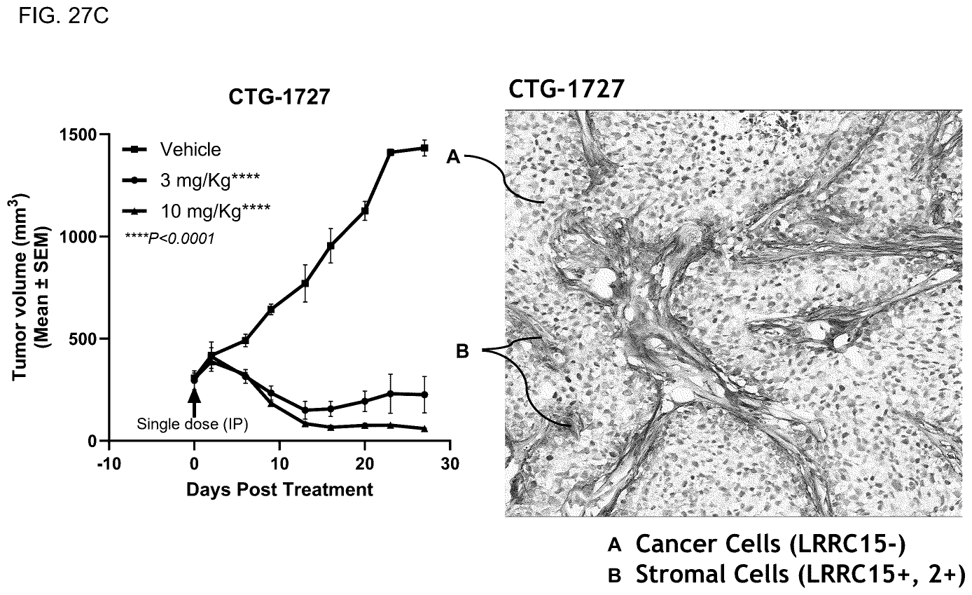

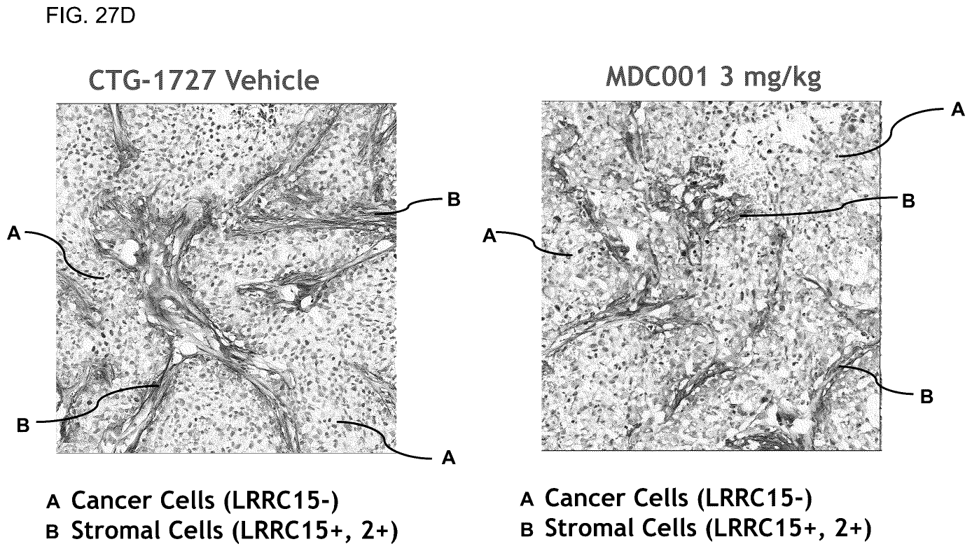

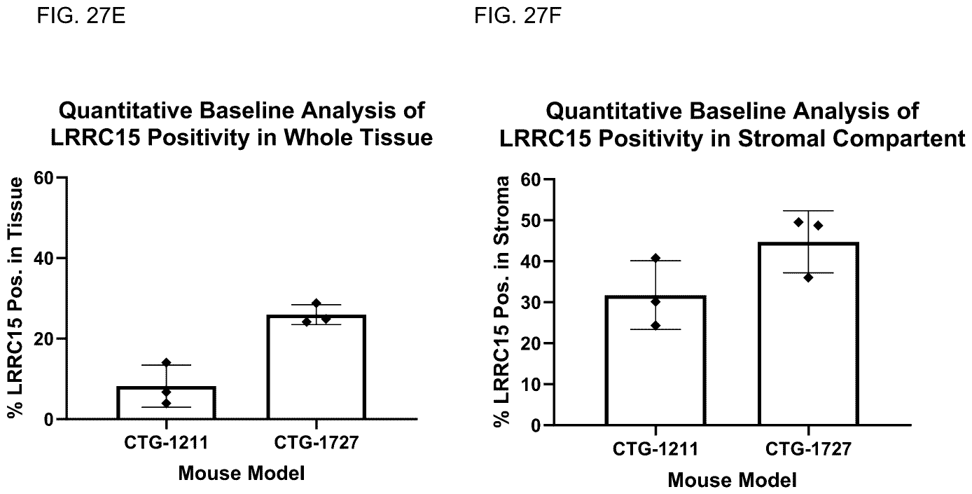

The antitumor efficacy of MDC001 was evaluated in two breast cancer PDX models, CTG-1211 and CTG-1727, at two dosages, 3 and 10 mg/kg, by a single IP injection. CTG-1211 and CTG-1727 were chosen based on the preliminary ISH analysis for LRRC15 expression. Mean tumor growth curves per treatment group and a representative IHC images and respective quantification of the LRRC15 staining for each model are displayed in FIGS. 27A-27F.

ISH methods: Breast cancer tumor microarrays (TMA) for the PDX models were obtained from Champions Oncology. FFPE TMA sections were assessed using RNAscope 2.5 LS Probe-Mouse LRRC15 from ACD. RNAscope was performed using the Leica Bond RX autostainer with the RNAscope 2.5 LSx Reagent Kit-Red using the RNAscope protocol provided by ACD. Stained slides were coverslipped using a xylene-based mounting medium and scanned on a Leica AT2 scanner. Analysis of LRRC15 RNAscope expression was analyzed with the RNAscope algorithm using HALO software ISH and TMA modules. The algorithm was trained to assess the number of dots per stromal cells. LRRC15 expression levels within the stromal cells of the these TMA sections were categorized as negative (0 dots per stromal cell), weak (average of 1 dot or 2 dots per stromal cell), moderate (average of 3 dots to 5 dots per stromal cell), and strong (average greater than 5 dots per stromal cell) based on the ISH signal, measured as dots per stromal cell. Annotations of stromal areas for each core were analyzed.

IHC methods: Frozen O.C.T. blocks of tumor samples from each PDX mouse model were sectioned at 5 μM. Prior to IHC staining, sections were washed with 10% NBF 3 times for 10 minutes each, followed by 3 washes with PBS for 5 minutes each. LRRC15 expression in breast PDX mouse model frozen tumors were then assessed by IHC using a tool mouse backbone LRRC15 antibody diluted in Leica BOND diluent to a concentration of 2 μg/mL to test on the prepped fresh frozen tumor samples. IHC was performed using the Leica BOND RX autostainer with peroxidase block, DAB chromogen, and hematoxylin reagents from the Leica BOND refine kit and Rodent Block M blocker and a Mouse-on-Mouse detection kit reagents from Biocare. Stained slides were coverslipped using a xylene-based mounting medium and scanned on the Leica AT2 scanner. Analysis of frozen mouse model tumors was analyzed with customized tissue classifiers and area quantification module using HALO. Classifiers were trained on tumor, stroma, and necrosis regions. Area quantification algorithm settings were based on a combination of DAB (positive) and hematoxylin (negative) optical density. Percentage positive of LRRC15 expression in the stromal region were quantified for each sample. HALO image analysis results were exported and graphed in GraphPad Prism.

In vivo methods: When sufficient stock animals' tumor volume reached 1,000 mm³ to 1,500 mm³, study mice were subcutaneously implanted unilaterally on the left flank with tumor fragments harvested from stock animals (CTG-1211 and CTG-1727 fragments). The mice were staged according to tumor volume and then randomly assigned to 3 groups, each with 3 mice. At the time of randomization, the average tumor volumes were 241 mm³ for the CTG-1211 model and 303 mm³ for the CTG-1727 model. Mice were dosed with a single IP injection of vehicle, or MDC001 at 3 or 10 mg/kg in a 10 mL/kg volume of vehicle.

In the CTG-1211 model (stromal cell LRRC15 1+, cancer cell LRRC15-negative), mice treated with 3 mg/kg or 10 mg/kg MDC001 showed a statistically significant decrease in mean tumor volume (P=0.0292 and P<0.0001, respectively) compared to the vehicle control group (FIG. 27A). Twenty-seven days post single dose, the treatment resulted in a 75% tumor growth inhibition (TGI) (3 mg/kg) and a 49% tumor volume regression (TVR) (10 mg/kg).

In the CTG-1727 model (stromal cell LRRC15 2+, cancer cell LRRC15-negative), mice treated with 3 mg/kg or 10 mg/kg MDC001 showed a statistically significant decrease in mean tumor volume (P<0.0001 and P<0.0001, respectively) compared to the vehicle control group (FIG. 27B). Twenty-seven days post single dose, the treatment resulted in a 24% TVR (3 mg/kg) and 80% TVR (10 mg/kg).

MDC001 demonstrated potent antitumor activity even in models where the LRRC15 staining intensity was as low as 1+. In both of the breast PDX models tested, MDC001 treatment was tolerated at 3 and 10 mg/kg dose levels. No mice were sacrificed due to weight loss following treatment with the MDC001 compound.

### Example 29: LRRC15 Expression in Breast Cancer PDX is Maintained Post-Treatment with MDC001

Mice from the CTG-1211 and CTG-1727 breast tumor models were allocated to the 2 different study groups, vehicle (n=1) and MDC001 3 mg/kg (n=3). Four days post treatment study animals were terminated to collect serum and tumor samples. Representative IHC images for CTG-1211 and CTG-1727 are shown in FIGS. 27B and 27D, respectively. IHC image quantification is summarized in Table 7.

In vivo methods: In a subset of animals, when the tumor reached approximately 184 mm³ to 224 mm³ (actual tumor volume during randomization), animals were matched by tumor size and assigned into control (vehicle, n=1) or treatment group (n=3/group). Mice were dosed with a single IP injection of vehicle or MDC001 3 mg/kg in a volume of 10 mL/kg. Tumor size and body weight were measured on day of dosing, and the study endpoint was 96 hours post-dose. Tumor and blood samples were collected at the end of the study.

LRRC15 IHC expression was quantified by digital image analysis of whole-slide scanned images. Using the HALO AI software, an image analysis algorithm was developed detecting LRRC15 staining at negative, low, medium, and high expression intensity. A tissue classifier was also developed to identify regions for analysis (tumor and stroma) and regions to exclude (necrosis and glass). The classifier was attached to the image analysis algorithm. The tissues were annotated to exclude folds and necrosis areas. LRRC15 expression was quantified by 4 different image analysis reporting: a) total tissue percent LRRC15 positive area, b) total stroma percent LRRC15 positive area, c) low, medium, and high expression of LRRC15 in total tissue area, and d) low, medium, and high expression of LRRC15 in stroma area.

TABLE 7
Quantitative Metrics for the LRRC15
Expression in Breast PDX Models

| | CTG-1211 | CTG-1727 |
|---|---|---|
| % TGI 3 mg/kg of BMS-986517 | 75 | 107 |
| % TGI 10 mg/kg of BMS-986517 | 136 | 122 |
| Preliminary ISH-based LRRC15 Expression | Low | Medium |
| Stromal LRRC15 Intensity | 1+ to 2+ | 2+ |
| % LRRC15 in Whole Tissue | 8.2 | 25.9 |
| LRRC15 H-score in Whole Tissue | 11 | 40 |
| Stromal % by Halo | 25 | 60 |
| % LRRC15 in Stromal Compartment | 31.7 | 44.7 |
| Stromal Compartment by H-score | 42 | 68 |

The IHC analysis indicated that tumors collected post-MDC001 treatment maintained LRRC15 stromal expression. These results indicate that MDC001 induced tumor regression through bystander killing while having minimal impact on LRRC15-expressing fibroblasts.

It is to be appreciated that the Detailed Description section, and not the Summary and Abstract sections, is intended to be used to interpret the claims. The Summary and Abstract sections may set forth one or more but not all exemplary aspects or embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects or embodiments will so fully reveal the general nature of the disclosure that others can, by applying knowledge within the skill of the art, readily modify and/or adapt for various applications such specific aspects or embodiments, without undue experimentation, without departing from the general concept of the present disclosure. Therefore, such adaptations and modifications are intended to be within the meaning and range of equivalents of the disclosed aspects or embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects or embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1.-124. (canceled)

125. An antibody drug conjugate (ADC) having the formula (II): 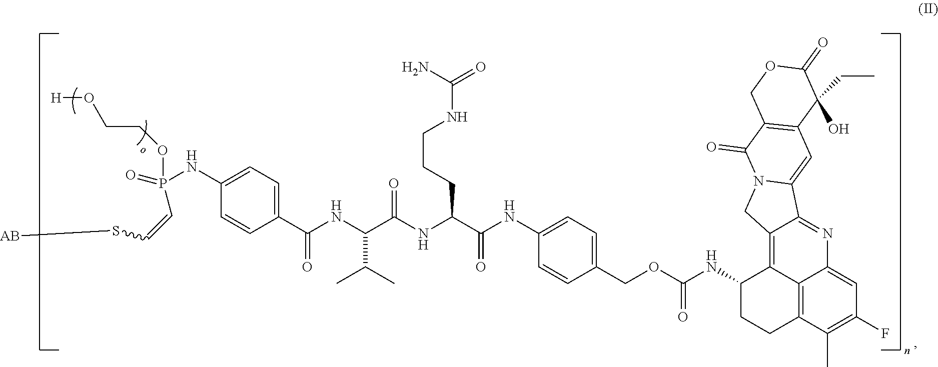
or a pharmaceutically acceptable salt, a stereoisomer or a solvate thereof, wherein: [symbol]
indicates that the configuration of the double bond is E or Z;
o ranges from 10 to 30;
n ranges from 1 to 20; and
AB is an anti-Leucine Rich Repeat Containing 15 (LRRC15) antibody or an antigen binding portion thereof, which specifically binds to LRRC15, comprising:
(a) a heavy chain variable region (VH) which comprises complementarity determining region (CDR)1, CDR2, and CDR3 regions comprising the amino acid sequences set forth in SEQ ID NOs: 3, 4, and 5, respectively, and (b) a light chain variable region (VL) which comprises CDR1, CDR2, and CDR3 regions comprising the amino acid sequences set forth in SEQ ID NOs: 9, 10, and 11, respectively.

126. The ADC of claim 125, wherein the VH comprises the amino acid sequence set forth in SEQ ID NO: 6 or SEQ ID NO: 7.

127. The ADC of claim 125, wherein the VL comprises the amino acid sequence set forth in SEQ ID NO: 12.

128. The ADC of claim 125, wherein the VH and the VL comprise the amino acid sequence set forth in SEQ ID NO: 6 or 7 and the amino acid sequence set forth in SEQ ID NO: 12, respectively.

129. The ADC of claim 125, wherein the VH and the VL comprise:
(a) the amino acid sequence set forth in SEQ ID NO: 6 and the amino acid sequence set forth in SEQ ID NO: 12, respectively; or
(b) the amino acid sequence set forth in SEQ ID NO: 7 and the amino acid sequence set forth in SEQ ID NO: 12, respectively.

130. The ADC of claim 125, wherein o is 22, 23, 24, 25, or 26.

131. The ADC of claim 125, wherein n ranges from 4 to 8.

132. An antibody drug conjugate (ADC) having the formula (II): 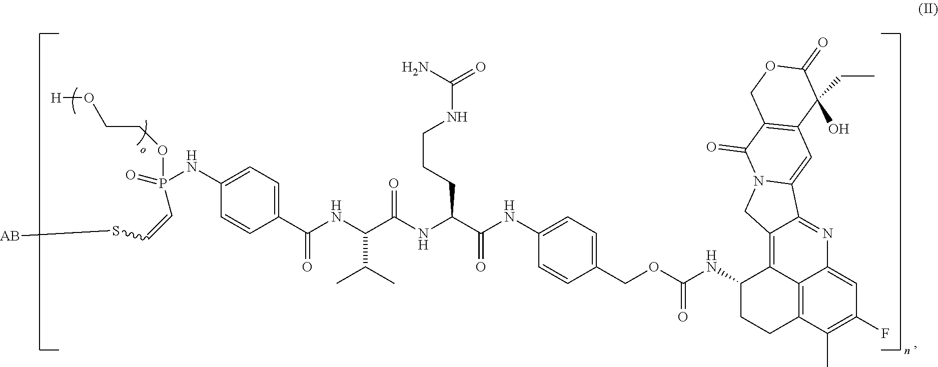
or a pharmaceutically acceptable salt, a stereoisomer, or a solvate thereof, wherein [symbol] indicates that the configuration of the double bond is E or Z; n is 8; o is 24; and AB is an anti-LRRC15 antibody or antigen binding portion thereof comprising a heavy chain variable region (VH) and a light chain variable region (VL) which comprise the amino acid sequence set forth in SEQ ID NO: 6 or 7 and the amino acid sequence set forth in SEQ ID NO: 12, respectively.

133. The ADC of claim 125, wherein the anti-LRRC15 antibody comprises a heavy chain and a light chain comprising the amino acid sequence set forth in SEQ ID NO: 14, 15, or 16 and the amino acid sequence set forth in SEQ ID NO: 17, respectively.

134. The ADC of claim 125, wherein the anti-LRRC15 antibody comprises an IgG1 constant region, an IgG2 constant region, an IgG3 constant region, an IgG4 constant region, or a variant thereof.

135. A pharmaceutical composition comprising the ADC of claim 125, and a pharmaceutically acceptable carrier.

136. A kit comprising the ADC of claim 125, and instructions for use.

137. A method of producing the ADC of claim 125 comprising conjugating a thiol-containing compound of AB-(SH)ₙ with Compound A: 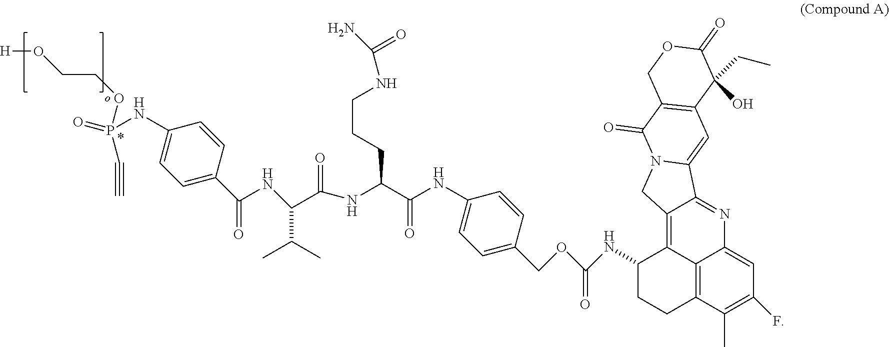
wherein AB is the anti-LRRC15 antibody or antigen binding portion thereof, and n ranges from 1 to 20.

138. A method of treating a cancer that expresses LRRC15 in a subject in need thereof comprising administering to the subject a therapeutically effective amount of the ADC of claim 125.

139. The method of claim 138, wherein the cancer comprises colorectal cancer, breast cancer, lung cancer including non-small cell lung cancer, ovarian cancer, pancreatic cancer, bladder cancer, uterine/cervical cancer, prostate cancer, testicular cancer, esophageal cancer, gastric cancer, gastrointestinal cancer, colon cancer, kidney cancer, head and neck cancer, stomach cancer, germ cell cancer, bone cancer, liver cancer, thyroid cancer, skin cancer, neoplasm of the central nervous system, lymphoma, leukemia, myeloma, sarcoma, or myelodysplastic syndromes.

140. The method of claim 138, wherein the cancer comprises: non-small cell lung cancer (NSCLC), squamous non-small cell lung cancer (SqNSCLC), non-squamous NSCLC, breast cancer, triple negative breast cancer (TNBC), HR⁺/HER⁻ breast cancer, head and neck squamous cell carcinoma (HNSCC), colorectal cancer, pancreatic cancer, ovarian cancer, HER2+ breast cancer, or prostate cancer.

141. An antibody drug conjugate (ADC) having the formula [ADC 102]: 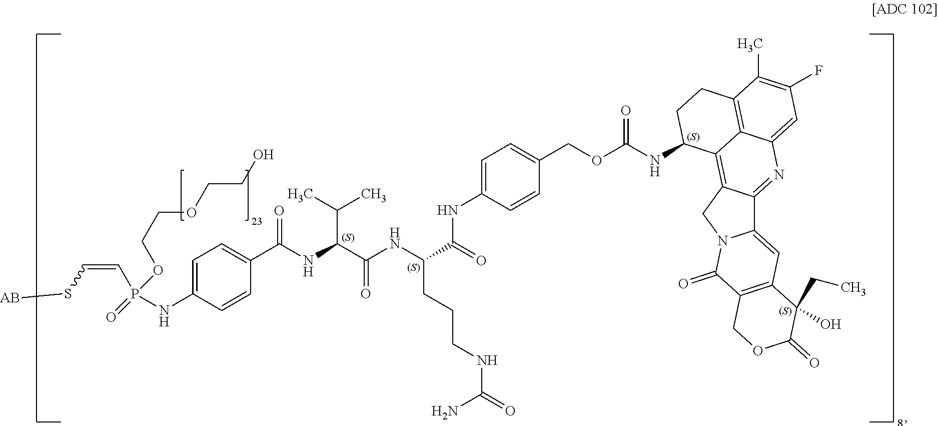
wherein [symbol] indicates that the configuration of the double bond is E or Z; AB is an anti-LRRC15 antibody comprising a heavy chain and a light chain which comprise the amino acid sequence set forth in SEQ ID NO: 14 and the amino acid sequence set forth in SEQ ID NO: 17, respectively.

142. An antibody drug conjugate (ADC) having the formula [ADC 102]: 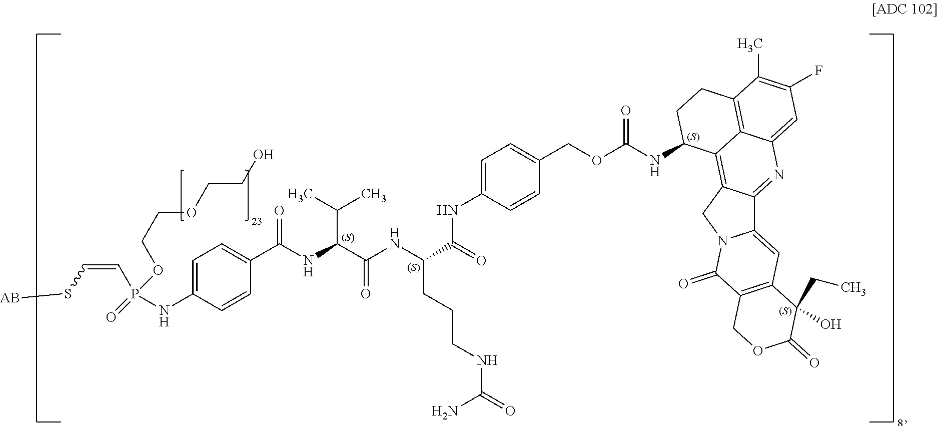
wherein [symbol] indicates that the configuration of the double bond is E or Z; AB is an anti-LRRC15 antibody comprising a heavy chain and a light chain which comprise the amino acid sequence set forth in SEQ ID NO: 16 and the amino acid sequence set forth in SEQ ID NO: 17, respectively.

143. A pharmaceutical composition comprising the ADC of claim 141, and a pharmaceutically acceptable carrier.

144. A pharmaceutical composition comprising the ADC of claim 142, and a pharmaceutically acceptable carrier.

145. A method of producing the ADC of claim 141 comprising conjugating a thiol-containing compound of AB-(SH)ₙ with Compound A′: 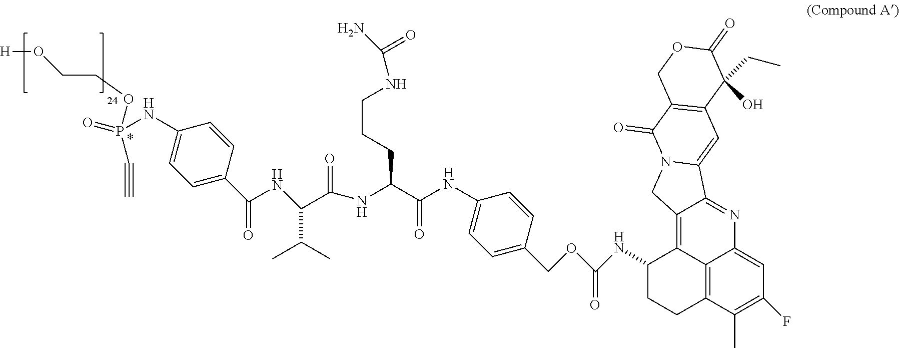
wherein AB is the anti-LRRC15 antibody or antigen binding portion thereof, and n is 8.

146. A method of producing the ADC of claim 142 comprising conjugating a thiol-containing compound of AB-(SH)ₙ with Compound A′: 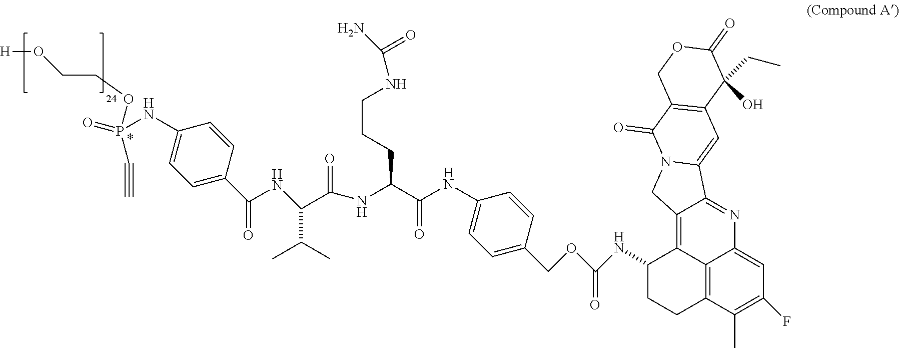
wherein AB is the anti-LRRC15 antibody or antigen binding portion thereof, and n is 8.

147. A method of treating a cancer that expresses LRRC15 in a subject in need thereof comprising administering to the subject a therapeutically effective amount of the ADC of claim 141.

148. A method of treating a cancer that expresses LRRC15 in a subject in need thereof comprising administering to the subject a therapeutically effective amount of the ADC of claim 142.

149. The method of claim 147, wherein the cancer comprises: non-small cell lung cancer (NSCLC), squamous non-small cell lung cancer (SqNSCLC), non-squamous NSCLC, breast cancer, triple negative breast cancer (TNBC), HR⁺/HER⁻ breast cancer, head and neck squamous cell carcinoma (HNSCC), colorectal cancer, pancreatic cancer, ovarian cancer, HIER2+ breast cancer, or prostate cancer.

150. The method of claim 148, wherein the cancer comprises: non-small cell lung cancer (NSCLC), squamous non-small cell lung cancer (SqNSCLC), non-squamous NSCLC, breast cancer, triple negative breast cancer (TNBC), HR⁺/HER⁻ breast cancer, head and neck squamous cell carcinoma (HNSCC), colorectal cancer, pancreatic cancer, ovarian cancer, HER2+ breast cancer, or prostate cancer.
